SEQUENCE LISTING (38 sequences)

SEQ ID NO: 1 - RNA, 52 nt, synthetic construct
  source 1..52: mol_type = other RNA
gaataaactagtattcttctggtccccacagactcagagagaacccgccacc

SEQ ID NO: 2 - RNA, 295 nt, synthetic construct
  source 1..295: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 3 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 4 - RNA, 45 nt, synthetic construct
  source 1..45: mol_type = other RNA
aataaactagtattcttctggtccccacagactcagagagaaccc

SEQ ID NO: 5 - RNA, 54 nt, synthetic construct
  source 1..54: mol_type = other RNA
gagaataaactagtattcttctggtccccacagactcagagagaacccgccacc

SEQ ID NO: 6 - RNA, 47 nt, synthetic construct
  source 1..47: mol_type = other RNA
agaataaactagtattcttctggtccccacagactcagagagaaccc

SEQ ID NO: 7 - RNA, 295 nt, synthetic construct; identical to SEQ ID NO: 2
  source 1..295: mol_type = other RNA

SEQ ID NO: 8 - RNA, 295 nt, synthetic construct; identical to SEQ ID NO: 2
  source 1..295: mol_type = other RNA

SEQ ID NO: 9 - protein, 12 aa, synthetic construct
PKSSWPNHETSL

SEQ ID NO: 10 - protein, 12 aa, synthetic construct
PRSSWPNHETSL

SEQ ID NO: 11 - protein, 11 aa, synthetic construct
PKRSWSNHTSS

SEQ ID NO: 12 - protein, 12 aa, synthetic construct
PKDSWSDHEASL

SEQ ID NO: 13 - protein, 12 aa, synthetic construct
PKSSWSSHEASL

SEQ ID NO: 14 - protein, 11 aa, synthetic construct
SNNAEEQTNLY

SEQ ID NO: 15 - protein, 11 aa, synthetic construct
SNNAAEQTNLY

SEQ ID NO: 16 - protein, 11 aa, synthetic construct
SNSAEEQTDLY

SEQ ID NO: 17 - protein, 11 aa, synthetic construct
PNDEAEQTKLY

SEQ ID NO: 18 - protein, 11 aa, synthetic construct
PNDEAEQTRLY

SEQ ID NO: 19 - protein, 11 aa, synthetic construct
PNDAAEQTKLY

SEQ ID NO: 20 - protein, 8 aa, synthetic construct
EFIRVPEW

SEQ ID NO: 21 - protein, 8 aa, synthetic construct
EFISVPEW

SEQ ID NO: 22 - protein, 7 aa, synthetic construct
EFTNPEW

SEQ ID NO: 23 - protein, 8 aa, synthetic construct
EFINVPEW

SEQ ID NO: 24 - protein, 12 aa, synthetic construct
PKSSWPNHETSL

SEQ ID NO: 25 - protein, 12 aa, synthetic construct
PRSSWPNHETSL

SEQ ID NO: 26 - protein, 11 aa, synthetic construct
PKRSWSNHTSS

SEQ ID NO: 27 - protein, 12 aa, synthetic construct
PKDSWSDHEASL

SEQ ID NO: 28 - protein, 12 aa, synthetic construct
PKSSWSSHEASL

SEQ ID NO: 29 - protein, 11 aa, synthetic construct
SNNAEEQTNLY

SEQ ID NO: 30 - protein, 11 aa, synthetic construct
SNNAAEQTNLY

SEQ ID NO: 31 - protein, 11 aa, synthetic construct
SNSAEEQTDLY

SEQ ID NO: 32 - protein, 11 aa, synthetic construct
PNDEAEQTKLY

SEQ ID NO: 33 - protein, 11 aa, synthetic construct
PNDEAEQTRLY

SEQ ID NO: 34 - protein, 11 aa, synthetic construct
PNDAAEQTKLY

SEQ ID NO: 35 - protein, 8 aa, synthetic construct
EFIRVPEW

SEQ ID NO: 36 - protein, 8 aa, synthetic construct
EFISVPEW

SEQ ID NO: 37 - protein, 7 aa, synthetic construct
EFTNPEW

SEQ ID NO: 38 - protein, 8 aa, synthetic construct
EFINVPEW

(19) United States
(12) Patent Application Publication
(10) Pub. No.: US 2025/0332245 A1
(43) Pub. Date: Oct. 30, 2025

(54) IMMUNOGENIC COMPOSITIONS AGAINST INFLUENZA

(71) Applicant: PFIZER INC., NEW YORK, NY (US)

(72) Inventors: Pirada Suphaphiphat Allen, Congers, NY (US); Ivna Padron De Souza, Nyack, NY (US); Teresa Marie Hauguel, Marriottsville, MD (US); Kelly Anne Lindert, Arlington, MA (US)

(73) Assignee: PFIZER INC., NEW YORK, NY (US)

(21) Appl. No.: 18/946,845

(22) Filed: Nov. 13, 2024

Related U.S. Application Data
(60) Provisional application No. 63/599,547, filed on Nov. 15, 2023.
(60) Provisional application No. 63/606,056, filed on Dec. 4, 2023.
(60) Provisional application No. 63/572,174, filed on Mar. 29, 2024.
(60) Provisional application No. 63/635,956, filed on Apr. 18, 2024.
(60) Provisional application No. 63/640,200, filed on Apr. 29, 2024.
(60) Provisional application No. 63/672,906, filed on Jul. 18, 2024.

(51) Int. Cl. A61K 39/145; A61K 39/00; A61P 37/04

(52) U.S. Cl. CPC A61K 39/145; A61P 37/04; A61K 2039/6018; A61K 2039/70

(57) ABSTRACT

The disclosure relates to compositions and methods for the preparation, manufacture and therapeutic use ribonucleic acid vaccines comprising polynucleotide molecules encoding one or more influenza antigens, such as hemagglutinin antigens.

### CROSS-REFERENCE TO RELATED APPLICATIONS

The present application claims priority from U.S. Provisional Application No. 63/599,547, filed Nov. 15, 2023; U.S. Provisional Application No. 63/606,056, filed Dec. 4, 2023; U.S.¶ Provisional Application No. 63/572,174, filed Mar. 29, 2024; U.S. Provisional Application No. 63/635,956, filed Apr. 18, 2024; U.S. Provisional Application No. 63/640,200, filed Apr. 29, 2024; and U.S. Provisional Application No. 63/672,906, filed Jul. 18, 2024; the contents of which are hereby incorporated by reference herein in their entirety.

### REFERENCE TO SEQUENCE LISTING

This application is being filed electronically via Patent Center and includes an electronically submitted sequence listing in .XML format. The XML file contains a sequence listing entitled, “PC073060A.xml,” created on Nov. 11, 2024, and has a size of 34 KB. The sequence listing contained in this XML file is part of the specification and is incorporated herein by reference in its entirety.

### SEQUENCE LISTING

The instant application contains a Sequence Listing which has been submitted electronically in ASCII format and is hereby incorporated by reference in its entirety.

### FIELD

The present disclosure relates to compositions and methods for the preparation, manufacture and therapeutic use of ribonucleic acid vaccines comprising polynucleotide molecules encoding one or more influenza antigens, such as hemagglutinin antigens. The present disclosure relates to influenza vaccine formulations and vaccination regimes for immunising against influenza disease, their use in medicine, including their use in augmenting immune responses to various antigens, and to methods of preparation. In additional aspects, the present disclosure relates to monovalent influenza immunogenic compositions comprising a polynucleotide molecule encoding one or more influenza antigens, such as a hemagglutinin antigen, or antigenic preparation thereof from an influenza virus strain that is associated with a pandemic or has the potential to be associated with a pandemic.

### BACKGROUND

Influenza viruses are members of the orthomyxoviridae family, and are classified into three types (A, B, and C), based on antigenic differences between their nucleoprotein (NP) and matrix (M) protein.

The genome of influenza A virus includes eight molecules (seven for influenza C virus) of linear, negative polarity, single-stranded RNAs, which encode several polypeptides including: the RNA-directed RNA polymerase proteins (PB2, PB1 and PA) and nucleoprotein (NP), which form the nucleocapsid; the matrix proteins (M1, M2, which is also a surface-exposed protein embedded in the virus membrane); two surface glycoproteins, which project from the lipoprotein envelope: hemagglutinin (HA) and neuraminidase (NA); and nonstructural proteins (NS1 and NS2). Hemagglutinin is the major envelope glycoprotein of influenza A and B viruses, and hemagglutinin-esterase (HE) of influenza C viruses is a protein homologous to HA.

An influenza virus strain that has the potential to be associated with a pandemic may continue a new hemagglutinin (HA) compared to the hemagglutinin in the currently circulating strains, which may or not be accompanied by a change in neuraminidase subtype; it may be capable of being transmitted horizontally in the human population; and it may be pathogenic for humans. A new haemagglutinin may be one which has not been evident in the human population for an extended period of time, or it may be a hemagglutinin that has not been circulating in the human population before, for example H5, H9, H7 or H6 which are found in birds. At least a large proportion of the population has not previously encountered the antigen of the influenza virus having the potential to be associated with a pandemic and the population may be immunologically naïve to it. A “pandemic” influenza strain as used herein is one that has caused or has capacity to cause pandemic infection of subject populations, such as human populations. In some embodiments, a pandemic strain has caused pandemic infection. In some embodiments, such pandemic infection involves epidemic infection across multiple territories; in some embodiments, pandemic infection involves infection across territories that are separated from one another (e.g., by mountains, bodies of water, as part of distinct continents, etc.) such that infections ordinarily do not pass between them.

Persons at risk in case of an influenza pandemic may be different from the defined risk-groups for complications due to seasonal influenza.

During a pandemic, the number of individuals at risk of influenza may be greater than in interpandemic periods. Accordingly, the development of a suitable vaccine with the potential to be produced in large amounts and with efficient distribution and administration potential is essential for addressing a pandemic. For these reasons, a monovalent instead of a multi-valent vaccine may be developed for pandemic purposes in an attempt to reduce vaccine volume while eliciting sufficient immune responses in subjects.

A challenge for therapy and prophylaxis against influenza and other infections using traditional vaccines is the limitation of vaccines in breadth, providing protection only against closely related subtypes. In addition, the length of time required to complete current standard influenza virus vaccine production processes inhibits the rapid development and production of an adapted vaccine in a pandemic situation.

There is a need for improved immunogenic compositions against influenza.

### SUMMARY

In a first aspect, there is disclosed an influenza immunogenic composition, such as a vaccine, comprising a ribonucleic acid (RNA) polynucleotide comprising an open reading frame encoding at least one influenza virus antigenic polypeptide or an immunogenic fragment thereof, wherein the RNA polynucleotide is formulated in a lipid nanoparticle (LNP), wherein the polypeptide is derived from an influenza virus strain that is associated with a pandemic or has the potential to be associated with a pandemic.

As used herein, a pandemic strain refers to an influenza strain that is associated with or susceptible to being associated with an outbreak of influenza disease, such as pandemic Influenza A strains. Suitable strains include avian (bird) influenza strains. Suitable pandemic strains are, but not limited to: H5N1 (the highly pathogenic avian H5N1 strain), H9N2, H7N7, H2N2, H7N1 and H1N1. Others suitable pandemic strains in humans include H7N3, H10N7 and H5N2.

In another aspect, the disclosure describes a method for the production of an influenza immunogenic composition, in particular a vaccine, for a pandemic situation or a pre-pandemic situation which method comprises formulating an RNA polynucleotide in a LNP and preparing a composition comprising a ribonucleic acid (RNA) polynucleotide comprising an open reading frame encoding at least one influenza virus antigenic polypeptide or an immunogenic fragment thereof, wherein the RNA polynucleotide is formulated in a lipid nanoparticle (LNP), wherein the polypeptide is derived from an influenza virus strain that is associated with a pandemic or has the potential to be associated with a pandemic.

In yet another aspect, the disclosure describes use of the composition(s) disclosed herein for inducing at least one of i) an improved CD4 T-cell immune response, ii) an improved B cell memory response, iii) an improved humoral response, against said virus antigen or antigenic composition in a human. Said immune response may be induced in an immuno-compromised individual or population, such as a high risk adult or an elderly. In a further embodiment, there is provided the use of an immunogenic composition described herein for revaccination of humans previously vaccinated with a monovalent influenza immunogenic composition comprising an RNA polynucleotide encapsulated in a LNP encoding an influenza antigen or antigenic preparation thereof from a single influenza virus strain which is associated with a pandemic or has the potential to be associated with a pandemic.

In some embodiments, the revaccination is made in subjects who have been vaccinated the previous season against influenza. In some embodiments, the revaccination is made with a vaccine comprising an influenza strain (e.g. H5N1 Vietnam) which is of the same subtype as that used for the first vaccination (e.g. H5N1 Vietnam). In some embodiments, the revaccination is made with a drift strain of the same sub-type, e.g. H5N1 Indonesia. In another embodiment, said influenza strain used for the revaccination is a shift strain, i.e. is different from that used for the first vaccination, e.g. it has a different HA or NA subtype, such as H5N2 (same HA subtype as H5N1 but different NA subtype) or H7N1 (different HA subtype from H5N1 but same NA subtype).

In some embodiments, the first administration (e.g., vaccination) is made at the declaration of a pandemic and revaccination is made later. Alternatively, the first administration is part of a pre-pandemic strategy and is made before the declaration of a pandemic, as a priming strategy, thus allowing the immune system to be primed, with the revaccination made subsequently. In this instance one or two doses of vaccine containing the same influenza strain are administered as part of the primo-vaccination. Revaccination, in particular with a variant (e.g. drift) strain, can be made at any time after the first course (one or two doses) of vaccination. Typically revaccination is made at least 1 month, suitably at least two months, suitably at least three months, or 4 months after the first vaccination, suitably 6 or 8 to 14 months after, suitably at around 10 to 12 months after or even longer. Suitable revaccination one year later or even more than one year later is potentially capable of boosting antibody and/or cellular immune response. This is especially important as further waves of infection may occur several months after the first outbreak of a pandemic. As needed, revaccination may be made more than once.

In a further aspect disclosed herein, there is provided the use of an antigen or antigenic preparation from a first pandemic influenza strain in the manufacture of an immunogenic composition as herein defined for protection against influenza infections caused by a variant influenza strain.

In a specific aspect, there is provided a method of vaccination of an immuno-compromised human individual or population such as high risk adults or elderly, said method comprising administering to said individual or population an influenza immunogenic composition described herein.

In still another embodiment, the disclosure describes a method for revaccinating humans previously vaccinated with a monovalent influenza immunogenic composition comprising an RNA polynucleotide encapsulated in a LNP encoding an influenza antigen or antigenic preparation thereof from a single pandemic influenza virus strain, said method comprising administering to said human a second immunogenic composition comprising an RNA polynucleotide encapsulated in a LNP encoding an influenza antigen or antigenic preparation thereof.

In a further embodiment there is provided a method for vaccinating a human population or individual against one pandemic influenza virus strain followed by revaccination of said human or population against a variant influenza virus strain, said method comprising administering to said human (i) a first composition comprising an RNA polynucleotide encapsulated in a LNP encoding an influenza virus or antigenic preparation thereof from a first pandemic influenza virus strain, and (ii) a second immunogenic composition comprising an RNA polynucleotide encapsulated in a LNP encoding an influenza virus strain variant of said first influenza virus strain. In a specific embodiment said variant strain is associated with a pandemic or has the potential to be associated with a pandemic. In another embodiment said variant strain is at least one circulating (seasonal) influenza virus strain.

The unmet needs for improved immunogenic compositions against influenza, among other things, are provided herein. In one aspect, the disclosure relates to an immunogenic composition including: (i) a first ribonucleic acid (RNA) polynucleotide having an open reading frame encoding a first antigen, said antigen including at least one influenza virus antigenic polypeptide or an immunogenic fragment thereof, and (ii) a second RNA polynucleotide having an open reading frame encoding a second antigen, said second antigen including at least one influenza virus antigenic polypeptide or an immunogenic fragment thereof, wherein the first and second RNA polynucleotides are formulated in a lipid nanoparticle (LNP). In some embodiments, the first and second antigens include hemagglutinin (HA), or an immunogenic fragment or variant thereof. In some embodiments, the first antigen includes an HA from a different subtype of influenza virus to the influenza virus antigenic polypeptide or an immunogenic fragment thereof of the second antigen. In some embodiments, the composition further includes (iii) a third antigen including at least one influenza virus antigenic polypeptide or an immunogenic fragment thereof, wherein the third antigen is from influenza virus but is from a different strain of influenza virus to both the first and second antigens. In some embodiments, the first, second and third RNA polynucleotides are formulated in a lipid nanoparticle.

In some embodiments, the composition further includes (iv) a fourth RNA polynucleotide having an open reading frame encoding a fourth antigen, said antigen including at least one influenza virus antigenic polypeptide or an immunogenic fragment thereof, wherein the fourth antigen is from influenza virus but is from a different strain of influenza virus to the first, second and third antigens. In some embodiments, the first, second, third, and fourth RNA polynucleotides are formulated in a lipid nanoparticle.

In some embodiments, each RNA polynucleotide includes a modified nucleotide. In some embodiments, the modified nucleotide is selected from the group consisting of pseudouridine, 1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-0-methyl uridine.

In some embodiments, each RNA polynucleotide includes a 5′terminal cap, a 5′ UTR, a 3′UTR, and a 3′ polyadenylation tail. In some embodiments, the 5′terminal cap includes:

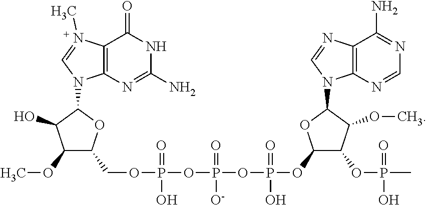

In some embodiments, the 5′ UTR includes SEQ ID NO: 1.

In some embodiments, the 3′ UTR includes SEQ ID NO: 2. In some embodiments, the 3′ polyadenylation tail includes SEQ ID NO: 3.

In some embodiments, the RNA polynucleotide has an integrity greater than 85%. In some embodiments, the RNA polynucleotide has a purity of greater than 85%.

In some embodiments, the lipid nanoparticle includes 20-60 mol % ionizable cationic lipid, 5-25 mol % neutral lipid, 25-55 mol % cholesterol, and 0.5-5 mol % PEG-modified lipid.

In some embodiments, the cationic lipid includes:

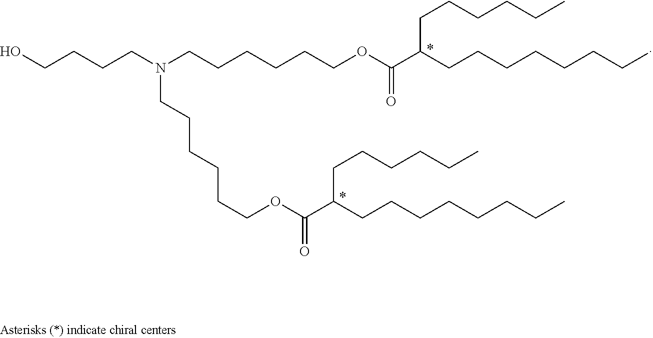

In some embodiments, the PEG-modified lipid includes:

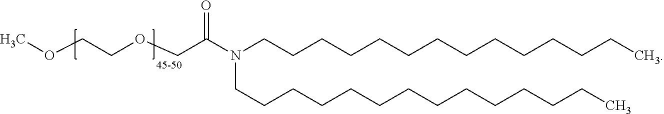

In some embodiments, the first antigen is HA from influenza A subtype H1 or an immunogenic fragment or variant thereof and the second antigen is HA from a different H1 strain to the first antigen or an immunogenic fragment or variant thereof. In some embodiments, the first and second antigens are HA from influenza A subtype H3 or an immunogenic fragment or variant thereof and wherein both antigens are derived from different strains of H3 influenza virus.

In some embodiments, the first and second antigens are HA from influenza A subtype H1 or an immunogenic fragment or variant thereof and the third and fourth antigens are from influenza A subtype H3 or an immunogenic fragment or variant thereof and wherein the first and second antigens are derived from different strains of H1 virus and the third and fourth antigens are from different strains of H3 influenza virus.

In some embodiments, at least the first and second RNA polynucleotides are formulated in a single lipid nanoparticle. In some embodiments, the first and second RNA polynucleotides are formulated in a single lipid nanoparticle. In some embodiments, the first, second, and third RNA polynucleotides are formulated in a single lipid nanoparticle. In some embodiments, the first, second, third, and fourth RNA polynucleotides are formulated in a single LNP.

In some embodiments, each of the RNA polynucleotides is formulated in a single LNP, wherein each single LNP encapsulates the RNA polynucleotide encoding one antigen. In some embodiments, the first RNA polynucleotide is formulated in a first LNP; and the second RNA polynucleotide is formulated in a second LNP. In some embodiments, the first RNA polynucleotide is formulated in a first LNP; the second RNA polynucleotide is formulated in a second LNP; and the third RNA polynucleotide is formulated in a third LNP. In some embodiments, the first RNA polynucleotide is formulated in a first LNP; the second RNA polynucleotide is formulated in a second LNP; the third RNA polynucleotide is formulated in a third LNP; and the fourth RNA polynucleotide is formulated in a fourth LNP.

In another aspect, the disclosure relates to any of the immunogenic compositions described herein, for use in the eliciting an immune response against influenza.

In another aspect, the disclosure relates to a method of eliciting an immune response against influenza disease, including administering an effective amount of any of the immunogenic compositions described herein.

In another aspect, the disclosure relates to a method of purifying an RNA polynucleotide synthesized by in vitro transcription. The method includes ultrafiltration and diafiltration. In some embodiments, the method does not comprise a chromatography step. In some embodiments, the purified RNA polynucleotide is substantially free of contaminants comprising short abortive RNA species, long abortive RNA species, double-stranded RNA (dsRNA), residual plasmid DNA, residual in vitro transcription enzymes, residual solvent and/or residual salt. In some embodiments, the residual plasmid DNA is ≤500 ng DNA/mg RNA. In some embodiments, the yield of the purified mRNA is about 70% to about 99%. In some embodiments, purity of the purified mRNA is between about 60% and about 100%. In some embodiments, purity of the purified mRNA is between about 85%-95%.

### DESCRIPTION OF THE DRAWINGS

The following drawings form part of the present specification and are included to further demonstrate certain aspects described herein.

FIG. 1. Functional Anti-HA Antibodies Elicited by Immunization of Mice With Monovalent or Quadrivalent LNP-Formulated modRNA Encoding Influenza HA as Measured By MNT.

FIG. 2 Flow Cytometry Detection of Antigens in Cells Transfected with LNP-formulated pandemic influenza modRNA vaccine candidates. LNP-formulated modRNA vaccine candidates encoding H5 (clade 2.3.4.4b) antigens were 2-fold serially titrated and added to a HEK-293T cell monolayer (from 500 to 0.97 ng/well). HA protein expression was measured using the HA stem-specific, broadly neutralizing human monoclonal antibody FI6. Prior to analysis by flow cytometry, cells were labeled with fluorescent antibodies, and the percentage of live cells expressing the HA protein is enumerated. Expression was measured by quantifying the number of cells that had a positive signal for bound anti-HA antibody.

FIG. 3. Functional Antibodies Elicited by Immunization of Mice With a Multivalent Versus Monovalent Formulation of Influenza modRNA Encoding HA and NA. HA- and NA-expressing modRNA at 2 μg dose either as monovalent or octavalent vaccine formulation in LNPs were administered to mice as prime and boost. The multivalent vaccine groups consisted of an octavalent vaccine of a mixture of separately formulated modRNA-LNP complexes each expressing HA or NA (Sep/M) or an octavalent vaccine of all HA and NA modRNAs pre-mixed and then co-formulated in LNPs (Co-f). A/Michigan/45/2015 (H1N1), B/Colorado/06/2017 (Victoria), and B/Phuket/3073/2013 (Yamagata) strains were used. The licensed vaccine comparator (Fluad) is an adjuvanted, trivalent seasonal influenza vaccine that did not contain a B Yamagata component. Day 49 sera were assessed for HAI titers depicted as GMTs with 95% confidence interval. One-way ANOVA Sidak's multiple comparison test *, p<0.05; **, p<0.01; ***, p<0.001. Blue asterisks above the multivalent groups indicate statistical significance between the multivalent group versus the monovalent control group.

FIG. 4. Virus Neutralization Elicited by Immunization of Mice With a Multivalent Versus Monovalent Formulation of Influenza modRNA Encoding HA and NA. HA- and NA-expressing modRNA at 2 μg dose either as monovalent or octavalent vaccine formulation in LNPs were administered to mice as prime and boost. A/Michigan/45/2015 (H1N1) and A/Singapore/INFIMH-16-0019/2016 (H3N2) strains were used. Day 49 sera were assessed for neutralizing titers depicted as GMTs with 95% confidence interval. There was no statistically significant difference between the monovalent and the multivalent groups.

FIG. 5. NA Antibodies Elicited by Immunization of Mice with a Multivalent vs Monovalent Formulation of Influenza modRNA Encoding HA and NA. HA and NA-expressing modRNA at 2 μg dose either as monovalent or octavalent vaccine formulation in LNPs were administered to mice as prime and boost. A/Michigan/45/2015 (H1N1) and B/Colorado/06/2017 (Victoria) strains were used. Day 49 sera were assessed for neuraminidase inhibition titers depicted as geometric mean titers with 95% confidence interval. One-way ANOVA Sidak's multiple comparison test *, p<0.05; **, p<0.01; ***, p<0.001. Asterisks indicate statistical significance between the multivalent groups and the monovalent control group.

FIG. 6. T-cell Responses Elicited by Immunization of Mice With a Multivalent Versus Monovalent Formulation of Influenza modRNA Encoding HA and NA. HA- and NA-mRNA at 2 μg dose either as a monovalent or multivalent vaccine formulation in LNPs were administered to mice as prime and boost. T-cell response was assessed by quantifying IFNγ-producing cells using Mabtech ELISpot PLUS kits. Results expressed as geometric mean with 95% confidence interval for IFNγ+ spots per million cells. One-way ANOVA Sidak's multiple comparison test *, p<0.05; **, p<0.01; ***, p<0.001. Blue asterisks above the multivalent groups indicate statistical significance between the multivalent group and the monovalent control group.

FIG. 7A-C. Virus Neutralization Elicited by Immunization of Mice with Pandemic Influenza modRNA Vaccine Candidates. Female BALB/c mice were immunized IM with 0.2, 1, or 10 μg of each LNP-formulated modRNA-HA preparation. Virus neutralization responses against the matched virus strains (FIG. 7A) A/Astrakhan/3212/2020 (H5N8), or (FIG. 7B) A/Wisconsin/588/2019 (seasonal H1N1 benchmark) were measured by a 1-Day MNT on Day 22 (3 weeks post-Dose 1 (PD1)) and Day 42 (2 weeks post-Dose 2 (PD2)). LOD=limit of detection. FIG. 7C shows neutralization titers against H9N2 post dose 1 (PD1) and post dose 2 (PD2).

FIG. 8A-D. Virus Neutralization Elicited by Immunization of Mice with Pandemic Influenza modRNA Vaccine Candidates Encoding HA and NA. Female BALB/c mice received two IM immunizations 4 weeks apart with LNP-formulated modRNA vaccine preparations encoding HA and/or NA antigens derived from (FIG. 8A) A/Astrakhan/3212/2020 H5N8 or (FIG. 8B) A/Wisconsin/588/2019 H1N1 virus (seasonal influenza modRNA benchmark). Splenocytes were also harvested on Day 43 and stimulated with a HA (H5 or H1) or NA (N8 or N1) peptide pool to measure the HA- or NA-specific T-cell responses as a percentage of cytokine-expressing CD4+ and CD8+ T-cells (FIG. 8A, FIG. 8B, FIG. 8C (H5N8 virus neutralization—post dose 2), and FIG. 8D (H1N1 virus neutralization—post dose 2)). Virus neutralization responses against the matched virus strains were measured by a 1-Day MNT at 2 weeks post-Dose 2 (PD2). LOD=limit of detection.

FIG. 9A-F. HA-Specific T-cell Responses to LNP-Formulated Pandemic modRNA Vaccine Candidates as Measured by Intracellular Cytokine Staining. Female BALB/c mice received two IM immunizations 4 weeks apart with LNP-formulated modRNA vaccine preparations encoding HA and/or NA antigens derived from the A/Astrakhan/3212/202 H5N8, or A/Wisconsin/588/2019 H1N1 virus (seasonal influenza modRNA benchmark). T-cell responses were analyzed by ICS assay using H5-specific (FIG. 9A to FIG. 9C) or H1-specific (FIG. 9D to FIG. 9F) peptide pools (2 μg/mL per peptide) to stimulate T cells in the spleens harvested on Day 43 (2 weeks post Dose 2). ICS assay results expressed as mean±SD of percentage of antigen-specific IFN-γ-expressing (FIG. 9A, FIG. 9D) or polyfunctional (IFN-γ+, IL-2+, and TNF-α+) CD4+ T cells (FIG. 9B, FIG. 9E) and IFN-γ-expressing CD8+ T cells (FIG. 9C, FIG. 9F).

FIG. 10A-F. NA-Specific T-cell Responses to LNP-Formulated Pandemic modRNA Vaccine Candidates as Measured by Intracellular Cytokine Staining. Female BALB/c mice received two IM immunizations 4 weeks apart with LNP-formulated modRNA vaccine preparations encoding HA and/or NA antigens derived from the A/Astrakhan/3212/202 H5N8, or A/Wisconsin/588/2019 H1N1 virus (seasonal influenza modRNA benchmark). T-cell responses were analyzed by ICS assay using N8-specific (FIG. 10A to FIG. 10C) or N1-specific (FIG. 10D to FIG. 10F) peptide pools (2 μg/mL per peptide) to stimulate Tcells in the spleens harvested on Day 43 (2 weeks post Dose 2). ICS assay results expressed as mean±SD of percentage of antigen-specific IFN-γ-expressing (FIG. 10A, FIG. 10D) or polyfunctional (IFN-γ+, IL-2+, and TNF-α+) CD4+ T cells (FIG. 10B, FIG. 10E) and IFN-γ-expressing CD8+ T cells (FIG. 10C, FIG. 10F).

FIG. 11A-B. Functional HA Antibodies and Virus Neutralization Titers Elicited by Immunization of Mice with an Influenza modRNA Encoding HA. Female Balb/c mice were immunized IM with 0.2 μg of LNP-Formulated modRNA-HA or 2.4 μg of a licensed adjuvanted quadrivalent inactivated vaccine (QIV), Fluad. Functional antibody and virus neutralization responses against A/Wisconsin/588/2019 (H1N1) were measured by an HAI (FIG. 11A) and 1-Day MNT (FIG. 11B) assay on Days 21 (3 weeks post-prime) and 42 (2 weeks post-boost).

FIG. 12A-E. HA-Specific T-Cell Responses to LNP-Formulated modRNA Encoding Influenza HA by Intracellular Cytokine Staining. Female BALB/c mice received two IM immunizations 4 weeks apart with 0.2 μg of modRNA encoding influenza A/Wisconsin/588/2019 (H1N1) HA or a licensed adjuvanted quadrivalent inactivated influenza vaccine (QIV), Fluad. T-cell response was analyzed by ICS using an HA peptide pool (2 μg/mL per peptide) to stimulate T-cells in the spleens harvested on Day 42 (2 weeks post-dose 2). ICS assay results expressed as median with interquartile range of percentage of IFN-γ, IL-4, IL-2 expressing or polyfunctional CD4+ T-cells (FIG. 12A to FIG. 12C); and ICS assay results expressed as median with interquartile range of percentage of IFN-γ expressing CD8+ T-cells (FIG. 12D) or polyfunctional (IFN-γ, TNF-α, CD107a) CD8+ T-cells (FIG. 12E).

FIG. 13. Functional Antibodies Elicited by Immunization of Rats with Influenza modRNA Encoding HA. Functional Antibodies Elicited by Immunization of Rats with Influenza modRNA Encoding HA. Four Wistar-Han female rats received two IM immunizations 2 weeks apart with 30 μg of an LNP-formulated modRNA encoding influenza A/Wisconsin/588/2019 (H1N1) HA. Functional antibody responses against A/Wisconsin/588/2019 (H1N1) were measured by an HAI assay on Days 16 and 21 (2 and 7 days post-dose 2).

FIG. 14A-D. Functional Antibody and Virus Neutralization Titers Elicited by Immunization of Nonhuman Primates with Influenza modRNA Encoding HA. Three rhesus macaques and three cynomolgus macaques received two IM immunizations 4 weeks apart (Days 0 and 28) with 30 μg of an LNP-formulated modRNA encoding influenza A/Wisconsin/588/2019 (H1N1) HA. Functional antibody responses against A/Wisconsin/588/2019 (H1N1) were measured by an HAI (FIG. 14A, FIG. 14B) and a 1-Day MNT (FIG. 14C, FIG. 14D) assay using sera collected on Day −7 (pre-immunization) and Days 7, 21, 28, 35, 42, 77, 105, 133 and 168 after primary immunization.

FIG. 15A-B. CD4+ T-Cell Responses Elicited Following Immunization of NHPs with LNP-Formulated modRNA Encoding Influenza HA. Three rhesus macaques and three cynomolgus macaques received two IM immunizations 4 weeks apart (Days 0 and 28) with 30 μg of an LNP-formulated modRNA encoding influenza A/Wisconsin/588/2019 (H1N1) HA. PBMCs were isolated at different timepoints and stimulated ex vivo with HA-specific peptide pools followed by intracellular cytokine staining (ICS). ICS assay results are plotted as the percentage of IFN-γ-expressing cells within CD4+ T cell subsets (FIG. 15A, depicting HA-specific IFN-γ-expressing cells within CD4+ T cell subsets of Rhesus monkeys); (FIG. 15B, depicting HA-specific IFN-γ-expressing cells within CD4+ T cell subsets of Cynomolgus monkeys). Connecting lines show animal-specific kinetics over time. Each thin line represents an individual animal and thick line depicts the median of 3 animals per group.

FIG. 16. Virus Neutralization Titers Elicited by Immunization of Mice with a Monovalent Versus Quadrivalent Influenza modRNA Vaccine Encoding HA. Female Balb/c mice received two IM immunizations 4 weeks apart (Days 0 and 28) with 1 μg of an LNP-formulated monovalent modRNA-HA or 4 μg total dose of an LNP-formulated quadrivalent modRNA-HA vaccine preparation (containing 1 μg of each modRNA-HA). The quadrivalent vaccine groups consisted of a vaccine made by combining the 4 modRNA-HAs in equal ratios and then coformulating into LNPs (“pre-mix”) or a mixture of separately formulated modRNA-LNP complexes each expressing a single HA (“post-mix”). Virus neutralization responses against A/Wisconsin/588/2019 (H1N1), A/Cambodia/e0826360/2020 (H3N2), B/Washington/02/2019 (B/Victoria-lineage), and B/Phuket/3073/2013 (B/Yamagata-lineage) virus strains were measured by a 1-Day MNT assay on Day 42 (2 weeks post-dose 2).

FIG. 17A-B. Functional Antibody and Virus Neutralization Titers Elicited by Immunization of Mice With a Quadrivalent Influenza modRNA Vaccine Encoding 4 Has. Female Balb/c mice received two IM immunizations 4 weeks apart (Days 0 and 28) with 0.8 μg of LNP-formulated quadrivalent modRNA-HA vaccine or 2.4 μg of a licensed adjuvanted quadrivalent inactivated vaccine (QIV), Fluad. Functional antibody and virus neutralization titers against A/Wisconsin/588/2019 (H1N1), A/Cambodia/e0826360/2020 (H3N2), B/Washington/02/2019 (B/Victoria-lineage), and B/Phuket/3073/2013 (B/Yamagata-lineage) virus strains were measured by an HAI (FIG. 17A) and a 1-Day MNT (FIG. 17B) assay on Day 42 (2 weeks post-dose 2).

FIG. 18A-C. Functional Antibody and Virus Neutralization Titers Elicited by Immunization of Mice with Influenza modRNA Vaccines Encoding HA and NA. Female Balb/c mice received two IM immunizations 4 weeks apart (Days 0 and 28) with 1.6 μg of LNP-formulated quadrivalent-HA/NA (qIRV-HA/NA) modRNA vaccine or 0.8 μg of LNP-formulated quadrivalent-HA (qIRV-HA) or quadrivalent-NA (qIRV-NA) modRNA vaccines (0.2 μg of each modRNA construct). Functional antibody and virus neutralization titers against A/Wisconsin/588/2019 (H1N1) and B/Austria/1359417/2021 (Victoria-lineage) virus strains were measured by an HAI (FIG. 18A), a 1-Day MNT (FIG. 18B), and an NAI (FIG. 18C) assay on Day 42 (2 weeks post-boost).

FIG. 19A-H. Virus Neutralization Titers Elicited by Immunization of Mice with a Pandemic Influenza modRNA H5 Vaccine. Female BALB/c mice were immunized IM with 10 μg of LNP-formulated modRNA encoding HA from A/Astrakhan/3212/2020 (H5N8) on Days 0 and 28. Virus neutralization responses against the matched virus strain (FIG. 19A) A/Astrakhan/3212/2020 (H5N8) and heterologous strains (FIG. 19B) A/American wigeon/South Carolina/22-000345-001/2021 (H5N1), (FIG. 19C) A/gyrfalcon/Washington/41088-6/2014 (H5N8), (FIG. 19D) A/chicken/Vietnam/RAHO4-CD-20-421/2020 (H5N6), A/chicken/Ghana/20/2015 (H5N1)(FIG. 19E), A/chicken/Guangdong/18SF020/2018 (H5N6)(FIG. 19F), A/duck/Bangladesh/17/D1012/2018 (H5N1)(FIG. 19F), and A/Vietnam/1203/2004 (H5N1)(FIG. 19H) were measured by a 1-Day MNT on Day 42 (2 weeks post-Dose 2). LOD=limit of detection.

### DETAILED DESCRIPTION

Embodiments of the present disclosure provide RNA (e.g., mRNA) vaccines that include polynucleotide encoding an influenza virus antigen. Influenza virus RNA vaccines, as provided herein may be used to induce a balanced immune response, comprising both cellular and humoral immunity, without many of the risks associated with DNA vaccination. In some embodiments, the virus is a strain of Influenza A or Influenza B or combinations thereof.

In one aspect, the disclosure relates to an immunogenic composition including: (i) a first ribonucleic acid (RNA) polynucleotide having an open reading frame encoding a first antigen, said antigen including at least one influenza virus antigenic polypeptide or an immunogenic fragment thereof, and (ii) a second RNA polynucleotide having an open reading frame encoding a second antigen, said second antigen including at least one influenza virus antigenic polypeptide or an immunogenic fragment thereof, wherein the first and second RNA polynucleotides are formulated in a lipid nanoparticle (LNP). In some embodiments, the first and second antigens include hemagglutinin (HA), or an immunogenic fragment or variant thereof. In some embodiments, the first antigen includes an HA from a different subtype of influenza virus to the influenza virus antigenic polypeptide or an immunogenic fragment thereof of the second antigen. In some embodiments, the composition further includes (iii) a third antigen including at least one influenza virus antigenic polypeptide or an immunogenic fragment thereof, wherein the third antigen is from influenza virus but is from a different strain of influenza virus to both the first and second antigens. In some embodiments, the first, second and third RNA polynucleotides are formulated in a lipid nanoparticle.

In some embodiments, the composition further includes (iv) a fourth RNA polynucleotide having an open reading frame encoding a fourth antigen, said antigen including at least one influenza virus antigenic polypeptide or an immunogenic fragment thereof, wherein the fourth antigen is from influenza virus but is from a different strain of influenza virus to the first, second and third antigens. In some embodiments, the first, second, third, and fourth RNA polynucleotides are formulated in a lipid nanoparticle.

In some embodiments, the RNA polynucleotides are mixed in desired ratios in a single vessel and are subsequently formulated into lipid nanoparticles. The inventors surprisingly discovered that the initial input of different RNA polynucleotides at a known ratio to be formulated in a single LNP process surprisingly resulted in LNPs encapsulating the different RNA polynucleotides in about the same ratio as the input ratio. The results were surprising in view of the potential for the manufacturing process to favor one RNA polynucleotide to another when encapsulating the RNA polynucleotides into an LNP. Such embodiments may be referred herein as “pre-mix”. Accordingly, in some embodiments, first and second RNA polynucleotides are formulated in a single lipid nanoparticle. In some embodiments, the first, second, third, and fourth RNA polynucleotides are formulated in a single LNP. In some embodiments, the first, second, third, fourth, and fifth RNA polynucleotides are formulated in a single LNP. In some embodiments, the first, second, third, fourth, fifth, and sixth RNA polynucleotides are formulated in a single LNP. In some embodiments, the first, second, third, fourth, fifth, sixth, and seventh RNA polynucleotides are formulated in a single LNP. In some embodiments, the first, second, third, fourth, fifth, sixth, seventh, and eighth RNA polynucleotides are formulated in a single LN P.

In some embodiments, the molar ratio of the first RNA polynucleotide to the second RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the second RNA polynucleotide is greater than 1:1.

In some embodiments, the molar ratio of the first RNA polynucleotide to the third RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the third RNA polynucleotide is greater than 1:1.

In some embodiments, the molar ratio of the first RNA polynucleotide to the fourth RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the fourth RNA polynucleotide is greater than 1:1. In some embodiments, the molar ratio of the first RNA polynucleotide to the fifth RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the fifth RNA polynucleotide is greater than 1:1. In some embodiments, the molar ratio of the first RNA polynucleotide to the sixth RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the sixth RNA polynucleotide is greater than 1:1. In some embodiments, the molar ratio of the first RNA polynucleotide to the seventh RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the seventh RNA polynucleotide is greater than 1:1. In some embodiments, the molar ratio of the first RNA polynucleotide to the eighth RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the eighth RNA polynucleotide is greater than 1:1.

In alternative embodiments, each RNA polynucleotide encoding a particular antigen is formulated in an individual LNP, such that each LNP encapsulates an RNA polynucleotide encoding identical antigens. Such embodiments may be referred herein as “post-mix”. Accordingly, in some embodiments, the first RNA polynucleotide is formulated in a first LNP; the second RNA polynucleotide is formulated in a second LNP; the third RNA polynucleotide is formulated in a third LNP; the fourth RNA polynucleotide is formulated in a fourth LNP; the fifth RNA polynucleotide is formulated in a fifth LNP; the sixth RNA polynucleotide is formulated in a sixth LNP; the seventh RNA polynucleotide is formulated in a seventh LNP; and the eighth RNA polynucleotide is formulated in an eighth LNP.

In some embodiments, the molar ratio of the first LNP to the second LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the second LNP is greater than 1:1.

In some embodiments, the molar ratio of the first LNP to the third LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the third LNP is greater than 1:1.

In some embodiments, the molar ratio of the first LNP to the fourth LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the fourth LNP is greater than 1:1. In some embodiments, the molar ratio of the first LNP to the fifth LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the fifth LNP is greater than 1:1. In some embodiments, the molar ratio of the first LNP to the sixth LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the sixth LNP is greater than 1:1. In some embodiments, the molar ratio of the first LNP to the seventh LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the seventh LNP is greater than 1:1. In some embodiments, the molar ratio of the first LNP to the eighth LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the eighth LNP is greater than 1:1.

Surprisingly, the inventors discovered that regardless of the process, the resulting ratio of RNA polynucleotide was comparable whether the plurality of RNA polynucleotides are mixed prior to formulation in an LNP (pre-mixed) or whether the RNA polynucleotides encoding a particular antigen is formulated in an individual LNP and the plurality of LNPs for different antigens are mixed (post-mixed). As a result of the discovery, there may be an option for medical professionals to mix and administer different ratios of antigens depending on the influenza season, particularly when the individual LNPs encapsulate RNA for a single antigen.

In some embodiments, the antigenic polypeptide encodes a hemagglutinin protein or immunogenic fragment thereof. In some embodiments, the hemagglutinin protein is H1, H2, H3, H4, H5, H6, H7, H8, H9, H10, H11, H12, H13, H14, H15, H16, H17, H18, or an immunogenic fragment thereof. In some embodiments, the hemagglutinin protein does not comprise a head domain. In some embodiments, the hemagglutinin protein comprises a portion of the head domain. In some embodiments, the hemagglutinin protein does not comprise a cytoplasmic domain. In some embodiments, the hemagglutinin protein comprises a portion of the cytoplasmic domain. In some embodiments, the truncated hemagglutinin protein comprises a portion of the transmembrane domain.

Some embodiments provide influenza vaccines comprising one or more RNA polynucleotides having an open reading frame encoding a hemagglutinin protein and a pharmaceutically acceptable carrier or excipient, formulated within a cationic lipid nanoparticle. In some embodiments, the hemagglutinin protein is selected from H1, H7 and H10. In some embodiments, the RNA polynucleotide further encodes neuraminidase (NA) protein. In some embodiments, the hemagglutinin protein is derived from a strain of Influenza A virus or Influenza B virus or combinations thereof. In some embodiments, the Influenza virus is selected from H1N1, H3N2, H7N9, and H10N8.

In some embodiments, the virus is a strain of Influenza A or Influenza B or combinations thereof. In some embodiments, the strain of Influenza A or Influenza B is associated with birds, pigs, horses, dogs, humans, or non-human primates. In some embodiments, the antigenic polypeptide encodes a hemagglutinin protein or fragment thereof. In some embodiments, the hemagglutinin protein is H7 or H10 or a fragment thereof. In some embodiments, the hemagglutinin protein comprises a portion of the head domain (HA1). In some embodiments, the hemagglutinin protein comprises a portion of the cytoplasmic domain. In some embodiments, the truncated hemagglutinin protein. In some embodiments, the protein is a truncated hemagglutinin protein comprises a portion of the transmembrane domain. In some embodiments, the virus is selected from the group consisting of H7N9 and H10N8. Protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length.

In some embodiments, an Influenza RNA composition includes an RNA encoding an antigenic fusion protein. Thus, the encoded antigen or antigens may include two or more proteins (e.g., protein and/or protein fragment) joined together. Alternatively, the protein to which a protein antigen is fused does not promote a strong immune response to itself, but rather to the influenza antigen. Antigenic fusion proteins, in some embodiments, retain the functional property from each original protein.

Some embodiments provide methods of preventing or treating influenza viral infection comprising administering to a subject any of the vaccines described herein. In some embodiments, the antigen specific immune response comprises a T cell response. In some embodiments, the antigen specific immune response comprises a B cell response. In some embodiments, the antigen specific immune response comprises both a T cell response and a B cell response. In some embodiments, the method of producing an antigen specific immune response involves a single administration of the vaccine. In some embodiments, the vaccine is administered to the subject by intradermal, intramuscular injection, subcutaneous injection, intranasal inoculation, or oral administration.

In some embodiments, the RNA (e.g., mRNA) polynucleotides or portions thereof may encode one or more polypeptides or fragments thereof of an influenza strain as an antigen.

mRNA Vaccines of the Disclosure

The present disclosure relates to mRNA vaccines in general. Several mRNA vaccine platforms are available in the prior art. The basic structure of in vitro transcribed (IVT) mRNA closely resembles “mature” eukaryotic mRNA and includes (i) a protein-encoding open reading frame (ORF), flanked by (ii) 5′ and 3′ untranslated regions (UTRs), and at the end sides (iii) a 7-methyl guanosine 5′ cap structure and (iv) a 3′ poly(A) tail. The non-coding structural features play important roles in the pharmacology of mRNA and can be individually optimized to modulate the mRNA stability, translation efficiency, and immunogenicity. By incorporating modified nucleosides, mRNA transcripts referred to as “nucleoside-modified mRNA” can be produced with reduced immunostimulatory activity, and therefore an improved safety profile can be obtained. In addition, modified nucleosides allow the design of mRNA vaccines with strongly enhanced stability and translation capacity, as they can avoid the direct antiviral pathways that are induced by type IFNs and are programmed to degrade and inhibit invading mRNA. For instance, the replacement of uridine with pseudouridine in IVT mRNA reduces the activity of 2′-5′-oligoadenylate synthetase, which regulates the mRNA cleavage by RNase L. In addition, lower activities are measured for protein kinase R, an enzyme that is associated with the inhibition of the mRNA translation process.

Besides the incorporation of modified nucleotides, other approaches have been validated to increase the translation capacity and stability of mRNA. One example is the development of “sequence-engineered mRNA”. Here, mRNA expression can be strongly increased by sequence optimizations in the ORF and UTRs of mRNA, for instance by enriching the GC content, or by selecting the UTRs of natural long-lived mRNA molecules. Another approach is the design of “self-amplifying mRNA” constructs. These are mostly derived from alphaviruses and contain an ORF that is replaced by the antigen of interest together with an additional ORF encoding viral replicase. The latter drives the intracellular amplification of mRNA and can therefore significantly increase the antigen expression capacity.

Also, several modifications have been implemented at the end structures of mRNA. Anti-reverse cap (ARCA) modifications can ensure the correct cap orientation at the 5′ end, which yields almost complete fractions of mRNA that can efficiently bind the ribosomes. Other cap modifications, such as phosphorothioate cap analogs, can further improve the affinity towards the eukaryotic translation initiation factor 4E, and increase the resistance against the RNA decapping complex.

Conversely, by modifying its structure, the potency of mRNA to trigger innate immune responses can be further improved, but to the detriment of translation capacity. By stabilizing the mRNA with either a phosphorothioate backbone, or by its precipitation with the cationic protein protamine, antigen expression can be diminished, but stronger immune-stimulating capacities can be obtained.

In one aspect the disclosure describes to an immunogenic composition comprising an mRNA molecule that encodes one or more polypeptides or fragments thereof of an influenza strain as an antigen.

In some embodiments, the mRNA molecule comprises a nucleoside-modified mRNA. mRNA useful in the disclosure typically include a first region of linked nucleosides encoding a polypeptide of interest (e.g., a coding region), a first flanking region located at the 5′-terminus of the first region (e.g., a 5-UTR), a second flanking region located at the 3′-terminus of the first region (e.g., a 3-UTR), at least one 5′-cap region, and a 3′-stabilizing region. In some embodiments, the mRNA of the disclosure further includes a poly-A region or a Kozak sequence (e.g., in the 5′-UTR). In some cases, mRNA of the disclosure may contain one or more intronic nucleotide sequences capable of being excised from the polynucleotide. In some embodiments, mRNA of the disclosure may include a 5′ cap structure, a chain terminating nucleotide, a stem loop, a poly A sequence, and/or a polyadenylation signal. Any one of the regions of a nucleic acid may include one or more alternative components (e.g., an alternative nucleoside). For example, the 3′-stabilizing region may contain an alternative nucleoside such as an L-nucleoside, an inverted thymidine, or a 2′-0-methyl nucleoside and/or the coding region, 5′-UTR, 3′-UTR, or cap region may include an alternative nucleoside such as a 5-substituted uridine (e.g., 5-methoxyuridine), a 1-substituted pseudouridine (e.g., 1-methyl-pseudouridine), and/or a 5-substituted cytidine (e.g., 5-methyl-cytidine).

The compositions described herein comprise at least one RNA polynucleotide, such as a mRNA (e.g., modified mRNA). mRNA, for example, is transcribed in vitro from template DNA, referred to as an “in vitro transcription template.” In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide.

In preferred embodiments, the 5′ UTR comprises SEQ ID NO: 1.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

In preferred embodiments, the 3′ UTR comprises SEQ ID NO: 2.

An “open reading frame” is a continuous stretch of DNA beginning with a start codon (e.g., methionine (ATG)), and ending with a stop codon (e.g., TAA, TAG or TGA) and encodes a polypeptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, export of the mRNA from the nucleus and translation.

In preferred embodiments, the 3′ polyadenylation tail comprises SEQ ID NO: 3.

In some embodiments, a polynucleotide includes 200 to 3,000 nucleotides. For example, a polynucleotide may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

In some embodiments, a LNP includes one or more RNAs, and the one or more RNAs, lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 5.67:1, about 6.0:1, about 6.5:1, or about 7.0:1. For example, the N:P ratio may be about 5.67:1.

mRNA of the disclosure may include one or more naturally occurring components, including any of the canonical nucleotides A (adenosine), G (guanosine), C (cytosine), U (uridine), or T (thymidine). In one embodiment, all or substantially all of the nucleotides comprising (a) the 5′-UTR, (b) the open reading frame (ORF), (c) the 3′-UTR, (d) the poly A tail, and any combination of (a, b, c, or d above) comprise naturally occurring canonical nucleotides A (adenosine), G (guanosine), C (cytosine), U (uridine), or T (thymidine).

mRNA of the disclosure may include one or more alternative components, as described herein, which impart useful properties including increased stability and/or the lack of a substantial induction of the innate immune response of a cell into which the polynucleotide is introduced. For example, a modRNA may exhibit reduced degradation in a cell into which the modRNA is introduced, relative to a corresponding unaltered mRNA. These alternative species may enhance the efficiency of protein production, intracellular retention of the polynucleotides, and/or viability of contacted cells, as well as possess reduced immunogenicity.

mRNA of the disclosure may include one or more modified (e.g., altered or alternative) nucleobases, nucleosides, nucleotides, or combinations thereof. The mRNA useful in a LNP can include any useful modification or alteration, such as to the nucleobase, the sugar, or the internucleoside linkage (e.g., to a linking phosphate/to a phosphodiester linkage/to the phosphodiester backbone). In certain embodiments, alterations (e.g., one or more alterations) are present in each of the nucleobase, the sugar, and the internucleoside linkage. Alterations according to the present disclosure may be alterations of ribonucleic acids (RNAs), e.g., the substitution of the 2′-OH of the ribofuranosyl ring to 2′-H, threose nucleic acids (TNAs), glycol nucleic acids (GNAs), peptide nucleic acids (PNAs), locked nucleic acids (LNAs), or hybrids thereof. Additional alterations are described herein.

mRNA of the disclosure may or may not be uniformly altered along the entire length of the molecule. For example, one or more or all types of nucleotide (e.g., purine or pyrimidine, or any one or more or all of A, G, U, C) may or may not be uniformly altered in a mRNA, or in a given predetermined sequence region thereof. In some instances, all nucleotides X in a mRNA (or in a given sequence region thereof) are altered, wherein X may any one of nucleotides A, G, U, C, or any one of the combinations A+G, A+U, A+C, G+U, G+C, U+C, A+G+U, A+G+C, G+U+C or A+G+C.

Different sugar alterations and/or internucleoside linkages (e.g., backbone structures) may exist at various positions in a polynucleotide. One of ordinary skill in the art will appreciate that the nucleotide analogs or other alteration(s) may be located at any position(s) of a polynucleotide such that the function of the polynucleotide is not substantially decreased. An alteration may also be a 5′- or 3′-terminal alteration. In some embodiments, the polynucleotide includes an alteration at the 3′-terminus. The polynucleotide may contain from about 1% to about 100% alternative nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of a canonical nucleotide (e.g., A, G, U, or C).

Polynucleotides may contain at a minimum zero and at maximum 100% alternative nucleotides, or any intervening percentage, such as at least 5% alternative nucleotides, at least 10% alternative nucleotides, at least 25% alternative nucleotides, at least 50% alternative nucleotides, at least 80% alternative nucleotides, or at least 90% alternative nucleotides. For example, polynucleotides may contain an alternative pyrimidine such as an alternative uracil or cytosine. In some embodiments, at least 5%, at least 10%, at least 25%, at least 50%, at least 80%, at least 90% or 100% of the uracil in a polynucleotide is replaced with an alternative uracil (e.g., a 5-substituted uracil). The alternative uracil can be replaced by a compound having a single unique structure or can be replaced by a plurality of compounds having different structures (e.g., 2, 3, 4 or more unique structures). In some instances, at least 5%, at least 10%, at least 25%, at least 50%, at least 80%, at least 90% or 100% of the cytosine in the polynucleotide is replaced with an alternative cytosine (e.g., a 5-substituted cytosine). The alternative cytosine can be replaced by a compound having a single unique structure or can be replaced by a plurality of compounds having different structures (e.g., 2, 3, 4 or more unique structures).

In some instances, nucleic acids do not substantially induce an innate immune response of a cell into which the polynucleotide (e.g., mRNA) is introduced. Features of an induced innate immune response include 1) increased expression of pro-inflammatory cytokines, 2) activation of intracellular PRRs (RIG-I, MDA5, etc., and/or 3) termination or reduction in protein translation.

In some embodiments, the mRNA comprises one or more alternative nucleoside or nucleotides. The alternative nucleosides and nucleotides can include an alternative nucleobase. A nucleobase of a nucleic acid is an organic base such as a purine or pyrimidine or a derivative thereof. A nucleobase may be a canonical base (e.g., adenine, guanine, uracil, thymine, and cytosine). These nucleobases can be altered or wholly replaced to provide polynucleotide molecules having enhanced properties, e.g., increased stability such as resistance to nucleases. Non-canonical or modified bases may include, for example, one or more substitutions or modifications including but not limited to alkyl, aryl, halo, oxo, hydroxyl, alkyloxy, and/or thio substitutions; one or more fused or open rings; oxidation; and/or reduction.

In some embodiments, the nucleobase is an alternative uracil. Exemplary nucleobases and nucleosides having an alternative uracil include pseudouridine (iii), pyridin-4-one ribonucleoside, 5-aza-uracil, 6-aza-uracil, 2-thio-5-aza-uracil, 2-thio-uracil (s2U), 4-thio-uracil (s4U), 4-thio-pseudouridine, 2-thio-pseudouridine, 5-hydroxy-uracil (ho5U), 5-aminoallyl-uracil, 5-halo-uracil (e.g., 5-iodo-uracil or 5-bromo-uracil), 3-methyl-uracil (m U), 5-methoxy-uracil (mo5U), uracil 5-oxyacetic acid (cmo5U), uracil 5-oxyacetic acid methyl ester (mcmo5U), 5-carboxymethyl-uracil (cm5U), 1-carboxymethyl-pseudouridine, 5-carboxyhydroxymethyl-uracil (chm5U), 5-carboxyhydroxymethyl-uracil methyl ester (mchm5U), 5-methoxycarbonylmethyl-uracil (mcm5U), 5-methoxycarbonylmethyl-2-thio-uracil (mcm5s2U), 5-aminomethyl-2-thio-uracil (nmVu), 5-methylaminomethyl-uracil (mnm5U), 5-methylaminomethyl-2-thio-uracil (mnmVu), 5-methylaminomethyl-2-seleno-uracil (mnm5se2U), 5-carbamoylmethyl-uracil (ncm5U), 5-carboxymethylaminomethyl-uracil (cmnm5U), 5-carboxymethylaminomethyl-2-thio-uracil (cmnmVu), 5-propynyl-uracil, 1-propynyl-pseudouracil, 5-taurinomethyl-uracil (xm5U), 1-taurinomethyl-pseudouridine, 5-taurinomethyl-2-thio-uraciI(xm5s2U), 1-taurinomethyl-4-thio-pseudouridine, 5-methyl-uracil (m5U, i.e., having the nucleobase deoxythymine), 1-methyl-pseudouridine (mV), 5-methyl-2-thio-uracil (m5s2U), I-methyl-4-thio-pseudouridine (m xj/), 4-thio-1-methyl-pseudouridine, 3-methyl-pseudouridine (mψ), 2-thio-1-methyl-pseudouridine, 1-methyl-1-deaza-pseudouri dine, 2-thio-I-methyl-1-deaza-pseudouri dine, dihydrouracil (D), dihydropseudouridine, 5,6-dihydrouracil, 5-methyl-dihydrouracil (m5D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, N1-methyl-pseudouridine, 3-(3-amino-3-carboxypropyl)uracil (acp U), I-methyl-3-(3-amino-3-carboxypropyl)pseudouridine (acp ψ), 5-(isopentenylaminomethyl)uracil (inm5U), 5-(isopentenylaminomethyl)-2-thio-uracil (inm5s2U), 5,2′-0-dimethyl-uridine (m5Um), 2-thio-2′-O_methyl-uridine (s2Um), 5-methoxycarbonylmethyl-2′-0-methyl-uridine (mem Um), 5-carbamoylmethyl-2′-0-methyl-uridine (ncm5Um), 5-carboxymethylaminomethyl-2′-0-methyl-uridine (cmnm5Um), 3,2-0-dimethyl-uridine (m Um), and 5-(isopentenylaminomethyl)-2′-0-methyl-uridine (inm5Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(I-E-propenylamino)]uracil.

In some embodiments, the nucleobase is an alternative cytosine. Exemplary nucleobases and nucleosides having an alternative cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocy tidine, 4-thio-1-methy 1-pseudoisocy tidine, 4-thio-1-methyl-1-deaza-pseudoisocytidine, 1-methyl-1-deaza-pseudoisocyti dine, zebularine, 5-aza-zebularine, 5-methy 1-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-0-dimethyl-cytidine (m5Cm), N4-acetyl-2′-0-methyl-cytidine (ac4Cm), N4,2′-0-dimethyl-cytidine (m4Cm), 5-formyl-2′-0-methyl-cytidine (f5Cm), N4,N4,2′-0-trimethyl-cytidine (m42Cm), 1-thio-cytosine, 5-hydroxy-cytosine, 5-(3-azidopropyl)-cytosine, and 5-(2-azidoethyl)-cytosine.

In some embodiments, the nucleobase is an alternative adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methy 1-adenine (m1 A), 2-methyl-adenine (m2A), N6-methyl-adenine (m6A), 2-methylthio-N6-methyl-adenine (ms2m6A), N6-isopentenyl-adenine (i6A), 2-methylthio-N6-isopentenyl-adenine (ms2i6A), N6-(cis-hydroxyisopentenyl)adenine (io6A), 2-methylthio-N6-(cis-hydroxyisopentenyl)adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms2g6A), N6, N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-0-dimethyl-adenosine (m6Am), N6,N6,2′-0-trimethyl-adenosine (m62Am), 1,2′-0-dimethyl-adenosine (m1 Am), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some embodiments, the nucleobase is an alternative guanine. Exemplary nucleobases and nucleosides having an alternative guanine include inosine (I), 1-methyl-inosine (mil), wyosine (imG), methylwyosine (mimG), 4-demethyl-wyosine (imG-14), isowyosine (imG2), wybutosine (yW), peroxywybutosine (o2yVV), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQO), 7-aminomethyl-7-deaza-guanine (preQl), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (mIG), N2-methyl-guanine (m2G), N2,N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-0-methyl-guanosine (m2Gm), N2,N2-dimethyl-2′-0-methyl-guanosine (m22Gm), 1-methyl-2′-0-methyl-guanosine (mlGm), N2,7-dimethyl-2′-0-methyl-guanosine (m2,7Gm), 2′-0-methyl-inosine (Im), 1,2′-0-dimethyl-inosine (mllm), 1-thio-guanine, and 0-6-methyl-guanine.

The alternative nucleobase of a nucleotide can be independently a purine, a pyrimidine, a purine or pyrimidine analog. For example, the nucleobase can be an alternative to adenine, cytosine, guanine, uracil, or hypoxanthine. In another embodiment, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[I,5-a] 1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine. When the nucleotides are depicted using the shorthand A, G, C, T or U, each letter refers to the representative base and/or derivatives thereof, e.g., A includes adenine or adenine analogs, e.g., 7-deaza adenine).

The mRNA may include a 5′-cap structure. The 5′-cap structure of a polynucleotide is involved in nuclear export and increasing polynucleotide stability and binds the mRNA Cap Binding Protein (CBP), which is responsible for polynucleotide stability in the cell and translation competency through the association of CBP with poly-A binding protein to form the mature cyclic mRNA species. The cap further assists the removal of 5′-proximal introns removal during mRNA splicing.

Endogenous polynucleotide molecules may be 5′-end capped generating a 5′-ppp-5′-triphosphate linkage between a terminal guanosine cap residue and the 5′-terminal transcribed sense nucleotide of the polynucleotide. This 5′-guanylate cap may then be methylated to generate an N7-methyl-guanylate residue. The ribose sugars of the terminal and/or anteterminal transcribed nucleotides of the 5′ end of the polynucleotide may optionally also be 2′-0-methylated. 5′-decapping through hydrolysis and cleavage of the guanylate cap structure may target a polynucleotide molecule, such as an mRNA molecule, for degradation.

Alterations to polynucleotides may generate a non-hydrolyzable cap structure preventing decapping and thus increasing polynucleotide half-life. Because cap structure hydrolysis requires cleavage of 5′-ppp-5′ phosphorodiester linkages, alternative nucleotides may be used during the capping reaction. For example, a Vaccinia Capping Enzyme from New England Biolabs (Ipswich, MA) may be used with a-thio-guanosine nucleotides according to the manufacturer's instructions to create a phosphorothioate linkage in the 5′-ppp-5′ cap.

Additional alternative guanosine nucleotides may be used such as a-methyl-phosphonate and seleno-phosphate nucleotides. Additional alterations include, but are not limited to, 2′-0-methylation of the ribose sugars of 5′-terminal and/or 5′-anteterminal nucleotides of the polynucleotide (as mentioned above) on the 2′-hydroxy group of the sugar. Multiple distinct 5′-cap structures can be used to generate the 5′-cap of an mRNA molecule.

Cap analogs, which herein are also referred to as synthetic cap analogs, chemical caps, chemical cap analogs, or structural or functional cap analogs, differ from natural (i.e., endogenous, wild-type, or physiological) 5′-caps in their chemical structure, while retaining cap function. Cap analogs may be chemically (i.e., non-enzymatically) or enzymatically synthesized and/linked to a polynucleotide. For example, the Anti-Reverse Cap Analog (ARCA) cap contains two guanosines linked by a 5′-5′-triphosphate group, wherein one guanosine contains an N7-methyl group as well as a 3′-0-methyl group (i.e., N7, ′-0-dimethyl-guanosine-5′-triphosphate-5′-guanosine, m7G-3′mppp-G, which may equivalently be designated 3′ 0-Me-m7G(5′)ppp(5′)G). The 3′-0 atom of the other, unaltered, guanosine becomes linked to the 5′-terminal nucleotide of the capped polynucleotide (e.g., an mRNA). The N7- and 3′-0-methylated guanosine provides the terminal moiety of the capped polynucleotide (e.g., mRNA). Another exemplary cap is mCAP, which is similar to ARCA but has a 2′-0-methyl group on guanosine (i.e., N7,2′-0-dimethyl-guanosine-5′-triphosphate-5′-guanosine, m7Gm-ppp-G).

A cap may be a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the cap structures of which are herein incorporated by reference.

Alternatively, a cap analog may be a N7-(4-chlorophenoxy ethyl) substituted dinucleotide cap analog known in the art and/or described herein. Non-limiting examples of N7-(4-chlorophenoxy ethyl) substituted dinucleotide cap analogs include a N7-(4-chlorophenoxyethyl)-G(5)ppp(5′)G and a N7-(4-chlorophenoxyethyl)-m3′-OG(5)ppp(5′)G cap analog (see, e.g., the various cap analogs and the methods of synthesizing cap analogs described in Kore et al. Bioorganic & Medicinal Chemistry 2013 21:4570-4574; the cap structures of which are herein incorporated by reference). In other instances, a cap analog useful in the polynucleotides of the present disclosure is a 4-chloro/bromophenoxy ethyl analog.

While cap analogs allow for the concomitant capping of a polynucleotide in an in vitro transcription reaction, up to 20% of transcripts remain uncapped. This, as well as the structural differences of a cap analog from endogenous 5′-cap structures of polynucleotides produced by the endogenous, cellular transcription machinery, may lead to reduced translational competency and reduced cellular stability.

Alternative polynucleotides may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function, and/or structure as compared to synthetic features or analogs of the prior art, or which outperforms the corresponding endogenous, wild-type, natural, or physiological feature in one or more respects. Non-limiting examples of more authentic 5′-cap structures useful in the polynucleotides of the present disclosure are those which, among other things, have enhanced binding of cap binding proteins, increased half-life, reduced susceptibility to 5′-endonucleases, and/or reduced 5′-decapping, as compared to synthetic 5′-cap structures known in the art (or to a wild-type, natural or physiological 5′-cap structure). For example, recombinant Vaccinia Virus Capping Enzyme and recombinant 2′-0-methyltransferase enzyme can create a canonical 5′-5′-triphosphate linkage between the 5′-terminal nucleotide of a polynucleotide and a guanosine cap nucleotide wherein the cap guanosine contains an N7-methylation and the 5′-terminal nucleotide of the polynucleotide contains a 2′-0-methyl. Such a structure is termed the Capl structure. This cap results in a higher translational-competency, cellular stability, and a reduced activation of cellular pro-inflammatory cytokines, as compared, e.g., to other 5′ cap analog structures known in the art. Other exemplary cap structures include 7mG(5′)ppp(5′)N,pN2p (Cap 0), 7mG(5′)ppp(5′)NlmpNp (Cap 1), 7mG(5′)-ppp(5′)NlmpN2mp (Cap 2), and m(7)Gpppm(3)(6,6,2′)Apm(2′)Apm(2′)Cpm(2)(3,2′)Up (Cap 4).

Because the alternative polynucleotides may be capped post-transcriptionally, and because this process is more efficient, nearly 100% of the mRNA may be capped. This is in contrast to −80% when a cap analog is linked to a polynucleotide in the course of an in vitro transcription reaction. 5′-terminal caps may include endogenous caps or cap analogs. A 5′ terminal cap may include a guanosine analog. Useful guanosine analogs include inosine, N1-methyl-guanosine, 2′-fluoro-guanosine, 7-deaza-guanosine, 8-oxo-guanosine, 2-amino-guanosine, LNA-guanosine, and 2-azido-guanosine. In some cases, a polynucleotide contains a modified 5′-cap. A modification on the 5′-cap may increase the stability of polynucleotide, increase the half-life of the polynucleotide, and could increase the polynucleotide translational efficiency. The modified 5′-cap may include, but is not limited to, one or more of the following modifications: modification at the 2′- and/or 3′-position of a capped guanosine triphosphate (GTP), a replacement of the sugar ring oxygen (that produced the carbocyclic ring) with a methylene moiety (CH2), a modification at the triphosphate bridge moiety of the cap structure, or a modification at the nucleobase (G) moiety.

A 5′-UTR may be provided as a flanking region to the mRNA. A 5′-UTR may be homologous or heterologous to the coding region found in a polynucleotide. Multiple 5′-UTRs may be included in the flanking region and may be the same or of different sequences. Any portion of the flanking regions, including none, may be codon optimized and any may independently contain one or more different structural or chemical alterations, before and/or after codon optimization.

In one embodiment, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park Calif.) and/or proprietary methods. In some embodiments, the open reading frame (ORF) sequence is optimized using optimization algorithms. To alter one or more properties of an mRNA, 5′-UTRs which are heterologous to the coding region of an mRNA may be engineered. The mRNA may then be administered to cells, tissue or organisms and outcomes such as protein level, localization, and/or half-life may be measured to evaluate the beneficial effects the heterologous 5′-UTR may have on the mRNA. Variants of the 5′-UTRs may be utilized wherein one or more nucleotides are added or removed to the termini, including A, T, C or G. 5′-UTRs may also be codon-optimized, or altered in any manner described herein.

mRNAs may include a stem loop such as, but not limited to, a histone stem loop. The stem loop may be a nucleotide sequence that is about 25 or about 26 nucleotides in length. The histone stem loop may be located 3′-relative to the coding region (e.g., at the 3′-terminus of the coding region). As a non-limiting example, the stem loop may be located at the 3′-end of a polynucleotide described herein. In some cases, an mRNA includes more than one stem loop (e.g., two stem loops). A stem loop may be located in a second terminal region of a polynucleotide. As a non-limiting example, the stem loop may be located within an untranslated region (e.g., 3′-UTR) in a second terminal region. In some cases, a mRNA which includes the histone stem loop may be stabilized by the addition of a 3′-stabilizing region (e.g., a 3′-stabilizing region including at least one chain terminating nucleoside). Not wishing to be bound by theory, the addition of at least one chain terminating nucleoside may slow the degradation of a polynucleotide and thus can increase the half-life of the polynucleotide. In other cases, a mRNA, which includes the histone stem loop may be stabilized by an alteration to the 3′-region of the polynucleotide that can prevent and/or inhibit the addition of oligio(U). In yet other cases, a mRNA, which includes the histone stem loop may be stabilized by the addition of an oligonucleotide that terminates in a 3′-deoxynucleoside, 2′,3′-dideoxynucleoside 3′-0-methylnucleosides, 3-0-ethylnucleosides, 3′-arabinosides, and other alternative nucleosides known in the art and/or described herein. In some instances, the mRNA of the present disclosure may include a histone stem loop, a poly-A region, and/or a 5′-cap structure. The histone stem loop may be before and/or after the poly-A region. The polynucleotides including the histone stem loop and a poly-A region sequence may include a chain terminating nucleoside described herein. In other instances, the polynucleotides of the present disclosure may include a histone stem loop and a 5′-cap structure. The 5′-cap structure may include, but is not limited to, those described herein and/or known in the art. In some cases, the conserved stem loop region may include a miR sequence described herein. As a non-limiting example, the stem loop region may include the seed sequence of a miR sequence described herein. In another non-limiting example, the stem loop region may include a miR-122 seed sequence.

mRNA may include at least one histone stem-loop and a poly-A region or polyadenylation signal. In certain cases, the polynucleotide encoding for a histone stem loop and a poly-A region or a polyadenylation signal may code for a pathogen antigen or fragment thereof. In other cases, the polynucleotide encoding for a histone stem loop and a poly-A region or a polyadenylation signal may code for a therapeutic protein. In some cases, the polynucleotide encoding for a histone stem loop and a poly-A region or a polyadenylation signal may code for a tumor antigen or fragment thereof. In other cases, the polynucleotide encoding for a histone stem loop and a poly-A region or a polyadenylation signal may code for an allergenic antigen or an autoimmune self-antigen.

An mRNA may include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of a nucleic acid. During RNA processing, a long chain of adenosine nucleotides (poly-A region) is normally added to messenger RNA (mRNA) molecules to increase the stability of the molecule. Immediately after transcription, the 3′-end of the transcript is cleaved to free a 3′-hydroxy. Then poly-A polymerase adds a chain of adenosine nucleotides to the RNA. The process, called polyadenylation, adds a poly-A region that is between 100 and 250 residues long. Unique poly-A region lengths may provide certain advantages to the alternative polynucleotides of the present disclosure. Generally, the length of a poly-A region of the present disclosure is at least 30 nucleotides in length. In another embodiment, the poly-A region is at least 35 nucleotides in length. In another embodiment, the length is at least 40 nucleotides. In another embodiment, the length is at least 45 nucleotides. In another embodiment, the length is at least 55 nucleotides. In another embodiment, the length is at least 60 nucleotides. In another embodiment, the length is at least 70 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1700 nucleotides. In another embodiment, the length is at least 1800 nucleotides. In another embodiment, the length is at least 1900 nucleotides. In another embodiment, the length is at least 2000 nucleotides. In another embodiment, the length is at least 2500 nucleotides. In another embodiment, the length is at least 3000 nucleotides. In some instances, the poly-A region may be 80 nucleotides, 120 nucleotides, 160 nucleotides in length on an alternative polynucleotide molecule described herein. In other instances, the poly-A region may be 20, 40, 80, 100, 120, 140 or 160 nucleotides in length on an alternative polynucleotide molecule described herein. In some cases, the poly-A region is designed relative to the length of the overall alternative polynucleotide. This design may be based on the length of the coding region of the alternative polynucleotide, the length of a particular feature or region of the alternative polynucleotide (such as mRNA) or based on the length of the ultimate product expressed from the alternative polynucleotide. When relative to any feature of the alternative polynucleotide (e.g., other than the mRNA portion which includes the poly-A region) the poly-A region may be 10, 20, 30, 40, 50, 60, 70, 80, 90 or 100% greater in length than the additional feature. The poly-A region may also be designed as a fraction of the alternative polynucleotide to which it belongs. In this context, the poly-A region may be 10, 20, 30, 40, 50, 60, 70, 80, or 90% or more of the total length of the construct or the total length of the construct minus the poly-A region.

In certain cases, engineered binding sites and/or the conjugation of mRNA for poly-A binding protein may be used to enhance expression. The engineered binding sites may be sensor sequences which can operate as binding sites for ligands of the local microenvironment of the mRNA. As a non-limiting example, the mRNA may include at least one engineered binding site to alter the binding affinity of poly-A binding protein (PABP) and analogs thereof. The incorporation of at least one engineered binding site may increase the binding affinity of the PABP and analogs thereof.

Additionally, multiple distinct mRNA may be linked together to the PABP (poly-A binding protein) through the 3′-end using alternative nucleotides at the 3′-terminus of the poly-A region. Transfection experiments can be conducted in relevant cell lines at and protein production can be assayed by ELISA at 12 hours, 24 hours, 48 hours, 72 hours, and day 7 post-transfection. As a non-limiting example, the transfection experiments may be used to evaluate the effect on PABP or analogs thereof binding affinity as a result of the addition of at least one engineered binding site. In certain cases, a poly-A region may be used to modulate translation initiation. While not wishing to be bound by theory, the poly-A region recruits PABP which in turn can interact with translation initiation complex and thus may be essential for protein synthesis. In some cases, a poly-A region may also be used in the present disclosure to protect against 3′-5′-exonuclease digestion. In some instances, an mRNA may include a polyA-G Quartet. The G-quartet is a cyclic hydrogen bonded array of four guanosine nucleotides that can be formed by G-rich sequences in both DNA and RNA. In this embodiment, the G-quartet is incorporated at the end of the poly-A region. The resultant mRNA may be assayed for stability, protein production and other parameters including half-life at various time points. It has been discovered that the polyA-G quartet results in protein production equivalent to at least 75% of that seen using a poly-A region of 120 nucleotides alone. In some cases, mRNA may include a poly-A region and may be stabilized by the addition of a 3′-stabilizing region. The mRNA with a poly-A region may further include a 5′-cap structure. In other cases, mRNA may include a poly-A-G Quartet. The mRNA with a poly-A-G Quartet may further include a 5′-cap structure. In some cases, the 3′-stabilizing region which may be used to stabilize mRNA includes a poly-A region or poly-A-G Quartet. In other cases, the 3′-stabilizing region which may be used with the present disclosure include a chain termination nucleoside such as 3′-deoxyadenosine (cordycepin), 3′-deoxyuridine, 3′-deoxycytosine, 3′-deoxyguanosine, 3′-deoxy thymine, 2′,3′-dideoxynucleosides, such as 2′,3′-dideoxyadenosine, 2′,3′-dideoxyuridine, 2′,3′-dideoxycytosine, 2′, 3′-dideoxyguanosine, 2′,3′-dideoxythymine, a 2′-deoxynucleoside, or an O-methylnucleoside. In other cases, mRNA which includes a polyA region or a poly-A-G Quartet may be stabilized by an alteration to the 3′-region of the polynucleotide that can prevent and/or inhibit the addition of oligio(U). In yet other instances, mRNA which includes a poly-A region or a poly-A-G Quartet may be stabilized by the addition of an oligonucleotide that terminates in a 3′-deoxynucleoside, 2,3′-dideoxynucleoside 3-0-methylnucleosides, 3′-0-ethylnucleosides, 3′-arabinosides, and other alternative nucleosides known in the art and/or described herein.

Embodiments of the present disclosure further include RNA polynucelotides that are self-amplifying RNA (saRNA) polynucleotides, wherein the saRNA encodes an influenza virus antigen, preferably an antigen from the pandemic influenza strain described herein. As used herein, mRNA encompasses saRNA polynucleotides, It is contemplated that any embodiment discussed in this specification may be implemented with respect to any method or composition of the disclosure, and vice versa. Furthermore, compositions of the disclosure may be used to achieve methods of the disclosure. In some embodiments, the RNA molecule, such as the first RNA molecule, is an saRNA. “saRNA,” “self-amplifying RNA,” and “replicon” refer to RNA with the ability to replicate itself. Selfamplifying RNA molecules may be produced by using replication elements derived from a virus or viruses, e.g., alphaviruses, and substituting the structural viral polypeptides with a nucleotide 30 sequence encoding a polypeptide of interest. A self-amplifying RNA molecule is typically a positive-strand molecule that may be directly translated after delivery to a cell, and this translation provides an RNA-dependent RNA polymerase which then produces both antisense and sense transcripts from the delivered RNA. The delivered RNA leads to the production of multiple daughter RNAs. These daughter RNAs, as well as collinear subgenomic transcripts, may be translated themselves to provide in situ expression of an encoded gene of interest, e.g., a viral antigen, or may be transcribed to provide further transcripts with the same sense as the delivered RNA which are translated to provide in situ expression of the protein of interest, e.g., an antigen. The overall result of this sequence of transcriptions is an amplification in the number of the introduced saRNAs and so the encoded gene of interest, e.g., a viral antigen, can become a major polypeptide product of the cells. In some embodiments, the self-amplifying RNA includes at least one or more genes selected from any one of viral replicases, viral proteases, viral helicases and other nonstructural viral proteins. In some embodiments, the self-amplifying RNA may also include 5′- and 3′-end tractive replication sequences, and optionally a heterologous sequence that encodes a desired amino acid sequence (e.g., an antigen of interest). A subgenomic promoter that directs expression of the heterologous sequence may be included in the self-amplifying RNA. Optionally, the heterologous sequence (e.g., an antigen of interest) may be fused in frame to other coding regions in the self-amplifying RNA and/or may be under the control of an internal ribosome entry site (IRES). In some embodiments, the self-amplifying RNA molecule is not encapsulated in a viruslike particle. Self-amplifying RNA molecules described herein may be designed so that the self-amplifying RNA molecule cannot induce production of infectious viral particles. This may be achieved, for example, by omitting one or more viral genes encoding structural proteins that are necessary to produce viral particles in the self-amplifying RNA. For example, when the selfamplifying RNA molecule is based on an alphavirus, such as Sinbis virus (SIN), Semliki forest virus and Venezuelan equine encephalitis virus (VEE), one or more genes encoding viral structural proteins, such as capsid and/or envelope glycoproteins, may be omitted. In some embodiments, a self-amplifying RNA molecule described herein encodes (i) an RNA-dependent RNA polymerase that may transcribe RNA from the self-amplifying RNA molecule and (ii) a polypeptide of interest, e.g., a viral antigen. In some embodiments, the polymerase may be an alphavirus replicase, e.g., including any one of alphavirus protein nsP1, nsP2, nsP3, nsP4, and any combination thereof. In some embodiments, the self-amplifying RNA molecules described herein may include one or more modified nucleotides (e.g., pseudouridine, N6-methyladenosine, 5-methylcytidine, 5-methyluridine). In some embodiments, the selfamplifying RNA molecules does not include a modified nucleotide (e.g., pseudouridine, N6-methyladenosine, 5-methylcytidine, 5-methyluridine). The saRNA construct may encode at least one non-structural protein (NSP), disposed 5′ or 3′ of the sequence encoding at least one peptide or polypeptide of interest. In some embodiments, the sequence encoding at least one NSP is disposed 5′ of the sequences encoding the peptide or polypeptide of interest. Thus, the sequence encoding at least one NSP may be disposed at the 5′ end of the RNA construct. In some embodiments, at least one non-structural protein encoded by the RNA construct may be the RNA polymerase nsP4. In some embodiments, the saRNA construct encodes nsP1, nsP2, nsP3 and, nsP4. As is known in the art, nsP1 is the viral capping enzyme and membrane anchor of the replication complex (RC). nsP2 is an RNA helicase and the protease responsible for the ns polyprotein processing. nsP3 interacts with several host proteins and may modulate protein poly- and mono-ADP-ribosylation. nsP4 is the core viral RNA-dependent RNA polymerase. In some embodiments, the polymerase may be an alphavirus replicase, e.g., comprising one or more of alphavirus proteins nsP1, nsP2, nsP3, and nsP4. Whereas natural alphavirus genomes encode structural virion proteins in addition to the non-structural replicase polypeptide, in some embodiments, the self-amplifying RNA molecules do not encode alphavirus structural proteins. In some embodiments, the self-amplifying RNA may lead to the production of genomic RNA copies of itself in a cell, but not to the production of RNA that includes virions. Without being bound by theory or mechanism, the inability to produce these virions means that, unlike a wild-type alphavirus, the self-amplifying RNA molecule cannot perpetuate itself in infectious form. The alphavirus structural proteins which are necessary for perpetuation in wild-type viruses can be absent from self-amplifying RNAs of the present disclosure and their place can be taken by gene(s) encoding the immunogen of interest, such that the subgenomic transcript encodes the immunogen rather than the structural alphavirus virion proteins. In some embodiments, the self-amplifying RNA molecule may have two open reading frames. The first (5′) open reading frame can encode a replicase; the second (3′) open reading frame can encode a polypeptide comprising an antigen of interest. In some embodiments the RNA may have additional (e.g., downstream) open reading frames, e.g., to encode further antigens or to encode accessory polypeptides. In some embodiments, the second RNA or the saRNA molecule further includes (1) an alphavirus 5′ replication recognition sequence, and (2) an alphavirus 3′ replication recognition sequence. In some embodiments, the 5′ sequence of the self-amplifying RNA molecule is selected to ensure compatibility with the encoded replicase. Optionally, self-amplifying RNA molecules described herein may also be designed to induce production of infectious viral particles that are attenuated or virulent, or to produce viral particles that are capable of a single round of subsequent infection. In some embodiments, the saRNA molecule is alphavirus-based. Alphaviruses include a set of genetically, structurally, and serologically related arthropod-borne viruses of the Togaviridae family. Exemplary viruses and virus subtypes within the alphavirus genus include Sindbis virus, Semliki Forest virus, Ross River virus, and Venezuelan equine encephalitis virus. As such, the self-amplifying RNA described herein may incorporate an RNA replicase derived from any one of semliki forest virus (SFV), sindbis virus (SIN), Venezuelan equine encephalitis virus (VEE), Ross-River virus (RRV), or other viruses belonging to the alphavirus family. In some embodiments, the self-amplifying RNA described herein may incorporate sequences derived from a mutant or wild-type virus sequence, e.g., the attenuated TC83 mutant of VEEV has been used in saRNAs. Alphavirus-based saRNAs are (+)-stranded saRNAs that may be translated after delivery to a cell, which leads to translation of a replicase (or replicase-transcriptase). The replicase is translated as a polyprotein which 5 auto-cleaves to provide a replication complex which creates genomic (−)-strand copies of the (+)-strand delivered RNA. These (−)-strand transcripts may themselves be transcribed to give further copies of the (+)-stranded parent RNA and also to give a subgenomic transcript which encodes the desired gene product. Translation of the subgenomic transcript thus leads to in situ expression of the desired gene product by the infected cell. Suitable alphavirus saRNAs may use a replicase from a sindbis virus, a semliki forest virus, an eastern equine encephalitis virus, a Venezuelan equine encephalitis virus, or mutant variants thereof. In some embodiments, the self-amplifying RNA molecule is derived from or based on a virus other than an alphavirus, such as a positive-stranded RNA virus, and in particular a picornavirus, flavivirus, rubivirus, pestivirus, hepacivirus, calicivirus, or coronavirus. Suitable wild type alphavirus sequences are well-known and are available from sequence depositories, such as the American Type Culture Collection, Rockville, Md. Representative examples of suitable alphaviruses include Aura (ATCC VR-368), Bebaru virus (ATCC VR-600, ATCC VR-1240), Cabassou (ATCC VR-922), Chikungunya virus (ATCC VR-64, ATCC VR-1241), Eastern equine encephalomyelitis virus (ATCC VR-65, ATCC VR-1242), Fort Morgan (ATCC VR-924), Getah virus (ATCC VR-369, ATCC VR-1243), Kyzylagach (ATCC VR-927), Mayaro (ATCC VR-66), Mayaro virus (ATCC VR-1277), Middleburg (ATCC VR-370), Mucambo virus (ATCC VR-580, ATCC VR-1244), Ndumu (ATCC VR-371), Pixuna virus (ATCC VR-372, ATCC VR-1245), Ross River virus (ATCC VR-373, ATCC VR-1246), Semliki Forest (ATCC VR-67, ATCC VR-1247), Sindbis virus (ATCC VR-68, ATCC VR-1248), Tonate (ATCC VR-925), Triniti (ATCC VR-469), Una (ATCC VR-374), Venezuelan equine encephalomyelitis (ATCC VR-69, ATCC VR-923, ATCC VR-1250 ATCC VR-1249, ATCC VR-532), Western equine encephalomyelitis (ATCC VR-70, ATCC VR-1251, ATCC VR-622, ATCC VR-1252), Whataroa (ATCC VR-926), and Y-62-33 (ATCC VR-375). In some aspects, one or more of the alphaviruses in the list may be excluded. In some embodiments, the self-amplifying RNA molecules described herein are larger than other types of RNA (e.g., saRNA). Typically, the self-amplifying RNA molecules described herein include at least about 4 kb. For example, the self-amplifying RNA may be equal to any one of, at least any one of, at most any one of, or between any two of 3 kb, 4 kb, 5 kb, 6 kb, 7 kb, 8 kb, 9 kb, 10 kb, 11 kb, 12 kb, 13 kb, 14 kb, 15 kb, 16 kb. In some instances the self-amplifying RNA may include at least about 5 kb, at least about 6 kb, at least about 7 kb, at least about 8 kb, at least about 9 kb, at least about 10 kb, at least about 11 kb, at least about 12 kb, or more than 12 kb. In certain examples, the self-amplifying RNA is about 4 kb to about 12 kb, about 5 kb to about 12 kb, about 6 kb to about 12 kb, about 7 kb to about 12 kb, about 8 kb to about 12 kb, about 9 kb to about 12 kb, about 10 kb to about 12 kb, about 11 kb to about 12 kb, about 5 kb to about 11 kb, about 5 kb to about 10 kb, about 5 kb to about 9 kb, about 5 kb to about 8 kb, about 5 kb to about 7 kb, about 5 kb to about 6 kb, about 6 kb to about 12 kb, about 6 kb to about 11 kb, about 6 kb to about 10 kb, about 6 kb to about 9 kb, about 6 kb to about 8 kb, about 6 kb to about 7 kb, about 7 kb to about 11 kb, about 75 kb to about 10 kb, about 7 kb to about 9 kb, about 7 kb to about 8 kb, about 8 kb to about 11 kb, about 8 kb to about 10 kb, about 8 kb to about 9 kb, about 9 kb to about 11 kb, about 9 kb to about 10 kb, or about 10 kb to about 11 kb. In some embodiments, the self-amplifying RNA molecule may encode a single polypeptide antigen or, optionally, two or more of polypeptide antigens linked together in a way that each of the sequences retains its identity (e.g., linked in series) when expressed as an amino acid sequence. The polypeptides generated from the self-amplifying RNA may then be produced as a fusion polypeptide or engineered in such a manner to result in separate polypeptide or peptide sequences. In some embodiments, the saRNA molecule may encode one polypeptide of interest or more, such as an antigen or more than one antigen, e.g., two, three, four, five, six, seven, eight, nine, ten, or more polypeptides. Alternatively, or in addition, one saRNA molecule may also encode more than one polypeptide of interest or more, such as an antigen, e.g., a bicistronic, or tricistronic RNA molecule that encodes different or identical antigens. The term “linked” as used herein refers to a first amino acid sequence or polynucleotide sequence covalently or non-covalently joined to a second amino acid sequence or polynucleotide sequence, respectively. The first amino acid or polynucleotide sequence can be directly joined or juxtaposed to the second amino acid or polynucleotide sequence or alternatively an intervening sequence can covalently join the first sequence to the second sequence. The term “linked” means not only a fusion of a first RNA molecule to a RNA molecule at the 5′-end or the 3′-end, but also includes insertion of the whole first RNA molecule into any two nucleotides in the second RNA molecule. The first second RNA molecule can be linked to a second RNA molecule by a phosphodiester bond or a linker. The linker can be, e.g., a polynucleotide. In some embodiments, the self-amplifying RNA described herein may encode one or more polypeptide antigens that include a range of epitopes. In some embodiments, the selfamplifying RNA described herein may encode epitopes capable of eliciting either a helper T-cell response or a cytotoxic T-cell response or both.

In some embodiments, the saRNA molecule is purified, e.g., such as by filtration that may occur via, e.g., ultrafiltration, diafiltration, or, e.g., tangential flow ultrafiltration/diafiltration. Some embodiments of the disclosure are directed to a composition comprising a selfamplifying RNA molecule comprising a 5′ Cap, a 5′ untranslated region, a coding region comprising a sequence encoding an RNA-dependent RNA polymerase (also referred to as a “replicase”), a subgenomic promoter, such as one derived from an alphavirus, an open reading frame encoding a gene of interest (e.g., an antigen derived from influenza virus), a 3′ untranslated region, and a 3′ poly A sequence. In some embodiments, at least 5% of a total population of a particular nucleotide in the saRNA molecule has been replaced with one or more modified or unnatural nucleotides. In some embodiments, the saRNA molecule does not include modified nucleotides, e.g., does not include modified nucleobases, 5 and all of the nucleotides in the RNA molecule are conventional standard ribonucleotides A, U, G and C, with the exception of an optional 5′ cap that may include, for example, 7-methylguanosine, which is further described below. In some embodiments, the RNA may include a 5′ cap comprising a 7′-methylguanosine, and the first 1, 2 or 3 5′ ribonucleotides may be methylated at the 2′ position of the ribose. The efficacy of the product is dependent on expression of the delivered saRNA, which requires a sufficiently intact RNA molecule. RNA integrity is a measure of RNA quality that quantitates intact RNA. The method is also capable of detecting potential degradation products. RNA integrity is preferably determined by capillary gel electrophoresis. The initial specification is set to ensure sufficient RNA integrity in drug product preparations. In some embodiments, the RNA polynucleotide has an integrity of at least about 80%, 85%, 90%, 92%, 94%, 95%, 96%, 97%, 98%, or 99%. In some embodiments, the RNA polynucleotide has an integrity of or greater than about 95%. In some embodiments, the RNA polynucleotide has an integrity of or greater than about 98%. In some embodiments, the RNA polynucleotide has an integrity of or greater than about 99%. In preferred embodiments, the saRNA polynucleotide has a clinical grade purity. In some embodiments, the purity of the RNA polynucleotide is between about 60% and about 100%. In some embodiments, the purity of the RNA polynucleotide is between about 80% and 99%. In some embodiments, the purity of the RNA polynucleotide is between about 90% and about 99%. In some embodiments, wherein the purified mRNA has a clinical grade purity without further purification. In some embodiments, the clinical grade purity is achieved through a method including tangential flow filtration (TFF) purification. In some embodiments, the clinical grade purity is achieved without the further purification selected from high performance liquid chromatography (H PLC) purification, ligand or binding based purification, and/or ion exchange chromatography. In some embodiments, the method of producing the RNA polynucleotides removes long abortive RNA species, double-stranded RNA (dsRNA), residual plasmid DNA residual solvent and/or residual salt. In some embodiments, the short abortive transcript contaminants comprise less than 15 bases. In some embodiments, the short abortive transcript contaminants comprise about 8-12 bases. In some embodiments, the method of the invention also removes RNAse inhibitor. In some embodiments, the purified saRNA polynucleotide comprises 5% or less, 4% or less, 3% or less, 2% or less, 1% or less or is substantially free of protein contaminants as determined by capillary electrophoresis. In some embodiments, the purified RNA polynucleotide comprises less than 5%, less than 4%, less than 3%, less than 2%, less than 1%, or is substantially free of salt contaminants determined by high performance liquid chromatography (HPLC). In some embodiments, the purified RNA polynucleotide comprises 5% or less, 4% or less, 3% or less, 2% or less, 1% or less or is substantially free of short abortive transcript 5 contaminants determined by known methods, such as, e.g., high performance liquid chromatography (HPLC). In some embodiments, the purified RNA polynucleotide has integrity of 60% or greater, 70% or greater, 80% or greater, 81% or greater, 82% or greater, 83% or greater, 84% or greater, 85% or greater, 86% or greater, 87% or greater, 88% or greater, 89% or greater, 90% or greater, 95% or greater, 96% or greater, 97% or greater, 98% or greater, or 99% or 10 greater as determined by a known method, such as, e.g., capillary electrophoresis.

In an embodiment, the mRNA vaccines of the disclosure comprise lipids. The lipids and modRNA can together form nanoparticles. The lipids can encapsulate the mRNA in the form of a lipid nanoparticle (LNP) to aid cell entry and stability of the RNA/lipid nanoparticles.

Lipid nanoparticles may include a lipid component and one or more additional components, such as a therapeutic and/or prophylactic. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combinations of elements. The efficacy and tolerability of a LNP formulation may be affected by the stability of the formulation.

Lipid nanoparticles may be designed for one or more specific applications or targets. For example, a LNP may be designed to deliver a therapeutic and/or prophylactic such as an RNA to a particular cell, tissue, organ, or system or group thereof in a mammal's body.

Physiochemical properties of lipid nanoparticles may be altered to increase selectivity for particular bodily targets. For instance, particle sizes may be adjusted based on the fenestration sizes of different organs. The therapeutic and/or prophylactic included in a LNP may also be selected based on the desired delivery target or targets. For example, a therapeutic and/or prophylactic may be selected for a particular indication, condition, disease, or disorder and/or for delivery to a particular cell, tissue, organ, or system or group thereof (e.g., localized or specific delivery). In certain embodiments, a LNP may include an mRNA encoding a polypeptide of interest capable of being translated within a cell to produce the polypeptide of interest. Such a composition may be designed to be specifically delivered to a particular organ. In some embodiments, a composition may be designed to be specifically delivered to a mammalian liver. In some embodiments, a composition may be designed to be specifically delivered to a lymph node. In some embodiments, a composition may be designed to be specifically delivered to a mammalian spleen.

A LNP may include one or more components described herein. In some embodiments, the LNP formulation of the disclosure includes at least one lipid nanoparticle component. Lipid nanoparticles may include a lipid component and one or more additional components, such as a therapeutic and/or prophylactic, such as a nucleic acid. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combination of elements. The efficacy and tolerability of a LNP formulation may be affected by the stability of the formulation.

In some embodiments, for example, a polymer may be included in and/or used to encapsulate or partially encapsulate a LNP. A polymer may be biodegradable and/or biocompatible. A polymer may be selected from, but is not limited to, polyamines, polyethers, polyamides, polyesters, poly carbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(gly colic acid) (PGA), poly(lactic acid-co-gly colic acid) (PLGA), poly(L-lactic acid-co-gly colic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcelIulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin 134, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A LNP may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, surface active agents, buffering agents, preservatives, and other species.

Surface active agents and/or emulsifiers may include, but are not limited to, natural emulsifiers (e.g., acacia, alginic acid, sodium alginate, cholesterol, and lecithin), sorbitan fatty acid esters (e.g., polyoxy ethylene sorbitan monolaurate [TWEEN®20], polyoxy ethylene sorbitan [TWEEN® 60], polyoxy ethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC® F 68, POLOXAMER® 188, cetrimonium bromide, cetylpyridinium chloride, benzalkonium chloride, docusate sodium, and/or combinations thereof.

Examples of preservatives may include, but are not limited to, antioxidants, chelating agents, free radical scavengers, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxy toluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxy toluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN® II, NEOLONE™, KATHON™, and/or EUXYL®. An exemplary free radical scavenger includes butylated hydroxytoluene (BHT or butylhydroxytoluene) or deferoxamine.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof.

In some embodiments, the formulation including a LNP may further include a salt, such as a chloride salt. In some embodiments, the formulation including a LNP may further includes a sugar such as a disaccharide. In some embodiments, the formulation further includes a sugar but not a salt, such as a chloride salt. In some embodiments, a LNP may further include one or more small hydrophobic molecules such as a vitamin (e.g., vitamin A or vitamin E) or a sterol. Carbohydrates may include simple sugars (e.g., glucose) and polysaccharides (e.g., glycogen and derivatives and analogs thereof).

The characteristics of a LNP may depend on the components thereof. For example, a LNP including cholesterol as a structural lipid may have different characteristics than a LNP that includes a different structural lipid. As used herein, the term “structural lipid” refers to sterols and also to lipids containing sterol moieties. As defined herein, “sterols” are a subgroup of steroids consisting of steroid alcohols. In some embodiments, the structural lipid is a steroid. In some embodiments, the structural lipid is cholesterol. In some embodiments, the structural lipid is an analog of cholesterol. In some embodiments, the structural lipid is alpha-tocopherol.

In some embodiments, the characteristics of a LNP may depend on the absolute or relative amounts of its components. For instance, a LNP including a higher molar fraction of a phospholipid may have different characteristics than a LNP including a lower molar fraction of a phospholipid. Characteristics may also vary depending on the method and conditions of preparation of the lipid nanoparticle. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Particular phospholipids can facilitate fusion to a membrane. In some embodiments, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue. Non-natural phospholipid species including natural species with modifications and substitutions including branching, oxidation, cyclization, and alkynes are also contemplated. In some embodiments, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye). Phospholipids include, but are not limited to, glycerophospholipids such as phosphatidylcholines, phosphatidyl-ethanolamines, phosphatidylserines, phosphatidylinositols, phosphatidy glycerols, and phosphatidic acids. Phospholipids also include phosphosphingolipid, such as sphingomyelin. In some embodiments, a phospholipid useful or potentially useful in the present invention is an analog or variant of DSPC.

Lipid nanoparticles may be characterized by a variety of methods. For example, microscopy (e.g., transmission electron microscopy or scanning electron microscopy) may be used to examine the morphology and size distribution of a LNP. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a LNP, such as particle size, polydispersity index, and zeta potential.

The mean size of a LNP may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a LNP may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a LNP may be from about 70 nm to about 100 nm. In a particular embodiment, the mean size may be about 80 nm. In other embodiments, the mean size may be about 100 nm.

A LNP may be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. For example, the zeta potential may describe the surface charge of a LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic describes the amount of therapeutic and/or prophylactic that is encapsulated or otherwise associated with a LNP after preparation, relative to the initial amount provided. The encapsulation efficiency is desirably high (e.g., close to 100%). The encapsulation efficiency may be measured, for example, by comparing the amount of therapeutic and/or prophylactic in a solution containing the lipid nanoparticle before and after breaking up the lipid nanoparticle with one or more organic solvents or detergents. Fluorescence may be used to measure the amount of free therapeutic and/or prophylactic (e.g., RNA) in a solution. For the lipid nanoparticles described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A LNP may optionally comprise one or more coatings. For example, a LNP may be formulated in a capsule, film, or tablet having a coating. A capsule, film, or tablet including a composition described herein may have any useful size, tensile strength, hardness, or density.

Formulations comprising amphiphilic polymers and lipid nanoparticles may be formulated in whole or in part as pharmaceutical compositions. Pharmaceutical compositions may include one or more amphiphilic polymers and one or more lipid nanoparticles. For example, a pharmaceutical composition may include one or more amphiphilic polymers and one or more lipid nanoparticles including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21st Edition, A. R. Gennaro; Lippincott, Williams & Wilkins, Baltimore, MD, 2006. Conventional excipients and accessory ingredients may be used in any pharmaceutical composition, except insofar as any conventional excipient or accessory ingredient may be incompatible with one or more components of a LNP or the one or more amphiphilic polymers in the formulation of the disclosure. An excipient or accessory ingredient may be incompatible with a component of a LNP or the amphiphilic polymer of the formulation if its combination with the component or amphiphilic polymer may result in any undesirable biological effect or otherwise deleterious effect.

In some embodiments, one or more excipients or accessory ingredients may make up greater than 50% of the total mass or volume of a pharmaceutical composition including a LNP. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia. Relative amounts of the one or more amphiphilic polymers, the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more lipid nanoparticles. As another example, a pharmaceutical composition may comprise between 0.1% and 15% (wt/vol) of one or more amphiphilic polymers (e.g., 0.5%, 1%, 2.5%, 5%, 10%, or 12.5% w/v).

In certain embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more amphiphilic polymers and one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles by adding an effective amount of an amphiphilic polymer and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.).

The chemical properties of the LNP, LNP suspension, lyophilized LNP composition, or LNP formulation of the present disclosure may be characterized by a variety of methods. In some embodiments, electrophoresis (e.g., capillary electrophoresis) or chromatography (e.g., reverse phase liquid chromatography) may be used to examine the mRNA integrity.

The efficacy of the product is dependent on expression of the delivered RNA, which requires a sufficiently intact RNA molecule. RNA integrity is a measure of RNA quality that quantitates intact RNA. The method is also capable of detecting potential degradation products. RNA integrity is preferably determined by capillary gel electrophoresis. The initial specification is set to ensure sufficient RNA integrity in drug product preparations. In some embodiments, the RNA polynucleotide has an integrity of at least about 80%, 85%, 90%, 92%, 94%, 95%, 96%, 97%, 98%, or 99%. In some embodiments, the RNA polynucleotide has an integrity of or greater than about 95%. In some embodiments, the RNA polynucleotide has an integrity of or greater than about 98%. In some embodiments, the RNA polynucleotide has an integrity of or greater than about 99%.

In preferred embodiments, the RNA polynucleotide has a clinical grade purity. In some embodiments, the purity of the RNA polynucleotide is between about 60% and about 100%. In some embodiments, the purity of the RNA polynucleotide is between about 80% and 99%. In some embodiments, the purity of the RNA polynucleotide is between about 90% and about 99%. In some embodiments, wherein the purified mRNA has a clinical grade purity without further purification. In some embodiments, the clinical grade purity is achieved through a method including tangential flow filtration (TFF) purification. In some embodiments, the clinical grade purity is achieved without the further purification selected from high performance liquid chromatography (H PLC) purification, ligand or binding based purification, and/or ion exchange chromatography. In some embodiments, the method of producing the RNA polynucleotides removes long abortive RNA species, double-stranded RNA (dsRNA), residual plasmid DNA residual solvent and/or residual salt. In some embodiments, the short abortive transcript contaminants comprise less than 15 bases. In some embodiments, the short abortive transcript contaminants comprise about 8-12 bases. In some embodiments, the method of the invention also removes RNAse inhibitor.

In some embodiments, the purified RNA polynucleotide comprises 5% or less, 4% or less, 3% or less, 2% or less, 1% or less or is substantially free of protein contaminants as determined by capillary electrophoresis. In some embodiments, the purified RNA polynucleotide comprises less than 5%, less than 4%, less than 3%, less than 2%, less than 1%, or is substantially free of salt contaminants determined by high performance liquid chromatography (HPLC). In some embodiments, the purified RNA polynucleotide comprises 5% or less, 4% or less, 3% or less, 2% or less, 1% or less or is substantially free of short abortive transcript contaminants determined by known methods, such as, e.g., high performance liquid chromatography (HPLC). In some embodiments, the purified RNA polynucleotide has integrity of 95% or greater, 96% or greater, 97% or greater, 98% or greater, or 99% or greater as determined by a known method, such as, e.g., capillary electrophoresis.

In some embodiments, the LNP integrity of the LNP, LNP suspension, lyophilized LNP composition, or LNP formulation of the present disclosure is about 20% or higher, about 25% or higher, about 30% or higher, about 35% or higher, about 40% or higher, about 45% or higher, about 50% or higher, about 55% or higher, about 60% or higher, about 65% or higher, about 70% or higher, about 75% or higher, about 80% or higher, about 85% or higher, about 90% or higher, about 95% or higher, about 96% or higher, about 97% or higher, about 98% or higher, or about 99% or higher.

In some embodiments, the LNP integrity of the LNP, LNP suspension, lyophilized LNP composition, or LNP formulation of the present disclosure is higher than the LNP integrity of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more, about 10 folds or more, about 20 folds or more, about 30 folds or more, about 40 folds or more, about 50 folds or more, about 100 folds or more, about 200 folds or more, about 300 folds or more, about 400 folds or more, about 500 folds or more, about 1000 folds or more, about 2000 folds or more, about 3000 folds or more, about 4000 folds or more, about 5000 folds or more, or about 10000 folds or more.

In some embodiments, the Txo % of the LNP, LNP suspension, lyophilized LNP composition, or LNP formulation of the present disclosure is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer.

In some embodiments, the Txo % of the LNP, LNP suspension, lyophilized LNP composition, or LNP formulation of the present disclosure is longer than the Txo % of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more.

In some embodiments, the T1/2 of the LNP, LNP suspension, lyophilized LNP composition, or LNP formulation of the present disclosure is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer.

In some embodiments, the T1/2 of the LNP, LNP suspension, lyophilized LNP composition, or LNP formulation of the present disclosure is longer than the T1/2 of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more

As used herein, “Tx” refers to the amount of time lasted for the nucleic acid integrity (e.g., mRNA integrity) of a LNP, LNP suspension, lyophilized LNP composition, or LNP formulation to degrade to about X of the initial integrity of the nucleic acid (e.g., mRNA) used for the preparation of the LNP, LNP suspension, lyophilized LNP composition, or LNP formulation. For example, “T80%” refers to the amount of time lasted for the nucleic acid integrity (e.g., mRNA integrity) of a LNP, LNP suspension, lyophilized LNP composition, or LNP formulation to degrade to about 80% of the initial integrity of the nucleic acid (e.g., mRNA) used for the preparation of the LNP, LNP suspension, lyophilized LNP composition, or LNP formulation. For another example, “T1/2” refers to the amount of time lasted for the nucleic acid integrity (e.g., mRNA integrity) of a LNP, LNP suspension, lyophilized LNP composition, or LNP formulation to degrade to about 1/2 of the initial integrity of the nucleic acid (e.g., mRNA) used for the preparation of the LNP, LNP suspension, lyophilized LNP composition, or LNP formulation.

Lipid nanoparticles may include a lipid component and one or more additional components, such as a therapeutic and/or prophylactic, such as a nucleic acid. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combination of elements. The efficacy and tolerability of a LNP formulation may be affected by the stability of the formulation.

The lipid component of a LNP may include, for example, a cationic lipid, a phospholipid (such as an unsaturated lipid, e.g., DOPE or DSPC), a PEG lipid, and a structural lipid. The elements of the lipid component may be provided in specific fractions.

In some embodiments, the LNP further comprises a phospholipid, a PEG lipid, a structural lipid, or any combination thereof. Suitable phospholipids, PEG lipids, and structural lipids for the methods of the present disclosure are further disclosed herein.

In some embodiments, the lipid component of a LNP includes a cationic lipid, a phospholipid, a PEG lipid, and a structural lipid. In certain embodiments, the lipid component of the lipid nanoparticle includes about 30 mol % to about 60 mol % cationic lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In some embodiments, the lipid component of the lipid nanoparticle includes about 35 mol % to about 55 mol % compound of cationic lipid, about 5 mol % to about 25 mol % phospholipid, about 30 mol % to about 40 mol % structural lipid, and about 0 mol % to about 10 mol % of PEG lipid. In a particular embodiment, the lipid component includes about 50 mol % said cationic lipid, about 10 mol % phospholipid, about 38.5 mol % structural lipid, and about 1.5 mol % of PEG lipid. In another embodiment, the lipid component includes about 40 mol % said cationic lipid, about 20 mol % phospholipid, about 38.5 mol % structural lipid, and about 1.5 mol % of PEG lipid. In some embodiments, the phospholipid may be DOPE or DSPC. In other embodiments, the PEG lipid may be PEG-DMG and/or the structural lipid may be cholesterol.

The amount of a therapeutic and/or prophylactic in a LNP may depend on the size, composition, desired target and/or application, or other properties of the lipid nanoparticle as well as on the properties of the therapeutic and/or prophylactic. For example, the amount of an RNA useful in a LNP may depend on the size, sequence, and other characteristics of the RNA. The relative amounts of a therapeutic and/or prophylactic (i.e. pharmaceutical substance) and other elements (e.g., lipids) in a LNP may also vary. In some embodiments, the wt/wt ratio of the lipid component to a therapeutic and/or prophylactic in a LNP may be from about 5:1 to about 60:1, such as 5:1, 6:1, 7:1,8:1,9:1, 10:1, 11:1, 12:1, 13:1, 14:1, 15:1, 16:1, 17:1, 18:1, 19:1, 20:1, 25:1,30:1,35:1, 40:1, 45:1, 50:1, and 60:1. For example, the wt/wt ratio of the lipid component to a therapeutic and/or prophylactic may be from about 10:1 to about 40:1. In certain embodiments, the wt/wt ratio is about 20:1. The amount of a therapeutic and/or prophylactic in a LNP may, for example, be measured using absorption spectroscopy (e.g., ultraviolet-visible spectroscopy).

In some embodiments, the ionizable lipid is a compound of Formula (I):

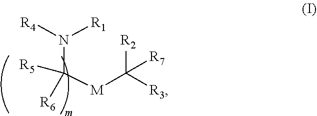

or their N-oxides, or salts or isomers thereof, wherein:
Ri is selected from the group consisting of C5-30 alkyl, C5-20 alkenyl, —R*YR″, —YR″, and —R″M′R′; R2 and R3 are independently selected from the group consisting of H, C1-14 alkyl, C2 14 alkenyl, —R*YR″, —YR″, and —R*OR″, or R2 and R3, together with the atom to which they are attached, form a heterocycle or carbocycle; R4 is selected from the group consisting of hydrogen, a C3-6 carbocycle, —(CH2)nQ, —(CH2)nCHQR, -CHQR, -CQ(R)2, and unsubstituted C₁₋₆ alkyl, where Q is selected from a carbocycle, heterocycle, —OR, —O(CH2)nN(R)2, —C(O)OR, —OC(O)R, —CX3, —CX2H, —CXH2, —ON, —N(R)2, —C(O)N(R)2, —N(R)C(O)R, —N(R)S(O)2R, —N(R)C(O)N(R)2, —N(R)C(S)N(R)2, —N(R)Re, N(R)S(O)2R8, —O(CH2)nOR, —N(R)C(═NR9)N(R)2, —N(R)C(═CHR9)N(R)2, —OC(O)N(R)2J-N(R)C(O)OR, —N(OR)C(O)R, —N(OR)S(O)2R, —N(OR)C(O)OR, N(OR)C(O)N(R)2, —N(OR)C(S)N(R)2, —N(OR)C(═NR9)N(R)2, —N(OR)C(═CHR9)N(R)2, —C(═NR9)N(R)2, —C(═NR9)R, —C(O)N(R)OR, and —C(R)N(R)2C(O)OR, and each n is independently selected from 1, 2, 3, 4, and 5; each R5 is independently selected from the group consisting of C1-3 alkyl, C2-3 alkenyl, and H; each Re is independently selected from the group consisting of C1-3 alkyl, C2-3 alkenyl, and H; M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)2-, —S—S—, an aryl group, and a heteroaryl group, in which M″ is a bond, C1-13 alkyl or C₂₋₁₃ alkenyl; R7 is selected from the group consisting of C1-3 alkyl, C2-3 alkenyl, and H; Re is selected from the group consisting of C3-6 carbocycle and heterocycle; R9 is selected from the group consisting of H, CN, NO2, C₁₋₆ alkyl, —OR, —S(O)2R, —S(O)2N(R)2, C2 6 alkenyl, C3-6 carbocycle and heterocycle; each R is independently selected from the group consisting of C1-3 alkyl, C2-3 alkenyl, and H; each R′ is independently selected from the group consisting of Ci-is alkyl, C2-is alkenyl, —R*YR″, —YR″, and H; each R″ is independently selected from the group consisting of C3-15 alkyl and C3-15 alkenyl; each R* is independently selected from the group consisting of Ci-i2 alkyl and C2-i2 alkenyl; each Y is independently a C3-6 carbocycle; each X is independently selected from the group consisting of F, Cl, Br, and I; and m is selected from 5, 6, 7, 8, 9, 10, 11, 12, and 13; and wherein when R4 is —(CH2)nQ, —(CH2)nCHQR, -CHQR, or -CQ(R)2, then (i) Q is not —N(R)2 when n is 1, 2, 3, 4 or 5, or (ii) Q is not 5, 6, or 7-membered heterocycloalkyl when n is 1 or 2. In some embodiments, the ionizable lipid is SM-102. In some embodiments, the ionizable lipid is ALC-0315. In some embodiments, the ionizable lipid is:

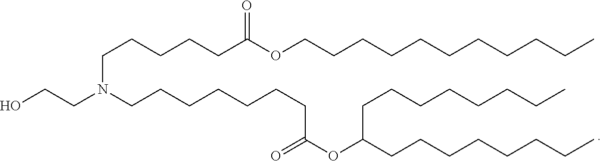 [SMILES: CCCCCCCCCCCOC(=O)CCCCCN(CCO)CCCCCCCC(=O)OC(CCCCCCCC)CCCCCCCC]

In some embodiments, the compounds have the following structure (I):

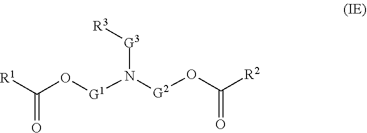

or a pharmaceutically acceptable salt, tautomer, prodrug or stereoisomer thereof, wherein: one of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa—, NRaC(═O)NRa—, —OC(═O)NRa—or —NRaC(═O)O—, and the other of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa—, NRaC(═O)NRa—, —OC(═O)NRa—or —NRaC(═O)O— or a direct bond; G1 and G2 are each independently unsubstituted C1-C12 alkylene or C1-C12 alkenylene; G3 is C1-C24 alkylene, C1-C24 alkenylene, C3-C8 cycloalkylene, C3-C8 cycloalkenylene; Ra is H or C1-C12 alkyl; R1 and R2 are each independently C6-C24 alkyl or C6-C24 alkenyl; R3 is H, OR5, CN, —C(═O)OR4, —OC(═O)R4 or —NR5C(═O)R4; R4 is C1-C12 alkyl; R5 is H or C1-C6 alkyl; and x is 0, 1 or 2. In a preferred embodiment, the ionizable lipid is:

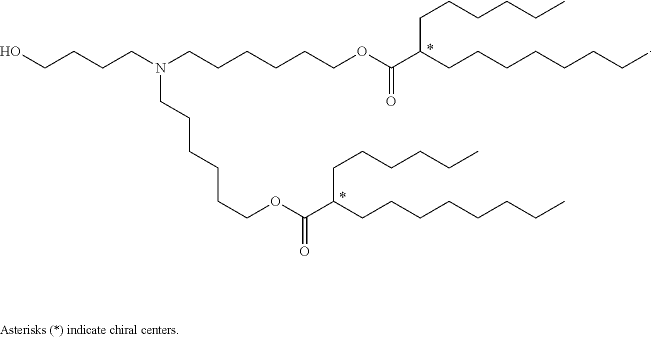

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. In some embodiments, a PEG lipid may be PEG-c-DOMG, PEG-DMG, PEG-DLPE, PEG-DMPE, PEG-DPPC, or a PEG-DSPE lipid. As used herein, the term “PEG lipid” refers to polyethylene glycol (PEG) -modified lipids. Non-limiting examples of PEG lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. In some embodiments, a PEG lipid can be PEG-c-DOMG, PEG-DMG, PEG-DLPE, PEG-DMPE, PEG-DPPC, or a PEG-DSPE lipid. In some embodiments, the PEG-modified lipids are a modified form of PEG DMG. In some embodiments, the PEG-modified lipid is PEG lipid with the formula (IV):

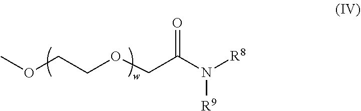

wherein R8 and R9 are each independently a straight or branched, saturated or unsaturated alkyl chain containing from 10 to 30 carbon atoms, wherein the alkyl chain is optionally interrupted by one or more ester bonds; and w has a mean value ranging from 30 to 60. The RNA (e.g., mRNA) vaccines may be utilized in various settings depending on the prevalence of the infection or the degree or level of unmet medical need. The RNA vaccines may be utilized to treat and/or prevent an influenza virus of various genotypes, strains, and isolates. The RNA vaccines typically have superior properties in that they produce much larger antibody titers and produce responses earlier than commercially available anti-viral therapeutic treatments. While not wishing to be bound by theory, it is believed that the RNA vaccines, as mRNA polynucleotides, are better designed to produce the appropriate protein conformation upon translation as the RNA vaccines co-opt natural cellular machinery. Unlike traditional vaccines, which are manufactured ex vivo and may trigger unwanted cellular responses, RNA (e.g., mRNA) vaccines are presented to the cellular system in a more native fashion.

In one aspect, a method of purifying an RNA polynucleotide synthesized by in vitro transcription is provided. The method includes ultrafiltration and diafiltration. In some embodiments, the method does not comprise a chromatography step. In some embodiments, the purified RNA polynucleotide is substantially free of contaminants comprising short abortive RNA species, long abortive RNA species, double-stranded RNA (dsRNA), residual plasmid DNA, residual in vitro transcription enzymes, residual solvent and/or residual salt. In some embodiments, the residual plasmid DNA is ≤500 ng DNA/mg RNA. In some embodiments, purity of the purified mRNA is between about 60% and about 100%. In another aspect, a method of producing an RNA polynucleotide-encapsulated LNP is provided. The method includes buffer exchanging the LNPs. The method further includes concentrating the LNPs via flat sheet cassette membranes. In preferred embodiments, the UFDF process does not utilize hollow fiber membranes.

There may be situations in which persons are at risk for infection with more than one strain of influenza virus. RNA (e.g., mRNA) therapeutic vaccines are particularly amenable to combination vaccination approaches due to a number of factors including, but not limited to, speed of manufacture, ability to rapidly tailor vaccines to accommodate perceived geographical threat, and the like. Moreover, because the vaccines utilize the human body to produce the antigenic protein, the vaccines are amenable to the production of larger, more complex antigenic proteins, allowing for proper folding, surface expression, antigen presentation, etc. in the human subject. To protect against more than one strain of influenza, a combination vaccine can be administered that includes RNA (e.g., mRNA) encoding at least one antigenic polypeptide protein (or antigenic portion thereof) of a first influenza virus or organism and further includes RNA encoding at least one antigenic polypeptide protein (or antigenic portion thereof) of a second influenza virus or organism. RNA (e.g., mRNA) can be co-formulated, for example, in a single lipid nanoparticle (LNP) or can be formulated in separate LNPs for co-administration.

Some embodiments of the present disclosure provide influenza virus (influenza) vaccines (or compositions or immunogenic compositions) that include at least one RNA polynucleotide having an open reading frame encoding at least one influenza antigenic polypeptide or an immunogenic fragment thereof (e.g., an immunogenic fragment capable of inducing an immune response to influenza).

In some embodiments, the at least one antigenic polypeptide is one of the defined antigenic subdomains of HA, termed HA1, HA2, or a combination of HA1 and HA2, and at least one antigenic polypeptide selected from neuraminidase (NA), nucleoprotein (NP), matrix protein 1 (M1), matrix protein 2 (M2), non-structural protein 1 (NS1) and non-structural protein 2 (NS2).

In some embodiments, the at least one antigenic polypeptide is HA or derivatives thereof comprising antigenic sequences from HA1 and/or HA2, and at least one antigenic polypeptide selected from NA, NP, M1, M2, NS1 and NS2.

In some embodiments, the at least one antigenic polypeptide is HA or derivatives thereof comprising antigenic sequences from HA1 and/or HA2 and at least two antigenic polypeptides selected from NA, NP, M1, M2, NS1 and NS2.

In some embodiments, a vaccine comprises at least one RNA (e.g., mRNA) polynucleotide having an open reading frame encoding an influenza virus protein, or an immunogenic fragment thereof.

In some embodiments, a vaccine comprises at least one RNA (e.g., mRNA) polynucleotide having an open reading frame encoding multiple influenza virus proteins, or immunogenic fragments thereof.

In some embodiments, a vaccine comprises at least one RNA (e.g., mRNA) polynucleotide having an open reading frame encoding a HA protein, or an immunogenic fragment thereof (e.g., at least one HA1, HA2, or a combination of both).

In some embodiments, a vaccine comprises at least one RNA (e.g., mRNA) polynucleotide having an open reading frame encoding a HA protein, or an immunogenic fragment thereof (e.g., at least one HA1, HA2, or a combination of both, of any one of or a combination of any or all of H1, H2, H3, H4, H5, H6, H7, H8, H9, H10, H11, H12, H13, H14, H15, H16, H17, and/or H18) and at least one other RNA (e.g., mRNA) polynucleotide having an open reading frame encoding a protein selected from a NP protein, a NA protein, a M1 protein, a M2 protein, a NS1 protein and a NS2 protein obtained from influenza virus.

In some embodiments, a vaccine comprises at least one RNA (e.g., mRNA) polynucleotide having an open reading frame encoding a HA protein, or an immunogenic fragment thereof (e.g., at least one any one of or a combination of any or all of H1, H2, H3, H4, H5, H6, H7, H8, H9, H10, H11, H12, H13, H14, H15, H16, H17, and/or H18) and at least two other RNAs (e.g., mRNAs) polynucleotides having two open reading frames encoding two proteins selected from a NP protein, a NA protein, a M1 protein, a M2 protein, a NS1 protein and a NS2 protein obtained from influenza virus.

In some embodiments, a vaccine comprises at least one RNA (e.g., mRNA) polynucleotide having an open reading frame encoding a HA protein, or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18) and at least three other RNAs (e.g., mRNAs) polynucleotides having three open reading frames encoding three proteins selected from a NP protein, a NA protein, a M protein, a M2 protein, a NS1 protein and a NS2 protein obtained from influenza virus.

In some embodiments, a vaccine comprises at least one RNA (e.g., mRNA) polynucleotide having an open reading frame encoding a HA protein, or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18) and at least four other RNAs (e.g., mRNAs) polynucleotides having four open reading frames encoding four proteins selected from a NP protein, a NA protein, a M1 protein, a M2 protein, a NS1 protein and a NS2 protein obtained from influenza virus.

In some embodiments, a vaccine comprises at least one RNA (e.g., mRNA) polynucleotide having an open reading frame encoding a HA protein, or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18) and at least five other RNAs (e.g., mRNAs) polynucleotides having five open reading frames encoding five proteins selected from a NP protein, a NA protein, a M1 protein, a M2 protein, a NS1 protein and a NS2 protein obtained from influenza virus.

In some embodiments, a vaccine comprises at least one RNA (e.g., mRNA) polynucleotide having an open reading frame encoding a HA protein or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18), a NP protein or an immunogenic fragment thereof, a NA protein or an immunogenic fragment thereof, a M1 protein or an immunogenic fragment thereof, a M2 protein or an immunogenic fragment thereof, a NS1 protein or an immunogenic fragment thereof and a NS2 protein or an immunogenic fragment thereof obtained from influenza virus.

Some embodiments of the present disclosure provide the following novel influenza virus polypeptide sequences: H1HA10-Foldon_ΔNgly1; H1HA10TM-PR8 (H1 A/Puerto Rico/8/34 HA); H1HA10-PR8-DS (H1 A/Puerto Rico/8/34 HA; pH1HA10-Cal04-DS (H1 A/California/04/2009 HA); Pandemic H1HA10 from California 04; pH1HA10-ferritin; HA10; Pandemic H1HA10 from California 04; Pandemic H1HA10 from California 04 strain/without foldon and with K68C/R76C mutation for trimerization; H1 HA10 from A/Puerto Rico/8/34 strain, without foldon and with Y94D/N95L mutation for trimerization; H1 HA10 from A/Puerto Rico/8/34 strain, without foldon and with K68C/R76C mutation for trimerization; H1N1 A/Viet Nam/850/2009; H3N2 A/Wisconsin/67/2005; H7N9 (A/Anhui/1/2013); H9N2 A/Hong Kong/1073/99; H10N8 A/JX346/2013.

Some embodiments of the present disclosure provide influenza virus (influenza) vaccines that include at least one RNA polynucleotide having an open reading frame encoding at least one influenza antigenic polypeptide or an immunogenic fragment of the novel influenza virus polypeptide sequences described above (e.g., an immunogenic fragment capable of inducing an immune response to influenza). In some embodiments, an influenza vaccine comprises at least one RNA (e.g., mRNA) polynucleotide having an open reading frame encoding at least one influenza antigenic polypeptide comprising a modified sequence that is at least 75% (e.g., any number between 75% and 100%, inclusive, e.g., 70%, 80%, 85%, 90%, 95%, 99%, and 100%) identity to an amino acid sequence of the novel influenza virus sequences described above. The modified sequence can be at least 75% (e.g., any number between 75% and 100%, inclusive, e.g., 70%, 80%, 85%, 90%, 95%, 99%, and 100%) identical to an amino acid sequence of the novel influenza virus sequences described above.

Some embodiments of the present disclosure provide an isolated nucleic acid comprising a sequence encoding the novel influenza virus polypeptide sequences described above; an expression vector comprising the nucleic acid; and a host cell comprising the nucleic acid. The present disclosure also provides a method of producing a polypeptide of any of the novel influenza virus sequences described above. A method may include culturing the host cell in a medium under conditions permitting nucleic acid expression of the novel influenza virus sequences described above, and purifying from the cultured cell or the medium of the cell a novel influenza virus polypeptide. The present disclosure also provides antibody molecules, including full length antibodies and antibody derivatives, directed against the novel influenza virus sequences.

In some embodiments, an open reading frame of a RNA (e.g., mRNA) vaccine is codon-optimized. In some embodiments, the open reading frame which the influenza polypeptide or fragment thereof is encoded is codon-optimized. Some embodiments provide use of an influenza vaccine that includes at least one ribonucleic acid (RNA) polynucleotide having an open reading frame encoding at least one influenza antigenic polypeptide or an immunogenic fragment thereof, wherein at least 80% (e.g., 85%, 90%, 95%, 98%, 99%, 100%) of the uracil in the open reading frame have a chemical modification, optionally wherein the vaccine is formulated in a lipid nanoparticle. In some embodiments, 100% of the uracil in the open reading frame have a chemical modification. In some embodiments, a chemical modification is in the 5-position of the uracil. In some preferred embodiments, a chemical modification is a N1-methyl pseudouridine.

In some embodiments, a RNA (e.g., mRNA) vaccine further comprising an adjuvant.

In some embodiments, at least one RNA polynucleotide encodes at least one influenza antigenic polypeptide that attaches to cell receptors.

In some embodiments, at least one RNA polynucleotide encodes at least one influenza antigenic polypeptide that causes fusion of viral and cellular membranes.

In some embodiments, at least one RNA polynucleotide encodes at least one influenza antigenic polypeptide that is responsible for binding of the virus to a cell being infected.

Some embodiments of the present disclosure provide a vaccine that includes at least one ribonucleic acid (RNA) (e.g., mRNA) polynucleotide having an open reading frame encoding at least one influenza antigenic polypeptide, at least one 5′ terminal cap and at least one chemical modification, formulated within a lipid nanoparticle.

In some embodiments, a 5′ terminal cap is 7mG(5′)ppp (5′)NlmpNp. In some preferred embodiments, the 5′ cap comprises:

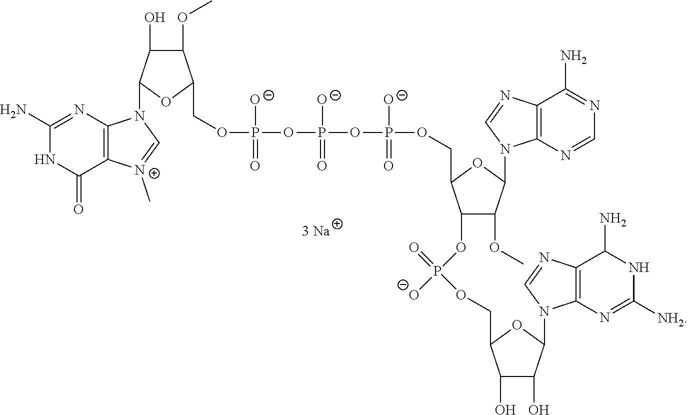 [SMILES: COC1C(COP(=O)([O-])OP(=O)([O-])OP(=O)([O-])OCC2OC(n3cnc4c(N)ncnc43)C(OC)C2OP(=O)([O-])OCC2OC(n3cnc4c3N=C(N)NC4N)C(O)C2O)OC(n2c[n+](C)c3c(=O)[nH]c(N)nc32)C1O]

In some embodiments, at least one chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-0-methyl uridine. In some embodiments, the chemical modification is in the 5-position of the uracil. In some embodiments, the chemical modification is a N1-m ethylpseudouridine. In some embodiments, the chemical modification is a N1-ethylpseudouridine.

In some embodiments, a lipid nanoparticle comprises a cationic lipid, a PEG-modified lipid, a sterol and a non-cationic lipid. In some embodiments, a cationic lipid is an ionizable cationic lipid and the non-cationic lipid is a neutral lipid, and the sterol is a cholesterol. In some embodiments, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy) heptadecanedioate (L319), (12Z,15Z)—N, N-dimethyl-2-nonylhenicosa-12, 15-dien-1-amine (L608), and N,N-dimethyl-1-[(1S,2R)-2-octylcyclopropyl]heptadecan-8-amine (L530).

Some embodiments of the present disclosure provide a vaccine that includes at least one RNA (e.g., mRNA) polynucleotide having an open reading frame encoding at least one influenza antigenic polypeptide, wherein at least 80% (e.g., 85%, 90%, 95%, 98%, 99%) of the uracil in the open reading frame have a chemical modification, optionally wherein the vaccine is formulated in a lipid nanoparticle (e.g., a lipid nanoparticle comprises a cationic lipid, a PEG-modified lipid, a sterol and a non-cationic lipid).

In some embodiments, 100% of the uracil in the open reading frame have a chemical modification. In some embodiments, a chemical modification is in the 5-position of the uracil. In some embodiments, a chemical modification is a N1-methyl pseudouridine. In some embodiments, 100% of the uracil in the open reading frame have a N1-methyl pseudouridine in the 5-position of the uracil.

In some embodiments, an open reading frame of a RNA (e.g., mRNA) polynucleotide encodes at least one influenza antigenic polypeptides. In some embodiments, the open reading frame encodes at least two, at least five, or at least ten antigenic polypeptides. In some embodiments, the open reading frame encodes at least 100 antigenic polypeptides. In some embodiments, the open reading frame encodes 1-100 antigenic polypeptides.

In some embodiments, a vaccine comprises at least two RNA (e.g., mRNA) polynucleotides, each having an open reading frame encoding at least one influenza antigenic polypeptide. In some embodiments, the vaccine comprises at least five or at least ten RNA (e.g., mRNA) polynucleotides, each having an open reading frame encoding at least one antigenic polypeptide or an immunogenic fragment thereof. In some embodiments, the vaccine comprises at least 100 RNA (e.g., mRNA) polynucleotides, each having an open reading frame encoding at least one antigenic polypeptide. In some embodiments, the vaccine comprises 2-100 RNA (e.g., mRNA) polynucleotides, each having an open reading frame encoding at least one antigenic polypeptide.

Also provided herein is an influenza RNA (e.g., mRNA) vaccine of any one of the foregoing paragraphs formulated in a nanoparticle (e.g., a lipid nanoparticle).

In some embodiments, the nanoparticle has a mean diameter of 50-200 nm. In some embodiments, the nanoparticle is a lipid nanoparticle. In some embodiments, the lipid nanoparticle comprises a cationic lipid, a PEG-modified lipid, a sterol and a non-cationic lipid. In some embodiments, the lipid nanoparticle comprises a molar ratio of about 20-60% cationic lipid, 0.5-15% PEG-modified lipid, 25-55% sterol, and 25% non-cationic lipid. In some embodiments, the cationic lipid is an ionizable cationic lipid and the non-cationic lipid is a neutral lipid, and the sterol is a cholesterol.

In some embodiments, the nanoparticle has a polydispersity value of less than 0.4 (e.g., less than 0.3, 0.2 or 0.1).

In some embodiments, the nanoparticle has a net neutral charge at a neutral pH value.

In some embodiments, the RNA (e.g., mRNA) vaccine is multivalent.

Some embodiments of the present disclosure provide methods of inducing an antigen specific immune response in a subject, comprising administering to the subject any of the RNA (e.g., mRNA) vaccine as provided herein in an amount effective to produce an antigen-specific immune response. In some embodiments, the RNA (e.g., mRNA) vaccine is an influenza vaccine. In some embodiments, the RNA (e.g., mRNA) vaccine is a combination vaccine comprising a combination of influenza vaccines (a broad spectrum influenza vaccine).

In some embodiments, an antigen-specific immune response comprises a T cell response or a B cell response.

In some embodiments, a method of producing an antigen-specific immune response comprises administering to a subject a single dose (no booster dose) of an influenza RNA (e.g., mRNA) vaccine of the present disclosure.

In some embodiments, a method further comprises administering to the subject a second (booster) dose of an influenza RNA (e.g., mRNA) vaccine. Additional doses of an influenza RNA (e.g., mRNA) vaccine may be administered.

In some embodiments, the subjects exhibit a seroconversion rate of at least 80% (e.g., at least 85%, at least 90%, or at least 95%) following the first dose or the second (booster) dose of the vaccine. Seroconversion is the time period during which a specific antibody develops and becomes detectable in the blood. After seroconversion has occurred, a virus can be detected in blood tests for the antibody. During an infection or immunization, antigens enter the blood, and the immune system begins to produce antibodies in response. Before seroconversion, the antigen itself may or may not be detectable, but antibodies are considered absent. During seroconversion, antibodies are present but not yet detectable. Any time after seroconversion, the antibodies can be detected in the blood, indicating a prior or current infection.

In some embodiments, an influenza RNA (e.g., mRNA) vaccine is administered to a subject by intradermal injection, intramuscular injection, or by intranasal administration. In some embodiments, an influenza RNA (e.g., mRNA) vaccine is administered to a subject by intramuscular injection.

Some embodiments, of the present disclosure provide methods of inducing an antigen specific immune response in a subject, including administering to a subject an influenza RNA (e.g., mRNA) vaccine in an effective amount to produce an antigen specific immune response in a subject. Antigen-specific immune responses in a subject may be determined, in some embodiments, by assaying for antibody titer (for titer of an antibody that binds to an influenza antigenic polypeptide) following administration to the subject of any of the influenza RNA (e.g., mRNA) vaccines of the present disclosure. In some embodiments, the anti-antigenic polypeptide antibody titer produced in the subject is increased by at least 1 log relative to a control. In some embodiments, the anti-antigenic polypeptide antibody titer produced in the subject is increased by 1-3 log relative to a control.

In some embodiments, the anti-antigenic polypeptide antibody titer produced in a subject is increased at least 2 times relative to a control. In some embodiments, the anti-antigenic polypeptide antibody titer produced in the subject is increased at least 5 times relative to a control. In some embodiments, the anti-antigenic polypeptide antibody titer produced in the subject is increased at least 10 times relative to a control. In some embodiments, the anti-antigenic polypeptide antibody titer produced in the subject is increased 2-10 times relative to a control.

In some embodiments, the control is an anti-antigenic polypeptide antibody titer produced in a subject who has not been administered a RNA (e.g., mRNA) vaccine of the present disclosure. In some embodiments, the control is an anti-antigenic polypeptide antibody titer produced in a subject who has been administered a live attenuated or inactivated influenza, or wherein the control is an anti-antigenic polypeptide antibody titer produced in a subject who has been administered a recombinant or purified influenza protein vaccine. In some embodiments, the control is an anti-antigenic polypeptide antibody titer produced in a subject who has been administered an influenza virus-like particle (VLP) vaccine.

A RNA (e.g., mRNA) vaccine of the present disclosure is administered to a subject in an effective amount (an amount effective to induce an immune response). In some embodiments, the effective amount is a dose equivalent to an at least 2-fold, at least 4-fold, at least 10-fold, at least 100-fold, at least 1000-fold reduction in the standard of care dose of a recombinant influenza protein vaccine, wherein the anti-antigenic polypeptide antibody titer produced in the subject is equivalent to an anti-antigenic polypeptide antibody titer produced in a control subject administered the standard of care dose of a recombinant influenza protein vaccine, a purified influenza protein vaccine, a live attenuated influenza vaccine, an inactivated influenza vaccine, or an influenza VLP vaccine. In some embodiments, the effective amount is a dose equivalent to 2-1000-fold reduction in the standard of care dose of a recombinant influenza protein vaccine, wherein the anti-antigenic polypeptide antibody titer produced in the subject is equivalent to an anti-antigenic polypeptide antibody titer produced in a control subject administered the standard of care dose of a recombinant influenza protein vaccine, a purified influenza protein vaccine, a live attenuated influenza vaccine, an inactivated influenza vaccine, or an influenza VLP vaccine.

In some embodiments, the control is an anti-antigenic polypeptide antibody titer produced in a subject who has been administered a virus-like particle (VLP) vaccine comprising structural proteins of influenza.

In some embodiments, the RNA (e.g., mRNA) vaccine is formulated in an effective amount to produce an antigen specific immune response in a subject.

In some embodiments, the effective amount is a total dose of 25 μg to 1000 μg, or 50 μg to 1000 μg. In some embodiments, the effective amount is a total dose of 100 μg. In some embodiments, the effective amount is a dose of 25 μg administered to the subject a total of two times. In some embodiments, the effective amount is a dose of 100 μg administered to the subject a total of two times. In some embodiments, the effective amount is a dose of 400 μg administered to the subject a total of two times. In some embodiments, the effective amount is a dose of 500 μg administered to the subject a total of two times.

In some embodiments, the efficacy (or effectiveness) of a RNA (e.g., mRNA) vaccine is greater than 60%. In some embodiments, the RNA (e.g., mRNA) polynucleotide of the vaccine at least one Influenza antigenic polypeptide.

Vaccine efficacy may be assessed using standard analyses. For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

Efficacy=(ARU−ARV)/ARU×100;

and

Efficacy=(1−RR)×100.

Likewise, vaccine effectiveness may be assessed using standard analyses. Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination: Effectiveness=(1−OR)×100. In some embodiments, the efficacy (or effectiveness) of a RNA (e.g., mRNA) vaccine is at least 65%, at least 70%, at least 75%, at least 80%, at least 85%, or at least 90%.

In some embodiments, the vaccine immunizes the subject against Influenza for up to 2 years. In some embodiments, the vaccine immunizes the subject against Influenza for more than 2 years, more than 3 years, more than 4 years, or for 5-10 years.

In some embodiments, the subject is about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger.

In some embodiments, the subject was born full term (e.g., about 37-42 weeks). In some embodiments, the subject was born prematurely, for example, at about 36 weeks of gestation or earlier (e.g., about 36, 35, 34, 33, 32, 31, 30, 29, 28, 27, 26 or 25 weeks). For example, the subject may have been born at about 32 weeks of gestation or earlier. In some embodiments, the subject was born prematurely between about 32 weeks and about 36 weeks of gestation. In such subjects, a RNA (e.g., mRNA) vaccine may be administered later in life, for example, at the age of about 6 months to about 5 years, or older.

In some embodiments, the subject is a young adult between the ages of about 20 years and about 50 years (e.g., about 20, 25, 30, 35, 40, 45 or 50 years old).

In some embodiments, the subject is an elderly subject about 60 years old, about 70 years old, or older (e.g., about 60, 65, 70, 75, 80, 85 or 90 years old).

In some embodiments, the subject has been exposed to influenza (e.g., C. trachomatis); the subject is infected with influenza (e.g., C. trachomatis); or subject is at risk of infection by influenza (e.g., C. trachomatis).

In some embodiments, the subject has been exposed to betacoronavirus (e.g., SARS-CoV-2); the subject is infected with betacoronavirus (e.g., SARS-CoV-2); or subject is at risk of infection by betacoronavirus (e.g., SARS-CoV-2).

In some embodiments, the subject has received at least one dose of an immunogenic composition against betacoronavirus (e.g., SARS-CoV-2), e.g., selected from any one of COMIRNATY®, the Pfizer-BioNTech COVID-19 vaccine, the Moderna mRNA-1273 COVID-19 vaccine, and the Janssen COVID-19 vaccine; the subject has received at least two doses of an immunogenic composition against betacoronavirus (e.g., SARS-CoV-2); the subject is receiving at least one dose of an immunogenic composition against betacoronavirus (e.g., SARS-CoV-2), e.g., selected from any one of COMIRNATY®, the Pfizer-BioNTech COVID-19 vaccine, the Moderna mRNA-1273 COVID-19 vaccine, and the Janssen COVID-19 vaccine; or the subject is being administered an immunogenic composition against betacoronavirus (e.g., SARS-CoV-2), e.g., selected from any one of COMIRNATY®, the Pfizer-BioNTech COVID-19 vaccine, the Moderna mRNA-1273 COVID-19 vaccine, and the Janssen COVID-19 vaccine at risk of infection by betacoronavirus (e.g., SARS-CoV-2) concomitantly, simultaneously, or within 12-48 hours of any one of the immunogenic compositions against influenza disclosed herein. In some embodiments, the subject is immunocompromised (has an impaired immune system, e.g., has an immune disorder or autoimmune disorder).

In some embodiments the nucleic acid vaccines described herein are chemically modified. In other embodiments the nucleic acid vaccines are unmodified.

Yet other aspects provide compositions for and methods of vaccinating a subject comprising administering to the subject a nucleic acid vaccine comprising one or more RNA polynucleotides having an open reading frame encoding a first virus antigenic polypeptide, wherein the RNA polynucleotide does not include a stabilization element, and wherein an adjuvant is not coformulated or co-administered with the vaccine.

In other aspects, the disclosure describes a composition for or method of vaccinating a subject comprising administering to the subject a nucleic acid vaccine comprising one or more RNA polynucleotides having an open reading frame encoding a first antigenic polypeptide wherein a dosage of between 10 μg/kg and 400 μg/kg of the nucleic acid vaccine is administered to the subject. In some embodiments the dosage of the RNA polynucleotide is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the nucleic acid vaccine is administered to the subject by intradermal or intramuscular injection. In some embodiments, the nucleic acid vaccine is administered to the subject on day zero. In some embodiments, a second dose of the nucleic acid vaccine is administered to the subject on day twenty-one.

In some embodiments, a dosage of 25 micrograms of the RNA polynucleotide is included in the nucleic acid vaccine administered to the subject. In some embodiments, a dosage of 100 micrograms of the RNA polynucleotide is included in the nucleic acid vaccine administered to the subject. In some embodiments, a dosage of 50 micrograms of the RNA polynucleotide is included in the nucleic acid vaccine administered to the subject. In some embodiments, a dosage of 75 micrograms of the RNA polynucleotide is included in the nucleic acid vaccine administered to the subject. In some embodiments, a dosage of 150 micrograms of the RNA polynucleotide is included in the nucleic acid vaccine administered to the subject. In some embodiments, a dosage of 400 micrograms of the RNA polynucleotide is included in the nucleic acid vaccine administered to the subject. In some embodiments, a dosage of 200 micrograms of the RNA polynucleotide is included in the nucleic acid vaccine administered to the subject. In some embodiments, the RNA polynucleotide accumulates at a 100-fold higher level in the local lymph node in comparison with the distal lymph node. In other embodiments the nucleic acid vaccine is chemically modified and in other embodiments the nucleic acid vaccine is not chemically modified.

Aspects of the disclosure provide a nucleic acid vaccine comprising one or more RNA polynucleotides having an open reading frame encoding a first antigenic polypeptide, wherein the RNA polynucleotide does not include a stabilization element, and a pharmaceutically acceptable carrier or excipient, wherein an adjuvant is not included in the vaccine. In some embodiments, the stabilization element is a histone stem-loop. In some embodiments, the stabilization element is a nucleic acid sequence having increased GC content relative to wild type sequence.

Aspects of the disclosure provide nucleic acid vaccines comprising one or more RNA polynucleotides having an open reading frame encoding a first antigenic polypeptide, wherein the RNA polynucleotide is present in the formulation for in vivo administration to a host, which confers an antibody titer superior to the criterion for seroprotection for the first antigen for an acceptable percentage of human subjects. In some embodiments, the antibody titer produced by the mRNA vaccines of the disclosure is a neutralizing antibody titer. In some embodiments the neutralizing antibody titer is greater than a protein vaccine. In other embodiments the neutralizing antibody titer produced by the mRNA vaccines of the disclosure is greater than an adjuvanted protein vaccine. In yet other embodiments the neutralizing antibody titer produced by the mRNA vaccines of the disclosure is 1,000-10,000, 1,200-10,000, 1,400-10,000, 1,500-10,000, 1,000-5,000, 1,000-4,000, 1,800-10,000, 2000-10,000, 2,000-5,000, 2,000-3,000, 2,000-4,000, 3,000-5,000, 3,000-4,000, or 2,000-2,500. A neutralization titer is typically expressed as the highest serum dilution required to achieve a 50% reduction in the number of plaques.

Also provided are nucleic acid vaccines comprising one or more RNA polynucleotides having an open reading frame encoding a first antigenic polypeptide, wherein the RNA polynucleotide is present in a formulation for in vivo administration to a host for eliciting a longer lasting high antibody titer than an antibody titer elicited by an mRNA vaccine having a stabilizing element or formulated with an adjuvant and encoding the first antigenic polypeptide. In some embodiments, the RNA polynucleotide is formulated to produce a neutralizing antibodies within one week of a single administration. In some embodiments, the adjuvant is selected from a cationic peptide and an immunostimulatory nucleic acid. In some embodiments, the cationic peptide is protamine.

Aspects provide nucleic acid vaccines comprising one or more RNA polynucleotides having an open reading frame comprising at least one chemical modification or optionally no modified nucleotides, the open reading frame encoding a first antigenic polypeptide, wherein the RNA polynucleotide is present in the formulation for in vivo administration to a host such that the level of antigen expression in the host significantly exceeds a level of antigen expression produced by an mRNA vaccine having a stabilizing element or formulated with an adjuvant and encoding the first antigenic polypeptide.

Other aspects provide nucleic acid vaccines comprising one or more RNA polynucleotides having an open reading frame comprising at least one chemical modification or optionally no modified nucleotides, the open reading frame encoding a first antigenic polypeptide, wherein the vaccine has at least 10-fold less RNA polynucleotide than is required for an unmodified mRNA vaccine to produce an equivalent antibody titer. In some embodiments, the RNA polynucleotide is present in a dosage of 25-100 micrograms.

Aspects of the disclosure also provide a unit of use vaccine, comprising between 10 μg and 400 ug of one or more RNA polynucleotides having an open reading frame comprising at least one chemical modification or optionally no modified nucleotides, the open reading frame encoding a first antigenic polypeptide, and a pharmaceutically acceptable carrier or excipient, formulated for delivery to a human subject. In some embodiments, the vaccine further comprises a cationic lipid nanoparticle.

Aspects of the disclosure provide methods of creating, maintaining or restoring antigenic memory to a virus strain in an individual or population of individuals comprising administering to said individual or population an antigenic memory booster nucleic acid vaccine comprising (a) at least one RNA polynucleotide, said polynucleotide comprising at least one chemical modification or optionally no modified nucleotides and two or more codon-optimized open reading frames, said open reading frames encoding a set of reference antigenic polypeptides, and (b) optionally a pharmaceutically acceptable carrier or excipient. In some embodiments, the vaccine is administered to the individual via a route selected from the group consisting of intramuscular administration, intradermal administration, and subcutaneous administration. In some embodiments, the administering step comprises contacting a muscle tissue of the subject with a device suitable for injection of the composition. In some embodiments, the administering step comprises contacting a muscle tissue of the subject with a device suitable for injection of the composition in combination with electroporation.

In some aspects, methods of inducing an antigen specific immune response in a subject are provided. The method includes administering to the subject an influenza RNA composition in an amount effective to produce an antigen specific immune response. In some embodiments, an antigen specific immune response comprises a T cell response or a B cell response. In some embodiments, an antigen specific immune response comprises a T cell response and a B cell response. In some embodiments, a method of producing an antigen specific immune response involves a single administration of the vaccine. In some embodiments, a method further includes administering to the subject a booster dose of the vaccine. In some embodiments, a vaccine is administered to the subject by intradermal or intramuscular injection.

Aspects of the disclosure provide methods of vaccinating a subject comprising administering to the subject a single dosage of between 25 ug/kg and 400 ug/kg of a nucleic acid vaccine comprising one or more RNA polynucleotides having an open reading frame encoding a first antigenic polypeptide in an effective amount to vaccinate the subject.

Other aspects provide nucleic acid vaccines comprising one or more RNA polynucleotides having an open reading frame comprising at least one chemical modification, the open reading frame encoding a first antigenic polypeptide, wherein the vaccine has at least 10-fold less RNA polynucleotide than is required for an unmodified mRNA vaccine to produce an equivalent antibody titer. In some embodiments, the RNA polynucleotide is present in a dosage of 25-100 micrograms.

Other aspects provide nucleic acid vaccines comprising an LNP formulated RNA polynucleotide having an open reading frame comprising no nucleotide modifications (unmodified), the open reading frame encoding a first antigenic polypeptide, wherein the vaccine has at least 10-fold less RNA polynucleotide than is required for an unmodified mRNA vaccine not formulated in a LNP to produce an equivalent antibody titer. In some embodiments, the RNA polynucleotide is present in a dosage of 25-100 micrograms.

The data presented in the Examples demonstrate significant enhanced immune responses using the formulations of the disclosure. Both chemically modified and unmodified RNA vaccines are useful according to the invention. Surprisingly, in contrast to prior art reports that it was preferable to use chemically unmodified mRNA formulated in a carrier to produce vaccines, it is described herein that chemically modified mRNA-LNP vaccines required a much lower effective mRNA dose than unmodified mRNA, i.e., tenfold less than unmodified mRNA when formulated in carriers other than LNP. Both the chemically modified and unmodified RNA vaccines of the disclosure produce better immune responses than mRNA vaccines formulated in a different lipid carrier.

In other aspects the disclosure describes a method of treating an elderly subject age 60 years or older comprising administering to the subject a nucleic acid vaccine comprising one or more RNA polynucleotides having an open reading frame encoding a virus antigenic polypeptide in an effective amount to vaccinate the subject.

In other aspects the disclosure describes a method of treating a young subject age 17 years or younger comprising administering to the subject a nucleic acid vaccine comprising one or more RNA polynucleotides having an open reading frame encoding a virus antigenic polypeptide in an effective amount to vaccinate the subject.

In other aspects the disclosure describes a method of treating an adult subject comprising administering to the subject a nucleic acid vaccine comprising one or more RNA polynucleotides having an open reading frame encoding a virus antigenic polypeptide in an effective amount to vaccinate the subject.

In some aspects the disclosure describes a method of vaccinating a subject with a combination vaccine including at least two nucleic acid sequences encoding antigens wherein the dosage for the vaccine is a combined therapeutic dosage wherein the dosage of each individual nucleic acid encoding an antigen is a sub therapeutic dosage. In some embodiments, the combined dosage is 25 micrograms of the RNA polynucleotide in the nucleic acid vaccine administered to the subject. In some embodiments, the combined dosage is 100 micrograms of the RNA polynucleotide in the nucleic acid vaccine administered to the subject. In some embodiments the combined dosage is 50 micrograms of the RNA polynucleotide in the nucleic acid vaccine administered to the subject. In some embodiments, the combined dosage is 75 micrograms of the RNA polynucleotide in the nucleic acid vaccine administered to the subject. In some embodiments, the combined dosage is 150 micrograms of the RNA polynucleotide in the nucleic acid vaccine administered to the subject. In some embodiments, the combined dosage is 400 micrograms of the RNA polynucleotide in the nucleic acid vaccine administered to the subject.

In preferred aspects, vaccines of the disclosure (e.g., LNP-encapsulated mRNA vaccines) produce prophylactically- and/or therapeutically efficacious levels, concentrations and/or titers of antigen-specific antibodies in the blood or serum of a vaccinated subject. As defined herein, the term antibody titer refers to the amount of antigen-specific antibody produces in s subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA). In exemplary embodiments, antibody titer is determined or measured by neutralization assay, e.g., by microneutralization assay. In certain aspects, antibody titer measurement is expressed as a ratio, such as 1:40, 1:100, etc.

In exemplary embodiments of the disclosure, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.) In exemplary aspects of the disclosure, antigen-specific antibodies are measured in units of pg/ml or are measured in units of IU/L (International Units per liter) or mIU/ml (milli International Units per ml). In exemplary embodiments of the disclosure, an efficacious vaccine produces >0.5 μg/ml, >0.1 μg/ml, >0.2 μg/ml, >0.35 μg/ml, >0.5 μg/ml, >1 μg/ml, >2 μg/ml, >5 μg/ml or >10 μg/ml. In exemplary embodiments of the disclosure, an efficacious vaccine produces >10 mIU/ml, >20 mIU/ml, >50 mIU/ml, >100 mIU/ml, >200 mIU/ml, >500 mIU/ml or >1000 mIU/ml. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.) In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay.

In some aspects, the disclosure provides a method comprising administering to a human subject a composition comprising: (a) a first messenger ribonucleic acid (mRNA) encoding a hemagglutinin (HA) antigen of a first influenza A virus and a second mRNA encoding an HA antigen of a second influenza A virus, wherein the influenza A HA antigens are of different subtypes; and (b) a third mRNA encoding an HA antigen of a first influenza B virus and a fourth mRNA encoding an HA antigen of a second influenza B virus, wherein the influenza B HA antigens are of different lineages, and wherein the composition further comprises a lipid nanoparticle comprising 40-55 mol % ionizable amino lipid; 5-15 mol % neutral lipid; 35-45 mol % sterol; and 1-5 mol % PEG-modified lipid, and wherein the composition comprises 50 or 25 μg to 200 μg of the mRNA in total. In some embodiments, the composition comprises 25 μg of the mRNA in total. In some embodiments, the composition comprises 50 μg of the mRNA in total. In some embodiments, the composition comprises 100 μg of the mRNA in total. In some embodiments, the composition comprises 200 μg of the mRNA in total. In preferred embodiments, the ratio of the first:second:third:fourth mRNA is not 1:1:1:1. In preferred embodiments, the ratio of the first:second:third:fourth mRNA is 1:1: greater than 1: greater than 1, influenza A:A:B:B respectively. In preferred embodiments, the ratio of the first:second:third:fourth mRNA is 1:1:2:2, influenza A:A:B:B respectively. In preferred embodiments, the ratio of the first:second:third:fourth mRNA is 1:1:4:4, influenza A:A:B:B respectively. In some embodiments, each mRNA comprises a 5′ cap analog. In some embodiments, the 5′ cap analog is a 5′ 7mG(5′)ppp(5′)NlmpNp cap. In some embodiments, each mRNA comprises a chemical modification. In some embodiments, the chemical modification is 1-methylpseudouridine. In some embodiments, the lipid nanoparticle comprises 40-50 mol % ionizable amino lipid, 35-45 mol % sterol, 10-15 mol % neutral lipid, and 2-4 mol % PEG-modified lipid. In some embodiments, the lipid nanoparticle comprises 45 mol %, 46 mol %, 47 mol %, 48 mol %, 49 mol %, or 50 mol % ionizable amino lipid. In some embodiments, the sterol is cholesterol. In some embodiments, the neutral lipid is 1,2 distearoyl-sn-glycero-3-phosphocholine (DSPC). In some embodiments, the PEG-modified lipid is 1,2 dimyristoyl-sn-glycerol, methoxypolyethyleneglycol (PEG2000 DMG). In some embodiments, the composition further comprises Tris buffer, sucrose, and sodium acetate. In some embodiments, the composition comprises 10 mM-30 mM Tris buffer, 75 mg/mL-95 mg/mL sucrose, and 5 mM-15 mM sodium acetate, optionally wherein the composition has a pH of 6-8. In some embodiments, the composition comprises 20 mM Tris buffer, 87 mg/mL sucrose, and 10.7 mM sodium acetate, optionally wherein the composition has a pH of 7.5. In some embodiments, the composition comprises 0.5 mg/mL of the mRNA.

In some embodiments, the composition is administered intramuscularly, optionally into a deltoid region of the human subject. In some embodiments, the human subject is 18 to 49 years of age. In some embodiments, the human subject is at least 50 years of age. In some embodiments, the human subject is 50-64 years of age. In some embodiments, the human subject is at least 65 years of age. In some embodiments, the human subject is seropositive for at least one of the HA antigens. In some embodiments, the human subject is seropositive for all of the HA antigens. In some embodiments, the subject is seronegative for all of the HA antigens. In some embodiments, the HA antigens are recommended by or selected according to standardized criteria used by World Health Organization's Global Influenza Surveillance and Response System (GISRS). In some embodiments, the HA antigen are selected using a hemagglutinin inhibition (HAI) assay to identify circulating influenza viruses that are antigenically similar to influenza viruses from a previous season's vaccine, optionally wherein influenza viruses are considered to be antigenically similar if their HAI titers differ by two dilutions or less. In some embodiments, the first mRNA encodes an influenza A HA antigen of the H1 subtype, the second mRNA encodes an influenza A HA antigen of the H3 subtype, the third mRNA encodes an influenza B HA antigen of the B/Yamagata lineage, and the fourth mRNA encodes an influenza B HA antigen of the B/Victoria lineage.

In some embodiments, the mRNA vaccine is administered in an amount effective to induce a neutralizing antibody response against influenza A H1N1, influenza A H3N2, influenza B/Yamagata, and influenza B/Victoria. In some embodiments, the mRNA vaccine is administered in an amount effective to induce a T cell response against influenza A H1N1, influenza A H3N2, influenza B/Yamagata, and influenza B/Victoria. The disclosure, in another aspect, provides a composition comprising: (a) a first messenger ribonucleic acid (mRNA) comprising an open reading frame (ORF) encoding a hemagglutinin (HA) antigen of a first influenza A virus and a second mRNA encoding an HA antigen of a second influenza A virus, wherein the influenza A HA antigens are of different subtypes; and (b) a third mRNA encoding an HA antigen of a first influenza B virus and a fourth mRNA encoding an HA antigen of a second influenza B virus, wherein the influenza B HA antigens are of different lineages, and wherein the composition further comprises a lipid nanoparticle comprising 40-55 mol % ionizable amino lipid; 5-15 mol % neutral lipid; 35-45 mol % sterol; and 1-5 mol % PEG-modified lipid; wherein the ratio of the first:second:third:fourth mRNA is 1:1:greater than 1: greater than 1, influenza A:A:B:B strains, respectively.

The disclosure, in another aspect, provides a composition comprising: (a) a first messenger ribonucleic acid (mRNA) comprising an open reading frame (ORF) encoding a hemagglutinin (HA) antigen of a first influenza A virus and a second mRNA encoding an HA antigen of a second influenza A virus, wherein the influenza A HA antigens are of different subtypes; and (b) a third mRNA encoding an HA antigen of a first influenza B virus and a fourth mRNA encoding an HA antigen of a second influenza B virus, wherein the influenza B HA antigens are of different lineages, and wherein the composition further comprises a lipid nanoparticle comprising 40-55 mol % ionizable amino lipid; 5-15 mol % neutral lipid; 35-45 mol % sterol; and 1-5 mol % PEG-modified lipid; wherein the ratio of the first:second:third:fourth mRNA is 1:1:greater than 1:greater than 1, influenza A:A:B:B strains, respectively.

Another aspect of the disclosure provides a composition comprising: (a) a first messenger ribonucleic acid (mRNA) comprising an open reading frame (ORF) encoding a hemagglutinin (HA) antigen of a first influenza A virus and a second mRNA encoding an HA antigen of a second influenza A virus, wherein the influenza A HA antigens are of different subtypes; and (b) a third mRNA encoding an HA antigen of a first influenza B virus and a fourth mRNA encoding an HA antigen of a second influenza B virus, wherein the influenza B HA antigens are of different lineages, and wherein the composition further comprises a lipid nanoparticle comprising 40-55 mol % ionizable amino lipid; 5-15 mol % neutral lipid; 35-45 mol % sterol; and 1-5 mol % PEG-modified lipid; wherein the ratio of the first:second:third:fourth mRNA is 1:1:greater than 1: greater than 1, influenza A:A:B:B strains, respectively, and wherein the composition comprises 100 μg of the mRNA in total.

The disclosure, in another aspect, provides a composition comprising: (a) a first messenger ribonucleic acid (mRNA) comprising an open reading frame (ORF) encoding a hemagglutinin (HA) antigen of a first influenza A virus and a second mRNA encoding an HA antigen of a second influenza A virus, wherein the influenza A HA antigens are of different subtypes; and (b) a third mRNA encoding an HA antigen of a first influenza B virus and a fourth mRNA encoding an HA antigen of a second influenza B virus, wherein the influenza B HA antigens are of different lineages, and wherein the composition further comprises a lipid nanoparticle comprising 40-55 mol % ionizable amino lipid; 5-15 mol % neutral lipid; 35-45 mol % sterol; and 1-5 mol % PEG-modified lipid; wherein the ratio of the first:second:third:fourth mRNA is 1:1:greater than 1:greater than 1, influenza A:A:B:B strains, respectively, and wherein the composition comprises 200 μg of the mRNA in total.

Another aspect of the disclosure includes a method for vaccinating a human subject, comprising administering an mRNA vaccine comprising mRNA encoding at least 4 influenza antigens, wherein the influenza antigens comprise at least 2 hemagglutinin (HA) A antigens, each of a different subtype and at least 2 HA B antigens, each of a different lineage, in an effective amount to produce an antigen specific immune response in the subject.

In some embodiments, the influenza antigens are encoded by one to four mRNAs. In some embodiments, the mRNA comprises a single mRNA encoding the at least 4 influenza antigens. In some embodiments, the mRNA comprises four mRNA each comprising a single open reading frame (ORF) encoding one of the 4 influenza antigens. In some embodiments, the 4 influenza antigens comprise an influenza A HA antigen of the H1 subtype, an influenza A HA antigen of the H3 subtype, an influenza B HA antigen of the B/Yamagata lineage, and an influenza B HA antigen of the B/Victoria lineage.

Another aspect of the disclosure provides a composition comprising an mRNA vaccine comprising mRNA encoding at least 4 influenza antigens, wherein the influenza antigens comprise at least 2 hemagglutinin (HA) A antigens, each of a different subtype and at least 2 HA B antigens, each of a different lineage, wherein the composition further comprises a lipid nanoparticle comprising 40-55 mol % ionizable amino lipid; 5-15 mol % neutral lipid; 35-45 mol % sterol; and 1-5 mol % PEG-modified lipid.

In some embodiments, the influenza antigens are encoded by one to four mRNAs. In some embodiments, the mRNA comprises a single mRNA encoding the at least 4 influenza antigens. In some embodiments, the mRNA comprises four mRNA each comprising a single open reading frame (ORF) encoding one of the 4 influenza antigens. In some embodiments, the 4 influenza antigens comprise an influenza A HA antigen of the H1 subtype, an influenza A HA antigen of the H3 subtype, an influenza B HA antigen of the B/Yamagata lineage, and an influenza B HA antigen of the B/Victoria lineage.

In some embodiments, the 4 mRNA are present in the composition in 1:1:greater than 1:greater than 1 ratio, influenza A:A:B:B strains, respectively. In some embodiments, the composition comprises 25 μg to 200 μg of the mRNA in total. In some embodiments, the percentage of subjects with seroconversion with respect to one of the four influenza antigens after a single dose at Day 29 is at least 40%, at least 50%, at least 60%, at least 70%, at least 80%, at least 90%, at least 95% or 100%. In some embodiments, the percentage of subjects with seroconversion with respect to one of the four influenza antigens after a single dose at Day 29 is 100%.

In some embodiments, the percentage of subjects with seroconversion with respect to two of the four influenza antigens after a single dose at Day 29 is at least 40%, at least 50%, at least 60%, at least 70%, at least 80%, at least 90%, at least 95% or 100%. In some embodiments, the percentage of subjects with seroconversion with respect to two of the four influenza antigens after a single dose at Day 29 is 100%.

In some embodiments, the percentage of subjects with seroconversion with respect to three of the four influenza antigens after a single dose at Day 29 is at least 40%, at least 50%, at least 60%, at least 70%, at least 80%, at least 90%, at least 95% or 100%. In some embodiments, the percentage of subjects with seroconversion with respect to three of the four influenza antigens after a single dose at Day 29 is 100%.

In some embodiments, the percentage of subjects with seroconversion with respect to all four of the influenza antigens after a single dose at Day 29 is at least 40%, at least 50%, at least 60%, at least 70%, at least 80%, at least 90%, at least 95% or 100%. In some embodiments, the percentage of subjects with seroconversion with respect to all four of the influenza antigens after a single dose at Day 29 is 100%.

In some embodiments, the percentage of subjects with at least a 2-fold rise in geometric mean fold rise (GMFR) after a single dose at Day 29 is at least 80%, at least 90%, at least 95% or 100%. In some embodiments, the percentage of subjects with a 2-fold rise in GMFR after a single dose at Day 29 is 100%. In some embodiments, the percentage of subjects with a 4-fold rise in GMFR after a single dose at Day 29 is at least 80%, at least 90%, at least 95% or 100%. In some embodiments, the percentage of subjects with a 4-fold rise in GMFR after a single dose at Day 29 is 100%.

In some embodiments, the GMFR comprises the H1N1 HAI titer GMFR. In some embodiments, the GMFR comprises the H3N2 HAI titer GMFR. In some embodiments, the GMFR comprises the B/Yamagata HAI titer GMFR. In some embodiments, the GMFR comprises the B/Victoria HAI titer GMFR.

In some embodiments, the serum antibody titers are increased 4-fold over baseline (Day 0) at Day 29 and Day 57 after a single dose. In some embodiments, the serum antibody titers are decreased at Day 181 over Day 29 titers after a single dose.

In some embodiments, the microneutralization titers are increased 4-fold over baseline (Day 0) at Day 29 and/or Day 57 after a single dose. In some embodiments, the microneutralization titers are decreased at Day 181 over Day 29 titers after a single dose.

In some embodiments, the composition comprises an approximately 25 μg to 250 μg dose of mRNA encoding four influenza HA proteins (e.g., HA proteins associated with the A/H1N1 strain, A/H3N2 strain, B/Victoria lineage and B/Yamagata lineage). In some embodiments, the composition comprises equal amounts of each of the mRNA encoding each of the four HA proteins (e.g., the mRNA are present in the composition at a 1:1:greater than 1: greater than 1 ratio, influenza A:A:B:B strains, respectively). In some embodiments, a composition comprises an approximately 25 μg dose of mRNA encoding four influenza HA proteins (e.g., HA proteins associated with the A/H1N1 strain, A/H3N2 strain, B/Victoria lineage and B/Yamagata lineage). In some embodiments, a composition comprises an approximately 50 μg dose of mRNA encoding four influenza HA proteins (e.g., HA proteins associated with the A/H1N1 strain, A/H3N2 strain, B/Victoria lineage and B/Yamagata lineage). In some embodiments, a composition comprises an approximately 100 μg dose of mRNA encoding four influenza HA proteins (e.g., HA proteins associated with the A/H1N1 strain, A/H3N2 strain, B/Victoria lineage and B/Yamagata lineage). In some embodiments, a composition comprises an approximately 150 μg dose of mRNA encoding four influenza HA proteins (e.g., HA proteins associated with the A/H1N1 strain, A/H3N2 strain, B/Victoria lineage and B/Yamagata lineage). In some embodiments, a composition comprises an approximately 200 μg dose of mRNA encoding four influenza HA proteins (e.g., HA proteins associated with the A/H1N1 strain, A/H3N2 strain, B/Victoria lineage and B/Yamagata lineage). In some embodiments, a composition comprises an approximately 250 μg dose of mRNA encoding four influenza HA proteins (e.g., HA proteins associated with the A/H1N1 strain, A/H3N2 strain, B/Victoria lineage and B/Yamagata lineage).

A composition may further comprise a buffer, for example a Tris buffer. For example, a composition may comprise 10 mM-30 mM, 10 mM-20 mM, or 20 mM-30 mM Tris buffer. In some embodiments, a composition comprises 10, 15, 20, 25, or 30 mM Tris buffer. In some embodiments, a composition comprises 20 mM Tris buffer.

In some embodiments, mRNA of a composition is formulated at a concentration of 0.1-1 mg/mL. In some embodiments, mRNA of a composition is formulated at a concentration of 0.1, 0.2, 0.3, 0.4, 0.5, 0.6, 0.7, 0.8, 0.9, or 1 mg/mL. In some embodiments, mRNA of a composition is formulated at a concentration of 0.5 mg/mL.

In some embodiments, a composition comprises sucrose. For example, a composition may comprise 75 mg/mL-95 mg/mL, 75 mg/mL-85 mg/mL, or 85 mg/mL-95 mg/mL sucrose. In some embodiments, a composition comprises 75, 80, 85, 86, 87, 88, 89, 90, or 95 mg/mL sucrose. In some embodiments, a composition comprises 87 mg/mL sucrose. In preferred embodiments, a composition does not include sodium acetate.

A composition may have a pH value of 6-8. In some embodiments, a composition has a pH value of 6, 6.5, 7, 7.5, or 8. In some embodiments, a composition has a pH value of 7.5.

In some embodiments, the composition further comprises a mixture of lipids. The mixture of lipids typically forms a lipid nanoparticle. The mRNA described herein, in some embodiments, is formulated with a lipid nanoparticle (e.g., for administration to a subject). In some embodiments, the lipid mixture, and thus the lipid nanoparticle, comprises: an ionizable amino lipid; a neutral lipid; a sterol; and a PEG-modified lipid.

For example, the lipid mixture/lipid nanoparticle may comprise: 20-60 mol % ionizable amino lipid; 5-25 mol % neutral lipid; 25-55 mol % sterol; and 0.5-15 mol % PEG-modified lipid. In some embodiments, the lipid nanoparticle comprises: 20-60 mol % ionizable amino lipid; 5-25 mol % neutral lipid; 25-55 mol % sterol; and 0.5-15 mol % PEG-modified lipid. In some embodiments, the lipid nanoparticle comprises: 40-55 mol % ionizable amino lipid; 5-15 mol % neutral lipid; 35-45 mol % sterol; and 1-5 mol % PEG-modified lipid. For example, the lipid nanoparticle may comprise: (a) 47 mol % ionizable amino lipid; 11.5 mol % neutral lipid; 38.5 mol % sterol; and 3.0 mol % PEG-modified lipid; (b) 48 mol % ionizable amino lipid; 11 mol % neutral lipid; 38.5 mol % sterol; and 2.5 mol % PEG-modified lipid; (c) 49 mol % ionizable amino lipid; 10.5 mol % neutral lipid; 38.5 mol % sterol; and 2.0 mol % PEG-modified lipid; (d) 50 mol % ionizable amino lipid; 10 mol % neutral lipid; 38.5 mol % sterol; and 1.5 mol % PEG-modified lipid; or (e) 51 mol % ionizable amino lipid; 9.5 mol % neutral lipid; 38.5 mol % sterol; and 1.0 mol % PEG-modified lipid. In some embodiments, the lipid mixture, and thus the lipid nanoparticle, comprises 20-55 mol %, 20-50 mol %, 20-45 mol %, 20-40 mol %, 25-60 mol %, 25-55 mol %, 25-50 mol %, 25-45 mol %, 25-40 mol %, 30-60 mol %, 30-55 mol %, 30-50 mol %, 30-45 mol %, 30-40 mol %, 35-60 mol %, 35-55 mol %, 35-50 mol %, 35-45 mol %, 35-40 mol %, 40-60 mol %, 40-55 mol %, 40-50 mol %, 40-45 mol %, 50-60 mol %, 50-55 mol %, or 55-60 mol % ionizable amino lipid. In some embodiments, the lipid mixture, and thus the lipid nanoparticle, comprises 5-20 mol %, 5-15 mol %, 5-10 mol %, 10-25 mol %, 10-20 mol %, 10-15 mol %, 15-25 mol %, 15-20 mol %, or 20-25 mol % neutral lipid. In some embodiments, the lipid mixture, and thus the lipid nanoparticle, comprises 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid mixture, and thus the lipid nanoparticle, comprises 0.5-10 mol %, 0.5-5 mol %, 0.5-1 mol %, 1-15%, 1-10 mol %, 1-5 mol %, 1.5-15%, 1.5-10 mol %, 1.5-5 mol %, 2-15%, 2-10 mol %, 2-5 mol %, 2.5-15%, 2.5-10 mol %, 2.5-5 mol %, 3-15%, 3-10 mol %, or 3-5 mol %, PEG-modified lipid. In some embodiments, the lipid mixture comprises: 50 mol % ionizable amino lipid; 10 mol % neutral lipid; 38.5 mol % sterol; and 1.5 mol % PEG-modified lipid. In some embodiments, the ionizable amino lipid is heptadecan-9-yl 8 ((2 hydroxyethyl)(6 oxo 6-(undecyloxy)hexyl)amino)octanoate. In some embodiments, the neutral lipid is 1,2 distearoyl sn glycero-3 phosphocholine (DSPC). In some embodiments, the sterol is cholesterol. In some embodiments, the PEG-modified lipid is 1-monomethoxypolyethyleneglycol-2,3-dimyristylglycerol with polyethylene glycol of average molecular weight 2000 (PEG2000 DMG).

A composition may further include a pharmaceutically-acceptable excipient, inert or active. A pharmaceutically acceptable excipient, after administered to a subject, does not cause undesirable physiological effects. The excipient in the pharmaceutical composition must be “acceptable” also in the sense that it is compatible with mRNA and can be capable of stabilizing it. One or more excipients (e.g., solubilizing agents) can be utilized as pharmaceutical carriers for delivery of the mRNA. Examples of a pharmaceutically acceptable excipients include, but are not limited to, biocompatible vehicles (e.g., LNPs), carriers, adjuvants, additives, and diluents to achieve a composition usable as a dosage form. Examples of other excipients include colloidal silicon oxide, magnesium stearate, cellulose, and sodium lauryl sulfate. Additional suitable pharmaceutical excipients, as well as pharmaceutical necessities for their use, are described in Remington's Pharmaceutical Sciences.

In some embodiments, an mRNA is formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with the RNA (e.g., for transplantation into a subject), hyaluronidase, nanoparticle mimics and combinations thereof. In some embodiments, a composition comprising mRNA does not include an adjuvant (the composition is adjuvant-free). Compositions may be sterile, pyrogen-free or both sterile and pyrogen-free. General considerations in the formulation and/or manufacture of pharmaceutical agents, such as compositions, may be found, for example, in Remington: The Science and Practice of Pharmacy 21st ed., Lippincott Williams & Wilkins, 2005. Formulations of the compositions described herein may be prepared by any method known or hereafter developed in the art of pharmacology. In general, such preparatory methods include the step of bringing the mRNA into association with an excipient (e.g., a mixture of lipids and/or a lipid nanoparticle), and then, if necessary and/or desirable, dividing, shaping and/or packaging the product into a desired single- or multi-dose unit. Relative amounts of the mRNA, the pharmaceutically-acceptable excipient, and/or any additional ingredients in a composition in accordance with the disclosure may vary, depending upon the identity, size, and/or condition of the subject treated and further depending upon the route by which the composition is to be administered.

In some embodiments, the present disclosure demonstrates that an improved immune response can be produced by increasing the relative amount of RNA encoding an antigen of a type B influenza virus as compared to RNA encoding an influenza type A virus (e.g., an immune response that comprises higher neutralization titers against an influenza type B virus (e.g., higher neutralization titers as compared to a composition comprising equal amounts of RNA encoding an influenza type A antigen and RNA encoding an influenza type B antigen (e.g., as determined by a pseudovirus neutralization assay described herein))). The present disclosure also provides exemplary doses of RNA that can produce strong immune responses against both types of influenza viruses (e.g., neutralizing titers and/or seroconversion rates that are at clinically relevalent levels (e.g., (i) neutralizing titers that are comparable or superior to those previously shown to prevent influenza symptoms, and/or (ii) neutralizing titers and/or seroconversion rates that are comparable or superior to those induced by a relevant comparator (e.g., a commercially approved influenza vaccine or an influenza RNA vaccine))). In some embodiments, a composition comprising a greater amount of RNA encoding influenza B antigens as compared to RNA encoding influenza A antigens produces an immune response against each of an influenza type B virus and influenza type A virus that is comparable or superior to that induced by a non-RNA influenza vaccine (e.g., an approved vaccine) and/or an RNA vaccine comprising equal amounts of RNA encoding influenza A antigens and RNA encoding influenza B antigens.

In some embodiments, the concentration of RNA in a pharmaceutical RNA preparation is about 0.1-0.2 mg/ml. In some embodiments, the concentration of RNA in a pharmaceutical RNA preparation is about 0.1 mg/ml. In some embodiments, the concentration of RNA in a pharmaceutical RNA preparation is about 0.12 mg/ml. In some embodiments, the concentration of RNA in a pharmaceutical RNA preparation is about 0.14 mg/ml. In some embodiments, the concentration of RNA in a pharmaceutical RNA preparation is about 0.16 mg/ml. In some embodiments, the concentration of RNA in a pharmaceutical RNA preparation is about 0.18 mg/ml. In some embodiments about 30 μg of RNA is administered by administering about 200 μL of RNA preparation. In some embodiments, the RNA in a pharmaceutical RNA preparation is diluted prior to administration (e.g., diluted to a concentration of about 0.05 mg/ml). In some embodiments, administration volumes are between about 200 μl and about 300 μl. In some embodiments, the RNA in a pharmaceutical RNA preparation is formulated in about 10 mM Tris buffer, and about 10% sucrose.

In some embodiments, a pharmaceutical RNA preparation comprises RNA in a concentration of about 0.1 mg/ml, and is formulated in about 10 mM Tris buffer, and about 10% sucrose. In some embodiments, a pharmaceutical RNA preparation comprises RNA in a concentration of about 0.12 mg/ml, and is formulated in about 10 mM Tris buffer, and about 10% sucrose. In some embodiments, a pharmaceutical RNA preparation comprises RNA in a concentration of about 0.14 mg/ml, and is formulated in about 10 mM Tris buffer, and about 10% sucrose. In some embodiments, a pharmaceutical RNA preparation comprises RNA in a concentration of about 0.16 mg/ml, and is formulated in about 10 mM Tris buffer, and about 10% sucrose. In some embodiments, a pharmaceutical RNA preparation comprises RNA in a concentration of about 0.18 mg/ml, and is formulated in about 10 mM Tris buffer, and about 10% sucrose. Such a formulation can be diluted as needed prior to administration to administer different doses of RNA while keeping total injection volume relatively constant. For example, a dose of RNA of about 10 μg can be administered by diluting such a pharmaceutical RNA preparation by about 1:1 and administering about 200 μl of diluted pharmaceutical RNA preparation.

In some embodiments, a vaccine is formulated in a vial (e.g., a glass vial). In some embodiments, a glass vial is sealed with a bromobutyl elastomeric stopper and an aluminum seal with flip-off plastic cap.

In some embodiments, a composition comprises an RNA encoding an antigen (e.g., an HA protein) of an influenza virus that is recommended by a relevant health authority for inclusion in a seasonally-adapted vaccine (e.g., a cell-based, recombinant, or live attenuated virus). In some embodiments a composition comprises a plurality of RNAs, encoding antigens (e.g., HA proteins) of each influenza virus recommended by a relevant health authority for inclusion in a seasonally-adapted vaccine (e.g., a cell-based, recombinant, or live attenuated virus).

In some embodiments, the influenza virus is an influenza A, influenza B, or influenza C virus. In some embodiments, the influenza A virus is an H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, H10N7, or H10N8 virus. In some embodiments, the influenza A virus is an H1N1, H3N2, H5N1, or H5N8 virus. In some embodiments, the influenza A virus is an H1N1 virus (e.g., A/Wisconsin/588/2019 or A/Sydney/5/2021). In some embodiments the influenza A virus is an H3N2 virus. In some embodiments the H3N2 virus is A/Cambodia/e0826360/2020 or A/Darwin/6/2021. In some embodiments, the influenza B virus is of a B/Yamagata or B/Victoria lineage. In some embodiments, the B/Victoria lineage influenza virus is B/Washington/02/2019. In some embodiments, the B/Victoria lineage virus is B/Austria/1359417/2021. In some embodiments, the B/Yamagata lineage influenza virus is B/Phuket/3073/2013.

In some embodiments, a composition described herein comprises a multivalent influenza vaccine. In some embodiments, a multivalent influenza vaccine comprises 2 to 50 RNA distinct molecules (e.g., 2 to 40, 2 to 30, or 2 to 20 RNA molecules), each of which, in some embodiments, may encode a different antigenic polypeptide (or a different version of a particular antigenic polypeptide) associated with influenza, e.g., as described in Arevalo, Claudia P., et al. “A multivalent nucleoside-modified mRNA vaccine against all known influenza virus subtypes.” Science 378.6622 (2022): 899-904. In some embodiments, a composition described herein comprises a trivalent influenza vaccine. In some embodiments, a trivalent influenza vaccine comprises RNAs encoding an antigenic polypeptide associated with two type A viruses and one type B virus that are predicted to be prevalent in a relevant jurisdiction. In some embodiments, the trivalent composition includes a modRNA suspension for injection, e.g., 0.09 mg/mL, having a ratio of 1:1:4 of influenza HA A:A:B strain, respectively. In some embodiments, the trivalent composition comprises 0.015 mg/ml modRNA encoding HA from an influenza A strain, 0.015 mg/ml modRNA encoding HA from an influenza A strain, and 0.060 mg/ml of an influenza B strain. The composition may further comprise a cationic lipid, a pegylated lipid, phospholipid, and sterol, sucrose, tromethamine, and Tris-HCl. A trivalent modRNA HA (B/Austria, A/Wisconsin, A/Darwin) 0.6ug composition elicited an immune response in mice, wherein the composition included 0.2 ug of each of the 3 HA. The dose volume and immunization route was 50 ul/IM, administered on day 0 and 28. Bleed occurred on day 21 and 42. See Example 9.

In some embodiments, a composition described herein comprises a tetravalent influenza vaccine. In some embodiments, a tetravalent influenza vaccine comprises RNAs encoding an antigenic polypeptide associated with two type A viruses and two type B viruses that are predicted to be prevalent in a relevant jurisdiction. In some embodiments, a composition described herein comprises an octavalent influenza vaccine. In some embodiments, an octavalent influenza vaccine comprises RNAs encoding two antigenic polypeptides associated with each of two type A viruses and two type B viruses that are predicted to be prevalent in a relevant jurisdiction (e.g., an HA protein and an NA protein associated with each virus, or immunogenic fragments thereof). In some embodiments, a composition disclosed herein comprises a tetravalent influenza vaccine comprising an RNA comprising a nucleotide sequence encoding an HA protein associated with an H1N1 virus (e.g., A/Wisconsin/588/2019), an RNA comprising a nucleotide sequence encoding an HA protein associated with an H3N2 virus (e.g., A/Cambodia/e0826360/2020), an RNA comprising a nucleotide sequence encoding an HA protein associated with a B/Victoria lineage influenza virus (e.g., B/Washington/02/2019), and an HA protein associated with a B/Yamagata lineage influenza virus (e.g., B/Phuket/3073/2013). In some embodiments, the tetravalent influenza RNA composition comprises 60 μg of total RNA (e.g., 5 μg of an RNA encoding an HA protein of an influenza A strain, 5 μg of an RNA encoding an HA protein of an influenza A strain, 25 μg of an RNA encoding an HA protein of an influenza B strain, and 25 μg of an RNA encoding an HA protein of an influenza B strain). In some embodiments, the tetravalent influenza RNA composition comprises 60 μg of total RNA (e.g., 5 μg of an RNA encoding an HA protein of an H1N1 influenza strain, 5 μg of an RNA encoding an HA protein of an H3N2 influenza strain, 25 μg of an RNA encoding an HA protein of a B/Victoria influenza lineage, and 25 μg of an RNA encoding an HA protein of a B/Yamagata influenza lineage). In some embodiments, the tetravalent influenza RNA composition described herein comprises 45 μg of total RNA (e.g., 11.25 μg of an RNA encoding an HA protein of a first influenza A strain, 11.25 μg of an RNA encoding an HA protein of a second influenza A strain, 11.25 μg of an RNA encoding an HA protein of an influenza B strain, and 11.25 μg of an RNA encoding an HA protein of a B/Yamagata influenza lineage). In some embodiments, the tetravalent influenza RNA composition described herein comprises 45 μg of total RNA (e.g., 11.25 μg of an RNA encoding an HA protein of an H1N1 influenza strain, 11.25 μg of an RNA encoding an HA protein of an H3N2 influenza strain, 11.25 μg of an RNA encoding an HA protein of a B/Victoria influenza lineage, and 11.25 μg of an RNA encoding an HA protein of a B/Yamagata influenza lineage). In some embodiments, the tetravalent influenza RNA composition comprises 30 μg of total RNA (e.g., 2.5 μg of an RNA encoding an HA protein of an H1N1 influenza strain, 2.5 μg of an RNA encoding an HA protein of an H3N2 influenza strain, 12.5 μg of an RNA encoding an HA protein of a B/Victoria influenza lineage, and 12.5 μg of an RNA encoding an HA protein of a B/Yamagata influenza lineage).

In some embodiments, a composition comprises a tetravalent influenza vaccine comprises RNA encoding an antigenic polypeptide associated with two type A viruses and two type B viruses that are predicted to be prevalent in a relevant jurisdiction. In some embodiments, a tetravalent influenza vaccine comprises RNA encoding an antigenic polypeptide associated with an H1N1 influenza virus, RNA encoding an antigenic polypeptide associated with an H3N2 influenza virus, RNA encoding an antigenic polypeptide associated with a Victoria lineage influenza virus, and RNA encoding an antigenic polypeptide associated with a Yamagata lineage influenza virus. In some embodiments, the tetravalent influenza vaccine comprises RNA associated with influenza types that are predicted to be prevalent in a relevant jurisdiction (e.g., HA polypeptides associated with the H1N1, H3N2, B/Victoria, and B/Yamagata influenza viruses that are predicted to be prevalent in a relevant jurisdiction).

In some embodiments, each of the RNAs in a composition disclosed herein encodes an antigenic polypeptide associated with an infectious agent that is predicted to be prevalent in a relevant jurisdiction. Such compositions can reduce the number of vaccinations needed. In some embodiments, a nucleic acid containing particle comprises two or more RNA molecules, each comprising a nucleotide sequence encoding an antigen (e.g., an HA protein) associated with a different influenza virus. In some embodiments, a nucleic acid containing particle comprises three or more RNA molecules, each comprising a nucleotide sequence encoding an antigen (e.g., an HA protein) associated with a different influenza virus. In some embodiments, a nucleic acid containing particle comprises four or more RNA molecules, each comprising a nucleotide sequence encoding an antigen (e.g., an HA protein) associated with a different influenza virus. In some embodiments, a nucleic acid containing particle comprises an RNA molecule comprising a nucleotide sequence encoding an antigenic polypeptide associated with an H1N1 influenza virus, an RNA molecule comprising a nucleotide sequence encoding an antigenic polypeptide associated with an H3N2 influenza virus, an RNA molecule comprising a nucleotide sequence encoding an antigenic polypeptide associated with a B/Victoria lineage influenza virus, and an RNA molecule comprising a nucleotide sequence encoding an antigenic polypeptide associated with a B/Yamagata influenza virus. In some embodiments, each RNA in a composition comprising a nucleotide sequence encoding an antigenic polypeptide associated with an influenza virus is formulated in the same nucleic acid containing particle. In some embodiments, each RNA in a composition comprising a nucleotide sequence encoding an antigenic polypeptide associated with an influenza virus is formulated in separate nucleic acid containing particles.

In some embodiments, a nucleic acid containing particle (e.g., in some embodiments an LNP as described herein) comprising two or more RNA molecules, comprises each RNA molecule in the same amount (i.e., at a 1:1 ratio).

In some embodiments, a nucleic acid containing particle (e.g., in some embodiments an LNP as described herein) comprising two or more RNA molecules, comprises a different amount of each RNA molecule. For example, in some embodiments, a nucleic acid containing particle comprises a first RNA molecule and a second RNA molecule, where the first RNA molecule is present in an amount that is 0.01 to 100 times that of the second RNA molecule (e.g., wherein the amount of the first RNA molecule is 0.01 to 50, 0.01 to 4, 0.01 to 30, 0.01 to 25, 0.01 to 20, 0.01 to 15, 0.01 to 10, 0.01 to 9, 0.01 to 8, 0.01 to 7, 0.01 to 6, 0.01 to 5, 0.01 to 4, 0.01 to 3, 0.01 to 2, 0.01 to 1.5, 1 to 50, 1 to 4, 1 to 30, 1 to 25, 1 to 20, 1 to 15, 1 to 10, 1 to 9, 1 to 8, 1 to 7, 1 to 6, 1 to 5, 1 to 4, 1 to 3, 1 to 2, or 1 to 1.5 times higher than the second RNA molecule).

In some embodiments, a nucleic acid containing particle comprises a first RNA molecule and a second RNA molecule, wherein the concentration of the first RNA molecule is 1 to 10 times that of the second RNA molecule. In some embodiments, a nucleic acid containing particle comprises a first RNA molecule and a second RNA molecule, wherein the concentration of the first RNA molecule is 1 to 5 times that of the second RNA molecule. In some embodiments, a nucleic acid containing particle comprises a first RNA molecule and a second RNA molecule, wherein the concentration of the first RNA molecule is 1 to 3 times that of the second RNA molecule. In some embodiments, a nucleic acid containing particle comprises a first RNA molecule and a second RNA molecule, wherein the concentration of the first RNA molecule is 2 times that of the second RNA molecule. In some embodiments, a nucleic acid containing particle comprises a first RNA molecule and a second RNA molecule, wherein the concentration of the first RNA molecule is 3 times that of the second RNA molecule.

In some embodiments, a nucleic acid containing particle (e.g., in some embodiments an LNP as described herein) comprising three RNA molecules, comprises each RNA molecule in the same amount (i.e., at a 1:1:1 ratio).

In some embodiments, a nucleic acid containing particle (e.g., in some embodiments an LNP as described herein) comprising three RNA molecules, comprises a different amount of each RNA molecule. For example, in some embodiments, the ratio of first RNA molecule: second RNA molecule: third RNA molecule is 1:0.01-100:0.01-100 (e.g., 1:0.01-50:0.01-50; 1:0.01-40:0.01-40; 1:0.01-30:0.01-25; 1:0.01-25:0.01-25; 1:0.01-20:0.01-20; 1:0.01-15:0.01-15; 1:0.01-10:0.01-9; 1:0.01-9:0.01-9; 1:0.01-8:0.01-8; 1:0.01-7:0.01-7; 1:0.01-6:0.01-6; 1:0.01-5:0.01-5; 1:0.01-4:0.01-4; 1:0.01-3:0.01-3; 1:0.01-2:0.01-2; or 1:0.01-1.5:0.01-1.5). In some embodiments, the ratio of first RNA molecule: second RNA molecule: third RNA molecule is 1:1:3. In some embodiments, the ratio of first RNA molecule: second RNA molecule: third RNA molecule is 1:3:3.

The term “dose” as used herein refers in general to a “dose amount” which relates to the amount of RNA administered per administration, i.e., per dosing.

In some embodiments, administration of an immunogenic composition or vaccine of the present disclosure may be performed by single administration or boosted by multiple administrations. In some embodiments, a regimen described herein includes at least one dose. In some embodiments, a regimen includes a first dose and at least one subsequent dose. In some embodiments, the first dose is the same amount as at least one subsequent dose. In some embodiments, the first dose is the same amount as all subsequent doses. In some embodiments, the first dose is a different amount as at least one subsequent dose. In some embodiments, the first dose is a different amount than all subsequent doses. In some embodiments, a regimen comprises two doses. In some embodiments, a provided regimen consists of two doses. In some embodiments, a regimen comprises three doses.

In one embodiment, the disclosure envisions administration of a single dose. In one embodiment, the disclosure envisions administration of a priming dose followed by one or more booster doses. The booster dose or the first booster dose may be administered 7 to 28 days or 14 to 24 days following administration of the priming dose. In some embodiments, a first booster dose may be administered 1 week to 3 months (e.g., 1 week, 2 weeks, 3 weeks, 4 weeks, 5 weeks, 6 weeks, 7 weeks, 8 weeks, 9 weeks, 10 weeks, 11 weeks, 12 weeks) following administration of a priming dose. In some embodiments, a subsequent booster dose may be administered at least 1 week or longer, including, e.g., at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, or longer, following a preceding booster dose. In some embodiments, subsequent booster doses may be administered about 5-9 weeks or 6-8 weeks apart. In some embodiments, at least one subsequent booster dose (e.g., after a first booster dose) may be administered at least 3 months or longer, including, e.g., at least 4 months, at least 5 months, at least 6 months, at least 7 months, at least 8 months, at least 9 months, at least 10 months, or longer, following a preceding dose.

In some embodiments, a dose comprises a total amount of RNA of 0.1 μg to 300 μg, 0.5 μg to 200 μg, or 1 μg to 100 μg, such as about 1 μg, about 2 μg, about 3 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, or about 100 μg. In some embodiments, a dose comprises a total amount of RNA (e.g., modRNA) of up to about 100 μg. In some embodiments, a dose comprises 0.1 μg to 100 μg of one or more first RNAs and 0.1 μg to 100 μg of one or more second RNAs, wherein the one or more first RNAs each comprise a nucleotide sequence encoding an antigenic polypeptide associated with a first infectious agent (e.g., a coronavirus), and the one or more second RNAs each comprise a nucleotide sequence encoding an antigenic polypeptide associated with a second infectious agent (e.g., influenza). In some embodiments, a dose comprises 3 to 60 μg of one or more first RNAs and 3 to 90 μg of one or more second RNAs. In some embodiments, a dose comprises 3 to 60 μg of one or more first RNAs and 3 to 90 μg of one or more second RNAs, wherein the dose comprises up to 100 μg of RNA total. In some embodiments, a dose comprises 3 to 30 μg of one or more first RNAs and 3 to 60 μg of one or more second RNAs, wherein the dose comprises up to 100 μg of RNA total. In some embodiments, a dose comprises 3 μg of one or more first RNAs and 3 μg of one or more second RNAs. In some embodiments, a dose comprises 3 μg of one or more first RNAs and 6 μg of one or more second RNAs. In some embodiments, a dose comprises 10 μg of one or more first RNAs and 10 μg of one or more second RNAs. In some embodiments, a dose comprises 10 μg of one or more first RNAs and 20 μg of one or more second RNAs. In some embodiments, a dose comprises 30 μg of one or more first RNAs and 30 μg of one or more second RNAs. In some embodiments, a dose comprises 30 μg of one or more first RNAs and 60 μg of one or more second RNAs. In some embodiments, a dose comprises 60 μg of one or more first RNAs and 30 μg of one or more second RNAs.

In some embodiments, a subsequent dose given to an individual (e.g., as part of a primary regimen or booster regimen) can have the same amount of RNA as previously given to the individual. In some embodiments, a subsequent dose given to an individual (e.g., as part of a primary regimen or booster regimen) can differ in the amount of RNA, as compared to the amount previously given to the individual. For example, in some embodiments, a subsequent dose can be higher or lower than the prior dose, for example, based on consideration of various factors, including, e.g., immunogenicity and/or reactogenicity induced by the prior dose, prevalence of the disease, etc. In some embodiments, a subsequent dose can be higher than a prior dose by at least 30%, at least 40%, at least 50%, at least 60%, at least 70%, at least 80%, at least 90%, or higher. In some embodiments, a subsequent dose can be higher than a prior dose by at least 1.5-fold, at least 2-fold, at least 2.5 fold, at least 3-fold, or higher. In some embodiments, a subsequent dose can be higher than a prior dose by at least 30%, at least 40%, at least 50%, at least 60%, at least 70%, at least 80%, at least 90%, or higher. In some embodiments, a subsequent dose can be lower than a prior dose by at least 10%, at least 20%, at least 30%, at least 40%, at least 50%, at least 60%, at least 70% or lower. In some embodiments, an amount the RNA described herein from 0.1 μg to 300 μg, 0.5 μg to 200 μg, or 1 μg to 100 μg, such as about 1 μg, about 2 μg, about 3 μg, about 10 μg, about 15 μg, about 20 μg, about 25 μg, about 30 μg, about 35 μg, about 40 μg, about 45 μg, about 50 μg, about 55 μg, about 60 μg, about 70 μg, about 80 μg, about 90 μg, or about 100 μg may be administered per dose (e.g., in a given dose).

In some embodiments, an amount of the RNA described herein of 60 μg or lower, 55 μg or lower, 50 μg or lower, 45 μg or lower, 40 μg or lower, 35 μg or lower, 30 μg or lower, 25 μg or lower, 20 μg or lower, 15 μg or lower, 10 μg or lower, 5 μg or lower, 3 μg or lower, 2.5 μg or lower, or 1 μg or lower may be administered per dose (e.g., in a given dose).

In some embodiments, an amount of the RNA described herein of at least 0.25 μg, at least 0.5 μg, at least 1 μg, at least 2 μg, at least 3 μg, at least 4 μg, at least 5 μg, at least 10 μg, at least 15 μg, at least 20 μg, at least 25 μg, at least 30 μg, at least 40 μg, at least 50 μg, or at least 60 μg may be administered per dose (e.g., in a given dose). In some embodiments, an amount of the RNA described herein of at least 3 ug may be administered in at least one of given doses. In some embodiments, an amount of the RNA described herein of at least 10 ug may be administered in at least one of given doses. In some embodiments, an amount of the RNA described herein of at least 15 ug may be administered in at least one of given doses. In some embodiments, an amount of the RNA described herein of at least 20 ug may be administered in at least one of given doses. In some embodiments, an amount of the RNA described herein of at least 25 ug may be administered in at least one of given doses. In some embodiments, an amount of the RNA described herein of at least 30 ug may be administered in at least one of given doses. In some embodiments, an amount of the RNA described herein of at least 50 ug may be administered in at least one of given doses. In some embodiments, an amount of the RNA described herein of at least 60 ug may be administered in at least one of given doses. In some embodiments, combinations of aforementioned amounts may be administered in a regimen comprising two or more doses (e.g., a prior dose and a subsequent dose can be of different amounts as described herein). In some embodiments, combinations of aforementioned amounts may be administered in a primary regimen and a booster regimen (e.g., different doses can be given in a primary regimen and a booster regimen). In some embodiments, an amount of an RNA described herein of 0.25 μg to 60 μg, 0.5 μg to 55 μg, 1 μg to 50 μg, 5 μg to 40 μg, or 10 μg to 30 μg may be administered per dose. In some embodiments, an amount of the RNA described herein of 3 μg to 30 μg may be administered in at least one of given doses. In some embodiments, an amount of the RNA described herein of 3 μg to 20 μg may be administered in at least one of given doses. In some embodiments, an amount of the RNA described herein of 3 μg to 15 μg may be administered in at least one of given doses. In some embodiments, an amount of the RNA described herein of 3 μg to 10 μg may be administered in at least one of given doses. In some embodiments, an amount of the RNA described herein of 10 μg to 30 μg may be administered in at least one of given doses. In some embodiments, a regimen administered to a subject may comprise a plurality of doses (e.g., at least two doses, at least three doses, or more). In some embodiments, a regimen administered to a subject may comprise a first dose and a second dose, which are given at least 2 weeks apart, at least 3 weeks apart, at least 4 weeks apart, or more. In some embodiments, such doses may be at least 1 month, at least 2 months, at least 3 months, at least 4 months, at least 5 months, at least 6 months, at least 7 months, at least 8 months, at least 9 months, at least 10 months, at least 11 months, at least 12 months, or more apart. In some embodiments, doses may be administered days apart, such as 1, 2, 3, 4, 5, 6, 7, 8, 9,10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more days apart. In some embodiments, doses may be administered about 1 to about 3 weeks apart, or about 1 to about 4 weeks apart, or about 1 to about 5 weeks apart, or about 1 to about 6 weeks apart, or about 1 to more than 6 weeks apart. In some embodiments, doses may be separated by a period of about 7 to about 60 days, such as for example about 14 to about 48 days, etc. In some embodiments, a minimum number of days between doses may be about 1, 2, 3, 4, 5, 6, 7, 8, 9, 10, 11, 12, 13, 14, 15, 16, 17, 18, 19, 20, 21 or more. In some embodiments, a maximum number of days between doses may be about 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or fewer. In some embodiments, doses may be about 21 to about 28 days apart. In some embodiments, doses may be about 19 to about 42 days apart. In some embodiments, doses may be about 7 to about 28 days apart. In some embodiments, doses may be about 14 to about 24 days. In some embodiments, doses may be about 21 to about 42 days.

In some embodiments, a vaccination regimen comprises a first dose and a second dose. In some embodiments, a first dose and a second dose are administered by at least 21 days apart. In some embodiments, a first dose and a second dose are administered by at least 28 days apart. In some embodiments, a vaccination regimen comprises a first dose and a second dose, wherein the amount of RNA administered in the first dose is the same as the amount of RNA administered in the second dose. In some embodiments, a vaccination regimen comprises a first dose and a second dose wherein the amount of RNA administered in the first dose differs from that administered in the second dose. In some embodiments, a vaccination regimen comprises a first dose and a second dose, wherein the amount of RNA administered in the first dose is less than that administered in the second dose. In some embodiments, the amount of RNA administered in the first dose is 10%-90% of the second dose. In some embodiments, the amount of RNA administered in the first dose is 10%-50% of the second dose. In some embodiments, the amount of RNA administered in the first dose is 10%-20% of the second dose. In some embodiments, the first dose and the second dose are administered at least 2 weeks apart, including, at least 3 weeks apart, at least 4 weeks apart, at least 5 weeks apart, at least 6 weeks apart or longer. In some embodiments, the first dose and the second dose are administered at least 3 weeks apart. In some embodiments, a first dose comprises less than about 30 μg of RNA and a second dose comprises at least about 30 μg of RNA. In some embodiments, a first dose comprises about 1 to less than about 30 μg of RNA (e.g., about 0.1, about 1, about 3, about 5, about 10, about 15, about 20, about 25, or less than about 30 μg of RNA) and a second dose comprises about 30 to about 100 μg of RNA (e.g., about 30, about 40, about 50, or about 60 μg of RNA). In some embodiments, a first dose comprises about 1 to about 20 μg of RNA, about 1 to about 10 μg of RNA, or about 1 to about 5 μg of RNA and a second dose comprises about 30 to about 60 μg of RNA. In some embodiments, a first dose comprises about 1 to about 10 μg of RNA (e.g., about 1, about 2, about 3, about 4, about 5, about 6, about 7, about 8, about 9, or about 10 μg of RNA) and a second dose comprises about 30 to about 60 μg of RNA (e.g., about 30, about 35, about 40, about 45, about 50, about 55, or about 60 μg of RNA). In some embodiments, a first dose comprises about 1 μg of RNA and a second dose comprises about 30 μg of RNA. In some embodiments, a first dose comprises about 3 μg of RNA and a second dose comprises about 30 μg of RNA. In some embodiments, a first dose comprises about 5 μg of RNA and a second dose comprises about 30 μg of RNA. In some embodiments, a first dose comprises about 10 μg of RNA and a second dose comprises about 30 μg of RNA. In some embodiments, a first dose comprises about 15 μg of RNA and a second dose comprises about 30 μg of RNA.

In some embodiments, a first dose comprises about 1 μg of RNA and a second dose comprises about 60 μg of RNA. In some embodiments, a first dose comprises about 3 μg of RNA and a second dose comprises about 60 μg of RNA. In some embodiments, a first dose comprises about 5 μg of RNA and a second dose comprises about 60 μg of RNA. In some embodiments, a first dose comprises about 6 μg of RNA and a second dose comprises about 60 μg of RNA. In some embodiments, a first dose comprises about 10 μg of RNA and a second dose comprises about 60 μg of RNA. In some embodiments, a first dose comprises about 15 μg of RNA and a second dose comprises about 60 μg of RNA. In some embodiments, a first dose comprises about 20 μg of RNA and a second dose comprises about 60 μg of RNA. In some embodiments, a first dose comprises about 25 μg of RNA and a second dose comprises about 60 μg of RNA.

In some embodiments, a first dose comprises about 30 μg of RNA and a second dose comprises about 60 μg of RNA.

In some embodiments, a first dose comprises less than about 10 μg of RNA and a second dose comprises at least about 10 μg of RNA. In some embodiments, a first dose comprises about 0.1 to less than about 10 μg of RNA (e.g., about 0.1, about 0.5, about 1, about 2, about 3, about 4, about 5, about 6, about 7, about 8, or less than about 10 μg of RNA) and a second dose comprises about 10 to about 30 μg of RNA (e.g., about 10, about 15, about 20, about 25, or about 30 μg of RNA). In some embodiments, a first dose comprises about 0.1 to about 10 μg of RNA, about 1 to about 5 μg of RNA, or about 0.1 to about 3 μg of RNA and a second dose comprises about 10 to about 30 μg of RNA.

In some embodiments, a first dose comprises about 0.1 to about 5 μg of RNA (e.g., about 0.1, about 0.5, about 1, about 2, about 3, about 4, about 5ug of RNA) and a second dose comprises about 10 to about 20 μg of RNA (e.g., about 10, about 12, about 14, about 16, about 18, about 20ug of RNA).

In some embodiments, a first dose comprises about 0.1 ug of RNA and a second dose comprises about 10 μg of RNA. In some embodiments, a first dose comprises about 0.3 ug of RNA and a second dose comprises about 10 μg of RNA. In some embodiments, a first dose comprises about 1 μg of RNA and a second dose comprises about 10 μg of RNA. In some embodiments, a first dose comprises about 3 μg of RNA and a second dose comprises about 10 μg of RNA.

In some embodiments, a first dose comprises less than about 3 μg of RNA and a second dose comprises at least about 3 μg of RNA. In some embodiments, a first dose comprises about 0.1 to less than about 3 μg of RNA (e.g., about 0.1, about 0.2, about 0.3, about 0.5, about 0.6, about 0.7, about 0.8, about 0.9, about 1.0, about 1.5, about 2.0, or about 2.5 ug of RNA) and a second dose comprises about 3 to about 10 μg of RNA (e.g., about 3, about 4, about 5, about 6, or about 7, about 8, about 9, or about 10 μg of RNA). In some embodiments, a first dose comprises about 0.1 to about 3 μg of RNA, about 0.1 to about 1 μg of RNA, or about 0.1 to about 0.5 ug of RNA and a second dose comprises about 3 to about 10 μg of RNA.

In some embodiments, a first dose comprises about 0.1 to about 1.0 ug of RNA (e.g., about 0.1, about 0.2, about 0.3, about 0.4, about 0.5, about 0.6, about 0.7, about 0.8, about 0.9, or about 1.0 ug of RNA) and a second dose comprises about 1 to about 3 μg of RNA (e.g., about 1.0, about 1.5, about 2.0, about 2.5, or about 3.0 ug of RNA).

In some embodiments, a first dose comprises about 0.1 ug of RNA and a second dose comprises about 3 μg of RNA. In some embodiments, a first dose comprises about 0.3 ug of RNA and a second dose comprises about 3 μg of RNA. In some embodiments, a first dose comprises about 0.5 ug of RNA and a second dose comprises about 3 μg of RNA. In some embodiments, a first dose comprises about 1 μg of RNA and a second dose comprises about 3 μg of RNA.

In some embodiments, a vaccination regimen comprises a first dose and a second dose, wherein the amount of RNA administered in the first dose is greater than that administered in the second dose. In some embodiments, the amount of RNA administered in the second dose is 10%-90% of the first dose. In some embodiments, the amount of RNA administered in the second dose is 10%-50% of the first dose. In some embodiments, the amount of RNA administered in the second dose is 10%-20% of the first dose. In some embodiments, the first dose and the second dose are administered at least 2 weeks apart, including, at least 3 weeks apart, at least 4 weeks apart, at least 5 weeks apart, at least 6 weeks apart or longer. In some embodiments, the first dose and the second dose are administered at least 3 weeks apart In some embodiments, a first dose comprises at least about 30 μg of RNA and a second dose comprises less than about 30 μg of RNA. In some embodiments, a first dose comprises about 30 to about 100 μg of RNA (e.g., about 30, about 40, about 50, or about 60 μg of RNA) and a second dose comprises about 1 to about 30 μg of RNA (e.g., about 0.1, about 1, about 3, about 5, about 10, about 15, about 20, about 25, or about 30 μg of RNA). In some embodiments, a second dose comprises about 1 to about 20 μg of RNA, about 1 to about 10 μg of RNA, or about 1 to 5 μg of RNA. In some embodiments, a first dose comprises about 30 to about 60 μg of RNA and a second dose comprises about 1 to about 20 μg of RNA, about 1 to about 10 μg of RNA, or about 0.1 to about 3 μg of RNA.

In some embodiments, a first dose comprises about 30 to about 60 μg of RNA (e.g., about 30, about 35, about 40, about 45, about 50, about 55, or about 60 μg of RNA) and a second dose comprises about 1 to about 10 μg of RNA (e.g., about 1, about 2, about 3, about 4, about 5, about 6, about 7, about 8, about 9, or about 10 μg of RNA).

In some embodiments, a first dose comprises about 30 μg of RNA and a second dose comprises about 1 μg of RNA. In some embodiments, a first dose comprises about 30 μg of RNA and a second dose comprises about 3 μg of RNA. In some embodiments, a first dose comprises about 30 μg of RNA and a second dose comprises about 5 μg of RNA. In some embodiments, a first dose comprises about 30 μg of RNA and a second dose comprises about 10 μg of RNA. In some embodiments, a first dose comprises about 30 μg of RNA and a second dose comprises about 15 μg of RNA.

In some embodiments, a first dose comprises about 60 μg of RNA and a second dose comprises about 1 μg of RNA. In some embodiments, a first dose comprises about 60 μg of RNA and a second dose comprises about 3 μg of RNA. In some embodiments, a first dose comprises about 60 μg of RNA and a second dose comprises about 5 μg of RNA. In some embodiments, a first dose comprises about 60 μg of RNA and a second dose comprises about 6 μg of RNA. In some embodiments, a first dose comprises about 60 μg of RNA and a second dose comprises about 10 μg of RNA. In some embodiments, a first dose comprises about 60 μg of RNA and a second dose comprises about 15 μg of RNA. In some embodiments, a first dose comprises about 60 μg of RNA and a second dose comprises about 20 μg of RNA. In some embodiments, a first dose comprises about 60 μg of RNA and a second dose comprises about 25 μg of RNA. In some embodiments, a first dose comprises about 60 μg of RNA and a second dose comprises about 30 μg of RNA.

In some embodiments, a first dose comprises at least about 10 μg of RNA and a second dose comprises less than about 10 μg of RNA. In some embodiments, a first dose comprises about 10 to about 30 μg of RNA (e.g., about 10, about 15, about 20, about 25, or about 30 μg of RNA) and a second dose comprises about 0.1 to less than about 10 μg of RNA (e.g., about 0.1, about 0.5, about 1, about 2, about 3, about 4, about 5, about 6, about 7, about 8, or less than about 10 μg of RNA). In some embodiments, a first dose comprises about 10 to about 30 μg of RNA, or about 0.1 to about 3 μg of RNA and a second dose comprises about 1 to about 10 μg of RNA, or about 1 to about 5 μg of RNA.

In some embodiments, a first dose comprises about 10 to about 20 μg of RNA (e.g., about 10, about 12, about 14, about 16, about 18, about 20 μg of RNA) and a second dose comprises about 0.1 to about 5 μg of RNA (e.g., about 0.1, about 0.5, about 1, about 2, about 3, about 4, or about 5 μg of RNA).

In some embodiments, a first dose comprises about 10 μg of RNA and a second dose comprises about 0.1 ug of RNA. In some embodiments, a first dose comprises about 10 μg of RNA and a second dose comprises about 0.3 ug of RNA. In some embodiments, a first dose comprises about 10 μg of RNA and a second dose comprises about 1 μg of RNA. In some embodiments, a first dose comprises about 10 μg of RNA and a second dose comprises about 3 μg of RNA.

In some embodiments, a first dose comprises at least about 3 μg of RNA and a second dose comprises less than about 3 μg of RNA. In some embodiments, a first dose comprises about 3 to about 10 μg of RNA (e.g., about 3, about 4, about 5, about 6, or about 7, about 8, about 9, or about 10 μg of RNA) and a second dose comprises 0.1 to less than about 3 μg of RNA (e.g., about 0.1, about 0.2, about 0.3, about 0.5, about 0.6, about 0.7, about 0.8, about 0.9, about 1.0, about 1.5 about 2.0, or about 2.5 ug of RNA). In some embodiments, a first dose comprises about 3 to about 10 μg of RNA and a second dose comprises about 0.1 to about 3 μg of RNA, about 0.1 to about 1 μg of RNA, or about 0.1 to about 0.5 ug of RNA.

In some embodiments, a first dose comprises about 1 to about 3 μg of RNA (e.g., about 1, about 1.5, about 2.0, about 2.5, or about 3.0 ug of RNA) and a second dose comprises about 0.1 to 0.3 ug of RNA (e.g., about 0.1, about 0.2, about 0.3, about 0.4, about 0.5, about 0.6, about 0.7, about 0.8, about 0.9, or about 1.0 ug of RNA).

In some embodiments, a first dose comprises about 3 μg of RNA and a second dose comprises about 0.1 ug of RNA. In some embodiments, a first dose comprises about 3 μg of RNA and a second dose comprises about 0.3 ug of RNA. In some embodiments, a first dose comprises about 3 μg of RNA and a second dose comprises about 0.6 ug of RNA. In some embodiments, a first dose comprises about 3 μg of RNA and a second dose comprises about 1 μg of RNA.

In some embodiments, a vaccination regimen comprises at least two doses, including, e.g., at least three doses, at least four doses or more. In some embodiments, a vaccination regimen comprises three doses. In some embodiments, the time interval between the first dose and the second dose can be the same as the time interval between the second dose and the third dose. In some embodiments, the time interval between the first dose and the second dose can be longer than the time interval between the second dose and the third dose, e.g., by days or weeks (including, e.g., at least 3 days, at least 4 days, at least 5 days, at least 6 days, at least 1 week, at least 2 weeks, at least 3 weeks, at least 4 weeks, at least 5 weeks, at least 6 weeks, or longer). In some embodiments, the time interval between the first dose and the second dose can be shorter than the time interval between the second dose and the third dose, e.g., by days or weeks (including, e.g., at least 3 days, at least 4 days, at least 5 days, at least 6 days, at least 1 week, at least 2 weeks, at least 3 weeks, at least 4 weeks, at least 5 weeks, at least 6 weeks, or longer). In some embodiments, the time interval between the first dose and the second dose can be shorter than the time interval between the second dose and the third dose, e.g., by at least 1 month (including, e.g., at least 2 months, at least 3 months, at least 4 months, at least 5 months, at least 6 months, at least 7 months, at least 8 months, at least 9 months, at least 10 months, at least 11 months, at least 12 months, or longer).

In some embodiments, a last dose of a primary regimen and a first dose of a booster regimen are given at least 2 months, at least 3 months, at least 4 months, at least 5 months, at least 6 months, at least 7 months, at least 8 months, at least 9 months, at least 10 months, at least 11 months, at least 12 months, or more apart. In some embodiments, a primary regimen may comprises two doses. In some embodiments, a primary regimen may comprises three doses.

In some embodiments, a first dose and a second dose (and/or other subsequent dose) may be administered by intramuscular injection. In some embodiments, a first dose and a second dose (and/or other subsequent dose) may be administered in the deltoid muscle. In some embodiments, a first dose and a second dose (and/or other subsequent dose) may be administered in the same arm.

In some embodiments, an mRNA composition described herein is administered (e.g., by intramuscular injection) as a series of two doses (e.g., 0.3 mL each) 21 days apart. In some embodiments, an mRNA composition described herein is administered (e.g., by intramuscular injection) as a series of two doses (e.g., 0.2 mL each) 21 days apart. In some embodiments, an mRNA composition described herein is administered (e.g., by intramuscular injection) as a series of three doses (e.g., 0.3 mL or lower including, e.g., 0.2 mL), wherein doses are given at least 3 weeks apart. In some embodiments, the first and second doses may be administered 3 weeks apart, while the second and third doses may be administered at a longer time interval than that between the first and the second doses, e.g., at least 4 weeks apart or longer (including, at least 5 weeks, at least 6 weeks, at least 7 weeks, at least 8 weeks, at least 9 weeks, or longer). In some embodiments, each dose is about 60 ug. In some embodiments, each dose is about 50 ug. In some embodiments, each dose is about 30 ug. In some embodiments, each dose is about 25 ug. In some embodiments, each dose is about 20 ug. In some embodiments, each dose is about 15 ug. In some embodiments, each dose is about 10 ug. In some embodiments, each dose is about 3 ug.

In some embodiments, at least one dose given in a vaccination regimen (e.g., a primary vaccination regimen and/or a booster vaccination regimen) is about 60 ug. In some embodiments, at least one dose given in a vaccination regimen (e.g., a primary vaccination regimen and/or a booster vaccination regimen) is about 50 ug. In some embodiments, at least one dose given in a vaccination regimen (e.g., a primary vaccination regimen and/or a booster vaccination regimen) is about 30 ug. In some embodiments, at least one dose given in a vaccination regimen (e.g., a primary vaccination regimen and/or a booster vaccination regimen) is about 25 ug. In some embodiments, at least one dose given in a vaccination regimen (e.g., a primary vaccination regimen and/or a booster vaccination regimen) is about 20 ug. In some embodiments, at least one dose given in a vaccination regimen (e.g., a primary vaccination regimen and/or a booster vaccination regimen) is about 15 ug. In some embodiments, at least one dose given in a vaccination regimen (e.g., a primary vaccination regimen and/or a booster vaccination regimen) is about 10 ug. In some embodiments, at least one dose given in a vaccination regimen (e.g., a primary vaccination regimen and/or a booster vaccination regimen) is about 3 ug.

In one embodiment, an amount of the RNA described herein of about 60 μg is administered per dose. In one embodiment, an amount of the RNA described herein of about 50 μg is administered per dose. In one embodiment, an amount of the RNA described herein of about 30 μg is administered per dose. In one embodiment, an amount of the RNA described herein of about 25 μg is administered per dose. In one embodiment, an amount of the RNA described herein of about 20 μg is administered per dose. In one embodiment, an amount of the RNA described herein of about 15 μg is administered per dose. In one embodiment, an amount of the RNA described herein of about 10 μg is administered per dose. In one embodiment, an amount of the RNA described herein of about 5 μg is administered per dose. In one embodiment, an amount of the RNA described herein of about 3 μg is administered per dose. In one embodiment, at least two of such doses are administered. For example, a second dose may be administered about 21 days following administration of the first dose.

In some embodiments, the efficacy of the RNA vaccine described herein (e.g., administered in two doses, wherein a second dose may be administered about 21 days following administration of the first dose, and administered, for example, in an amount of about 30 μg per dose) is at least 70%, at least 80%, at least 90, or at least 95% beginning 7 days after administration of the second dose (e.g., beginning 28 days after administration of the first dose if a second dose is administered 21 days following administration of the first dose). In some embodiments, such efficacy is observed in populations of age of at least 50, at least 55, at least 60, at least 65, at least 70, or older. In some embodiments, the efficacy of the RNA vaccine described herein (e.g., administered in two doses, wherein a second dose may be administered about 21 days following administration of the first dose, and administered, for example, in an amount of about 30 μg per dose) beginning 7 days after administration of the second dose (e.g., beginning 28 days after administration of the first dose if a second dose is administered 21 days following administration of the first dose) in populations of age of at least 65, such as 65 to 80, 65 to 75, or 65 to 70, is at least 90%, at least 91%, at least 92%, at least 93%, at least 94%, or at least 95%. Such efficacy may be observed over time periods of up to 1 month, 2 months, 3 months, 6 months or even longer.

In one embodiment, vaccine efficacy is defined as the percent reduction in the number of subjects with evidence of infection (vaccinated subjects vs. non-vaccinated subjects). In one embodiment, methods and agents described herein are administered to a paediatric population. In various embodiments, the paediatric population comprises or consists of subjects under 18 years, e.g., 5 to less than 18 years of age, 12 to less than 18 years of age, 16 to less than 18 years of age, 12 to less than 16 years of age, 5 to less than 12 years of age, or 6 months to less than 12 years of age. In various embodiments, the paediatric population comprises or consists of subjects under 5 years, e.g., 2 to less than 5 years of age, 12 to less than 24 months of age, 7 to less than 12 months of age, or less than 6 months of age. In some such embodiments, an mRNA composition described herein is administered to subjects of less than 2 years old, for example, 6 months to less than 2 years old. In some such embodiments, an mRNA composition described herein is administered to subjects of less than 6 months old, for example, 1 month to less than 4 months old. In some embodiments, a dosing regimen (e.g., doses and/or dosing schedule) for a paediatric population may vary for different age groups. For example, in some embodiments, a subject 6 months through 4 years of age may be administered according to a primary regimen comprising at least three doses, in which the initial two doses are administered at least 3 weeks (including, e.g., at least 4 weeks, at least 5 weeks, at least 6 weeks, or longer) apart followed by a third dose administered at least 8 weeks (including, e.g., at least 9 weeks, at least 10 weeks, at least 11 weeks, at least 12 weeks, or longer) after the second dose. In some such embodiments, at least one dose administered is 3 ug RNA described herein. In some embodiments, a subject 5 years of age and older may be administered according to a primary regimen comprising at least two doses, in which the two doses are administered at least 3 weeks (including, e.g., at least 3 weeks, at least 4 weeks, at least 5 weeks, at least 6 weeks, or longer) apart. In some such embodiments, at least one dose administered is 10 ug RNA described herein. In some embodiments, a subject 5 years of age and older who are immunocompromised (e.g., in some embodiments subjects who have undergone solid organ transplantation, or who are diagnosed with conditions that are considered to have an equivalent of immunocompromise) may be administered according to a primary regimen comprising at least three doses, in which the initial two doses are administered at least 3 weeks (including, e.g., at least 3 weeks, at least 4 weeks, at least 5 weeks, at least 6 weeks, or longer) apart, followed by a third dose administered at least 4 weeks (including, e.g., at least 5 weeks, at least 6 weeks, at least 7 weeks, at least 8 weeks, at least 9 weeks, at least 10 weeks, at least 11 weeks, at least 12 weeks, or longer) after the second dose.

In some embodiments, an mRNA composition described herein is administered to subjects of age 12 or older and each dose is about 30 ug. In some embodiments, an mRNA composition described herein is administered to subjects of age 12 or older (including, e.g., age 18 or older) and each dose is higher than 30 ug, including, e.g., 35 ug, 40 ug, 45 ug, 50 ug, 55 ug, 60 ug, 65 ug, 70 ug, or higher. In some such embodiments, an mRNA composition described herein is administered to subjects of age 12 or older and each dose is about 60 ug. In some such embodiments, an mRNA composition described herein is administered to subjects of age 12 or older and each dose is about 50 ug. In one embodiment, the paediatric population comprises or consists of subjects 12 to less than 18 years of age including subjects 16 to less than 18 years of age and/or subjects 12 to less than 16 years of age. In this embodiment, treatments may comprise 2 vaccinations 21 days apart, wherein, in one embodiment, the vaccine is administered in an amount of 30 μg RNA per dose, e.g., by intramuscular administration. In some embodiments, higher doses are administered to older pediatric patients and adults, e.g., to patients 12 years or older, compared to younger children or infants, e.g. 2 to less than 5 years old, 6 months to less than 2 years old, or less than 6 months old. In some embodiments, higher doses are administered to children who are 2 to less than 5 years old, as compared to toddlers and/or infants, e.g., who are 6 months to less than 2 years old, or less than 6 months old.

In one embodiment, the paediatric population comprises or consists of subjects 5 to less than 18 years of age including subjects 12 to less than 18 years of age and/or subjects 5 to less than 12 years of age. In this embodiment, treatments may comprise 2 vaccinations 21 days apart, wherein, in various embodiments, the vaccine is administered in an amount of 10 μg, 20 μg, or 30 μg RNA per dose, e.g., by intramuscular administration. In some such embodiments, an mRNA composition described herein is administered to subjects of age 5 to 11 and each dose is about 10 ug.

In one embodiment, the paediatric population comprises or consists of subjects less than 5 years of age including subjects 2 to less than 5 years of age, subjects 12 to less than 24 months of age, subjects 7 to less than 12 months of age, subjects 6 to less than 12 months of age and/or subjects less than 6 months of age. In this embodiment, treatments may comprise 2 vaccinations, e.g., 21 to 42 days apart, e.g., 21 days apart, wherein, in various embodiments, the vaccine is administered in an amount of 3 μg, 10 μg, 20 μg, or 30 μg RNA per dose, e.g., by intramuscular administration. In some such embodiments, an mRNA composition described herein is administered to subjects of age 2 to less than 5 and each dose is about 3 ug. In some such embodiments, an mRNA composition described herein is administered to subjects of about 6 months to less than about 5 years and each dose is about 3 ug.

In some embodiments, an mRNA composition described herein is administered to subjects of age 12 or older and at least one dose given in a vaccination regimen (e.g., a primary vaccination regimen and/or a booster vaccination regimen) is about 60 ug. In some embodiments, an mRNA composition described herein is administered to subjects of age 12 or older and at least one dose given in a vaccination regimen (e.g., a primary vaccination regimen and/or a booster vaccination regimen) is about 30 ug. In some embodiments, an mRNA composition described herein is administered to subjects of age 12 or older and at least one dose given in a vaccination regimen (e.g., a primary vaccination regimen and/or a booster vaccination regimen) is about 15 ug. In some embodiments, an mRNA composition described herein is administered to subjects of age 5 to less than 12 years of age and at least one dose given in a vaccination regimen (e.g., a primary vaccination regimen and/or a booster vaccination regimen) is about 10 ug. In some embodiments, an mRNA composition described herein is administered to subjects of age 2 to less than 5 and at least one dose given in a vaccination regimen (e.g., a primary vaccination regimen and/or a booster vaccination regimen) is about 3 ug. In some embodiments, an mRNA composition described herein is administered to subjects of 6 months to less than age 2 and at least one dose given in a vaccination regimen (e.g., a primary vaccination regimen and/or a booster vaccination regimen) is about 3 μg or lower, including, e.g., 2 ug, 1 ug, or lower). In some embodiments, an mRNA composition described herein is administered to infants of less than 6 months and at least one dose given in a vaccination regimen (e.g., a primary vaccination regimen and/or a booster vaccination regimen) is about 3 μg or lower, including, e.g., 2 ug, 1 ug, 0.5 ug, or lower).

In some embodiments, a dose administered to subjects in need thereof may comprise administration of a single mRNA composition described herein.

In some embodiments, a dose administered to subjects in need thereof may comprise administration of at least two or more (including, e.g., at least three or more) different drug products/formulations. For example, in some embodiments, at least two or more different drug products/formulations may comprise at least two different mRNA compositions described herein (e.g., in some embodiments each comprising a different RNA construct).

In some embodiments, a subject is administered two or more RNAs (e.g., as part of either a primary regimen or a booster regimen), wherein the two or more RNAs are administered on the same day or same visit. In some embodiments, the two or more RNAs are administered in separate compositions, e.g., by administering each RNA to a separate part of the subject (e.g., by intramuscular administration to different arms of the subject or to different sites of the same arm of the subject). In some embodiments, the two or more RNAs are mixed prior to administration (e.g., mixed immediately prior to administration, e.g., by the administering practitioner). In some embodiments, the two or more RNAs are formulated together (e.g., by (a) mixing separate populations of LNPs, each population comprising a different RNA; or (b) by mixing two or more RNAs prior to LNP formulation, so that each LNP comprises two or more RNAs).

In some embodiments, a subject is administered or a composition comprises one or more first RNAs and one or more second RNAs, each in the same amount (i.e., at a 1:1 ratio).

In some embodiments, a subject is administered or a composition comprises one or more first RNAs and one or more second RNAs, each in a different amount. For example, in some embodiments, a subject is administered or a composition comprises one or more first RNAs in an amount that is 0.01 to 100 times that of one or more second RNAs (e.g., wherein the amount of the one or more first RNAs is 0.01 to 50, 0.01 to 4, 0.01 to 30, 0.01 to 25, 0.01 to 20, 0.01 to 15, 0.01 to 10, 0.01 to 9, 0.01 to 8, 0.01 to 7, 0.01 to 6, 0.01 to 5, 0.01 to 4, 0.01 to 3, 0.01 to 2, 0.01 to 1.5, 1 to 50, 1 to 4, 1 to 30, 1 to 25, 1 to 20, 1 to 15, 1 to 10, 1 to 9, 1 to 8, 1 to 7, 1 to 6, 1 to 5, 1 to 4, 1 to 3, 1 to 2, or 1 to 1.5 times that of the one or more second RNAs). In some embodiments, a subject is administered or a composition comprises one or more first RNAs and one or more second RNAs, wherein the concentration of the one or more first RNAs is 1 to 10 times that of the one or more second RNAs. In some embodiments, a subject is administered or a composition comprises one or more first RNAs and one or more second RNAs, wherein the amount of the one or more first RNAs is 1 to 5 times that of the one or more second RNAs. In some embodiments, a subject is administered or a composition comprises one or more first RNAs and one or more second RNAs, wherein the concentration of the one or more first RNAs is 1 to 3 times that of the one or more second RNAs. In some embodiments, a subject is administered or a composition comprises one or more first RNAs and one or more second RNAs, wherein the amount of the one or more first RNAs is 2 times that of the one or more second RNAs. In some embodiments, a subject is administered or a composition comprises one or more first RNAs and one or more second RNAs, wherein the concentration of the one or more first RNAs is 3 times that of the one or more second RNAs.

In some embodiments, a subject is administered or a composition comprises two first RNAs, each encoding an antigen derived from an influenza strain or variant, wherein the amount of each RNA is not the same. For example, in some embodiments, the ratio of the two first RNAs is 1:0.01-100 (e.g., 1:0.01-50; 1:0.01-40; 1:0.01-30; 1:0.01-25; 1:0.01-20; 1:0.01-15; 1:0.01-10; 1:0.01-9; 1:0.01-8; 1:0.01-7; 1:0.01-6; 1:0.01-5; 1:0.01-4; 1:0.01-3; 1:0.01-2; 1:0.01-1.5, 1:0.1-10, 1:0.1-5, 1:0.1-3, 1:2-10, 1:2-5, or 1: 2-3). In some embodiments, a subject is administered or a composition comprises two first RNAs at a ratio of 1:3. In some embodiments, a subject is administered or a composition comprises two first RNAs at a ratio of 1:2.

For example, in some embodiments, the ratio of the three first RNAs is 1:0.01-100:0.01-100 (e.g., 1:0.01-50:0.01-50; 1:0.01-40:0.01-40; 1:0.01-30:0.01-30; 1:0.01-25:0.01-25; 1:0.01-20:0.01-20; 1:0.01-15:0.01-15; 1:0.01-10:0.01-10; 1:0.01-9:0.01-9; 1:0.01-8:0.01-8; 1:0.01-7:0.01-7; 1:0.01-6:0.01-6; 1:0.01-5:0.01-5; 1:0.01-4:0.01-4; 1:0.01-3:0.01-3; 1:0.01-2:0.01-2; 1:0.01-1.5:0.01-1.5; 1:0.1-10:0.1-10, 1:0.1-5:0.1-5, 1:0.1-3:0.1-3, 1:2-10: 2-10, 1:2-5: 2-5, or 1: 2-3: 2-3). In some embodiments, a subject is administered or a composition comprises three first RNAs at a ratio of 1:1:3. In some embodiments, a subject is administered or a composition comprises three first RNAs at a ratio of 1:3:3.

In some embodiments, a subject is administered or a composition comprises two or more second RNAs, one or more of which encode an HA protein of a Type A influenza virus, and one or more of which encode an HA protein of a Type B influenza virus. In some embodiments, the one or more second RNAs that encode an HA protein of a Type A influenza virus and the one or more second RNAs that encode an HA protein of a Type B influenza virus are present or are administered in the same amount (i.e., at a ratio of 1:1). In some embodiments, the one more second RNAs that encode an HA protein of a Type A influenza virus and the one or more second RNAs that encode an HA protein of a Type B influenza virus are administered in different amounts (e.g., in a ratio of between 1:10 and 10:1, or in a ratio of 1:2, 1:3, 1:4, 1:5, 2:1, 3:1, 4:1, or 5:1 (total RNA encoding an A antigen:total RNA encoding a B antigen).

In some embodiments, a subject is administered or a composition comprises two second RNAs, each encoding an HA protein of a different influenza virus type (e.g., a second RNA encoding an HA protein of a Type A influenza virus and a second RNA encoding an HA protein of a Type B influenza virus). In some embodiments, the second RNAs are administered or are present in the same amount (i.e., at a 1:1 ratio). In some embodiments, the second RNAs are administered or are present in different amounts (e.g., in a ratio of between 1:10 and 10:1, or in a ratio of 1:2, 1:3, 1:4, 1:5, 2:1, 3:1, 4:1, or 5:1 (A: B)).

In some embodiments, a subject is administered or a composition comprises three second RNAs, each encoding an HA protein of a different influenza virus subtype (e.g., an HA protein of an A/Wisconsin (H1N1) virus, an A/Darwin (H3N2) virus, and a B/Austria (Victoria) virus). In some embodiments, a subject is administered or a composition comprises each of the three second RNAs in the same amount (i.e., at a 1:1:1 ratio). In some embodiments, a subject is administered or a composition comprises a different amount of one or more of the three second RNAs (e.g., in a ratio of between 1:1:2 and 1:1:10 (e.g., in a ratio of 1:1:2, 1:1:3, 1:1:4, or 1:1:5), or in a ratio of between 2:2:1 and 2:2:10, (e.g., in a ratio of 2:2:1, 3:3:1, 4:4:1, or 5:5:1). In some embodiments, a subject is administered or a composition comprises three second RNAs, two of which encode HA proteins of different influenza type A virus, and one of which encodes an HA protein of an influenza type B virus. In some such embodiments, the second RNA encoding an HA protein of an influenza type B virus is present or is administered in a higher amount as compared to either second RNA encoding an HA protein from a type A virus (e.g., in some embodiments, the ratios of the two second RNAs encoding HA proteins from type A influenza viruses relative to the second RNA encoding an HA protein from a type B influenza virus is 1:1:1-10, 1:1:2, 1:1:3, 1:1:4, or 1:1:5 (A:A:B)). In some embodiments, a subject is administered or a composition comprises three second RNAs, two encoding an HA protein of an influenza type A virus and one encoding an HA protein of an influenza type B virus, wherein the ratio of the three second RNAs 1:1:4 (A:A:B). In some embodiments, the two second RNAs encoding an HA protein of an influenza type A viruses are each present or are each administered in a higher amount as compared to the second RNA encoding an HA protein from a type B virus (e.g., in some embodiments, the ratios of the two second RNAs encoding HA proteins from type A influenza viruses relative to the second RNA encoding an HA protein from a type B influenza virus is 1-10:1-10:1, 2:2:1, 3:3:1, 4:4:1, or 5:5:1 (A:A:B)).

In some embodiments, a subject is administered or a composition comprises four second RNAs, each encoding an HA protein of a different influenza virus subtype. In some such embodiments, the four second RNAs comprise two second RNAs encoding HA proteins of different influenza type A viruses and two second RNAs encoding HA proteins of different influenza type B virus (e.g., an HA protein of an H1N1 virus, an HA protein of an H3N2 virus, an HA protein of a B/Victoria lineage virus, and an HA protein of a B/Yamagata lineage virus). In some embodiments, each of the two second RNAs encoding an HA protein of an influenza type A virus and each of the two second RNAs encoding an HA protein of an influenza type B virus are present in the same amount (i.e., the ratio of the four second RNAs is 1:1:1:1). In some embodiments, the two second RNAs encoding an HA protein of an influenza type B virus are each administered or are each present in a higher amount as compared to either second RNA encoding an HA protein from a type A virus (e.g., in some embodiments, the ratios of the two second RNAs encoding HA proteins from type A influenza viruses relative to the two second RNAs encoding an HA protein from a type B influenza virus is 1:1:2-10:2-10, 1:1:2-5:2-5, 1:1:2:2, 1:1:3:3, 1:1:4:4, 1:1:5:5, 1:1:6:6, 1:1:7:7, 1:1:8:8, 1:1:9:9, 1:1:10:10 (A:A:B:B)). In some embodiments, a subject is administered or a composition comprises four second RNAs, two encoding an HA protein of an influenza type A virus and two encoding an HA protein of an influenza type B virus, wherein the ratio of the four second RNAs 1:1:5:5 (A:A:B:B). In some embodiments, the two second RNAs encoding an HA protein of an influenza type A virus are each administered or are each present in a higher amount as compared to either second RNA encoding an HA protein from a type B virus (e.g., in some embodiments, the ratios of the two second RNAs encoding HA proteins from type A influenza viruses relative to the two second RNAs encoding an HA protein from a type B influenza virus is 2-10:2-10:1:1, 2-5:2-5:1:1, 2:2:1:1, 3:3:1:1, 4:4:1:1, 5:5:1:1, 6:6:1:1, 7:7:1:1, 8:8:1:1, 9:9:1:1, 10:10:1:1 (A:A:B:B)).

In some embodiments, a composition comprises or a subject is administered four second RNAs, comprising three second RNAs that encode HA proteins of different influenza type A viruses and one second RNA encoding an HA protein of an influenza type B virus (e.g., A/Wisconsin (H1N1), A/Darwin (H3N2), A/Cambodia (H3N2), and B/Austria (Victoria)). In some such embodiments, each of the four second RNAs is administered or is present in the same amount (i.e., at a 1:1:1:1 ratio). In some embodiments, the amount of second RNA encoding an HA protein of an influenza type B virus is higher than any one of the second RNAs encoding an HA protein of an influenza type A virus (e.g., in some embodiments, the ratio of second RNAs is 1:1:1:1-10, 1:1:1:1-5,1:1:1:2, 1:1:1:3, 1:1:1:4, or 1:1:1:5 (A:A:A:B)). In some embodiments, the ratio of second RNAs administered or in a composition is 1:1:1:5 (A:A:A:B). In some embodiments, the amount of each of the second RNAs encoding an HA protein of an influenza type A virus is higher than that of the second RNA encoding an HA protein of an influenza type B virus (e.g., in some embodiments, the ratio of second RNAs is 1-10:1-10:1-10:1, 1-5:1-5:1-5:1, 2:2:2:1, 3:3:3:1, 4:4:4:1, or 5:5:5:1 (A:A:A:B)).

In some embodiments, a subject is administered or a composition comprises one or more second RNAs encoding an HA protein of an influenza virus (e.g., two second RNAs, three second RNAs, or four second RNAs, each encoding an HA protein of a different influenza virus) in a total amount of 0.1 to 100 μg (e.g., 1 to 90 μg, 3 to 90 μg, 1 to 60 μg, 3 to 60 μg, 5 to 60 μg, 10 to 60 μg, 30 to 60 μg, 3 to 30 μg). In some embodiments, a subject is administered or a composition comprises one or more second RNAs encoding an HA protein of an influenza virus in a total amount of 3 μg, 5 μg, 6 μg, 10 μg, 15 μg, 20 μg, 25 μg, 30 μg, 45 μg, 60 μg, 75 μg, or 90 μg.

In some embodiments, a subject is administered or a composition comprises three or four second RNAs, each encoding an HA antigen of a different influenza strain, in one of the amounts listed in the below Table C (each “Influenza Component” corresponding to a second RNA encoding an HA antige (e.g., a second RNA as described herein).

TABLE C
Exemplary Amounts of Second RNAs Encoding HA Antigens

| Combination | Influenza | Influenza | Influenza | Influenza | |
|---|---|---|---|---|---|
| # | Component 1 | Component 2 | Component 3 | Component 4 | Total |
| 1 | 7.5 μg (A type) | 7.5 μg (A type) | 7.5 μg (B type) | 7.5 μg (B type) | 30 μg |
| 2 | 15 μg (A type) | 15 μg (A type) | 15 μg (B type) | 15 μg (B type) | 60 μg |
| 3 | 11.25 μg (A type) | 11.25 μg (A type) | 11.25 μg (B type) | 11.25 μg (B type) | 45 μg |
| 4 | 5 μg (A type) | 5 μg (A type) | 25 μg (B type) | 25 μg (B type) | 60 μg |
| 5 | 2.5 μg (A type) | 2.5 μg (A type) | 12.5 μg (B type) | 12.5 μg (B type) | 30 μg |
| 6 | 7.5 μg (A type) | 7.5 μg (A type) | 30 μg (B type) | — | 45 μg |
| 7 | 7.5 μg (A type) | 7.5 μg (A type) | 7.5 μg (A type) | 7.5 μg (B type) | 30 μg |

In some embodiments, a composition described herein is characterized in that it produces influenza neutralizing antibody titers that are within at least two fold of those produced by a reference vaccine for each influenza virus that it encodes antigens of (e.g., wherein the reference vaccine is a quadrivalent influenza RNA vaccine administered alone, or an approved (non-RNA) influenza vaccine).

In some embodiments, the influenza vaccine is an alphainfluenza virus, a betainfluenza virus, a gammainfluenza virus or a deltainfluenza virus vaccine. In some embodiments the vaccine is an Influenza A virus, an Influenza B virus, an Influenza C virus, or an Influenza D virus vaccine. In some embodiments, the influenza A virus vaccine comprises a hemagglutinin selected from H1, H2, H3, H4, H5, H6, H7, H8, H9, H10, H11, H12, H13, H14, H15, H16, H17, and H18, or an immunogenic fragment or variant of the same, or a nucleic acid (e.g., RNA) encoding any one of the same. In some embodiments the influenza A vaccine comprises or encodes a neuraminidase (NA) selected from N1, N2, N3, N4, N5, N6, N7, N8, N9, N10, and N11, or an immunogenic fragment or variant of the same, or a nucleic acid (e.g., RNA) encoding any one of the same. In some embodiments, the influenza vaccine comprises at least one Influenza virus hemagglutinin (HA), neuraminidase (NA), nucleoprotein (NP), matrix protein 1 (M1), matrix protein 2 (M2), non-structural protein 1 (NS1), non-structural protein 2 (NS2), nuclear export protein (NEP), polymerase acidic protein (PA), polymerase basic protein PB1, PB1-F2, and/or polymerase basic protein 2 (PB2), or an immunogenic fragment or variant thereof, or a nucleic acid (e.g., RNA) encoding any of one of the same.

The saRNA compositions may be utilized to treat and/or prevent an influenza virus of various genotypes, strains, and isolates. Some embodiments provide methods of preventing or treating influenza viral infection comprising administering to a subject any of the saRNA compositions described herein. In some embodiments, the antigen specific immune response comprises a T cell response. In some embodiments, the antigen specific immune response comprises a B cell response. In some embodiments, the antigen specific immune response comprises both a T cell response and a B cell response. In some embodiments, the method of producing an antigen specific immune response involves a single administration of the saRNA composition. In some embodiments, the saRNA composition is administered to the subject by intradermal, intramuscular injection, subcutaneous injection, intranasal inoculation, or oral administration.

In some embodiments, the RNA (e.g., saRNA) polynucleotides or portions thereof may encode one or more polypeptides or fragments thereof of an influenza strain as an antigen.

Some aspects of the disclosure are directed to a method of inducing an immune response in a subject, comprising administering to the subject in need thereof an effective amount of a composition as disclosed herein. Some aspects of the disclosure are directed to a method of vaccinating a subject, comprising administering to the subject in need thereof an effective amount of a composition as disclosed herein. Some aspects of the disclosure are directed to a method comprising administering to the subject in need thereof an effective amount of a composition as disclosed herein. In some embodiments, a composition as disclosed herein elicits an immune response comprising an antibody response. In some embodiments, a composition as disclosed herein elicits an immune response comprising a T cell response.

Some embodiments of the present disclosure provide methods of inducing an antigen specific immune response in a subject, comprising administering to the subject any of the RNA (e.g., saRNA) composition as provided herein in an amount effective to produce an antigen-specific immune response. In some embodiments, the RNA (e.g., saRNA) composition is an influenza vaccine. In some embodiments, the RNA (e.g., saRNA) composition is a combination vaccine comprising a combination of influenza vaccines (a broad spectrum influenza vaccine).

In some embodiments, an antigen-specific immune response comprises a T cell response or a B cell response. In some embodiments, a method of producing an antigen-specific immune response comprises administering to a subject a single dose (no booster dose) of an influenza RNA (e.g., saRNA) composition of the present disclosure. In some embodiments, a method further comprises administering to the subject a second (booster) dose of an influenza RNA (e.g., saRNA) composition. Additional doses of an influenza RNA (e.g., saRNA) composition may be administered.

In some embodiments, the subjects exhibit a seroconversion rate of at least 80% (e.g., at least 85%, at least 90%, or at least 95%) following the first dose or the second (booster) dose of the vaccine. Seroconversion is the time period during which a specific antibody develops and becomes detectable in the blood. After seroconversion has occurred, a virus can be detected in blood tests for the antibody. During an infection or immunization, antigens enter the blood, and the immune system begins to produce antibodies in response. Before seroconversion, the antigen itself may or may not be detectable, but antibodies are considered absent. During seroconversion, antibodies are present but not yet detectable. Any time after seroconversion, the antibodies can be detected in the blood, indicating a prior or current infection.

In some embodiments, an influenza RNA (e.g., saRNA) composition is administered to a subject by intradermal injection, intramuscular injection, or by intranasal administration. In some embodiments, an influenza RNA (e.g., saRNA) composition is administered to a subject by intramuscular injection.

Some embodiments, of the present disclosure provide methods of inducing an antigen specific immune response in a subject, including administering to a subject an influenza RNA (e.g., saRNA) composition in an effective amount to produce an antigen specific immune response in a subject. Antigen-specific immune responses in a subject may be determined, in some embodiments, by assaying for antibody titer (for titer of an antibody that binds to an influenza antigenic polypeptide) following administration to the subject of any of the influenza RNA (e.g., saRNA) compositions of the present disclosure. In some embodiments, the anti-antigenic polypeptide antibody titer produced in the subject is increased by at least 1 log relative to a control. In some embodiments, the anti-antigenic polypeptide antibody titer produced in the subject is increased by 1-3 log relative to a control.

In some embodiments, the anti-antigenic polypeptide antibody titer produced in a subject is increased at least 2 times relative to a control. In some embodiments, the anti-antigenic polypeptide antibody titer produced in the subject is increased at least 5 times relative to a control. In some embodiments, the anti-antigenic polypeptide antibody titer produced in the subject is increased at least 10 times relative to a control. In some embodiments, the anti-antigenic polypeptide antibody titer produced in the subject is increased 2-10 times relative to a control.

In some embodiments, the control is an anti-antigenic polypeptide antibody titer produced in a subject who has not been administered a RNA (e.g., saRNA) composition of the present disclosure. In some embodiments, the control is an anti-antigenic polypeptide antibody titer produced in a subject who has been administered a live attenuated or inactivated influenza, or wherein the control is an anti-antigenic polypeptide antibody titer produced in a subject who has been administered a recombinant or purified influenza protein vaccine.

In some embodiments, the RNA (e.g., saRNA) composition is formulated in an effective amount to produce an antigen specific immune response in a subject.

In some embodiments, the effective amount is a total dose of 1 μg to 1000 μg, or 1 μg to 100 μg of saRNA. In some embodiments, the effective amount is a total dose of 30 μg. In some embodiments, the effective amount is a dose of 10 μg administered to the subject a total of two times. In some embodiments, the effective amount is a dose of 10 μg administered to the subject a total of two times. In some embodiments, the effective amount is a dose of 15 μg administered to the subject a total of two times. In some embodiments, the effective amount is a dose of 30 μg administered to the subject a total of two times.

In some embodiments, the method includes administering to the subject a saRNA composition described herein at dosage of between 10 μg/kg and 400 μg/kg is administered to the subject. In some embodiments the dosage of the saRNA polynucleotide is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the saRNA composition is administered to the subject by intradermal or intramuscular injection. In some embodiments, the saRNA composition is administered to the subject on day zero. In some embodiments, a second dose of the saRNA composition is administered to the subject on day twenty-one.

In some embodiments, the subject is about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger.

In some embodiments, the subject was born full term (e.g., about 37-42 weeks). In some embodiments, the subject was born prematurely, for example, at about 36 weeks of gestation or earlier (e.g., about 36, 35, 34, 33, 32, 31, 30, 29, 28, 27, 26 or 25 weeks). For example, the subject may have been born at about 32 weeks of gestation or earlier. In some embodiments, the subject was born prematurely between about 32 weeks and about 36 weeks of gestation. In such subjects, a RNA (e.g., mRNA) vaccine may be administered later in life, for example, at the age of about 6 months to about 5 years, or older.

In some embodiments, the subject is a young adult between the ages of about 20 years and about 50 years (e.g., about 20, 25, 30, 35, 40, 45 or 50 years old).

In some embodiments, the subject is an elderly subject about 60 years old, about 70 years old, or older (e.g., about 60, 65, 70, 75, 80, 85 or 90 years old).

In some embodiments, the subject has been exposed to influenza (e.g., C. trachomatis); the subject is infected with influenza (e.g., C. trachomatis); or subject is at risk of infection by influenza (e.g., C. trachomatis).

In some embodiments, the subject has been exposed to betacoronavirus (e.g., SARS-CoV-2); the subject is infected with betacoronavirus (e.g., SARS-CoV-2); or subject is at risk of infection by betacoronavirus (e.g., SARS-CoV-2).

In some embodiments, the subject has received at least one dose of an immunogenic composition against betacoronavirus (e.g., SARS-CoV-2), e.g., selected from any one of COMIRNATY®, the Pfizer-BioNTech COVID-19 vaccine, the Moderna mRNA-1273 COVID-19 vaccine, and the Janssen COVID-19 vaccine; the subject has received at least two doses of an immunogenic composition against betacoronavirus (e.g., SARS-CoV-2); the subject is receiving at least one dose of an immunogenic composition against betacoronavirus (e.g., SARS-CoV-2), e.g., selected from any one of COMIRNATY®, the Pfizer-BioNTech COVID-19 vaccine, the Moderna mRNA-1273 COVID-19 vaccine, and the Janssen COVID-19 vaccine; or the subject is being administered an immunogenic composition against betacoronavirus (e.g., SARS-CoV-2), e.g., selected from any one of COMIRNATY®, the Pfizer-BioNTech COVID-19 vaccine, the Moderna mRNA-1273 COVID-19 vaccine, and the Janssen COVID-19 vaccine at risk of infection by betacoronavirus (e.g., SARS-CoV-2) concomitantly, simultaneously, or within 12-48 hours of any one of the immunogenic compositions against influenza disclosed herein.

In some embodiments, the subject is immunocompromised (has an impaired immune system, e.g., has an immune disorder or autoimmune disorder).

Aspects of the disclosure provide saRNA compositions comprising one or more saRNA polynucleotides having an open reading frame encoding a first antigenic polypeptide, wherein the saRNA polynucleotide is present in the formulation for in vivo administration to a host, which confers an antibody titer superior to the criterion for seroprotection for the first antigen (e.g., HA) for an acceptable percentage of human subjects. In some embodiments, the antibody titer produced by the saRNA composition of the disclosure is a neutralizing antibody titer. In some embodiments the neutralizing antibody titer is greater than a protein vaccine. In other embodiments the neutralizing antibody titer produced by the saRNA composition is greater than an adjuvanted protein vaccine. In yet other embodiments the neutralizing antibody titer produced by the saRNA compositionis 1,000-10,000, 1,200-10,000, 1,400-10,000, 1,500-10,000, 1,000-5,000, 1,000-4,000, 1,800-10,000, 2000-10,000, 2,000-5,000, 2,000-3,000, 2,000-4,000, 3,000-5,000, 3,000-4,000, or 2,000-2,500. A neutralization titer is typically expressed as the highest serum dilution required to achieve a 50% reduction in the number of plaques.

### EXAMPLES

### Example 1: Drug Product Composition

The drug product composition is an influenza modRNA drug substance targeting the Wisconsin 2021/2022 hemagglutinin.

TABLE 1
Formulation composition of the ready-to-use (RTU)
presentation of Flu vaccine drug product

| | | Concentration, |
|---|---|---|
| Components | Function | mg/mL |
| PF-07829855 Drug | Active | 0.1 |
| substance (mRNA) | | |
| ALC-0315 | Functional lipid | 1.43 |
| ALC-0159 | Functional lipid | 0.18 |
| DSPC | Structural lipid | 0.31 |
| Cholesterol | Structural lipid | 0.62 |
| Sucrose | Cryoprotectant/Tonicifier | 1.3 |
| Tris/Tromethamine | Buffer, pH 7.4 | 0.18 |
| Tris HCl | | 1.34 |
| Water for injection | Solvent/Vehicle | q.s. |

In some embodiments, the immunogenic composition comprising one lipid nanoparticle encapsulated mRNA molecule encoding HA is monovalent and has a dose selected from any one of 1 μg mRNA, 2 μg RNA, 5 μg RNA, and 20 μg RNA.

In some embodiments, the immunogenic composition comprising one lipid nanoparticle encapsulated mRNA molecule encoding HA, a second lipid nanoparticle encapsulated mRNA molecule encoding HA, a third lipid nanoparticle encapsulated mRNA molecule encoding NA, and a fourth lipid nanoparticle encapsulated mRNA molecule encoding NA, wherein the total dose is up to 20 μg RNA.

In some embodiments, the subject is aged 30-50 years.

### Example 2: Shipping and Container Closure Information

The Drug Product is shipped frozen on dry ice. The primary container closure is a 2 mL glass Type 1 vial with 13 mm stopper. The drug product should be stored at −60 to −90° C.

### Example 3: Dosage Forms

The PF-07252220 influenza modRNA immunogenic composition candidates include one of 3 different dosage forms, selected from 2 monovalent forms and one quadrivalent form, each of which incorporate different constructs of mRNA.

Four Constructs of modRNA:
Wisconsin modRNA (Wisc2019 HA)
Phuket modRNA (Phuk2013 HA)
Washington modRNA (Wash2019 HA)
Cambodia modRNA (Camb2020 HA)

Accordingly, there are 2 monovalent immunogenic compositions (also referred to herein as drug products (DPs)) and one quadrivalent immunogenic composition.
1. Monovalent including Wisconsin modRNA
2. Monovalent including Phuket modRNA
3. Quadrivalent, which includes Wisconsin modRNA, Phuket modRNA, Washington modRNA, and Cambodia modRNA

The immunogenic composition is supplied in a 2 mL glass vial sealed with a chlorobutyl rubber stopper and an aluminum seal with flip-off plastic cap (nominal volume of 0.3 mL).

### 4.2. Components of the Immunogenic Composition

The immunogenic composition includes modRNA encoding a strain-specific full length, codon-optimized HA envelope glycoprotein which is responsible for viral binding to target cells and mediating cell entry.

The immunogenic composition is a preservative-free, sterile dispersion of LNPs in aqueous cryoprotectant buffer for IM administration. The immunogenic composition is formulated at 0.1 mg/mL RNA in 10 mM Tris buffer, 300 mM sucrose, pH 7.4 as a single-dose vial with 0.5 mL/vial fill volume, and 0.3 mL nominal volume.

### 4.2.1. Drug Substance

The specific constructs (i.e., Wisconsin modRNA [Wisc2019 HA] and Phuket modRNA [Phuk2013 HA]) or constructs (quadrivalent: Wisconsin modRNA, Phuket modRNA, Washington modRNA, and Cambodia modRNA, in the drug substance (modRNA) are the only active ingredient(s) in the DP. The drug substance is formulated in 10 mM HEPES buffer, 0.1 mM EDTA at pH 7.0 and stored at 20±5° C. in HDPE bottles EVA flexible containers.

In addition to the codon-optimized sequence encoding the antigen, the RNA contains common structural elements optimized for mediating high RNA stability and translational efficiency (5′-cap, 5′UTR, 3′-UTR, poly(A)-tail; see table and sequences below). Furthermore, an intrinsic signal peptide (sec) is part of the open reading frame and is translated as an N-terminal peptide. The RNA does not contain any uridines; instead of uridine the modified N1-methylpseudouridine is used in RNA synthesis.

The specific constructs each comprise the following elements:

5′-cap analog (m2^{7,3′-O}Gppp(m₁^{2′O})ApG) for Production of RNA Containing a Cap1 Structure is Shown Below

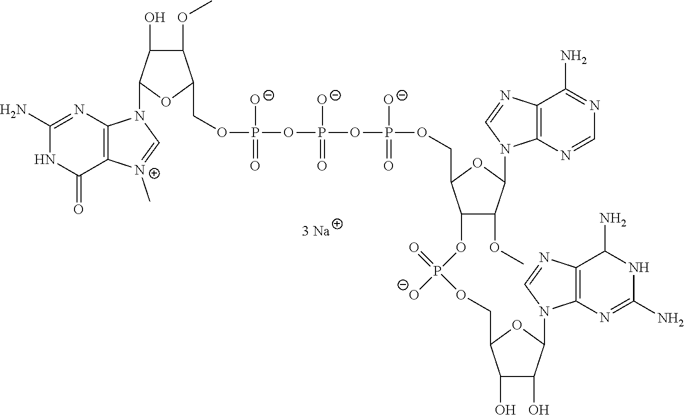 [SMILES: COC1C(COP(=O)([O-])OP(=O)([O-])OP(=O)([O-])OCC2OC(n3cnc4c(N)ncnc43)C(OC)C2OP(=O)([O-])OCC2OC(n3cnc4c3N=C(N)NC4N)C(O)C2O)OC(n2c[n+](C)c3c(=O)[nH]c(N)nc32)C1O]

The cap1 structure (i.e., containing a 2′-0-methyl group on the penultimate nucleoside of the 5′-end of the RNA chain) is incorporated into the drug substance by using a respective cap analog during in vitro transcription. For RNAs with modified uridine nucleotides, the cap1 structure is superior to other cap structures, since cap1 is not recognized by cellular factors such as IFIT1 and, thus, cap1-dependent translation is not inhibited by competition with eukaryotic translation initiation factor 4E. In the context of IFIT1 expression, mRNAs with a cap1 structure give higher protein expression levels.

TABLE 2
Table of elements

| Element | Description | Position |
|---|---|---|
| cap | A modified 5′-cap1 structure (m⁷G + m³′-5′-ppp-5′-Am) | 1-2 |
| 5′-UTR | 5′-untranslated region derived from human alpha-globin | 3-54 |
| | RNA with an optimized Kozak sequence | |
| 3′-UTR | The 3′ untranslated region comprises two sequence | 3880-4174 |
| | elements derived from the amino-terminal enhancer of | |
| | split (AES) mRNA and the mitochondrial encoded 12S | |
| | ribosomal RNA to confer RNA stability and high total | |
| | protein expression. | |
| poly(A) | A 110-nucleotide poly(A)-tail consisting of a stretch of 30 | 4175-4284 |
| | adenosine residues, followed by a 10-nucleotide linker | |
| | sequence and another 70 adenosine residues. | |

### Sequence

| GA | |
|---|---|
| (SEQ ID NO: 1) | |
| GAAΨAAAC ΨAGΨAΨΨCΨΨ CΨGGΨCCCCA CAGACΨCAGA GAGAACCCG | 50 |
| CACC | 54 |
| (SEQ ID NO: 2) | |
| C ΨCGAGCΨGGΨ ACΨGCAΨGCA | 3900 |
| CGCAAΨGCΨA GCΨGCCCCΨΨ ΨCCCGΨCCΨG GGΨACCCCGA GΨCΨCCCCCG | 3950 |
| ACCΨCGGGΨC CCAGGΨAΨGC ΨCCCACCΨCC ACCΨGCCCCA CΨCACCACCΨ | 4000 |
| CΨGCΨAGΨΨC CAGACACCΨC CCAAGCACGC AGCAAΨGCAG CΨCAAAACGC | 4050 |
| ΨΨAGCCΨAGC CACACCCCCA CGGGAAACAG CAGΨGAΨΨAA CCΨΨΨAGCAA | 4100 |
| ΨAAACGAAAG ΨΨΨAACΨAAG CΨAΨACΨAAC CCCAGGGΨΨG GΨCAAΨΨΨCG | 4150 |
| ΨGCCAGCCAC ACCCΨGGAGC ΨAGC | |
| (SEQ ID NO: 3) | |
| AAAAAA AAAAAAAAAA AAAAAAAAAA | 4200 |
| AAAAGCAΨAΨ GACΨAAAAAA AAAAAAAAAA AAAAAAAAAA AAAAAAAAAA | 4250 |
| AAAAAAAAAA AAAAAAAAAA AAAAAAAAAA AAAA | 4284 |
| Ψ = 1-methyl-3′-pseudouridylyl | |

The manufacturing process comprises RNA synthesis via in vitro transcription (IVT) step followed by DNase I and proteinase K digestion steps, purification by ultrafiltration/diafiltration (UFDF), and final filtration and dispense. A platform approach to the IVT, digestion, and purification process steps was used in the production of the four modRNA drug substances. The mRNA drug substance clinical batches were prepared at a scale of 37.6 L starting volume for IVT. The primary objective of the DNase I digestion step is to reduce the size of linear DNA template to enable subsequent removal across the ultrafiltration/diafiltration step. A DNase solution are added at the end of the final IVT incubation. Temperature and agitation rate from IVT step are maintained during this step. The primary objective of the proteinase K digestion step is to reduce the size of proteins in the reaction mixture for subsequent removal across the ultrafiltration/diafiltration step. Proteinase K solution is added to the reaction vessel and incubated for a predetermined amount of time. Temperature and agitation rate implemented during IVT and DNase digestion steps are maintained during this step. All the material is purified by a single 2-stage Ultrafiltration (UF) and diafiltration (DF) (UFDF) to produce the RNA drug substance. Flat sheet cassette membranes were used as part of the UFDF process. Preferably, the UFDF process does not utilize hollow fiber membranes. The UFDF step removes small process-related impurities and concentrates, and buffer exchanges the RNA into the final DS formulation.

Based on the retentate RNA concentration determined after diafiltration 2, the diafiltered retentate is then concentrated, if needed, and recovered through a dual-layer filter into a flexible container. The UFDF system is subsequently rinsed and added to the retentate pool through the same dual-layer filter. Formulation buffer may be added. The final pool is then filtered through a second dual-layer filter into HDPE bottle(s).

TABLE 3
Batch Results for Influenza modRNA Vaccine Wisconsin Drug Substance

| Quality | Analytical | Acceptance | | Developmental | Developmental | | Clinical Drug | |
|---|---|---|---|---|---|---|---|---|
| Attribute | Procedure | Criteria | | Material | Material | | Substance | |
| Appearance | Clarity | ≤6 | NTU | NT | ≤3 | NTU | ≤1 | NTU |

| (Clarity) | | | | | | | |
|---|---|---|---|---|---|---|---|
| Appearance | Coloration | Not more | NT | ≤B9 | | ≤B9 | |
| (Coloration) | | intensely | | | | | |
| | | colored than | | | | | |
| | | level 7 of the | | | | | |
| | | brown (B) color | | | | | |
| | | standard. | | | | | |
| pH | Potentiometry | 7.0 ± 0.5 | NT | 6.8 | | 6.8 | |
| Content | UV | 2.25 ± 0.25 | 2.40 mg/mL | 2.28 | mg/mL | 2.17 | mg/mL |

| (RNA | spectroscopy | mg/mL | | | | | |
|---|---|---|---|---|---|---|---|
| concentration) | | | | | | | |
| RT-PCR | Identity of | Identity | | NT | Identity | | Confirmed |
| | Encoded RNA | confirmed | | | confirmed | | |
| | Sequence | | | | | | |
| RNA | Capillary gel | ≥60% | | 87% | 86% | | 88% |
| integrity | electrophoresis | | | | | | |
| RP-HPLC | 5′-Cap | Report Results | | 82% | 82% | | 83% |
| Residual | qPCR | ≤500 ng | | NT | 23 | ng/mg | 112 ng |
| DNA | | DNA/mg RNA | | | | | DNA/mg RNA |
| template | | | | | | | |
| Endotoxin | Endotoxin | ≤12.5 | EU/mL | NT | 0.35 | EU/mL | NMT 1.00 EU/mL |

| | | | | | |
|---|---|---|---|---|---|
| | (LAL) | | | | |
| Bioburden | Bioburden | ≤1 CFU/10 mL | NT | 0 CFU/10 mL | 0 CFU/10 mL |

Specifications only apply to clinical supplies
Abbreviations:
NTU = nephelometric turbidity units;
NT = not tested;
ddPCR = digital droplet polymerase chain reaction;
RP-HPLC = reversed phased high performance liquid chromatography;
qPCR = quantitative polymerase chain reaction;
LAL = limulus amebocyte lysate;
EU = Endotoxin unit;
CFU = Colony forming unit

TABLE 4
Table 4 Batch Analyses for Wisconsin Clinical Drug Product

| | | | |
|---|---|---|---|
| Analytical | Quality | Acceptance | Batch |
| Procedure | Attributes | Criteria | Results |

Composition and Strength

| Appearance (Visual) | Appearance | White to off-white | | White to off-white | |
|---|---|---|---|---|---|
| | | suspension | | suspension | |
| Appearance | Appearance (Visible | May contain white to | | Meets | |
| (Particles) | Particulates) | off-white opaque, | | | |
| | | amorphous particles | | | |
| Subvisible particulate | Subvisible particles | Particles ≥10 | | 21 Particles/ | |
| matter | | μm: ≤6000 per container | | container | |
| | | Particles ≥25 | | 1 Particles/ | |
| | | μm: ≤600 per container | | container | |
| Potentiometry | pH | 7.4 ± 0.5 | | 7.4 | |
| Osmometry | Osmolality | 240-400 | mOsmol/kg | 364 | mOsm/kg |
| Dynamic Light | LNP Size | 40-120 | nm | 67 | nm |
| Scattering (DLS) | LNP Polydispersity | ≤0.3 | | 0.2 | |
| Fluorescence Assay | RNA Content | 0.074-0.126 | mg/mL | 0.104 | mg/mL |
| | RNA Encapsulation | ≥80% | | 97% | |
| HPLC-CAD | ALC-0315 Content | 0.90-1.85 | mg/mL | 1.39 | mg/mL |
| | ALC-0159 Content | 0.11-0.24 | mg/mL | 0.18 | mg/ml |
| | DSPC Content | 0.18-0.41 | mg/mL | 0.32 | mg/mL |
| | Cholesterol Content | 0.36-0.78 | mg/mL | 0.61 | mg/mL |

| | | | |
|---|---|---|---|
| Container content | Vial content (volume) | Not less than 0.30 | Not less than labeled |
| | | mL | volume |

Identity

| | | | |
|---|---|---|---|
| HPLC-CAD | Lipid identities | Retention times | Retention times |
| | | consistent with | consistent with |
| | | references (ALC- | references (ALC- |
| | | 0315, ALC-0159, | 0315, ALC-0159, |
| | | Cholesterol, DSPC) | Cholesterol, DSPC) |
| RT-PCR | Identity of encoded | Identity confirmed | Confirmed |
| | RNA sequence(s) | | |

Purity

| | | | |
|---|---|---|---|
| Capillary Gel | RNA Integrity | ≥55% intact RNA | 87% |
| Electrophoresis | | (release) | |
| | | ≥50% intact RNA | |
| | | (stability) | |

Safety

| | | | | |
|---|---|---|---|---|
| Endotoxin (LAL) | Endotoxin (LAL) | ≤12.5 | EU/mL | NMT 5.0 EU/mL |

| | | | |
|---|---|---|---|
| Sterility | Sterility | No growth detected | No growth detected |

Specifications only apply to clinical supplies
Abbreviations:
NTU = nephelometric turbidity units;
NT = not tested;
ddPCR = digital droplet polymerase chain reaction;
RP-HPLC = reversed phased high performance liquid chromatography;
qPCR = quantitative polymerase chain reaction;
LAL = limulus amebocyte lysate;
EU = Endotoxin unit;
CFU = Colony forming unit

TABLE 5
Batch Results for Influenza modRNA Vaccine Phuket Drug Substance

| Quality | Analytical | Acceptance | | Developmental | Clinical Drug | |
|---|---|---|---|---|---|---|
| Attribute | Procedure | Criteria | | Material | Substance | |
| Appearance | Clarity | ≤6 | NTU | NT | ≤0 | NTU |

| (Clarity) | | | | | |
|---|---|---|---|---|---|
| Appearance | Coloration | Not more | NT | ≤B9 | |
| (Coloration) | | intensely colored | | | |
| | | than level 7 of the | | | |
| | | brown (B) color | | | |
| | | standard. | | | |
| pH | Potentiometry | 7.0 ± 0.5 | NT | 6.8 | |
| Content (RNA | UV | 2.25 ± | 2.42 mg/mL | 2.20 | mg/mL |

| concentration) | spectroscopy | 0.25 mg/mL | | | |
|---|---|---|---|---|---|
| RT-PCR | Identity of | Identity | | NT | Confirmed |
| | Encoded RNA | confirmed | | | |
| | Sequence | | | | |
| RNA integrity | Capillary gel | ≥60% | | 88% | 87% |
| | electrophoresis | | | | |
| RP-HPLC | 5′-Cap | Report Results | | 85% | 88% |
| Residual DNA | qPCR | ≤500 ng DNA/mg | | NT | 156 ng DNA/mg |
| template | | RNA | | | RNA |
| Endotoxin | Endotoxin | ≤12.5 | EU/mL | NT | NMT 1.00 EU/mL |
| | (LAL) | | | | |

| | | | | |
|---|---|---|---|---|
| Bioburden | Bioburden | ≤1 CFU/10 mL | NT | 0 CFU/10 mL |

Specifications only apply to clinical supplies
Abbreviations:
NTU = nephelometric turbidity units;
NT = not tested;
ddPCR = digital droplet polymerase chain reaction;
RP-HPLC = reversed phased high performance liquid chromatography;
qPCR = quantitative polymerase chain reaction;
LAL = limulus amebocyte lysate;
EU = Endotoxin unit;
CFU = Colony forming unit

TABLE 6
Batch Analyses for Phuket Clinical Drug Product

| | | | |
|---|---|---|---|
| Analytical | Quality | Acceptance | Batch |
| Procedure | Attributes | Criteria | Results |

Composition and Strength

| Appearance | Appearance | White to off-white | | White to off-white | |
|---|---|---|---|---|---|
| (Visual) | | suspension | | suspension | |
| Appearance | Appearance (Visible | May contain white to | | Meets | |
| (Particles) | Particulates) | off-white opaque, | | | |
| | | amorphous particles | | | |
| Subvisible | Subvisible particles | Particles ≥10 | | 46 Particles/ | |
| particulate | | μm: ≤6000 per container | | container | |
| matter | | Particles ≥25 | | <1 Particles/ | |
| | | μm: ≤600 per container | | container | |
| Potentiometry | pH | 7.4 ± 0.5 | | 7.3 | |
| Osmometry | Osmolality | 240-400 | mOsmol/kg | 360 | mOsm/kg |
| Dynamic Light | LNP Size | 40-120 | nm | 80 | nm |
| Scattering (DLS) | LNP Polydispersity | ≤0.3 | | 0.2 | |
| Fluorescence | RNA Content | 0.074-0.126 | mg/mL | 0.086 | mg/mL |
| Assay | RNA Encapsulation | ≥80% | | 94% | |
| HPLC-CAD | ALC-0315 Content | 0.90-1.85 | mg/mL | 1.39 | mg/mL |
| | ALC-0159 Content | 0.11-0.24 | mg/mL | 0.17 | mg/mL |
| | DSPC Content | 0.18-0.41 | mg/mL | 0.29 | mg/mL |
| | Cholesterol Content | 0.36-0.78 | mg/mL | 0.59 | mg/mL |

| | | | |
|---|---|---|---|
| Container | Vial content | Not less than 0.30 mL | Not less than labeled |
| content | (volume) | | volume |

Identity

| | | | |
|---|---|---|---|
| HPLC-CAD | Lipid identities | Retention times | Retention times |
| | | consistent with | consistent with |
| | | references (ALC- | references (ALC-0315, |
| | | 0315, ALC-0159, | ALC-0159, Cholesterol, |
| | | Cholesterol, DSPC) | DSPC) |
| RT-PCR | Identity of encoded | Identity confirmed | Confirmed |
| | RNA sequence(s) | | |

Purity

| | | | |
|---|---|---|---|
| Capillary Gel | RNA Integrity | ≥55% intact RNA | 85% |
| Electrophoresis | | (release) | |

Safety

| | | | | |
|---|---|---|---|---|
| Endotoxin (LAL) | Endotoxin (LAL) | ≤12.5 | EU/mL | NMT 5.0 EU/mL |

| | | | |
|---|---|---|---|
| Sterility | Sterility | No growth detected | No growth detected |

TABLE 7
Batch Results for Influenza modRNA Vaccine Cambodia Drug Substance

| Quality | Analytical | Acceptance | | Developmental | Clinical Drug | |
|---|---|---|---|---|---|---|
| Attribute | Procedure | Criteria | | Material | Substance | |
| Appearance | Clarity | ≤6 | NTU | NT | ≤1 | NTU |
| (Clarity) | | | | | | |

| Appearance | Coloration | Not more | NT | ≤B9 | |
|---|---|---|---|---|---|
| (Coloration) | | intensely colored | | | |
| | | than level 7 of the | | | |
| | | brown (B) color | | | |
| | | standard. | | | |
| pH | Potentiometry | 7.0 ± 0.5 | NT | 6.8 | |
| Content (RNA | UV | 2.25 ± 0.25 | 2.31 mg/mL | 2.18 | mg/mL |

| concentration) | spectroscopy | mg/mL | | | |
|---|---|---|---|---|---|
| ddPCR | Identity of | Identity confirmed | | NT | Confirmed |
| | Encoded RNA | | | | |
| | Sequence | | | | |
| RNA integrity | Capillary gel | ≥60% | | 90% | 75% |
| | electrophoresis | | | | |
| RP-HPLC | 5′-Cap | Report Results | | 80% | 86% |
| Residual DNA | qPCR | ≤1500 ng | | NT | 221 ng |
| template | | DNA/mg RNA | | | DNA/mg RNA |
| Endotoxin | Endotoxin | ≤12.5 | EU/mL | NT | NMT 1.00 EU/mL |

| | | | | |
|---|---|---|---|---|
| | (LAL) | | | |
| Bioburden | Bioburden | ≤1 CFU/10 mL | NT | 0 CFU/10 mL |

Specifications only apply to clinical supplies
Abbreviations:
NTU = nephelometric turbidity units;
NT = not tested;
ddPCR = digital droplet polymerase chain reaction;
RP-HPLC = reversed phased high performance liquid chromatography;
qPCR = quantitative polymerase chain reaction;
LAL = limulus amebocyte lysate;
EU = Endotoxin unit;
CFU = Colony forming unit

TABLE 8
Batch Results for Influenza modRNA Vaccine Washington Drug Substance

| Quality | Analytical | Acceptance | | Developmental | Clinical Drug | |
|---|---|---|---|---|---|---|
| Attribute | Procedure | Criteria | | Material | Substance | |
| Appearance | Clarity | ≤6 | NTU | NT | ≤1 | NTU |
| (Clarity) | | | | | | |

| Appearance | Coloration | Not more | NT | ≤B9 | |
|---|---|---|---|---|---|
| (Coloration) | | intensely colored | | | |
| | | than level 7 of the | | | |
| | | brown (B) color | | | |
| | | standard. | | | |
| pH | Potentiometry | 7.0 ± 0.5 | NT | 6.9 | |
| Content (RNA | UV | 2.25 ± 0.25 | 2.41 mg/mL | 2.22 | mg/mL |

| concentration) | spectroscopy | mg/mL | | | |
|---|---|---|---|---|---|
| RT-PCR | Identity of | Identity confirmed | | NT | Confirmed |
| | Encoded RNA | | | | |
| | Sequence | | | | |
| RNA integrity | Capillary gel | ≥60% | | 87% | 83% |
| | electrophoresis | | | | |
| RP-HPLC | 5′-Cap | Report Results | | 86% | 87% |
| Residual DNA | qPCR | ≤1500 ng | | NT | 364 ng |
| template | | DNA/mg RNA | | | DNA/mg RNA |
| Endotoxin | Endotoxin | ≤12.5 | EU/mL | NT | NMT 1.00 EU/mL |

| | | | | |
|---|---|---|---|---|
| | (LAL) | | | |
| Bioburden | Bioburden | ≤1 CFU/10 mL | NT | 0 CFU/10 mL |

Specifications only apply to clinical supplies
Abbreviations:
NTU = nephelometric turbidity units;
NT = not tested;
ddPCR = digital droplet polymerase chain reaction;
RP-HPLC = reversed phased high performance liquid chromatography;
qPCR = quantitative polymerase chain reaction;
LAL = limulus amebocyte lysate;
EU = Endotoxin unit;
CFU = Colony forming unit

The process parameters for formation and stabilization of lipid nanoparticles are summarized in Table 10.

Table 10. Process Parameters for Formation and Stabilization of LNPs

### Process Parameter Acceptable Range

Temperature of aqueous phase 15-25° C.
Temperature of organic phase 15-25° C.
Flow rate ratio of citrate buffer to diluted drug substance for preparation of aqueous phase 4:1^{a}
Flow rate ratio of LNP suspension to citrate buffer for stabilization 2:1^{a}
LNP collection vessel temperature 2-25° C.

^{a}Target set-point during LNP formation. Ratios may be calculated from input flow rates.

### Lipid Nanoparticle (LNP) Formation and Stabilization

To form the LNPs, the citrate buffer is combined in-line with the diluted drug substance in a 4:1 flowrate ratio to create the aqueous phase. The organic and aqueous phases are fed into one or more T-mixer(s) to form the LNPs. Post formation of the LNP suspension, the LNPs are stabilized via in-line dilution with citrate buffer in a 2:1 ratio of LNP suspension to citrate buffer and then collected in a vessel which is maintained at 2-25° C.

### Buffer Exchange and Concentration

To prepare for the Buffer Exchange and Concentration operation, the tangential flow filtration (TFF) membranes are flushed with Tris buffer for equilibration.

The LNPs are processed through a tangential flow filtration (TFF) unit operation where they are concentrated and then buffer exchanged with 2 diavolumes of tris buffer to remove ethanol from the suspension. The LNPs are then concentrated further and buffer exchanged with ≥8 additional diavolumes of Tris buffer.

TABLE 9
In-Process Controls During Drug Product Manufacturing

| | | Acceptance |
|---|---|---|
| Description | In-process control | criteria |
| LNP formation and | pH of citrate buffer | 4.0 ± 0.1 |
| stabilization | | |
| Buffer exchange, | pH of Tris buffer | 7.5 ± 0.2 |
| concentration and filtration | | |
| Concentration adjustment and | pH of Sucrose/Tris | 7.5 ± 0.2 |
| addition of cryoprotectant | buffer | |
| Concentration adjustment and | RNA content prior to | ≥0.133 mg/mL |
| addition of cryoprotectant | Tris addition | (Action limit) |
| Sterile filtration | Bioburden prior to | ≤2 CFU/20 mL |
| | sterile filtration | |
| Sterile filtration | Filter integrity | Pass |
| | pre-use/post-use | |
| | sterile filtration | |
| Aseptic filling | Fill weight | 0.5 mL (0.52 |
| | (measurement) | g) ± 4% |

### 4.2.2. Excipients

The excipients Tromethamine (Tris base) and Tris Hydrochloride (HCl) present in the LNP drug product are buffer components used in pharmaceuticals and suitable to achieve the desired product pH. Sucrose is also included and was selected for its stabilizing effect to enable storage as a frozen composition prior to distribution and refrigeration at point of use. The 4 lipid excipients in the immunogenic composition are both functional and structural lipids utilized as part of the modRNA platform.

### 4.3. Dosage and Administration

The immunogenic composition is diluted as needed with normal saline, either by in-vial dilution or syringe to syringe mixing, prior to administration of the monovalent compositions or combination for the bivalent compositions.

For monovalent dosing, the immunogenic composition is dosed in the range of 3.75 to 30 μg per dose with an injection volume of 0.3 mL. Except for the 30-μg dose, dilution with sterile 0.9% sodium chloride (normal saline) is required for dosing. The 4 dose levels are:
3.75 μg mRNA
7.5 μg mRNA
15 μg mRNA
30 μg mRNA

The Wisconsin immunogenic composition is also dosed as a bivalent vaccine in combination with the Phuket immunogenic composition in a total delivered volume of 0.3 mL. The proposed dosing range (total RNA) and ratios of Wisconsin (W) immunogenic composition to Phuket (P) immunogenic composition in the bivalent immunogenic composition are:
15 μg at 1W:1P(7.5 μg A+7.5 μg B)
30 μg at 1W:1P(15 μg A+15 μg B)
22.5 μg at 1W:2P (7.5 μg A+15 μg B)
18.75 μg at 1 W:4P (3.75 μg A+15 μg B)

For quadrivalent dosing, the immunogenic composition is dosed with an injection volume of 0.3 mL containing each of the 4 modRNA sequences for a total dose of up to 30 μg. No dilution is required for administration of the quadrivalent immunogenic composition Container Closure System.

The type I borosilicate glass vials meet USP <660>, Ph. Eur. 3.2.1, and JP 7.01 compendial requirements for hydrolytic resistance for Type I glass containers. The chlorobutyl elastomeric stoppers meet USP <381>, Ph. Eur. 3.2.9 and JP 7.03 compendial chemical testing requirements for elastomeric closures.

### 4.4. Storage and Transport, Label and Pack of the Drug Product

The immunogenic composition is frozen and stored at ultralow temperature (ULT) (−90° C. to 60° C.) for long-term storage.

The influenza modRNA immunogenic composition is comprised of one or more nucleoside-modified mRNAs that encode the full-length HA glycoprotein derived from seasonal human influenza strains. The modRNA is formulated with 2 functional and 2 structural lipids, which protect the modRNA from degradation and enable transfection of the modRNA into host cells after IM injection. Influenza HA is the most abundant envelope glycoprotein on the surface of influenza A and B virions.

The primary pharmacology of the influenza modRNA immunogenic composition was evaluated in nonclinical studies in vitro and in vivo. In vitro and in vivo studies demonstrated the mechanism-of-action for the influenza modRNA immunogenic composition, which is to encode influenza HA that induces an immune response characterized by both a strong functional antibody responses and a Th1-type CD4+ and an IFNg+CD8+ T-cell response. Efficient in vitro expression of the HA glycoprotein from influenza modRNA vaccines was demonstrated in cultured cells. Mouse and rat immunogenicity studies demonstrated that influenza modRNA vaccines elicited strong functional and neutralizing antibody responses and CD4+ and CD8+ T-cell responses. Immunogenicity studies in mice, benchmarked against a licensed, adjuvanted inactivated influenza vaccine, also support the potential use of a multivalent influenza modRNA immunogenic composition formulation to target 4 different influenza virus strains.

### A Lipid Nanoparticle Encapsulated RNA Immunogenic Composition Encoding the Influenza HA as a Vaccine Antigen

The influenza modRNA immunogenic composition is based on a modRNA platform technology. The single-stranded, 5′-capped modRNA contains an open reading frame encoding the HA vaccine antigen and features structural elements optimized for high efficacy of the RNA. The modRNA also contains a substitution of 1-methyl-pseudouridine for each uridine to decrease recognition of the vaccine RNA by innate immune sensors, such as TLRs 7 and 8, resulting in decreased innate immune activation and increased protein translation. The modRNA is encapsulated in a LNP for delivery into target cells. The formulation contains 2 functional lipids, ALC-0315 and ALC-0159, and 2 structural lipids DSPC (1,2-distearoyl-sn-glycero-3-phosphocholine) and cholesterol. The physicochemical properties and the structures of the 4 lipids are shown in the Table below.

TABLE 10
Lipids in the Formulation

| | Molec- | | Physical | |
|---|---|---|---|---|
| | ular | | State and | |
| | Weight | Molecular | Storage | Chemical Name (Synonyms) and |
| Lipid | [Da] | Formula | Condition | Structure |
| ALC-- | 766 | C₄₈H₉₅NO₅ | Liquid | ((4-hydroxybutyl)azanediyl)bis(hexane- |
| 0315 | | | (oil) | 6,1-diyl)bis(2-hexyldecanoate) |
| | | | −20° C. | 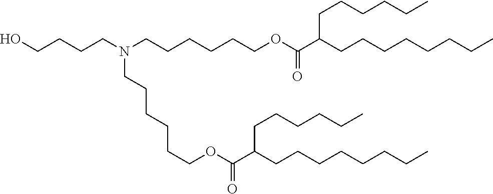 [SMILES: CCCCCCCCC(CCCCCC)C(=O)OCCCCCCN(CCCCO)CCCCCCOC(=O)C(CCCCCC)CCCCCCCC] |
| ALC- | ~2400- | C₃₀H₆₀NO | Solid | 2-[(polyethylene glycol)-2000]-N,N- |
| 0159 | 2600 | (C₂H₄O)ₙ | −20° C. | ditetradecylacetamide |
| | | OCH₃ | | 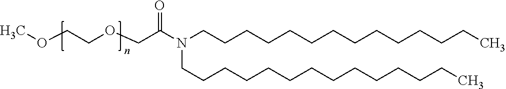 |
| DSPC | 790 | C₄₄H₈₈NO₈P | Solid | 1,2-Distearoyl-sn-glycero-3- |
| | | | −20° C. | phosphocholine |
| | | | | 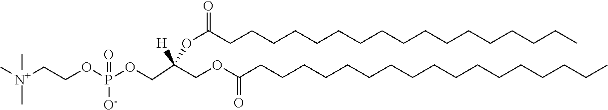 [SMILES: CCCCCCCCCCCCCCCCCC(=O)OC[C@H](COP(=O)([O-])OCC[N+](C)(C)C)OC(=O)CCCCCCCCCCCCCCCCC] |
| Choles- terol | 387 | C₂₇H₄₆O | Solid −20° C. | 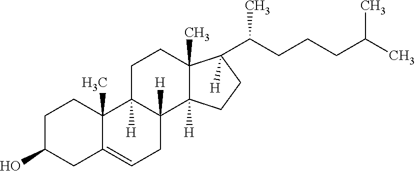 |

CAS = Chemical Abstracts Service;
DSPC = 1,2-Distearoyl-sn-glycero-3-phosphocholine

Influenza modRNA vaccine candidates selected for initial clinical testing will contain the full-length, codon-optimized coding sequence for the HA glycoprotein from the 4 cell-based virus strains recommended for use in the 2021-2022 Northern Hemisphere influenza season.
A/Wisconsin/588/2019 (H1N1)
A/Cambodia/e0826360/2020 (H3N2)
B/Phuket/3073/2013 (B Yamagata)
B/Washington/02/2019 (B Victoria)

In another embodiment, PF-07252220 (IRV) vaccine for Suspension for Injection is supplied as a white to off-white sterile frozen liquid, packaged in a 2 mL clear glass vial with a rubber stopper, aluminum overseal and flip off cap. The solution is a white to off-white opalescent liquid which may contain white to off white opaque, amorphous particles. The vial contains 0.5 mL with an extractable volume of 0.3 mL for further dilution via syringe mixing. For in-vial dilution, the vial contents (0.5 mL) should be accounted for the final dosing solution. Each vial includes the 0.1 mg/mL of PF-07252220 in a Lipid Nanoparticle (LNP) construct in 300 mM sucrose and 10 mM Tris, pH 7.4. There is no microbiological growth inhibitor in the formulation. PF-07252220 consists of five variations; four monovalent strain presentations and a quadrivalent strain presentation. The monovalent presentations may be further mixed to bivalent and quadrivalent dosing solutions at the point of use. The stability data presented below applies to all presentations and mixtures.
PF-07836259 (Phuket) Influenza mod RNA Suspension for Injection, 0.1 mg/mL
PF-07829855 (Wisconsin) Influenza mod RNA Suspension for Injection, 0.1 mg/mL
PF-07836261 (Washington) Influenza mod RNA Suspension for Injection, 0.1 mg/ml
PF-07836258 (Cambodia) Influenza mod RNA Suspension for Injection, 0.1 mg/ml
PF-07841697 Quadrivalent Influenza mod RNA Suspension for Injection, 0.1 mg/mL

The active investigational product must be stored at −90 to −60° C. (−130 to −76° F.) prior to use. Vials should be thawed at room temperature (no more than 30° C./86° F.) for approximately 30 minutes and then mixed by gently inverting the vial(s) 10 times. The investigational product will be administered intramuscularly.

TABLE 11
MONOVALENT DOSE PREPARATIONS USING 0.5 ML FILLED VOLUME
VIALS OF MONOVALENT INFLUENZA MOD RNA VACCINE

| | | | | | | | | Final Dosing | | | | Max |
|---|---|---|---|---|---|---|---|---|---|---|---|---|
| | | | | Volume | | Final | | Solution | | | | number |
| | | | | of 0.9% | | Volume of | | concentration | | Final | | of Doses |
| | Dilution | Volume of | | Sodium | | Dosing | | (in Diluted | | Injection | | per DP |
| Dose | Type | PF-07252220 | | Chloride | | solution | | Syringe/Vial) | | Volume | | vial |
| 3.75 mcg | Syringe to | 0.3 | mL | 2.1 | mL | 2.4 | mL | 12.5 | mcg/mL | 0.3 | mL | 5 |
| 7.5 | In-Vial | 0.5 | mL | 1.5 | mL | 2 | mL | 25 | mcg/mL | 0.3 | mL | 4 |
| 15 | In-Vial | 0.5 | mL | 0.5 | mL | 1 | mL | 50 | mcg/mL | 0.3 | mL | 2 |
| 30 | None | 0.5 | mL | 0 | mL | 0.5 | mL | 100 | mcg/mL | 0.3 | mL | 1 |

* Dilutions of Influenza mod RNA PF-07252220 are not limited to the preparations described in this table. The preparation instructions provided in this document are intended to support a specific clinical design, however dose preparation is not limited to these specific instruction sets. Active doses in the verified concentration range are acceptable.

TABLE 12
BIVALENT DOSE PREPARATIONS USING 0.5 ML FILLED VOLUME VIALS OF
MONOVALENT INFLUENZA MOD RNA VACCINE AND SYRINGE TO SYRINGE MIX

| | | | Volume | | | Final Dosing | |
|---|---|---|---|---|---|---|---|
| | | | of 0.9% | | Final Volume | solution | |
| | DP | Volume of | Sodium | Syringe to | of Dosing | Concentration | Final |
| Strain: | Vial | PF-07252220 | Chloride | Syringe Mix | Solution in | (total active | Injection |
| Dose (mcg) | Strain | in Vial | into Vial | (1:1) | Diluted | content) | Volume |
| 1: 7.5 mcg | 1 | 0.5 mL × | 0.5 mL | 0.3 mL | 0.6 mL | 50 mcg/mL | 0.3 mL |
| 2: 7.5 mcg | 2 | 0.5 mL × 1 vial | 0.5 mL | 0.3 mL | | | |
| 1: 15 mcg | 1 | 0.5 mL × | 0 | 0.3 mL | 0.6 mL | 100 mcg/mL | 0.3 mL |
| 2: 15 mcg | 2 | 0.5 mL × 1 vial | 0 | 0.3 mL | | | |
| 1: 30 mcg | 1 | 0.5 mL × 2 vials | 0 | 0.5 mL | 1 mL | 100 mcg/mL | 0.6 mL |
| 2: 30 mcg | 2 | 0.5 mL × 2 vials | 0 | 0.5 mL | | | |

*Bivalent doses can be made of any 2 monovalent strains, designated as Strain 1 and Strain 2

TABLE 13
BIVALENT DOSE PREPARATIONS USING 0.5 ML FILLED VOLUME VIALS
OF MONOVALENT INFLUENZA MOD RNA VACCINE AND IN-VIAL MIX
(VOLUME OF VIAL STRAIN 1 TRANSFERRED TO VIAL STRAIN 2)

| | | | Volume | In- Vial Mix: | | Final Dosing | |
|---|---|---|---|---|---|---|---|
| | | | of 0.9% | Volume of | Final Volume | solution | |
| | DP | Volume of | Sodium | Vial Strain 1 | of Dosing | Concentration | Final |
| Strain: | Vial | PF-07252220 | Chloride | transferred to | Solution in | (total active | Injection |
| Dose (mcg) | Strain | in Vial | into Vial | Vial Strain 2 | Diluted Vial | content) | Volume |
| 1: 7.5 mcg | 1 | 0.5 mL | 0.5 mL | 0.5 mL | 1 mL in | 75 mcg/mL | 0.3 mL |
| 2: 15 mcg | 2 | 0.5 mL | 0 | N/A | Vial 2 | | |
| 1: 3.75 mcg | 1 | 0.5 mL | 1.5 mL | 0.5 mL | 1 mL in | 62.5 mcg/mL | 0.3 mL |
| 2: 15 mcg | 2 | 0.5 mL | 0 | N/A | Vial 2 | | |

*Bivalent doses can be made of any 2 monovalent strains, designated as Strain 1 and Strain 2

TABLE 14
QUADRIVALENT DOSE PREPARATIONS USING 100 MCG/ML
QUADRIVALENT INFLUENZA MOD RNA VACCINE

| | | | Volume | Final Dosing | | Max |
|---|---|---|---|---|---|---|
| | Dilution | Volume of | of 0.9% | Solution | Injection | number |
| Dose | Type | PF-0725222 | Sodium | concentratio | Volum | of |
| 7.5 mcg per strain | None | 0.5 mL | N/A | 100 mcg/mL | 0.3 mL | 1 |
| 15 mcg per strain | None | 0.5 mL × 2 vials | N/A | 100 mcg/mL | 0.6 mL | 1 |

TABLE 15
QUADRIVALENT DOSE PREPARATIONS USING 0.5 ML FILLED VOLUME VIALS OF
MONOVALENT INFLUENZA MOD RNA VACCINE AND SYRINGE TO SYRINGE MIX

| | | | Step 1: | | Step 2: | Final Dosing | |
|---|---|---|---|---|---|---|---|
| | | | Volume from | | Volume from | solution | |
| | | Volume | Vial for | Volume of | Step 1 for | Concentration | |
| Strain: | DP | of PF- | syringe to | 0.9% | syringe to | (total | Final |
| Dose | Vial | 07252220 | syringe | Sodium | syringe | active | Injection |
| (mcg) | Strain | in Vial | mix | Chloride | mix | content) | Volume |
| 1: 7.5 mcg | 1 | 0.5 mL | 0.3 mL | 1.6 mL | 1.1 mL | 60 mcg/mL | 1 mL |
| 2: 7.5 mcg | 2 | 0.5 mL | 0.3 mL | | from | | |
| 3: 22.5 | | | | | syringe A | | |
| 4: 22.5 | 3 | 0.5 mL × | 0.6 mL | N/A | 0.9 mL | | |
| | | 2 vials | | | from | | |
| | 4 | 0.5 mL × | 0.6 mL | | syringe B | | |
| | | 2 vials | | | | | |
| 1: 7.5 mcg | 1 | 0.5 mL | 0.3 mL | 0.4 mL | 0.3 mL | 90 mcg/mL | 1 mL |
| 2: 7.5 mcg | 2 | 0.5 mL | 0.3 mL | | from | | |
| 3: 37.5 | | | | | syringe A | | |
| 4: 37.5 | 3 | 0.5 mL × | 0.6 mL | N/A | 0.9 mL | | |
| | | 2 vials | | | from | | |
| | 4 | 0.5 mL × | 0.6 mL | | syringe B | | |
| | | 2 vials | | | | | |

*Quadrivalent doses can be made of any 4 monovalent strains, designated as Strain 1, 2, 3, and 4

### Example 4: Nonclinical Studies

An initial mouse immunogenicity study was conducted using an influenza modRNA immunogenic composition encoding the HA sequence from A/California/07/2009 (H1N1). This HA sequence differs from the H1N1 HA antigen that will be used in the clinical study due to strain differences, but the modRNA was formulated with the same clinical LNP composition and provides supportive data for the platform.

BALB/c mice were immunized IM with 1 μg of the LNP-formulated influenza modRNA vaccine on Days 0 and 28. ELISA of sera obtained on Days 28 and 49 showed high levels of HA-binding IgG. Sera obtained as early as 14 days after the first dose had high neutralization titers against A/California/07/2009 influenza virus, and by Day 49 (21 days after the second dose) serum influenza neutralization titers exceeded 1×104. The HAI titers against A/California/07/2009 measured in sera drawn on Day 49 greatly exceeded the titer of 40 that is generally accepted as protective against influenza in humans. BALB/c mice were immunized twice IM with 1 μg of the vaccine candidate. HA-specific IgG was measured by ELISA. The functionality of the antibodies was measured by influenza virus microneutralization. IFNγ ELISpot using splenocytes harvested on Day 49 and stimulated with antigen-specific peptides showed strong CD4+ and CD8+ T-cell responses. These data confirmed that modRNA formulated with LNPs elicited Th1 phenotype T-cell responses. BALB/c mice received 2 IM immunizations with 1 μg of modRNA encoding influenza HA. The T-cell response was analyzed using antigen-specific peptides to stimulate T cells recovered from the spleen. IFNγ release was measured after peptide stimulation using an ELISpot assay.

The primary serological assay used to measure vaccine-induced immune responses to influenza is the hemagglutinin inhibition assay, or HAI. The HAI quantitatively measures functional antibodies in serum that prevent HA-mediated agglutination of red blood cells in reactions containing receptor-destroying enzyme pretreated serum samples, influenza virus and red blood cells derived from turkey or guinea pig. The HAI titer is the reciprocal of the highest serum dilution resulting in loss of HA activity, visualized as a teardrop shape when the microtiter plate is tilted. Titers from multiple determinations per sample are reported as geometric mean titers (GMT). A HAI titer of ≥1:40 is generally accepted as protective in humans. HAI assays have been developed for each of the 4 influenza strains, A/Wisconsin/588/2019 (H1N1), A/Cambodia/e0826360/2020 (H3N2), B/Phuket/3073/2013 (B Yamagata) and B/Washington/02/2019 (B Victoria).

The influenza virus microneutralization assay, or MNT, quantitatively measures functional antibodies in serum that neutralize influenza virus activity, preventing productive infection of a host cell monolayer. A neutralization reaction occurs when influenza virus is incubated with serum samples; this reaction mixture is then applied to a monolayer of Madin-Darby Canine Kidney (MDCK) cells to measure the extent of neutralization. MNT titers are reported as the reciprocal of the dilution that results in 50% reduction in infection when compared to a no serum control. MNT assays have been developed for each of the virus strains that match the HA antigens encoded by the influenza modRNA vaccine candidates.

Study to Evaluate the Feasibility of Bi-Valent modRNA HA Flu Vaccine with Pre-Mixed Drug Substance (RNA) to Form an LNP and Post-Mixed LNP Arms

As used herein unless stated otherwise, a “pre-mixed” drug substance refers to a composition wherein RNA expressing either HA or NA is mixed in a desired ratio, followed by a single formulation into an LNP. A “post-mixed” drug product refers to a composition wherein each RNA expressing either HA or NA is encapsulated in an LNP and the resulting RNA-encapsulated LNPs are then mixed in a desired ratio.

Hemagglutination-inhibition (HAI) antibody titers were examined in mice administered with a formulation as described in the following table.

### Study Design Table:

TABLE 16

| | | RNA drug product (DP, i.e., RNA | Dose | Dose Vol/ | Vax | Bleed |
|---|---|---|---|---|---|---|
| Gp# | Mice | encapsulated LNP) Description | (μg) | Route | (Day) | (Day) |
| 1 | 10 | Saline | — | 50 μl/IM | 0, 28 | 21, 42 |
| 2 | 10 | LNP modRNA HA mono-valent - Wisc | 1 | 50 μl/IM | 0, 28 | 21, 42 |
| | | Strain | | | | |
| | | 10 mM Tris and 300 mM Sucrose | | | | |
| 3 | 10 | LNP modRNA HA mono-valent - Wisc | 0.2 | 50 μl/IM | 0, 28 | 21, 42 |
| | | Strain | | | | |
| | | 10 mM Tris and 300 mM Sucrose | | | | |
| 4 | 10 | LNP modRNA HA mono-valent - Phuket | 1 | 50 μl/IM | 0, 28 | 21, 42 |
| | | Strain | | | | |
| | | 10 mM Tris and 300 mM Sucrose | | | | |
| 5 | 10 | LNP modRNA HA mono-valent - Phuket | 0.2 | 50 μl/IM | 0, 28 | 21, 42 |
| | | Strain | | | | |
| | | 10 mM Tris and 300 mM Sucrose | | | | |
| 6 | 10 | LNP modRNA HA pre-mix bi-valent, i.e., | 2 | 50 μl/IM | 0, 28 | 21, 42 |
| | | Bivalent Wisconsin and Phuket (DS mixed | | | | |
| | | prior to LNP formation) | | | | |
| | | 10 mM Tris and 300 mM Sucrose | | | | |
| 7 | 10 | LNP modRNA HA pre-mix bi-valent, i.e., | 0.4 | 50 μl/IM | 0, 28 | 21, 42 |
| | | Bivalent Wisconsin and Phuket (DS mixed | | | | |
| | | prior to LNP formation) | | | | |
| | | 10 mM Tris and 300 mM Sucrose | | | | |
| 8 | 10 | LNP modRNA HA post-mix bi-valent, i.e., | 2 | 50 μl/IM | 0, 28 | 21, 42 |
| | | Bivalent Wisconsin and Phuket (LNP mix) | | | | |
| | | 10 mM Tris and 300 mM Sucrose | | | | |
| 9 | 10 | LNP modRNA HA post-mix bi-valent, i.e., | 0.4 | 50 μl/IM | 0, 28 | 21, 42 |
| | | Bivalent Wisconsin and Phuket (LNP mix) | | | | |
| | | 10 mM Tris and 300 mM Sucrose | | | | |

High HAI Titers were induced by Wisconsin HA modRNA at 3 wks post-dose 1.

Slightly Higher HAI in Bi-Valent Groups.

Higher HAI in pre-mix Formulation for Bi-valent at 0.2ug Dose. See tables 17-18 below.

TABLE 17
GMTs 3 weeks post-dose 1 (Wisconsin)

| | GMT: | | | | |
|---|---|---|---|---|---|
| | 10 | 422 | 260 | 453 | 130 |
| | Sample: | | | | |
| | Saline | bi-val. | bi-val. | bi-val. | bi-val. |
| | (Group 1) | pre-mix. | pre-mix. | post-mix. | post-mix. |
| RNA Dose | — | 2 | 0.4 | 2 | 0.4 |
| (ug) | | | | | |

TABLE 18
GMTs 3 weeks post-dose 1 (Phuket)

| | GMT: | | | | |
|---|---|---|---|---|---|
| | 10 | 25 | 13 | 28 | 10 |
| | Sample: | | | | |
| | Saline | bi-val. | bi-val. | bi-val. | bi-val. |
| | (Group 1) | pre-mix. | pre-mix. | post-mix. | post-mix. |
| RNA Dose | — | 2 | 0.4 | 2 | 0.4 |
| (ug) | | | | | |

It was also observed that 50% Neutralizing Ab Titers Were Comparable Between Pre-Mix and Post-Mix Drug Product. See Tables 19-22 below

TABLE 19
at 3 weeks post-dose 1 (against Wisconsin)

| | GMT | | | | |
|---|---|---|---|---|---|
| | 165 | 14319 | 9393 | 24043 | 5221 |
| | Sample: | | | | |
| | | bi-val. | bi-val. | bi-val. | bi-val. |
| | Saline | pre-mix. | pre-mix. | post-mix. | post-mix. |
| RNA Dose (ug) | — | 2 | 0.4 | 2 | 0.4 |

TABLE 20
at 2 weeks post-dose 2 (against Wisconsin)

| | GMT | | | | |
|---|---|---|---|---|---|
| | 169 | 1286052 | 290731 | 1187870 | 278031 |
| | Sample: | | | | |
| | | bi-val. | bi-val. | bi-val. | bi-val. |
| | Saline | pre-mix. | pre-mix. | post-mix. | post-mix. |
| RNA Dose (ug) | — | 2 | 0.4 | 2 | 0.4 |

TABLE 21
at 3 weeks post-dose 1 (against Phuket)

| | GMT | | | | |
|---|---|---|---|---|---|
| | 60 | 730 | 1051 | 1089 | 265 |
| | Sample: | | | | |
| | | bi-val. | bi-val. | bi-val. | bi-val. |
| | Saline | pre-mix. | pre-mix. | post-mix. | post-mix. |
| RNA Dose (ug) | — | 2 | 0.4 | 2 | 0.4 |

TABLE 22
at 2 weeks post-dose 2 (against Phuket)

| | GMT | | | | |
|---|---|---|---|---|---|
| | 103 | 31818 | 6800 | 29035 | 8186 |
| | Sample: | | | | |
| | | bi-val. | bi-val. | bi-val. | bi-val. |
| | Saline | pre-mix. | pre-mix. | post-mix. | post-mix. |
| RNA Dose (ug) | — | 2 | 0.4 | 2 | 0.4 |

HAI Titers Were Comparable Between Pre-Mix Versus Post-Mix Drug Product. See tables 23-26 below.

TABLE 23
at 3 weeks post-dose 1 (against Wisconsin)

| | GMT | | | | |
|---|---|---|---|---|---|
| | 10 | 422 | 260 | 453 | 130 |
| | Sample: | | | | |
| | | bi-val. | bi-val. | bi-val. | bi-val. |
| | Saline | pre-mix. | pre-mix. | post-mix. | post-mix. |
| RNA Dose (ug) | — | 2 | 0.4 | 2 | 0.4 |

TABLE 24
at 2 weeks post-dose 2 (against Wisconsin)

| | GMT | | | | |
|---|---|---|---|---|---|
| | 10 | 2986 | 2389 | 3152 | 2229 |
| | Sample: | | | | |
| | | bi-val. | bi-val. | bi-val. | bi-val. |
| | Saline | pre-mix. | pre-mix. | post-mix. | post-mix. |
| RNA Dose (ug) | — | 2 | 0.4 | 2 | 0.4 |

TABLE 25
at 3 weeks post-dose 1 (against Phuket)

| | GMT | | | | |
|---|---|---|---|---|---|
| | 10 | 25 | 13 | 28 | 10 |
| | Sample: | | | | |
| | | bi-val. | bi-val. | bi-val. | bi-val. |
| | Saline | pre-mix. | pre-mix. | post-mix. | post-mix. |
| RNA Dose (ug) | — | 2 | 0.4 | 2 | 0.4 |

TABLE 26
at 2 weeks post-dose 2 (against Phuket)

| | GMT | | | | |
|---|---|---|---|---|---|
| | 10 | 1040 | 243 | 844 | 184 |
| | Sample: | | | | |
| | | bi-val. | bi-val. | bi-val. | bi-val. |
| | Saline | pre-mix. | pre-mix. | post-mix. | post-mix. |
| RNA Dose (ug) | — | 2 | 0.4 | 2 | 0.4 |

### Example 5: Description of Quadrivalent Drug Product

The quadrivalent drug product is a preservative-free, sterile dispersion of liquid nanoparticles (LNP) in aqueous cryoprotectant buffer for intramuscular administration. The drug product is formulated at 0.1 mg/mL RNA in 10 mM Tris buffer, 300 mM sucrose, pH 7.4.

The drug product is supplied in a 2 mL glass vial sealed with a chlorobutyl rubber stopper and an aluminum seal with flip-off plastic cap (maximum nominal volume of 0.3 mL).

TABLE 27
Composition of Quadrivalent Drug Product

| | | | | | Nominal |
|---|---|---|---|---|---|
| | | | | | Amount or |
| | | | | Filled | Net |
| | | | Unit | Amount | Quantity |
| | Grade/Quality | | Formula | (Total | (Net |
| Name of Ingredient | Standard | Function | (mg/mL) | mg/vial) | mg/vial) |
| PF-07829855 Drug | In-house | Active | 0.025 | 0.013 | 0.008 |
| Substance (Wisconsin) | specification | ingredient | | | |
| PF-07836258 Drug | In-house | Active | 0.025 | 0.013 | 0.008 |
| Substance (Cambodia) | specification | ingredient | | | |
| PF-07836259 Drug | In-house | Active | 0.025 | 0.013 | 0.008 |
| Substance (Phuket) | specification | ingredient | | | |
| PF-07836261 Drug | In-house | Active | 0.025 | 0.013 | 0.008 |
| Substance | specification | ingredient | | | |
| (Washington) | | | | | |
| ALC-0315^{a} | In-house | Functional | 1.43 | 0.72 | 0.43 |
| | specification | lipid | | | |
| ALC-0159^{b} | In-house | Functional | 0.18 | 0.09 | 0.05 |
| | specification | lipid | | | |
| DSPC^{c} | In-house | Structural | 0.31 | 0.16 | 0.09 |
| | specification | lipid | | | |
| Cholesterol | Ph. Eur., NF | Structural | 0.62 | 0.31 | 0.2 |
| | | lipid | | | |
| Sucrose | Ph. Eur., NF | Cryoprotectant | 102.69 | 51.35 | 30.81 |
| Tromethamine (Tris | Ph. Eur., USP | Buffer | 0.20 | 0.10 | 0.06 |
| base) | | component | | | |
| Tris (hydroxymethyl) | In-house | Buffer | 1.32 | 0.66 | 0.40 |
| aminomethane | specification | component | | | |
| hydrochloride (Tris HCl) | | | | | |
| Water for Injection | Ph. Eur., USP, | Solvent | q.s. ^{d} to | q.s. ^{d} to | q.s. ^{d} to |
| | JP | | 1.00 mL | 0.50 mL | 0.30 mL |

^{a}ALC-0315 = ((4-hydroxybutyl)azanediyl)bis(hexane-6,1-diyl)bis(2-hexyldecanoate)
^{b}ALC-0159 = 2-[(polyethylene glycol)-2000]-N,N-ditetradecylacetamide
^{c}DSPC = 1,2-Distearoyl-sn-glycero-3-phosphocholine
^{d} q.s. is an abbreviation for quantum satis meaning as much as is sufficient.

The recommended storage temperature of the FIH drug substance is −20±5° C.

The recommended long term storage temperature of the FIH drug product is −60 to −90° C.

The drug product may be stored at 2-8° C. at Point of Use.

TABLE 28
Batch Analyses for Quadrivalent Clinical Drug Product

| | | |
|---|---|---|
| Analytical | Quality | Acceptance |
| Procedure | Attributes | Criteria |

Composition and Strength

| Appearance | Appearance | White to off-white | |
|---|---|---|---|
| (Visual) | | suspension | |
| Appearance | Appearance (Visible | May contain white to off- | |
| (Particles) | Particulates) | white opaque, | |
| | | amorphous particles | |
| Subvisible | Subvisible particles | Particles ≥10 μm: ≤6000 | |
| particulate matter | | per container | |
| | | Particles ≥25 μm: ≤600 | |
| | | per container | |
| Potentiometry | pH | 7.4 ± 0.5 | |
| Osmometry | Osmolality | 240-400 | mOsmol/kg |
| Dynamic Light | LNP Size | 40-120 | nm |
| Scattering (DLS) | LNP Polydispersity | ≤0.3 | |
| Fluorescence Assay | RNA Content | 0.074-0.126 | mg/mL |
| | RNA Encapsulation | ≥80% | |
| HPLC-CAD | ALC-0315 Content | 0.90-1.85 | mg/mL |
| | ALC-0159 Content | 0.11-0.24 | mg/mL |
| | DSPC Content | 0.18-0.41 | mg/mL |
| | Cholesterol Content | 0.36-0.78 | mg/mL |

| | | |
|---|---|---|
| Container content | Vial content (volume) | Not less than 0.30 mL |

Identity

| | | |
|---|---|---|
| HPLC-CAD | Lipid identities | Retention times |
| | | consistent with |
| | | references (ALC-0315, |
| | | ALC-0159, Cholesterol, |
| | | DSPC) |
| RT-PCR | Identity of encoded | Identity confirmed |
| | RNA sequence(s) | |

Purity

| | | |
|---|---|---|
| Capillary Gel | RNA Integrity | ≥55% intact RNA |
| Electrophoresis | | (release) |
| | | ≥50% intact RNA |
| | | (stability) |

Safety

| | | | |
|---|---|---|---|
| Endotoxin (LAL) | Endotoxin (LAL) | ≤12.5 | EU/mL |

| | | |
|---|---|---|
| Sterility | Sterility | No growth detected |
| Dye incursion | Container Closure | Pass |
| | Integrity | |

TABLE 29
Batch Analyses for Quadrivalent Clinical Drug Product

| | | |
|---|---|---|
| Analytical | Quality | Acceptance |
| Procedure | Attributes | Criteria |

Composition and Strength

| Appearance | Appearance | White to off-white | |
|---|---|---|---|
| (Visual) | | suspension | |
| Appearance | Appearance (Visible | May contain white to | |
| (Particles) | Particulates) | off-white opaque, | |
| | | amorphous particles | |
| Subvisible | Subvisible particles | Particles ≥10 μm: ≤6000 | |
| particulate matter | | per container | |
| | | Particles ≥25 μm: ≤600 | |
| | | per container | |
| Potentiometry | pH | 7.4 ± 0.5 | |
| Osmometry | Osmolality | 240-400 | mOsmol/kg |
| Dynamic Light | LNP Size | 40-120 | nm |
| Scattering (DLS) | LNP Polydispersity | ≤0.3 | |
| Fluorescence | RNA Content | 0.074-0.126 | mg/mL |
| Assay | RNA Encapsulation | ≥80% | |
| HPLC-CAD | ALC-0315 Content | 0.90-1.85 | mg/mL |
| | ALC-0159 Content | 0.11-0.24 | mg/mL |
| | DSPC Content | 0.18-0.41 | mg/mL |
| | Cholesterol Content | 0.36-0.78 | mg/mL |

| | | |
|---|---|---|
| Container content | Vial content (volume) | Not less than 0.30 mL |

Identity

| | | |
|---|---|---|
| HPLC-CAD | Lipid identities | Retention times |
| | | consistent with |
| | | references (ALC-0315, |
| | | ALC-0159, Cholesterol, |
| | | DSPC) |
| RT-PCR | Identity of encoded | Identity confirmed |
| | RNA sequence(s) | |

Purity

| | | |
|---|---|---|
| Capillary Gel | RNA Integrity | ≥55% intact RNA |
| Electrophoresis | | (release) |
| | | ≥50% intact RNA |
| | | (stability) |

Safety

| | | | |
|---|---|---|---|
| Endotoxin (LAL) | Endotoxin (LAL) | ≤12.5 | EU/mL |

| | | |
|---|---|---|
| Sterility | Sterility | No growth detected |
| Dye incursion | Container Closure | Pass |
| | Integrity | |

### Example 6: LNP Flu HA modRNA Quadrivalent Study

The following example describes a study of LNP Flu HA modRNA Quadrivalent, in which mice were administered with different LNP_Flu HA modRNA materials as detailed in the table below. Sera collected at Day 21 post prime and at Day 42 (14 days post boost) were evaluated by serology testing (HAI, and neutralization).

TABLE 30

| | RNA DP | Dose | Dose Vol/ | Vax | Bleed |
|---|---|---|---|---|---|
| Mice | Description | (μg) | Route | (Day) | (Day) |
| 10 | Saline | — | 50 μl/IM | 0, 28 | 21, 42 |
| 10 | Quadrivalent | 4 | 50 μl/IM | 0, 28 | 21, 42 |
| | (modRNAs | | | | |
| | premixed & | | | | |
| | coformulate) | | | | |
| 10 | Quadrivalent | 0.8 | 50 μl/IM | 0, 28 | 21, 42 |
| | (modRNAs | | | | |
| | premixed & | | | | |
| | coformulate) | | | | |
| 10 | Quadrivalent | 4 (1 μg | 50 μl/IM | 0, 28 | 21, 42 |
| | (LNPs made | each) | | | |
| | separately & | | | | |
| | mixed), “post- | | | | |
| | mixed” | | | | |
| 10 | Quadrivalent | 0.8 (0.2 μg | 50 μl/IM | 0, 28 | 21, 42 |
| | (LNPs made | each) | | | |
| | separately & | | | | |
| | mixed) “post- | | | | |
| | mixed” | | | | |

HAI Titers were Comparable Between Pre-mixed Versus Post-mixed Drug Product at D21, see following tables 31-35.

TABLE 31
GMTs 3 weeks post-dose 1 (Wisconsin)

| | GMT: | | | | |
|---|---|---|---|---|---|
| | 10 | 686 | 343 | 485 | 299 |
| | Sample: | | | | |
| | Saline | Pre- | Pre- | Post- | Post- |
| | (Group 1) | mixed | mixed | mixed | mixed |
| RNA Dose | — | 4 | 0.8 | 4 | 0.8 |
| (ug) | | | | | |

TABLE 32
GMTs 3 weeks post-dose 1 (Cambodia)

| | GMT: | | | | |
|---|---|---|---|---|---|
| | 10 | 686 | 343 | 485 | 299 |
| | Sample: | | | | |
| | Saline | Pre- | Pre- | Post- | Post- |
| | (Group 1) | mixed | mixed | mixed | mixed |
| RNA Dose | — | 4 | 0.8 | 4 | 0.8 |
| (ug) | | | | | |

TABLE 33
GMTs 3 weeks post-dose 1 (Cambodia)

| | GMT: | | | | |
|---|---|---|---|---|---|
| | 28 | 40 | 49 | 46 | 53 |
| | Sample: | | | | |
| | Saline | Pre- | Pre- | Post- | Post- |
| | (Group 1) | mixed | mixed | mixed | mixed |
| RNA Dose | — | 4 | 0.8 | 4 | 0.8 |
| (ug) | | | | | |

TABLE 34
GMTs 3 weeks post-dose 1 (Washington)

| | GMT: | | | | |
|---|---|---|---|---|---|
| | 14 | 26 | 21 | 30 | 23 |
| | Sample: | | | | |
| | Saline | Pre- | Pre- | Post- | Post- |
| | (Group 1) | mixed | mixed | mixed | mixed |
| RNA Dose | — | 4 | 0.8 | 4 | 0.8 |
| (ug) | | | | | |

TABLE 35
GMTs 3 weeks post-dose 1 (Phuket)

| | GMT: | | | | |
|---|---|---|---|---|---|
| | 10 | 61 | 36 | — | 53 |
| | Sample: | | | | |
| | Saline | Pre- | Pre- | Post- | Post- |
| | (Group 1) | mixed | mixed | mixed | mixed |
| RNA Dose | — | 4 | 0.8 | 4 | 0.8 |
| (ug) | | | | | |

H1N1 A/Wisconsin: Comparable 50% Neutralization Titers Between Pre-mix and Post-Mix were observed. H3N2 A/Cambodia: Comparable 50% Neutralization Titers Between Pre-mix and Post-Mix were observed. By/Phuket: Comparable 50% Neutralization Titers Between Pre-mix and Post-Mix were observed. By/Washington: Comparable 50% Neutralization Titers Between Pre-mix and Post-Mix were also observed.

### Example 7: Immunogenicity Data in Mice of a Multivalent Influenza modRNA Vaccine, Cont'd

To evaluate the feasibility of a multivalent formulation of the modRNA influenza vaccine, modRNAs encoding 4 different HA proteins and 4 different neuraminidase (NA) proteins were generated. Immune responses elicited by mice vaccinated with LNP-formulated modRNA encoding a single strain-specific HA or NA were compared to groups vaccinated with an octavalent HA/NA modRNA formulation. Octavalent formulation methods were compared by separately formulating each modRNA expressing HA or NA in LNPs and then mixing the eight LNPs together in equal ratios, or by pre-mixing the eight modRNAs followed by a single co-formulation in LNPs.

BALB/c mice were immunized IM with 2 μg of each HA and NA-expressing modRNA either as a monovalent or octavalent vaccine formulation in LNPs on Days 0 and 28. Robust antibody and T cell responses were elicited by LNP-formulated modRNA to all HA and NA components, at levels similar to or greater than the licensed vaccine comparator. Similar HAI and neutralizing responses were observed on Day 49 (21 days after the second boost) for individual HA and octavalent formulations for influenza A strains. Antibodies measured against NA showed a similar trend as HA (data not shown). Immunogenicity studies in mice, benchmarked against a licensed, adjuvanted inactivated influenza vaccine, support the potential use of a multivalent influenza modRNA vaccine formulation to target at least four different influenza virus strains. Initial immunogenicity studies in mice of an octavalent HA/NA modRNA vaccine indicated no interference for influenza A strains and exhibited antibody responses for influenza B strains in comparison to monovalent control vaccines. These initial mouse immunogenicity data support the use of a multivalent modRNA formulation. The neuraminidase inhibition assay (NAI) quantitatively measures functional antibodies in serum that prevent NA-mediated cleavage of sialic acid in an enzyme-linked lectin assay. Briefly, antibody-containing serum is incubated with influenza virus, and the mixture is transferred to a fetuin lectin-coated plate. Cleavage of sialic acid from the fetuin is monitored through a colorimetric reaction following binding of horseradish peroxidase-conjugated peanut agglutinin to explosed galactose moieties and addition of substrate. The NAI titer is the reciprocal of the highest serum dilution resulting in reduction of NA activity by 50% compared to the no serum control. Titers from multiple determinations per sample are reported as geometric mean titers (GMT).

### Example 8: ModRNA Flu Quadrivalent Feasibility Study

This study was performed to evaluate the immunogenicity of a quadrivalent modRNA vaccine candidate encoding influenza hemagglutinin (HA) from the four strains recommended for the Northern Hemisphere 21-22 season (H1N1 A/Wisconsin/588/2019, H3N2 B/Cambodia/e0826360/2020, By/Phuket/3073/2013, By/Washington/02/2019) compared to a monovalent modRNA-HA vaccine of each strain. Historically, lower titers have been induced against the less immunogenic Flu B strains when mixed in a multivalent formulation. This study investigated whether that interference can be rescued if Flu B doses in the vaccine were increased 2× or 4× relative to Flu A doses. Intramuscular immunization of Balb/c mice with LNP-formulated modRNA-HA monovalent or quadrivalent vaccines induced functional antibody responses as measured by the Hemagglutination Inhibition Assay (HAI) and 1-day Microneutralization Assay (MNT) at Day 21 (3 weeks post dose 1) and Day 42 (2 weeks post dose 2). Monovalent and quadrivalent vaccines were similarly immunogenic against the two A strains at D21, with a robust boost effect observed 2 weeks after the second dose. Interference was observed in titers elicited by the quadrivalent modRNA vaccine only against B/Washington at D21. After the second dose, a modest increase in titers was observed for the B strains. At D42, interference was observed for both B/Phuket and B/Washington. This interference was rescued at the low dose for both strains by increasing the Flu B HA concentration 2× or 4× compared to the Flu A HA. An effective quadrivalent modRNA Influenza vaccine may potentially include an adjusted Flu B dose.

The purpose of this study was to evaluate the feasibility of a quadrivalent modRNA-HA influenza vaccine. The objectives were two-fold: 1) to compare the immunogenicity of a quadrivalent vs. monovalent modRNA formulation in mice to assess levels of interference and 2) to determine if altering the dose composition of Flu B can “rescue” any interference. The influenza modRNA composition comprises up to 4 nucleoside-modified mRNAs that encodes the full-length hemagglutinin (HA) glycoprotein derived from a seasonal human influenza strain. The modRNA is formulated with two functional and two structural lipids, which protect the modRNA from degradation and enable transfection of the modRNA into host cells after intramuscular (IM) injection. The modRNA in the quadrivalent vaccine and the monovalent comparators studied herein encode HA proteins from the four strains recommended for the 2021-2022 Northern Hemisphere Influenza season. These strains are A/Wisconsin/588/2019 (H1N1)pdm09; A/Cambodia/e0826360/2020 (H3N2); B/Phuket/3073/2013 (B/Yamagata/16/88 lineage); and B/Washington/02/2019 (B/Victoria/2/87 lineage).

Balb/c mice were immunized on Days 0 and 28 with either a monovalent modRNA-HA vaccine for one of the four recommended strains or a quadrivalent composition. Quadrivalent vaccines were mixed either as modRNA drug substances then coformulated into LNPs (pre-mix) or as LNPs after formulation of each drug substance (post-mix), as described in earlier Examples. Increased relative Flu B doses (either 2× or 4× higher than Flu A doses) were tested to determine optimal dose for the less immunogenic B strains. Serum was collected 21 days post prime and 14 days post boost. Anti-HA antibodies were measured by the Hemagglutination Inhibition Assay (HAI) and 1-day Microneutralization Assay (MNT) to determine immunogenicity.

This study was designed with 17 groups as shown in Table 36, each containing a total of 10 female mice (strain of mice: Balb/c). The modRNA drug products were evaluated at 0.05 mL dose volume.

TABLE 36
Study design

| Gp | | RNA DP | Dose | Dose Vol/ | Vax | Bleed |
|---|---|---|---|---|---|---|
| # | Mice | Description | (μg) | Route | (Day) | (Day) |
| 1 | 10 | Saline | — | 50 μL/IM | 0, 28 | 21, 42 |
| 2 | 10 | A/Wisconsin (H1N1) | 1 | 50 μL/IM | 0, 28 | 21, 42 |
| | | modRNA HA monovalent | | | | |
| 3 | 10 | A/Wisconsin (H1N1) | 0.2 | 50 μL/IM | 0, 28 | 21, 42 |
| | | modRNA HA monovalent | | | | |
| 4 | 10 | A/Cambodia (H3N2) | 1 | 50 μL/IM | 0, 28 | 21, 42 |
| | | modRNA HA monovalent | | | | |
| 5 | 10 | A/Cambodia (H3N2) | 0.2 | 50 μL/IM | 0, 28 | 21, 42 |
| | | modRNA HA monovalent | | | | |
| 6 | 10 | B/Phuket (By) | 1 | 50 μL/IM | 0, 28 | 21, 42 |
| | | modRNA HA monovalent | | | | |
| 7 | 10 | B/Phuket (By) | 0.2 | 50 μL/IM | 0, 28 | 21, 42 |
| | | modRNA HA monovalent | | | | |
| 8 | 10 | B/Washington (Bv) | 1 | 50 μL/IM | 0, 28 | 21, 42 |
| | | modRNA HA monovalent | | | | |
| 9 | 10 | B/Washington (Bv) | 0.2 | 50 μL/IM | 0, 28 | 21, 42 |
| | | modRNA HA monovalent | | | | |
| 10 | 10 | Quadrivalent (modRNAs | 4 | 50 μL/IM | 0, 28 | 21, 42 |
| | | premixed & coformulate) | | | | |
| 11 | 10 | Quadrivalent (modRNAs | 0.8 | 50 μL/IM | 0, 28 | 21, 42 |
| | | premixed & coformulate) | | | | |
| 12 | 10 | Quadrivalent (LNPs made | 4 (1 μg | 50 μL/IM | 0, 28 | 21, 42 |
| | | separately & mixed) | each) | | | |
| 13 | 10 | Quadrivalent (LNPs made | 0.8 (0.2 μg | 50 μL/IM | 0, 28 | 21, 42 |
| | | separately & mixed) | each) | | | |
| 14 | 10 | Quadrivalent (LNPs made | 4 (0.66 μg | 50 μL/IM | 0, 28 | 21, 42 |
| | | separately & mixed) - | H1, H3 & | | | |
| | | 2x Flu B dose | 1.32 μg Bv, By) | | | |
| 15 | 10 | Quadrivalent (LNPs made | 1.2 (0.2 μg | 50 μL/IM | 0, 28 | 21, 42 |
| | | separately & mixed) - | H1, H3 & | | | |
| | | 2x Flu B dose | 0.4 μg Bv, By) | | | |
| 16 | 10 | Quadrivalent (LNPs made | 4 (0.4 μg | 50 μL/IM | 0, 28 | 21, 42 |
| | | separately & mixed) - | H1, H3 & | | | |
| | | 4x Flu B dose | 1.6 μg Bv, By) | | | |
| 17 | 10 | Quadrivalent (LNPs made | 2 (0.2 μg | 50 μL/IM | 0, 28 | 21, 42 |
| | | separately & mixed) - | H1, H3 & | | | |
| | | 4x Flu B dose | 0.8 μg Bv, By) | | | |

One 0.3 mL syringe was filled to 0.05 mL, and vaccine was administered via the intramuscular route for each animal. Procedure was repeated on day 28 for the booster vaccination.

TABLE 37
Test Article and Diluent

| | Test articles/ | Formulation Matrix and | Gps to | Vials #/ |
|---|---|---|---|---|
| Item # | Diluent | information | be used | Storage |
| 1 | Saline | 0.9% NaCl in water | 1 and as diluent for | 2 bottles |
| | | | groups 2-17 | RT |
| 2 | LNP HA mono-valent | 0.086 mg/mL of modRNA | 2-3 and 12-17 | 5 × 0.5 mL |
| | plasmid | LNP in 10 mM Tris/300 mM | | vials −80° C. |
| | (Wisconsin) | Sucrose, pH 7.4 | | (2) prime, (2) boost, |
| | | | | and 1 extra |
| 3 | LNP HA mono-valent | 0.117 mg/mL of modRNA | 4-5 and 12-17 | 5 × 0.5 mL |
| | plasmid | LNP in 10 mM Tris/300 mM | | vials −80° C. |
| | (Cambodia) | Sucrose, pH 7.4 | | (2) prime, (2) boost, |
| | | | | and 1 extra |
| 4 | LNP HA mono-valent | 0.093 mg/mL of modRNA | 6-7 and 12-17 | 7 × 0.5 mL |
| | plasmid | LNP in 10 mM Tris/300 mM | | vials −80° C. |
| | (Phuket) | Sucrose, pH 7.4 | | (3) prime, (3) boost, |
| | | | | and 1 extra |
| 5 | LNP HA mono-valent | 0.085 mg/mL of modRNA | 8-9 and 12-17 | 9 × 0.5 mL |
| | plasmid | LNP in 10 mM Tris/300 mM | | vials −80° C. |
| | (Washington) | Sucrose, pH 7.4 | | (4) prime, (4) boost, |
| | | | | and 1 extra |
| 6 | LNP HA quad-valent | 0.100 mg/mL of modRNA | 10 and 11 | 6 × 0.5 mL |
| | plasmid pre-mix DS | LNP in 10 mM Tris/300 mM | | vials −80° C. |
| | (Wisconsin, Phuket, | Sucrose, pH 7.4 | | (2) prime, (2) boost, |
| | Cambodia, Washington) | | | and 2 extra |

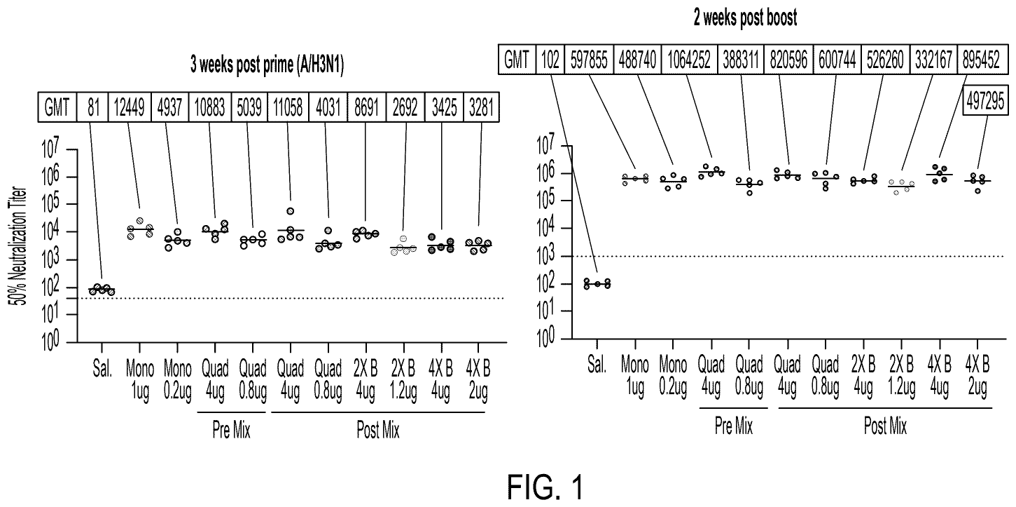

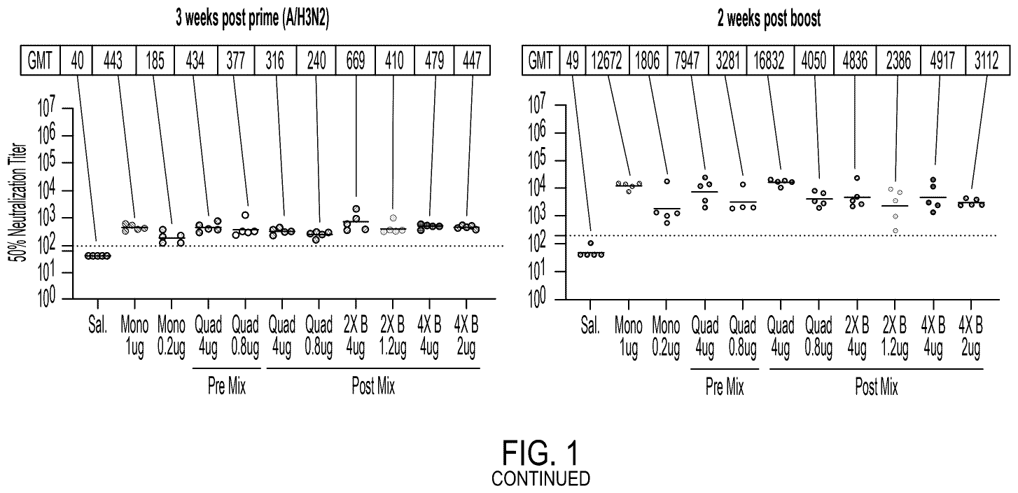

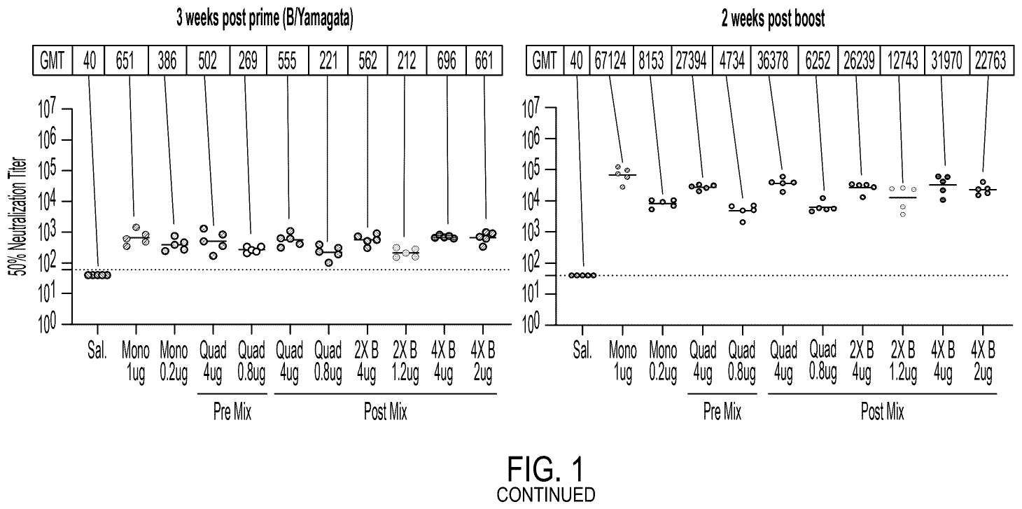

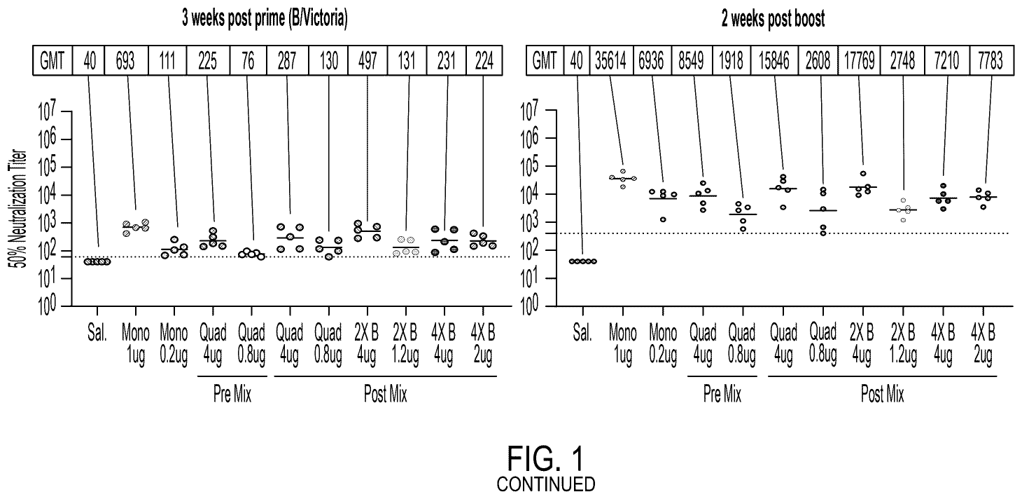

Intramuscular immunization of Balb/c mice with LNP formulated modRNA monovalent or quadrivalent vaccines encoding HA antigens from the four recommended NH 21-22 season strains (H1N1 A/Wisconsin/588/2019, H3N2 A/Cambodia/e0826360/2020, B/Phuket/3073/2013 (Yamagata), and B/Washington/02/2019 (Victoria)) induced functional antibody responses as measured by MNT (FIG. 1). HAI was performed but overall titers were low except for H1N1 A/Wisconsin, which made data interpretation challenging and therefore will not be included in this report.

MNT titers were induced by all the vaccine groups against the four strains with a robust boosting effect at Day 42. Comparable MNT titers were elicited by the quadrivalent modRNA mixes compared to each monovalent modRNA encoding H1N1 A/Wisconsin HA and H3N2 A/Cambodia HA at Day 21 and Day 42 (FIG. 1). Minimal difference in titers was observed between the pre-mix and post-mix quadrivalent formulations, although post mix formulations generally trended slightly higher at Day 42. In contrast to the Flu A strains, modest interference was observed for the Flu B strains, although the level of interference was dependent on dose level and time point. For example, MNT titers induced by the quadrivalent vaccine against B/Phuket were comparable to those elicited by the corresponding monovalent modRNA at Day 21, but trended lower at Day 42. Against B/Washington, MNT titers for the high dose (4 μg total) quadrivalent vaccines were reduced almost 3-fold compared to the 1 μg monovalent modRNA-HA control on Day 21, and slight interference for both the low and high dose groups were also observed on Day 42. Quadrivalent titers could be rescued when Flu B HA concentrations were increased 2× and 4× relative to Flu A HA, but mostly for the low dose vaccine groups.

FIG. 1—Female Balb/c mice were immunized IM on Day 0 and Day 28 with a high (1 μg or 4 μg) or low (0.2 μg or 0.8 μg) dose of either a monovalent LNP-formulated modRNA encoding HA from one of the four vaccine strains (H1N1 A/Wisconsin/588/2019, H3N2 A/Cambodia/e0826360/2020, By/Phuket/3073/2013, Bv/Washington/02/2019) or a modRNA (pre) or LNP (post) quadrivalent mix. Also tested were quadrivalent mixes with increased relative B strain concentrations at either 2× (4 μg=0.66 μg H1, H3/1.32 μg By, Bv; 1.2 μg=0.2 μg H1, H3/0.4 μg By, Bv) or 4× (4 μg=0.4 μg H1, H3/1.6 μg By, Bv; 2 μg=0.2 μg H1, H3/0.8 μg By, Bv) doses. Functional antibody responses against all four strains were measured by 1-day MNT on Day 21 (3 weeks post prime) and Day 42 (2 weeks post boost). 50% Neutralization titers are reported.

Functional antibody responses were produced against all four NH 21-22 strains (H1N1 A/Wisconsin/588/2019, H3N2 A/Cambodia/e0826360/2020, By/Phuket/3073/2013, By/Washington/02/2019) when Balb/c mice were vaccinated with a high (1 μg/4 μg) or low (0.2 μg/0.8 μg) dose of an LNP-formulated modRNA-HA monovalent or quadrivalent vaccine. Pre-mixed (modRNA) and post-mixed (LNPs) quad-valent constructs were similarly immunogenic for all four strains. At Day 21, MNT titers against H3N2 A/Cambodia, B/Phuket and B/Washington were lower than those against H1N1 A/Wisconsin, but a robust boost effect was observed for all four strains at two weeks after the second dose. Modest interference was detected in the quadrivalent titers against both B strains. However, this interference was counteracted at the low dose when concentrations of the B modRNA-HA were increased to 2 or 4 times the Flu A H1/H3 concentration. Importantly, the immunogenicity against the A strains was maintained at these modified doses. An effective quadrivalent modRNA Influenza vaccine may potentially include an adjusted Flu B dose.

### Example 9: In Vivo Mice Study PRL-Flu-Ms-2022-40

Mice aged 10-13 weeks were immunized with monovalent, bivalent, trivalent, or quadrivalent LNP_Flu modRNA-encapsulated-in-LNP compositions. Sera collected at Day 21 post prime and at Day 42 (14 days post boost). There were 10 mice per group.

### Study Design and Materials are Shown Below:

TABLE 38

| | | | Formulation Matrix | | Dose | | |
|---|---|---|---|---|---|---|---|
| | | RNA DP | and | Dose | Vol/ | Vax | Bleed |
| Gp# | Mice | Description | information | (μg) | Route | (Day) | (Day) |
| 1 | 10 | Saline | 0.9% NaCl in water | — | 50 μl/M | 0, 28 | 21, 42 |
| 2 | 10 | Monovalent | 0.123 mg/mL of | 0.2 | 50 μl/M | 0, 28 | 21, 42 |
| | | modRNA HA | modRNA LNP in 10 | | | | |
| | | (Bᵥ) (B/Austria) | mM Tris 10% Sucrose, | | | | |
| | | | pH 7.4 | | | | |
| 3 | 10 | Monovalent | 0.122 mg/mL of | 0.2 | 50 μl/M | 0, 28 | 21, 42 |
| | | modRNA HA | modRNA LNP in 10 | | | | |
| | | (B_{y}) (B/Phuket) | mM Tris 10% Sucrose, | | | | |
| | | | pH 7.4 | | | | |
| 4 | 10 | Monovalent modRNA | 0.126 mg/mL of | 0.2 | 50 μl/M | 0, 28 | 21, 42 |
| | | HA (A_{H1}) | modRNA LNP in 10 | | | | |
| | | (A/Wisconsin) | mM Tris 10% Sucrose, | | | | |
| | | | pH 7.4 | | | | |
| 5 | 10 | Monovalent modRNA | 0.114 mg/mL of | 0.2 | 50 μl/M | 0, 28 | 21, 42 |
| | | HA (A_{H1}) | modRNA LNP in 10 | | | | |
| | | (A/Sydney) | mM Tris 10% Sucrose, | | | | |
| | | | pH 7.4 | | | | |
| 6 | 10 | Monovalent modRNA | 0.118 mg/mL of | 0.2 | 50 μl/M | 0, 28 | 21, 42 |
| | | HA (A_{H3}) | modRNA LNP in 10 | | | | |
| | | (A/Darwin) | mM Tris 10% Sucrose, | | | | |
| | | | pH 7.4 | | | | |
| 7 | 10 | Monovalent modRNA | 0.140 mg/mL of | 0.2 | 50 μl/M | 0, 28 | 21, 42 |
| | | HA (A_{H3}) | modRNA LNP in 10 | | | | |
| | | (A/Cambodia) | mM Tris 10% Sucrose, | | | | |
| | | | pH 7.4 | | | | |
| 8 | 10 | Bivalent modRNA HA | 0.121 mg/mL of | 0.4 | 50 μl/M | 0, 28 | 21, 42 |
| | | (BᵥB_{y}) | modRNA LNP in 10 | (0.2 μg | | | |
| | | (B/Austria + | mM Tris 10% Sucrose, | ea) | | | |
| | | B/Phuket) | pH 7.4 | | | | |
| 9 | 10 | Bivalent modRNA HA | 0.128 mg/mL of | 0.4 | 50 μl/M | 0, 28 | 21, 42 |
| | | (BᵥA_{H1}) | modRNA LNP in 10 | (0.2 μg | | | |
| | | (B/Austria + | mM Tris 10% Sucrose, | ea) | | | |
| | | A/Wisconsin) | pH 7.4 | | | | |
| 10 | 10 | Trivalent modRNA | 0.140 mg/mL of | 0.6 | 50 μl/M | 0, 28 | 21, 42 |
| | | HA (BᵥA_{H1}A_{H3}) | modRNA LNP in 10 | (0.2 μg | | | |
| | | (B/Austria + | mM Tris 10% Sucrose, | ea) | | | |
| | | A/Wisconsin + | pH 7.4 | | | | |
| | | A/Darwin) | | | | | |
| 11 | 10 | Quadrivalent modRNA | 0.122 mg/mL of | 0.8 | 50 μl/M | 0, 28 | 21, 42 |
| | | HA (BᵥB_{y}A_{H1}A_{H3}) | modRNA LNP in 10 | (0.2 μg | | | |
| | | (B/Austria + | mM Tris 10% Sucrose, | ea) | | | |
| | | B/Phuket + | pH 7.4 | | | | |
| | | A/Wisconsin + | | | | | |
| | | A/Darwin) | | | | | |
| 12 | 10 | Quadrivalent modRNA | 0.116 mg/mL of | 0.8 | 50 μl/M | 0, 28 | 21, 42 |
| | | _{HA}(BᵥA_{H1}A_{H3}A_{H3}) | modRNA LNP in 10 | (0.2 μg | | | |
| | | (B/Austria + | mM Tris 10% Sucrose | ea) | | | |
| | | A/Wisconsin + | pH 7.4 | | | | |
| | | A/Darwin + | | | | | |
| | | A/Cambodia) | | | | | |
| 13 | 10 | Quadrivalent modRNA | 0.118 mg/mL of | 0.8 | 50 μl/M | 0, 28 | 21, 42 |
| | | _{HA}(BᵥA_{H1}A_{H1}A_{H3}) | modRNA LNP in 10 | (0.2 μg | | | |
| | | (B/Austria + | mM Tris 10% Sucrose, | ea) | | | |
| | | A/Wisconsin + | pH 7.4 | | | | |
| | | A/Sydney + A/Darwin) | | | | | |

A trivalent modRNA HA (B/Austria, A/Wisconsin, A/Darwin) 0.6 μg composition elicited an immune response in mice, wherein the composition included 0.2 μg of each of the 3 HA. The dose volume and immunization route was 50 μl/IM, administered on day 0 and 28. Bleed occurred on day 21 and 42.

Results following immunization according to Table 38 are shown below in 39 and and Table 40. Group 10 represents the trivalent modRNA HA-encapsulated LNP composition in Table39 and Table 40

TABLE 39

| Gp | RNA DP | 50% neutralization titer (10^{x}) | | | | | | | | | | | |
|---|---|---|---|---|---|---|---|---|---|---|---|---|---|
| # | Description | GMT01 | GMT02 | GMT03 | GMT04 | GMT05 | GMT06 | GMT07 | GMT08 | GMT09 | GMT10 | GMT11 | GMT12 |
| 1 | Saline | 45 | 311 | 30 | 148 | 125 | 92 | 176 | 115 | 54 | 45 | 23 | 20 |
| | 0.9% NaCl in | | | | | | | | | | | | |
| | water | | | | | | | | | | | | |
| 2 | Monovalent | 2204 | n/a | 63787 | n/a | n/a | n/a | n/a | n/a | n/a | n/a | n/a | n/a |
| | modRNA HA | | | | | | | | | | | | |
| | (Bᵥ) (B/Austria) | | | | | | | | | | | | |
| | 0.123 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 3 | Monovalent | n/a | 618 | n/a | 6972 | n/a | n/a | n/a | n/a | n/a | n/a | n/a | n/a |
| | modRNA HA | | | | | | | | | | | | |
| | (B_{y}) (B/Phuket) | | | | | | | | | | | | |
| | 0.122 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 4 | Monovalent | n/a | n/a | n/a | n/a | 1858 | n/a | 109388 | n/a | n/a | n/a | n/a | n/a |
| | modRNA HA | | | | | | | | | | | | |
| | (A_{H1}) | | | | | | | | | | | | |
| | (A/Wisconsin) | | | | | | | | | | | | |
| | 0.126 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 5 | Monovalent | n/a | n/a | n/a | n/a | n/a | 1886 | n/a | 78606 | n/a | n/a | n/a | n/a |
| | modRNA HA | | | | | | | | | | | | |
| | (A_{H1}) | | | | | | | | | | | | |
| | (A/Sydney) | | | | | | | | | | | | |
| | 0.114 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 6 | Monovalent | n/a | n/a | n/a | n/a | n/a | n/a | n/a | n/a | 1168 | n/a | 38942 | n/a |
| | modRNA HA | | | | | | | | | | | | |
| | (A_{H3}) | | | | | | | | | | | | |
| | (A/Darwin) | | | | | | | | | | | | |
| | 0.118 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 7 | Monovalent | n/a | n/a | n/a | n/a | n/a | n/a | n/a | n/a | n/a | 165 | n/a | 980 |
| | modRNA HA | | | | | | | | | | | | |
| | (A_{H3}) | | | | | | | | | | | | |
| | (A/Cambodia) | | | | | | | | | | | | |
| | 0.140 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 8 | Bivalent | 4335 | 595 | 33573 | 5279 | n/a | n/a | n/a | n/a | n/a | n/a | n/a | n/a |
| | modRNA HA | | | | | | | | | | | | |
| | (BᵥB_{y}) | | | | | | | | | | | | |
| | (B/Austria + | | | | | | | | | | | | |
| | B/Phuket) | | | | | | | | | | | | |
| | 0.121 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 9 | Bivalent | 3747 | n/a | 27236 | n/a | 3648 | n/a | 137595 | n/a | n/a | n/a | n/a | n/a |
| | modRNA HA | | | | | | | | | | | | |
| | (BᵥA_{H1}) | | | | | | | | | | | | |
| | (B/Austria + | | | | | | | | | | | | |
| | A/Wisconsin) | | | | | | | | | | | | |
| | 0.128 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 10 | Trivalent | 3378 | n/a | 27747 | n/a | 2431 | n/a | 140055 | n/a | 2019 | n/a | 42936 | n/a |
| | modRNA HA | | | | | | | | | | | | |
| | (BᵥA_{H1}A_{H3}) | | | | | | | | | | | | |
| | (B/Austria + | | | | | | | | | | | | |
| | A/Wisconsin + | | | | | | | | | | | | |
| | A/Darwin) | | | | | | | | | | | | |
| | 0.140 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 11 | Quadrivalent | 4595 | 663 | 35757 | 7540 | 3816 | n/a | 213292 | n/a | 4482 | n/a | 55402 | n/a |
| | modRNA HA | | | | | | | | | | | | |
| | (BᵥB_{y}A_{H1}A_{H3}) | | | | | | | | | | | | |
| | (B/Austria + | | | | | | | | | | | | |
| | B/Phuket + | | | | | | | | | | | | |
| | A/Wisconsin + | | | | | | | | | | | | |
| | A/Darwin) | | | | | | | | | | | | |
| | 0.122 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 12 | Quadrivalent | 3223 | n/a | 23872 | n/a | 2987 | n/a | 113132 | n/a | 4056 | 404 | 31726 | 2396 |
| | modRNA HA | | | | | | | | | | | | |
| | (BᵥA_{H1}A_{H3}A_{H3}) | | | | | | | | | | | | |
| | (B/Austria + | | | | | | | | | | | | |
| | A/Wisconsin + | | | | | | | | | | | | |
| | A/Darwin + | | | | | | | | | | | | |
| | A/Cambodia) | | | | | | | | | | | | |
| | 0.116 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 13 | Quadrivalent | 1553 | n/a | 1524 | n/a | 3371 | 2706 | 128563 | 92189 | 2172 | n/a | 44138 | n/a |
| | modRNA HA | | | | | | | | | | | | |
| | (BᵥA_{H1}A_{H1}A_{H3}) | | | | | | | | | | | | |
| | (B/Austria + | | | | | | | | | | | | |
| | A/Wisconsin + | | | | | | | | | | | | |
| | A/Sydney+ | | | | | | | | | | | | |
| | A/Darwin) | | | | | | | | | | | | |
| | 0.118 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 14 | Licensed flu | 2771 | 911 | 5374 | 4555 | 3041 | n/a | 21427 | n/a | 1964 | n/a | 24908 | n/a |
| | vaccine | | | | | | | | | | | | |

GMT 1 refers to geometric mean titers 3 weeks post dose 1 against By/Austria; GMT2 refers to geometric mean titers 3 weeks post dose 1 against By/Phuket; GMT3 refers to geometric mean titers 2 weeks post dose 2 against By/Austria; GMT4 refers to geometric mean titers 2 weeks post dose 2 against By/Phuket; GMT5 refers to geometric mean titers 3 weeks post dose 1 against H1N1 A/Wisconsin; GMT6 refers to geometric mean titers 3 weeks post dose 1 against H1N1 A/Sydney; GMT7 refers to geometric mean titers 2 weeks post dose 2 against H1N1 A/Wisconsin; GMT8 refers to geometric mean titers 2 weeks post dose 2 against H1N1 A/Sydney; GMT9 refers to geometric mean titers 3 weeks post dose 1 against H3N2 A/Darwin GMT10 refers to geometric mean titers 3 weeks post dose 1 against H3N2 A/Cambodia GMT11 refers to geometric mean titers 2 weeks post dose 2 against H3N2 A/Darwin GMT12 refers to geometric mean titers 2 weeks post dose 2 against H3N2 A/Cambodia

TABLE 40

| Gp | RNA DP | 90% neutralization titer (10^{x}) | | | | | | | | | | | |
|---|---|---|---|---|---|---|---|---|---|---|---|---|---|
| # | Description | GMT13 | GMT14 | GMT15 | GMT16 | GMT17 | GMT18 | GMT19 | GMT20 | GMT21 | GMT22 | GMT23 | GMT24 |
| 1 | Saline | 30 | 30 | 20 | 20 | 30 | 30 | 20 | 20 | 30 | 30 | 20 | 20 |
| | 0.9% NaCl in | | | | | | | | | | | | |
| | water | | | | | | | | | | | | |
| 2 | Monovalent | 446 | n/a | 11839 | n/a | n/a | n/a | n/a | n/a | n/a | n/a | n/a | n/a |
| | modRNA HA | | | | | | | | | | | | |
| | (Bᵥ) (B/Austria) | | | | | | | | | | | | |
| | 0.123 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 3 | Monovalent | n/a | 105 | n/a | 1213 | n/a | n/a | n/a | n/a | n/a | n/a | n/a | n/a |
| | modRNA HA | | | | | | | | | | | | |
| | (B_{y}) (B/Phuket) | | | | | | | | | | | | |
| | 0.122 mg/mL of | | | | | | | | | | | | |
| | modRNA LNP in | | | | | | | | | | | | |
| | 10 mM Tris | | | | | | | | | | | | |
| | 10% Sucrose, | | | | | | | | | | | | |
| | pH 7.4 | | | | | | | | | | | | |
| 4 | Monovalent | n/a | n/a | n/a | n/a | 462 | n/a | 25126 | n/a | n/a | n/a | n/a | n/a |
| | modRNA HA | | | | | | | | | | | | |
| | (A_{H1}) | | | | | | | | | | | | |
| | (A/Wisconsin) | | | | | | | | | | | | |
| | 0.126 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 5 | Monovalent | n/a | n/a | n/a | n/a | n/a | 483 | n/a | 18110 | n/a | n/a | n/a | n/a |
| | modRNA HA | | | | | | | | | | | | |
| | (A_{H1}) | | | | | | | | | | | | |
| | (A/Sydney) | | | | | | | | | | | | |
| | 0.114 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 6 | Monovalent | n/a | n/a | n/a | n/a | n/a | n/a | n/a | n/a | 234 | n/a | 7135 | n/a |
| | modRNA HA | | | | | | | | | | | | |
| | (A_{H3}) | | | | | | | | | | | | |
| | (A/Darwin) | | | | | | | | | | | | |
| | 0.118 mg/mL of | | | | | | | | | | | | |
| | modRNA LNP in | | | | | | | | | | | | |
| | 10 mM Tris | | | | | | | | | | | | |
| | 10% Sucrose, | | | | | | | | | | | | |
| | pH 7.4 | | | | | | | | | | | | |
| 7 | Monovalent | n/a | n/a | n/a | n/a | n/a | n/a | n/a | n/a | n/a | 30 | n/a | 113 |
| | modRNA HA | | | | | | | | | | | | |
| | (A_{H3}) | | | | | | | | | | | | |
| | (A/Cambodia) | | | | | | | | | | | | |
| | 0.140 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 8 | Bivalent | 867 | 104 | 7757 | 1019 | n/a | n/a | n/a | n/a | n/a | n/a | n/a | n/a |
| | modRNA HA | | | | | | | | | | | | |
| | (BᵥB_{y}) | | | | | | | | | | | | |
| | (B/Austria + | | | | | | | | | | | | |
| | B/Phuket) | | | | | | | | | | | | |
| | 0.121 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 9 | Bivalent | 834 | n/a | 6343 | n/a | 844 | n/a | 36325 | n/a | n/a | n/a | n/a | n/a |
| | modRNA HA | | | | | | | | | | | | |
| | (BᵥA_{H1}) | | | | | | | | | | | | |
| | (B/Austria + | | | | | | | | | | | | |
| | A/Wisconsin) | | | | | | | | | | | | |
| | 0.128 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 10 | Trivalent | 627 | n/a | 5679 | n/a | 642 | n/a | 35062 | n/a | 386 | n/a | 7702 | n/a |
| | modRNA HA | | | | | | | | | | | | |
| | (BᵥA_{H1}A_{H3}) | | | | | | | | | | | | |
| | (B/Austria + | | | | | | | | | | | | |
| | A/Wisconsin + | | | | | | | | | | | | |
| | A/Darwin) | | | | | | | | | | | | |
| | 0.140 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 11 | Quadrivalent | 885 | 95 | 8803 | 1481 | 820 | n/a | 55405 | n/a | 889 | n/a | 11846 | n/a |
| | modRNA HA | | | | | | | | | | | | |
| | (BᵥB_{y}A_{H1}A_{H3}) | | | | | | | | | | | | |
| | (B/Austria + | | | | | | | | | | | | |
| | B/Phuket + | | | | | | | | | | | | |
| | A/Wisconsin + | | | | | | | | | | | | |
| | A/Darwin) | | | | | | | | | | | | |
| | 0.122 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 12 | Quadrivalent | 702 | n/a | 5657 | n/a | 721 | n/a | 28379 | n/a | 703 | 60 | 6031 | 279 |
| | modRNA HA | | | | | | | | | | | | |
| | (BᵥA_{H1}A_{H3}A_{H3}) | | | | | | | | | | | | |
| | (B/Austria + | | | | | | | | | | | | |
| | A/Wisconsin + | | | | | | | | | | | | |
| | A/Darwin + | | | | | | | | | | | | |
| | A/Cambodia) | | | | | | | | | | | | |
| | 0.116 mg/mL of | | | | | | | | | | | | |
| | modRNA LNP in | | | | | | | | | | | | |
| | 10 mM Tris | | | | | | | | | | | | |
| | 10% Sucrose, | | | | | | | | | | | | |
| | pH 7.4 | | | | | | | | | | | | |
| 13 | Quadrivalent | 332 | n/a | 3067 | n/a | 791 | 647 | 34257 | 24363 | 461 | n/a | 7484 | n/a |
| | modRNA HA | | | | | | | | | | | | |
| | (BᵥA_{H1}A_{H1}A_{H3}) | | | | | | | | | | | | |
| | (B/Austria + | | | | | | | | | | | | |
| | A/Wisconsin + | | | | | | | | | | | | |
| | A/Sydney + | | | | | | | | | | | | |
| | A/Darwin) | | | | | | | | | | | | |
| | 0.118 mg/mL of | | | | | | | | | | | | |
| | modRNA LNP | | | | | | | | | | | | |
| | in 10 mM Tris | | | | | | | | | | | | |
| | 10% Sucrose, | | | | | | | | | | | | |
| | pH 7.4 | | | | | | | | | | | | |
| 14 | Licensed flu | 624 | 148 | 1570 | 629 | 708 | n/a | 5517 | n/a | 311 | n/a | 4039 | n/a |
| | vaccine | | | | | | | | | | | | |

GMT 13 refers to geometric mean titers 3 weeks post dose 1 against By/Austria; GMT14 refers to geometric mean titers 3 weeks post dose 1 against By/Phuket; GMT15 refers to geometric mean titers 2 weeks post dose 2 against By/Austria; GMT16 refers to geometric mean titers 2 weeks post dose 2 against By/Phuket; GMT17 refers to geometric mean titers 3 weeks post dose 1 against H1N1 A/Wisconsin; GMT18 refers to geometric mean titers 3 weeks post dose 1 against H1N1 A/Sydney; GMT19 refers to geometric mean titers 2 weeks post dose 2 against H1N1 A/Wisconsin; GMT20 refers to geometric mean titers 2 weeks post dose 2 against H1N1 A/Sydney; GMT21 refers to geometric mean titers 3 weeks post dose 1 against H3N2 A/Darwin; GMT22 refers to geometric mean titers 3 weeks post dose 1 against H3N2 A/Cambodia GMT23 refers to geometric mean titers 2 weeks post dose 2 against H3N2 A/Darwin GMT24 refers to geometric mean titers 2 weeks post dose 2 against H3N2 A/Cambodia

### Example 10: Supportive In Vitro Studies

5.1.1.3.1. In Vitro Expression of Antigens from the Influenza modRNA Vaccine

To demonstrate that the HA protein from the A/Wisconsin/588/2019 (H1N1) strain is efficiently expressed from modRNA, LNP-formulated modRNA encoding this HA antigen was added to HEK-293T cells and antigen expression measured by flow cytometry. The measured HA protein expression was dose-dependent, as reported by percent HA positive cells across vaccine input doses. Specifically, LNP-formulated modRNA encoding HA from 2 development lots was added to a HEK-293 T cell monolayer at 3 RNA dose levels (125 ng, 63 ng, and 31 ng) per well. Protein expression was measured using a rabbit polyclonal antibody raised against the A/Michigan/45/2015 (H1N1) strain. Prior to analysis by flow cytometry, cells were labeled with a Live/Dead dye, and the percentage of live cells expressing the H1N1 HA protein is enumerated. Expression was measured by quantifying the number of cells that had a positive signal for bound anti-HA antibody.

5.1.1.3.2. In Vitro Antigen Expression from Pandemic Influenza modRNA Vaccines

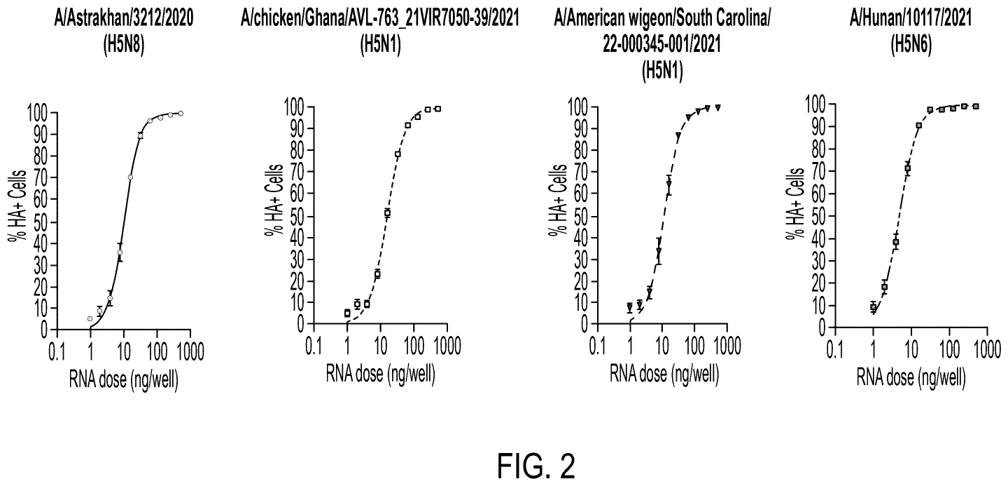

To demonstrate that the HA proteins from the A/Astrakhan/3212/2020 (H5N8) and related H5N1 and H5N6 (clade 2.3.4.4b) strains are efficiently expressed from modRNA, LNP formulated modRNA candidates encoding these HA antigens were added to HEK-293T cells and antigen expression was measured by flow cytometry (FIG. 2). With respect to FIG. 2, LNP-formulated modRNA vaccine candidates encoding H5 (clade 2.3.4.4b) antigens were 2-fold serially titrated and added to a HEK-293T cell monolayer (from 500 to 0.97 ng/well). HA protein expression was measured using the HA stem-specific, broadly neutralizing human monoclonal antibody F16. Prior to analysis by flow cytometry, cells were labeled with fluorescent antibodies, and the percentage of live cells expressing the HA protein is enumerated. Expression was measured by quantifying the number of cells that had a positive signal for bound anti-HA antibody.

### Example 11: (5.1.1.10.) Immunogenicity in Mice of a Multivalent Influenza modRNA Vaccine

Current licensed seasonal influenza vaccines are designed to protect against up to 4 different influenza viruses, including 2 influenza A viruses (H1N1 and H3N2 subtypes) and 2 influenza B viruses (B Yamagata and B Victoria lineages). To evaluate the feasibility of a multivalent formulation of the modRNA influenza vaccine, modRNAs encoding 4 different HA proteins and 4 different NA proteins were generated. Immune responses elicited by mice vaccinated with LNP-formulated modRNA encoding a single strain-specific HA or NA were compared to groups vaccinated with a multivalent HA/NA modRNA formulation. Multivalent formulation methods were compared by separately formulating each modRNA expressing HA or NA in LNPs and then mixing the 8 LNPs together in equal ratios, or by pre-mixing the 8 modRNAs followed by a single co formulation in LNPs. A licensed, adjuvanted trivalent inactivated influenza vaccine (FluAd®, Seqirus) was included as a benchmark in the study.

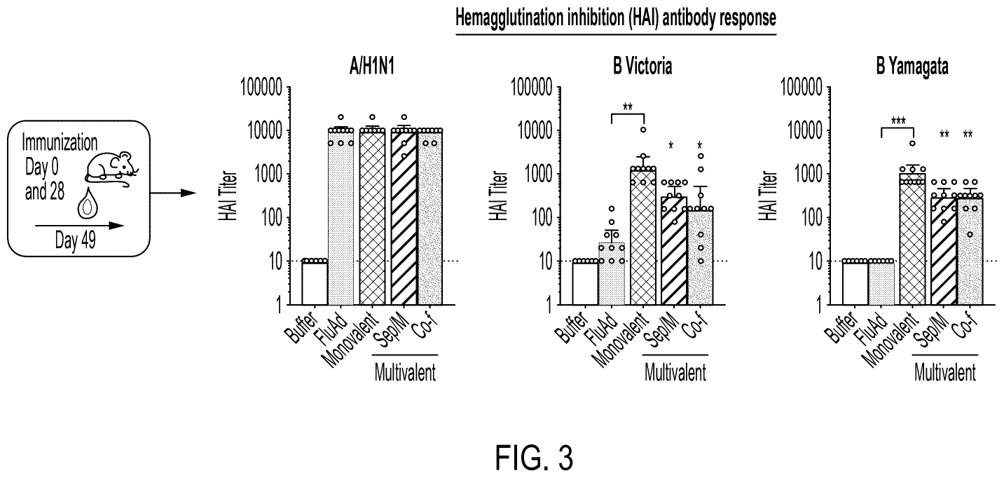

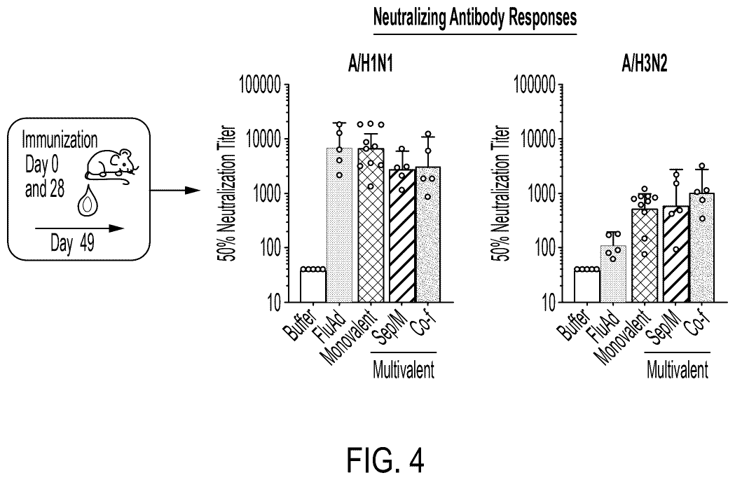

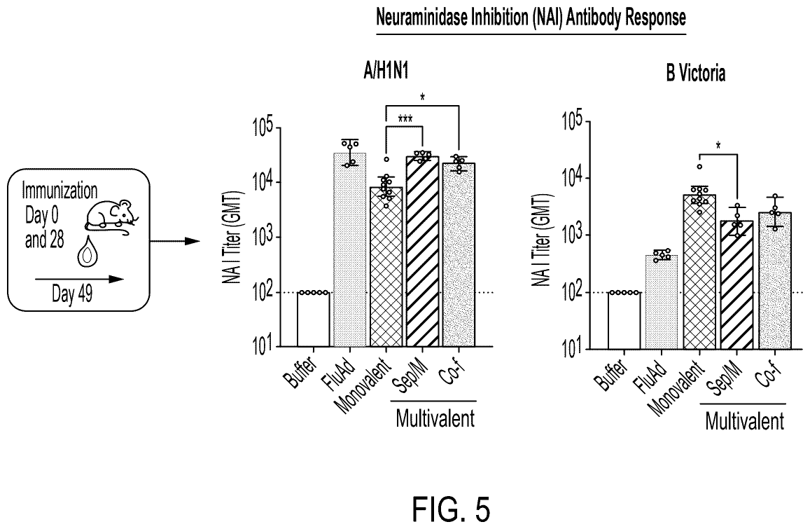

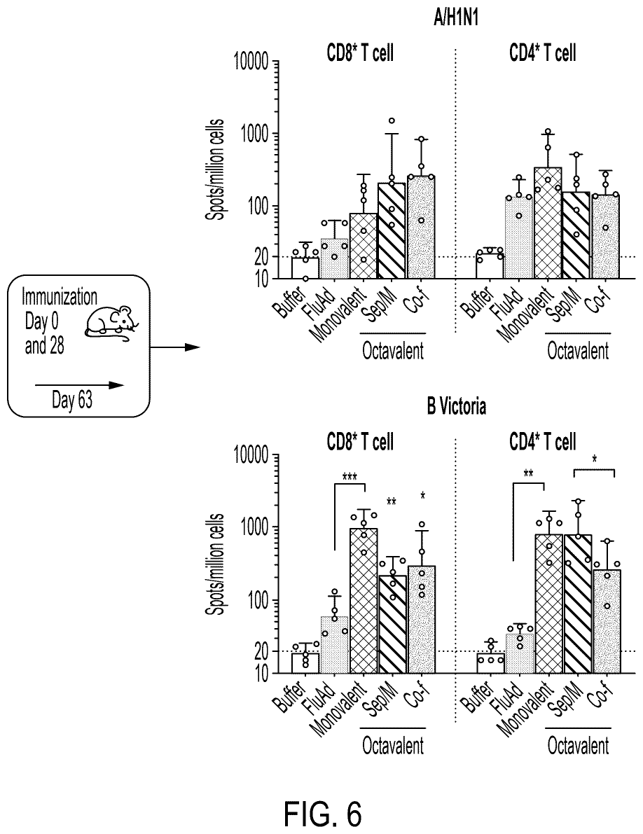

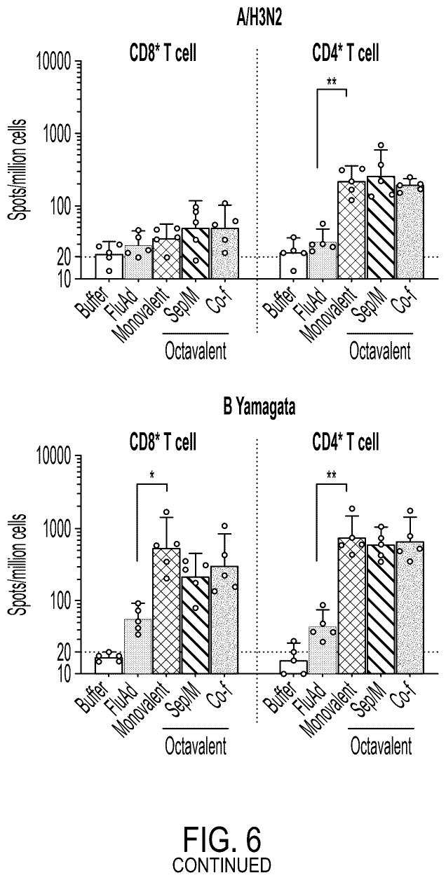

BALB/c mice were immunized IM with 2 μg of each HA- and NA-expressing modRNA either as a monovalent or multivalent vaccine formulation in LNPs on Days 0 and 28. Robust antibody and T-cell responses were elicited by LNP-formulated modRNA to all HA and NA components, at levels similar to or greater than the licensed vaccine comparator. Similar HAI and neutralizing responses were observed on Day 49 (21 days after the second boost) for individual HA and multivalent formulations for influenza A strains; however, some interference was observed with B strains in the multivalent formulation (FIG. 3 and FIG. 4). Antibodies measured against NA showed a similar trend as HA (FIG. 5). Similar CD8+ and CD4+ T-cell responses were observed for monovalent HA and multivalent formulations for influenza strains, with some interference also noted for the B strains (FIG. 6). No difference in immunogenicity was observed with the 2 different multivalent formulation methods. These data also demonstrated the potential to supplement or combine an influenza modRNA vaccine expressing HA proteins with modRNA expressing NA proteins. NA may serve as a desirable vaccine antigen due to evidence that NA plays a role in reducing disease severity and inducing cross-protection.

### Example 12: (5.1.1.11.) Immunogenicity in Mice of LNP-Formulated Pandemic Influenza modRNA Vaccine Candidates

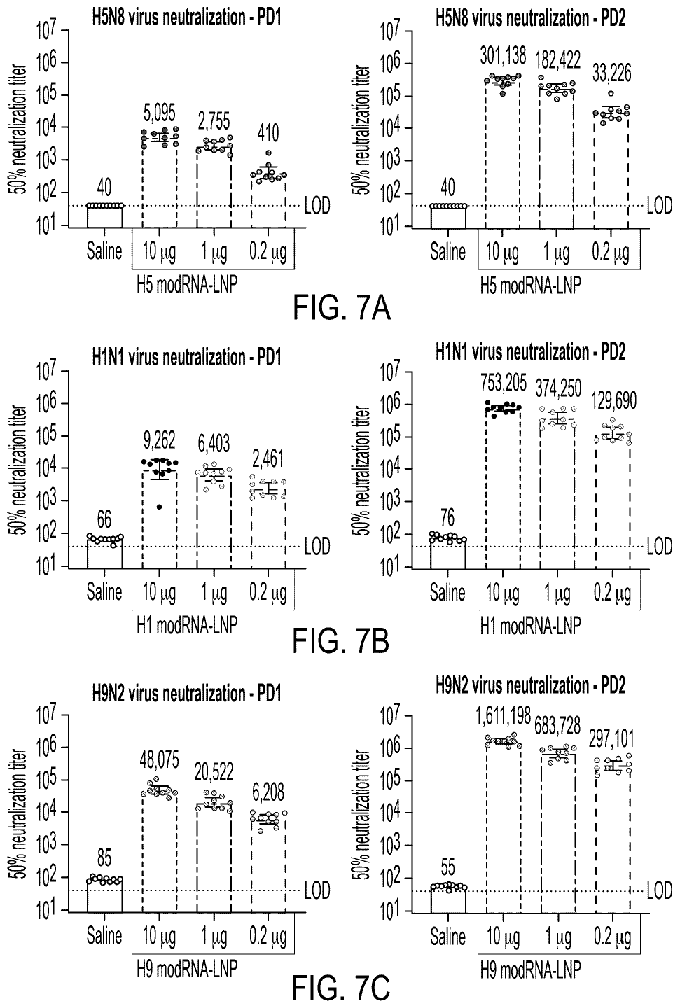

Robust virus neutralization responses were elicited by pdmFlu vaccine candidates at levels similar to the seasonal H1 modRNA vaccine benchmark. To evaluate immunogenicity at escalating doses, BALB/c mice were immunized IM with 0.2, 1, or 10 μg of each LNP-formulated influenza modRNA vaccine preparation encoding the HA antigen from A/Astrakhan/3212/2020 (H5N8) or A/Wisconsin/588/2019 (H1N1) on Days 0 and 28. Virus neutralization titers against the matched virus strains were measured using sera collected on Days 22 (3 weeks post prime) and 42 (2 weeks post boost) (FIGS. 7A and 7B). FIG. 7C shows neutralization titers against H9N2 post dose 1 (PD1) and post dose 2 (PD2). The study design for FIG. 7A-C is shown in Table 41. A dose-dependent neutralizing response was observed for the modRNA vaccines at both timepoints.

TABLE 41

| | | | Dos | Dose Volume/ | Vaccination |
|---|---|---|---|---|---|
| Gp # | N/G | RNA | (μg) | Route | (Day) |
| 1 | 10 | Saline | — | 50 mL/IM | 0, 28 |
| 2 | 10 | pdmFlu | 10 | | |
| | | modRNA-H5 | | | |
| | | (A/Astrakhan/ | | | |
| | | 3212/2020 H5) | | | |
| 3 | 10 | pdmFlu | 1 | | |
| | | modRNA-H5 | | | |
| | | (A/Astrakhan/ | | | |
| | | 3212/2020 H5) | | | |
| 4 | 10 | pdmFlu | 0.2 | | |
| | | modRNA-H5 | | | |
| | | (A/Astrakhan/ | | | |
| | | 3212/2020 H5) | | | |
| 5 | 10 | pdmFlu | 10 | | |
| | | modRNA-H9 | | | |
| | | (A/Anhui-Lujiang/ | | | |
| | | 39/2018 H9) | | | |
| 6 | 10 | pdmFlu | 1 | | |
| | | modRNA-H9 | | | |
| | | (A/Anhui-Lujiang/ | | | |
| | | 39/2018 H9) | | | |
| 7 | 10 | pdmFlu | 0.2 | | |
| | | modRNA-H9 | | | |
| | | (A/Anhui-Lujiang/ | | | |
| | | 39/2018 H9) | | | |
| 8 | 10 | modRNA-H1 | 10 | | |
| | | (A/Wisconsin/ | | | |
| | | 588/2019) | | | |
| 9 | 10 | modRNA-H1 | 1 | | |
| | | (A/Wisconsin/ | | | |
| | | 588/2019) | | | |
| 10 | 10 | modRNA-H1 | 0.2 | | |
| | | (A/Wisconsin/ | | | |
| | | 588/2019) | | | |

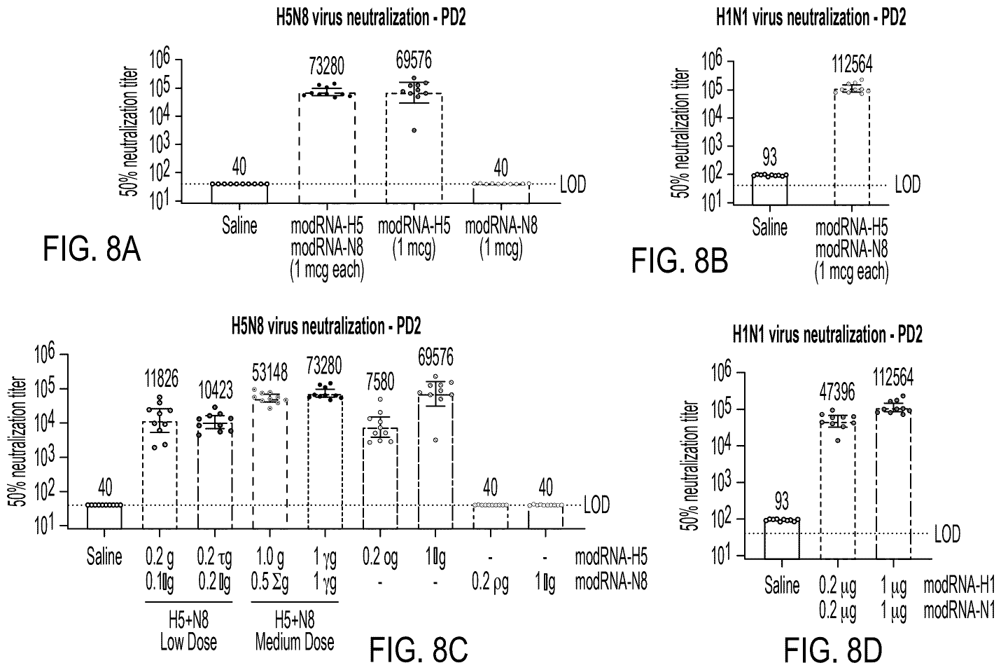

Virus neutralizing antibody titers and CD4+ and CD8+ T-cell responses induced by pdmFlu vaccine candidates encoding individual or both HA and NA antigens from A/Astrakhan/3212/2020 were also measured in a different mouse study, benchmarked against a modRNA vaccine encoding seasonal influenza HA (H1) and NA (N1) antigens. BALB/c mice were immunized IM with LNP-formulated influenza modRNA vaccine preparations containing 1 μg of each modRNA on Days 0 and 28. Virus neutralization titers against the matched virus strains were measured using sera collected on Day 43 (2 weeks post boost) (FIG. 8). The H5+N8 pdmFlu vaccine elicited similar levels of virus neutralizing antibodies in mice as the H5 pdmFlu vaccine alone.

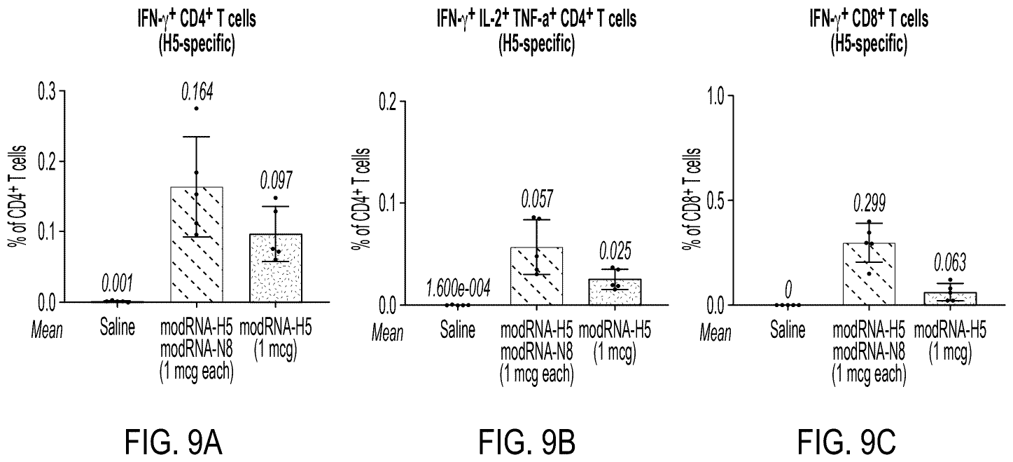

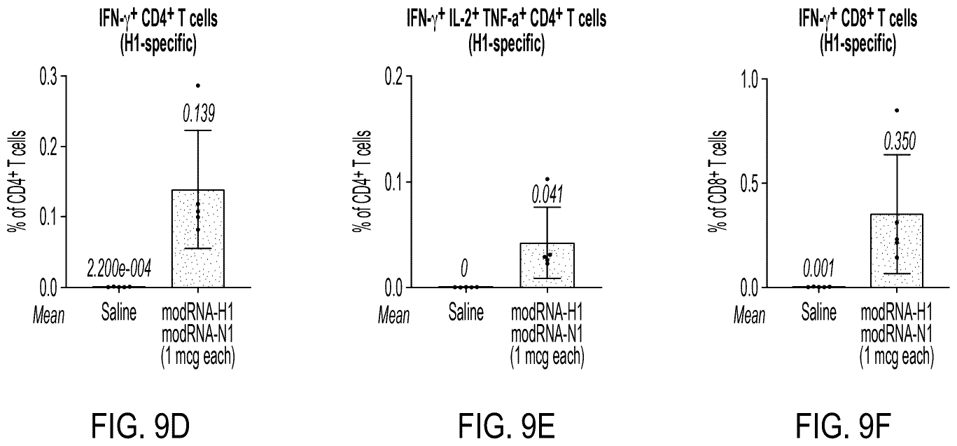

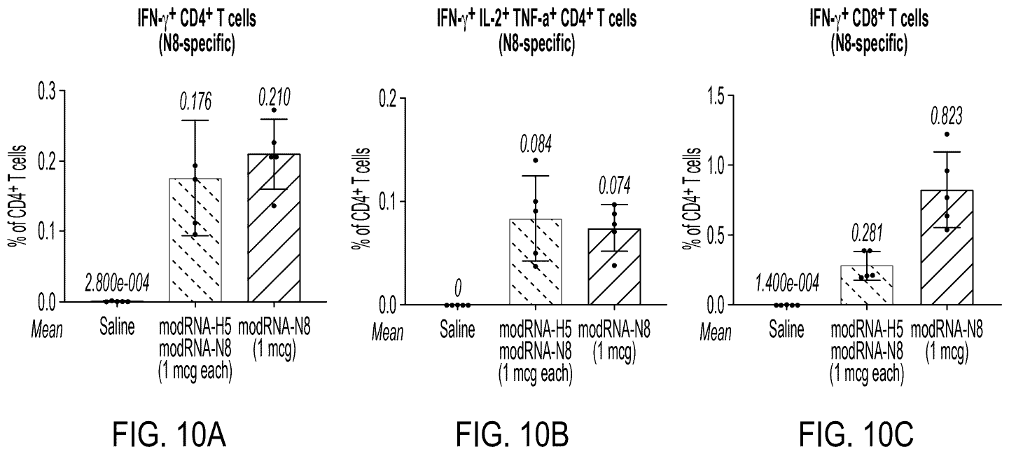

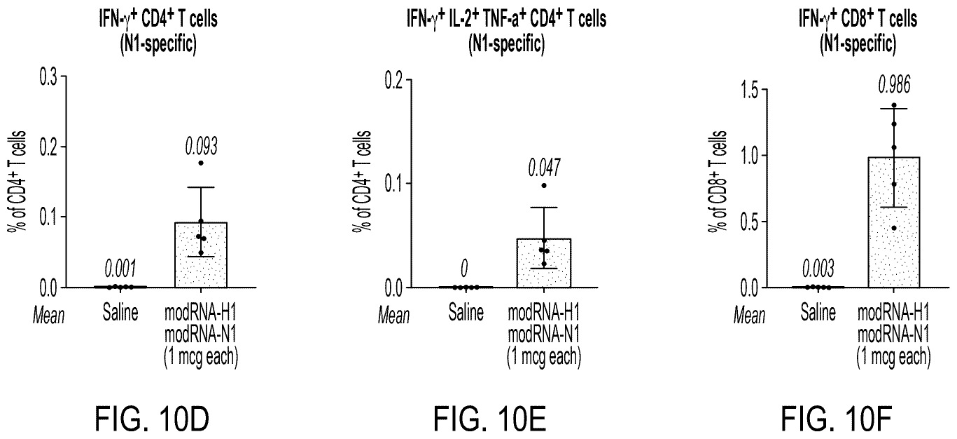

Splenocytes were also harvested on Day 43 and stimulated with a HA (H5 or H1) or NA (N8 or N1) peptide pool to measure the HA- or NA-specific T-cell responses as a percentage of cytokine-expressing CD4+ and CD8+ T-cells (FIG. 8A-D and FIG. 9). Vaccination with the pdmFlu vaccine candidates induced robust IFN-γ+CD4+ T-cell responses, including those with polyfunctional (IFN-γ+, IL-2+, TNF-α+) attributes, for both the HA and NA antigens. These data confirmed that the pandemic influenza modRNA candidates elicited Th1-biased T-cell responses, which is consistent with what was observed for the seasonal influenza modRNA construct. Notably, HA- and NA-specific CD4+ T-cell responses in the H5+N8 pdmFlu vaccine group are higher or similar in magnitude to the H5 or N8 only pdmFlu groups, respectively, suggesting lack of interference in elicitation of HA- and NA-specific CD4+ T-cell responses with the inclusion of both H5 and N8 antigens in the pdmFlu vaccine. The pdmFlu vaccine candidates also induced HA- and NA-specific IFN-γ-expressing CD8+ T-cell responses, similar to that of the seasonal influenza modRNA vaccine. Compared to the H5 or N8 only pdmFlu vaccine groups, inclusion of both H5 and N8 antigens in the pdmFlu vaccine resulted in higher H5 specific but lower N8-specific CD8+ T-cell responses, suggesting potential interference in elicitation of NA-specific CD8+ T-cell response by the H5+N8 pdmFlu vaccine. Overall, the pdmFlu vaccine candidates induced robust virus neutralizing titers against the matched virus strain as well as antigen-specific CD4+ as well as CD8+ T cell responses.

Pandemic Flu H5N8 HA+NA modRNA immunogenicity study in mice were studied according to Table 42 and data shown in FIG. 8A-D.

TABLE 42

| | | | | | Dose | Vacci- |
|---|---|---|---|---|---|---|
| Gp | | | Dosing | Dose | Volume/ | nation |
| # | N/Gr | RNA | Group | (μg) | Route | (Day) |
| 1 | 10 | Saline | — | — | 50 μL/IM | 0, 28 |
| 2 | 10 | pdmFlu | Low | 0.3 μg | | |
| | | modRNA-H5 + | Dose | (0.2 μg HA/ | | |
| | | pdmFlu | | 0.1 μg NA) | | |
| | | modRNA-N8 | | | | |
| | | (2:1) | | | | |
| 3 | 10 | pdmFlu | | 0.4 μg | | |
| | | modRNA-H5 + | | (0.2 μg HA/ | | |
| | | pdmFlu | | 0.2 μg NA) | | |
| | | modRNA-N8 | | | | |
| | | (1:1) | | | | |
| 4 | 10 | pdmFlu | Medium | 1.5 μg | | |
| | | modRNA-H5 + | Dose | (1 μg HA/ | | |
| | | pdmFlu | | 0.5 μg NA) | | |
| | | modRNA-N8 | | | | |
| | | (2:1) | | | | |
| 5 | 10 | pdmFlu | | 2 μg | | |
| | | modRNA-H5 + | | (1 μg HA/ | | |
| | | pdmFlu | | 1 μg NA) | | |
| | | modRNA-N8 | | | | |
| | | (1:1) | | | | |
| 6 | 10 | | — | 0.2 μg | | |
| 7 | 10 | pdmFlu | — | 1 μg | | |
| | | modRNA-H5 | | | | |
| 8 | 10 | pdmFlu | — | 0.2 μg | | |
| 9 | 10 | modRNA-N8 | — | 1 μg | | |
| 10 | 10 | modRNA-H1 + | Low | 0.4 μg | | |
| | | modRNA-N1 | Dose | (0.2 μg HA/ | | |
| | | (1:1) | | 0.2 μg NA) | | |
| 11 | 10 | | Medium | 2 μg | | |
| | | | Dose | (1 μg HA/ | | |
| | | | | 1 μg NA) | | |

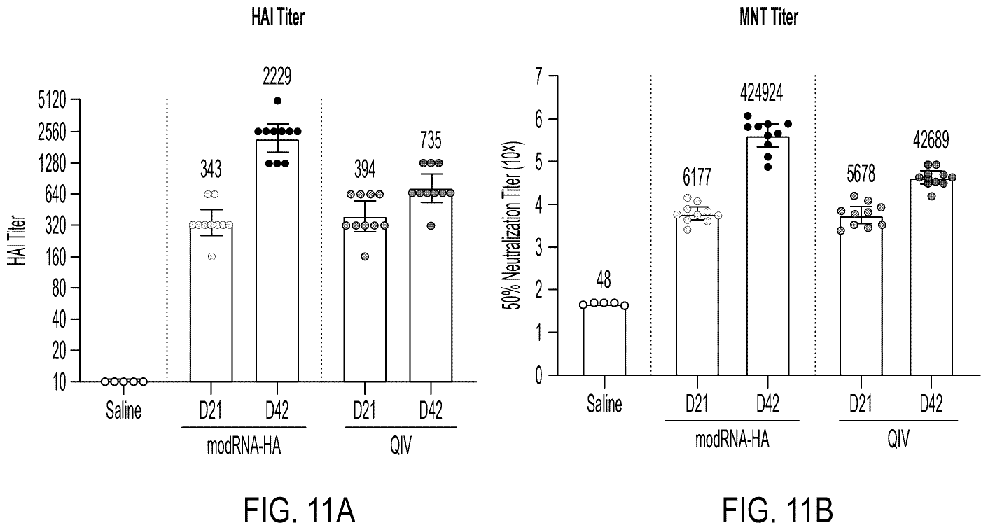

Immunogenicity in Mice of an Influenza modRNA Vaccine. Robust HAI and virus neutralization responses were elicited by LNP-formulated influenza modRNA at levels similar to or greater than the licensed QIV comparator. A mouse immunogenicity study was initially conducted using an influenza monovalent modRNA vaccine encoding the HA sequence from A/Wisconsin/588/2019 (H1N1) (VR-VTR-10907). BALB/c mice were immunized IM with 0.2 μg of an LNP-formulated influenza modRNA vaccine or 2.4 μg (1/25th of the human dose) of a licensed adjuvanted quadrivalent inactivated influenza vaccine (QIV, Fluad) on Days 0 and 28. Functional antibody and virus neutralization titers were measured by HAI and MNT, respectively, using sera collected on Days 21 (3 weeks post-prime) and 42 (2 weeks post-boost) (FIG. 11A-B). At 3 weeks post-dose 1, the influenza modRNA vaccine and the QIV comparator induced similar HAI and MNT titers, but the influenza modRNA vaccine was more effective than QIV at boosting these responses after the second immunization. For the influenza modRNA vaccine, 2 weeks after the second dose, HAI titers increased by more than 6× and MNT titers increased by 68×.

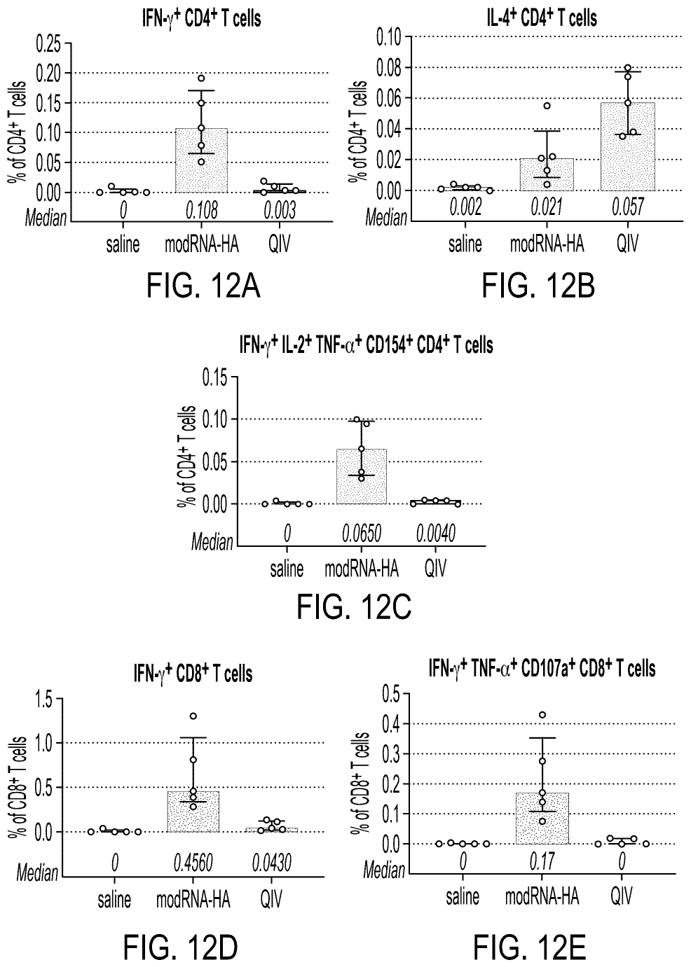

HA-specific CD4+ and CD8+ T-cell responses were also measured from splenocytes harvested on Day 42 (2 weeks post-dose 2) from this same mouse study. Splenocytes were stimulated with an HA peptide pool to measure the HA-specific T-cell response as a percentage of cytokine-expressing CD4+ and CD8+ T-cells (FIG. 12A-E). Vaccination with the influenza modRNA vaccine induced high Th1-type (IFN-γ) CD4+ T-cell responses compared to the licensed QIV comparator, while the licensed QIV induced higher Th2-type (IL-4) CD4+ T-cell responses. A polyfunctional (IFN-γ, IL-2, TNF-α) CD4+ T-cell response was also observed for the influenza modRNA vaccine. These data confirmed that influenza modRNA formulated with LNPs elicited Th1-biased T-cell responses, which is consistent with what was observed for BNT162b2. The influenza modRNA vaccine also induced much stronger IFN-γ expressing and polyfunctional (IFN-γ, TNF-α, CD107a) CD8+ T-cell responses compared to the licensed QIV comparator.

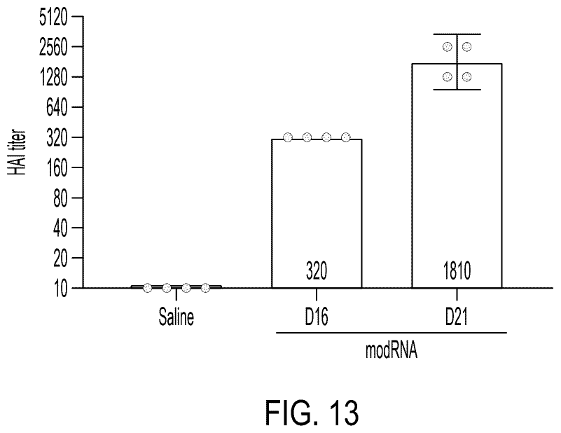

Immunogenicity in Rats of an Influenza modRNA Vaccine. Influenza modRNA vaccines induced functional antibody responses in rats. A rat immunogenicity study was performed to confirm the presence of a detectable antibody response to influenza modRNA LNP preparations in the selected toxicology species (VR-VTR-10794). Wistar-Han rats were immunized IM with 30 μg of an LNP-formulated influenza modRNA vaccine encoding A/Wisconsin/588/2019 (H1N1) HA on Days 0 and 14. Functional antibody responses against A/Wisconsin/588/2019 were detected on Days 16 and 21 (2 and 7 days post-boost, respectively) as measured by HAI (FIG. 13). A clear boosting effect was observed after the second immunization.

Immunogenicity in Nonhuman Primates of an Influenza modRNA Vaccine.

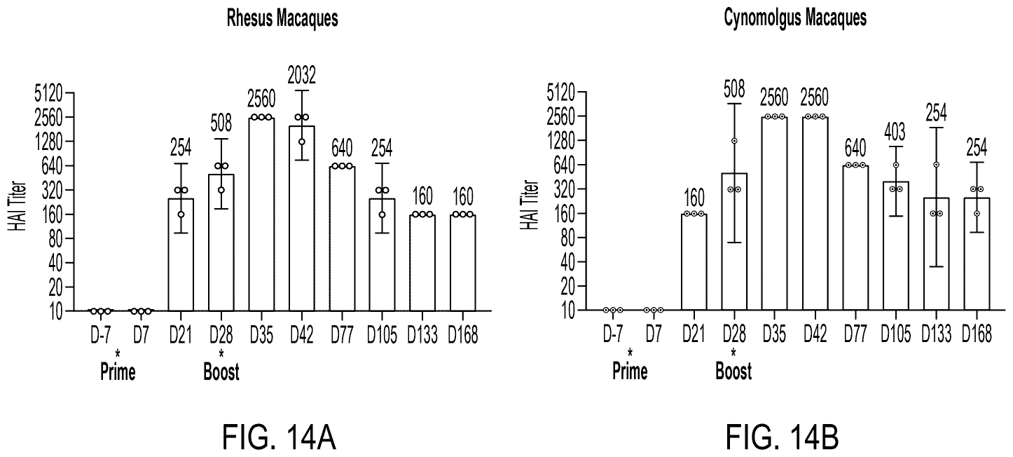

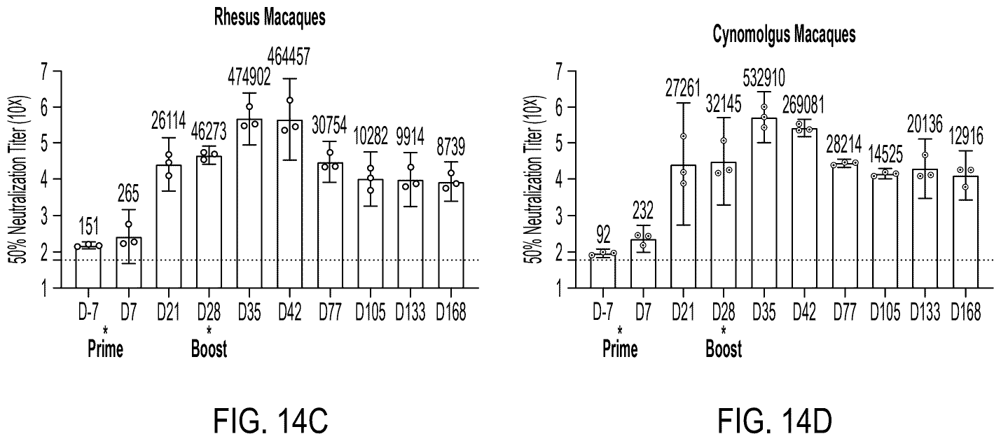

Influenza modRNA vaccines induced functional and neutralizing antibody responses in NHPs. A study was performed to test the immunogenicity of an LNP-formulated influenza modRNA vaccine preparation encoding the A/Wisconsin/588/2019 (H1N1) HA antigen in NHPs (VR-VTR-10948). Both rhesus and cynomolgus macaques were included in the study to evaluate the vaccine candidate in multiple species. Animals were immunized IM with 30 μg of an LNP-formulated influenza modRNA vaccine on Days 0 and 28. Functional antibody and virus neutralization titers were measured by HAI and MNT, respectively, using sera collected on Day −7 (pre-immunization baseline titer) and multiple time-points after immunization (Days 7, 21, 28, 33, 42, 77, 105, 133, and 168). IM injection of both rhesus and cynomolgus NHPs with influenza modRNA vaccine induced robust functional antibody responses when measured by HAI and MNT (FIG. 14A-D). Sera collected from NHPs 21 and 28 days following a primary vaccination with influenza modRNA vaccine contained high levels of both HAI and neutralizing antibodies. After a second dose of vaccine on Day 28, all animals had a rise in antibody levels at Day 35 (7 days post-dose 2), which eventually waned to levels comparable to those measured pre-boost on Day 28 and these levels were maintained up to at least Day 168.

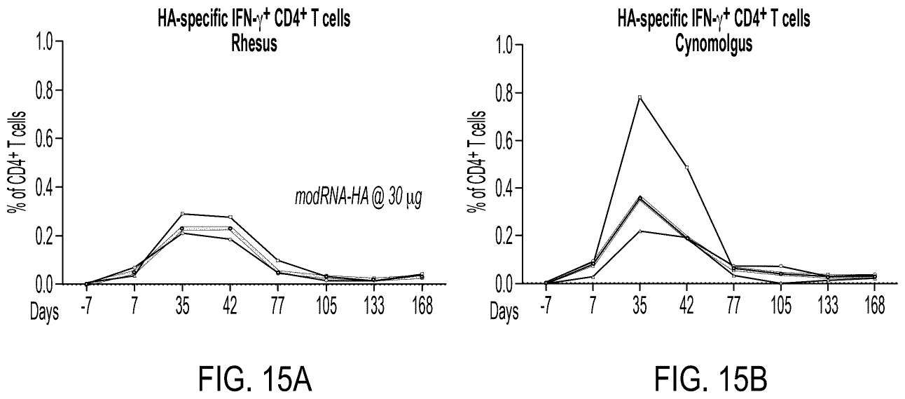

T-cell immunity induced by the influenza modRNA vaccine in the NHPs was quantified by measuring HA-specific IFN-γ+CD4+ and CD8+ T cells in PBMCs by ICS (FIG. 15A-B). The percentage of antigen-specific IFN-γ+CD4+ and CD8+ T cells before (Day −7) and after (Day 7) the first dose of each vaccine was negligible. Following the Day 28 booster dose, HA-specific CD4+ T-cells were detected in influenza modRNA-vaccinated NHPs, but little to no HA-specific CD8+ T-cells were detected in both NHP species.

Overall, these data provide evidence for the robust immunogenicity (antibody and CD4+ T cell responses) of influenza modRNA vaccines in two NHP species.

Immunogenicity in Mice of a Quadrivalent Influenza modRNA Vaccine. Current licensed seasonal influenza vaccines are designed to protect against up to four different influenza viruses, including two influenza A viruses (H1N1 and H3N2 subtypes) and two influenza B viruses (B/Yamagata and B/Victoria lineages). To evaluate the feasibility of a quadrivalent formulation of an influenza modRNA vaccine, immune responses elicited by mice vaccinated with an LNP-formulated modRNA encoding a single strain-specific HA was compared to groups vaccinated with the quadrivalent HA modRNA formulation. Two different quadrivalent formulation methods were also assessed:
1) a “pre-mix” approach where 4 modRNAs were combined in equal ratios followed by a single co-formulation into LNPs, and 2) a “post-mix” approach where each of the 4 modRNAs is separately formulated into LNPs and then the 4 LNPs are combined in equal ratios to make the final vaccine preparation (VR-VTR-10906).

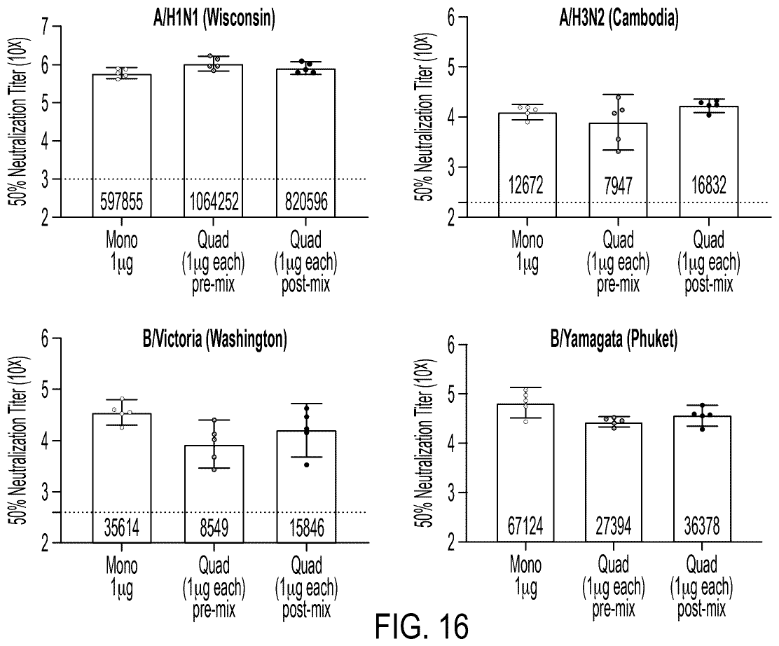

Robust immune responses were elicited by influenza modRNA to all HA components of the quadrivalent influenza modRNA vaccine. BALB/c mice were immunized IM with 1 μg of each HA-expressing modRNA either as a monovalent or quadrivalent vaccine formulation (4 μg total dose) in LNPs on Days 0 and 28. Similar virus neutralizing titers were obtained on Day 42 (2 weeks post-dose 2) for monovalent and quadrivalent formulations for influenza A strains; however, some interference was observed with B strains in the quadrivalent formulation. Comparable immunogenicity was observed for both “pre-mix” and “post-mix” formulation methods for the quadrivalent preparation (FIG. 16).

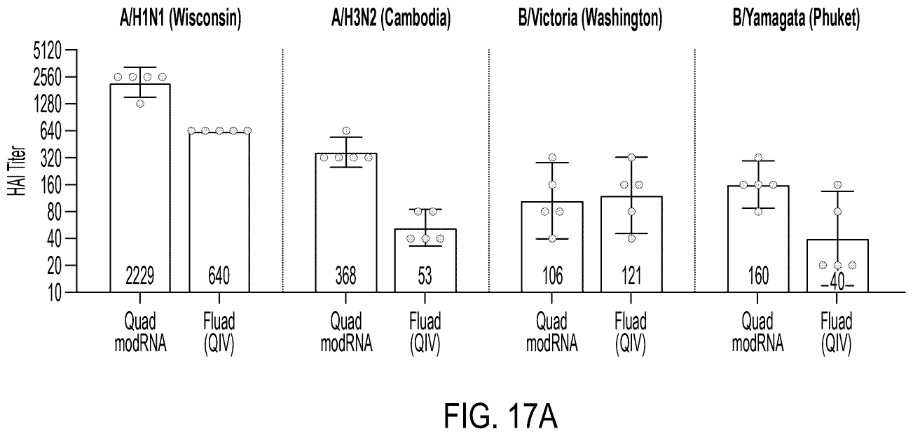

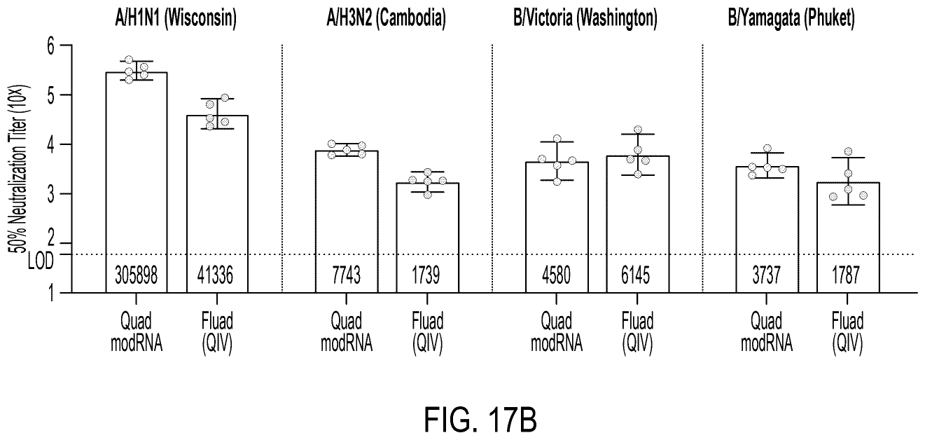

In a separate study, immune responses elicited by mice vaccinated with the quadrivalent influenza modRNA vaccine was also compared to responses induced by a licensed, adjuvanted quadrivalent inactivated influenza vaccine (FluAd®, Seqirus), which was used as a benchmark (VR-VTR-10991). BALB/c mice were immunized IM on Days 0 and 28 with a 0.8 μg total dose of the influenza quadrivalent modRNA vaccine (0.2 μg of each modRNA construct) or 2.4 μg of the licensed QIV comparator. Robust antibody responses were elicited by the quadrivalent influenza modRNA vaccine to all four HA components at levels similar to or greater than the licensed vaccine comparator, as measured by HAI and MNT assays (FIG. 17A-B).

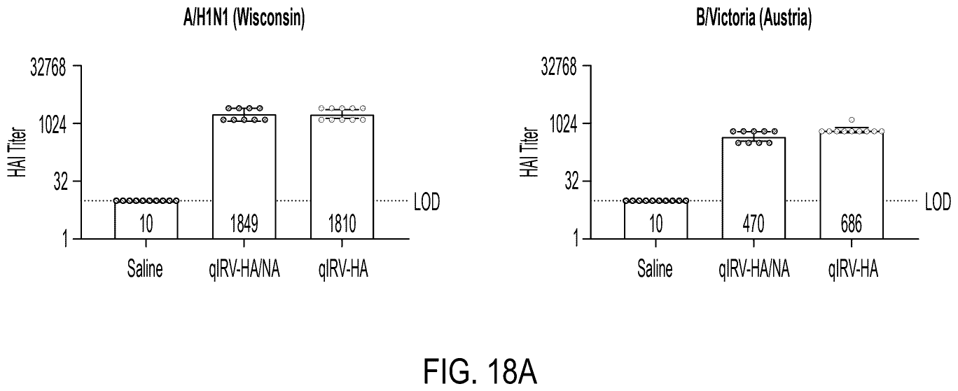

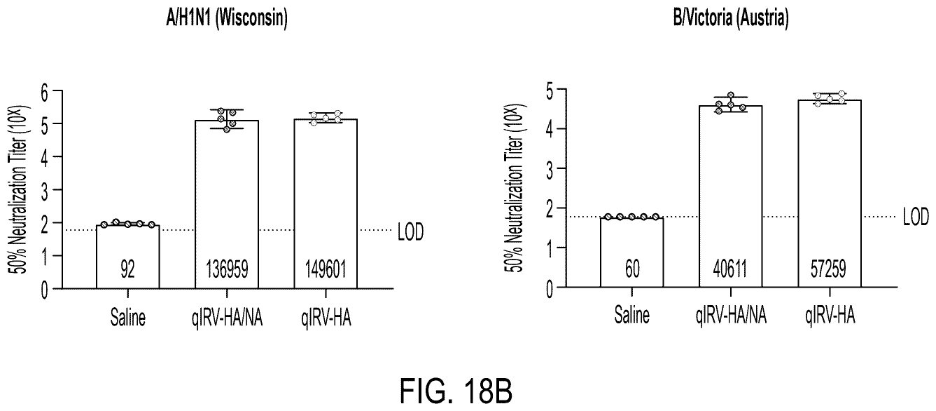

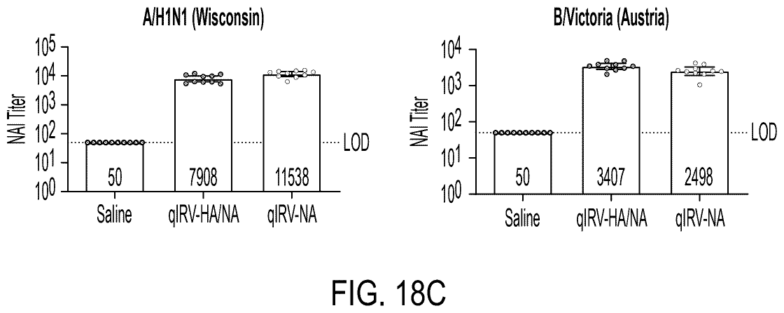

Immunogenicity in Mice of Quadrivalent Influenza modRNA Vaccines Encoding HA and NA. In addition to HA, the influenza NA glycoprotein is also immunogenic and antibodies against NA can contribute to protection against influenza infection (Eichelberger et al, 2015). Therefore, we evaluated the immunogenicity of a quadrivalent influenza modRNA vaccine encoding 4 modRNAs expressing HA and 4 modRNAs expressing NA. BALB/c mice were immunized IM on Days 0 and 28 with 1.6 μg of quadrivalent influenza modRNA-HA/NA, 0.8 μg of quadrivalent influenza modRNA-HA, or 0.8 μg of quadrivalent influenza modRNA-NA (0.2 μg of each construct) (FIG. 18A-C). Immune responses to a representative influenza A virus (H1N1) and an influenza B virus (B/Victoria) from the strains encoded in the vaccines were evaluated. The quadrivalent influenza modRNA-HA/NA vaccine elicited both influenza A and influenza B HA and NA antibodies. Functional antibody and virus neutralization titers were comparable to those elicited by quadrivalent influenza modRNA-HA and quadrivalent influenza modRNA-NA vaccines.

### Example 13: (6.) Effects in Humans

The influenza modRNA vaccine (PF-07252220) contains RNA encoding HA from up to 4 seasonal influenza viruses (selected according to official recommendations e.g. from WHO, FDA and EMA) formulated with LNPs. The pdmFlu vaccine (PF-07985819) contains RNA encoding HA for influenza strain A/H5N8 formulated with the same LNPs

The influenza modRNA vaccines (seasonal and pandemic) described herein are suspensions for injection containing RNA encapsulated in LNPs; the formulation contains 2 functional lipids, ALC-0315 and ALC-0159, and 2 structural lipids, DSPC (1,2-distearoyl-sn-glycero-3-phosphocholine) and cholesterol. During each influenza season, 1 or 2 doses totaling less than 100 μg is intended to be studied in individuals ≥18 years of age.

Influenza modRNA vaccine is administered via IM injection into the deltoid region of the upper arm. The current injection volume is 0.3 mL or 0.5 mL.

In participants 65 through 85 years of age, similar or higher antibody responses and T-cell responses were observed to all 4 strains elicited by a single dose of qIRV (60 μg) compared to qIRV (30 μg). Compared to licensed QIV, postvaccination HAI GMTs were higher in the qIRV (60 μg) group for both A strains, but lower with regard to titers against B strains. CD4+ and CD8+ T-cell responses in general, trended similar to or higher than licensed QIV for all 4 influenza strains. Seroconversion rates and GMRs (based on comparison of qIRV to licensed QIV) trended similar or higher in the 60 μg qIRV dose level as compared to the 30 μg dose level for all 4 influenza strains. Single doses of qIRV 30 μg and 60 μg were well tolerated in this age cohort. Considering the higher risk of severe complications of influenza as well as the improved immune response with the higher dose, a single dose of qIRV (60 μg) was selected for further evaluation in participants ≥65 years of age in the Phase 3 Study C4781004.

In participants 18 through 64 years of age, antibody and T-cell responses were similar overall for qIRV (30 μg) and qIRV (60 μg). A modest dose-dependent increase in reactogenicity was observed between the qIRV (30 μg) and qIRV (60 μg) groups. Based on the totality of data in this study, a single dose of qIRV (30 μg) was selected as the dose to be evaluated in participants 18 through 64 years of age in the Phase 3 Study.

The PdmFlu vaccine described herein is administered via IM injection into the deltoid region of the upper arm. The current injection volume is 0.25 mL, 0.5 mL or 0.75.

Table 43 summarizes the number of participants exposed to modRNA-based influenza vaccines by age and total dose of modRNA received in the ongoing influenza modRNA vaccine (PF-07252220) program. Exposure data includes participants from the Phase 1/2 study C4781001 (substudy A and B). A total of 706 participants 65 years of age and above and 262 participants 18 through 64 years of age received either monovalent or bivalent or quadrivalent modRNA based influenza vaccines with different strain combinations and doses. The Phase 3 study C4781004 is blinded and exposure data is not available at the time of this update—to-date, approximately 46,000participants received blinded therapy in the C4781004 study, with these participants being randomized 1:1 to received qIRV (30 μg for participants 18 through 64, and 60 μg for participants ≥65 years of age) or licensed comparator.

TABLE 43
a) Number of Participants Exposed to modRNA Influenza Vaccine by
Age and Total dose of modRNA received in C4781001 Substudy A and B

| | | | | | | | | | | Participants | | |
|---|---|---|---|---|---|---|---|---|---|---|---|---|
| | Participants ≥65 Years | | | | | | | | | 18-64 Years | | |
| | | | | | | | | | Any | | | Any |
| Exposure | 3.75 | 7.5 | 15 | 18.75 | 22.5 | 30 | 60 | 90 | modRNA | 30 | 60 | modRNA |
| (Vaccine Arm) | μg | μg | μg | μg | μg | μg | μg | μg | Vaccine | μg | μg | Vaccine |
| mIRV | 16 | 15 | 14 | | | 25 | | | 70 | | | |
| (Influenza A) | | | | | | | | | | | | |
| mIRV | 17 | 14 | 15 | | | 17 | | | 63 | | | |
| (Influenza B) | | | | | | | | | | | | |
| bIRV 3.75 μg | | | | 14 | | | | | 14 | | | |
| A + 15 μg B | | | | | | | | | | | | |
| bIRV 7.5 μg | | | 16 | | | | | | 16 | | | |
| A + 7.5 μg B | | | | | | | | | | | | |
| bIRV 7.5 μg | | | | | 15 | | | | 15 | | | |
| A + 15 μg B | | | | | | | | | | | | |
| bIRV 15 μg | | | | | | 15 | | | 15 | | | |
| A + 15 μg B | | | | | | | | | | | | |
| bIRV 2A 15 | | | | | | 43 | | | 43 | | | |
| μg/strain^{a} | | | | | | | | | | | | |
| bIRV 2A 30 | | | | | | | 30 | | 30 | | | |
| μg/strain^{a} | | | | | | | | | | | | |
| bIRV 2B 15 | | | | | | 14 | | | 14 | | | |
| μg/strain^{a} | | | | | | | | | | | | |
| qIRV 2A | | | | | | 262 | | | 262 | 131 | | 131 |
| 7.5 μg/strain, | | | | | | | | | | | | |
| 2B 7.5 | | | | | | | | | | | | |
| μg/strain^{b} | | | | | | | | | | | | |
| qIRV 2A, 2B | | | | | | | 133 | | 133 | | 131 | 131 |
| 15 μg/strain | | | | | | | | | | | | |
| x′qIRV 2A | | | | | | | 16 | | 16 | | | |
| 7.5 μg/strain, | | | | | | | | | | | | |
| 2B 22.5 | | | | | | | | | | | | |
| μg/strain | | | | | | | | | | | | |
| qIRV 2A | | | | | | | | 15 | 15 | | | |
| 7.5 μg/strain, | | | | | | | | | | | | |
| 2B 37.5 | | | | | | | | | | | | |
| μg/strain | | | | | | | | | | | | |
| Totals | 33 | 29 | 45 | 14 | 15 | 376 | 179 | 15 | 706 | 131 | 131 | 262 |

b) Number of Participants Exposed to qIRV in Combination Studies -
C5401001*** and C5261001**

modRNA Total Dose (qIRV)

| Study (Age) | 30 μg* | 60 μg* |
|---|---|---|
| C5401001 (≥60 years) | | 126 |
| C5261001 (≥65 years) | 33 | 33 |
| C5261001 (18-64 years) | 59 | 31 |
| Total qIRV exposure | 92 | 190 |

^{a}bIRV 2A (30 μg; 60 μg) and bIRV 2B (30 μg) vaccines were administered either as second dose (first dose was licensed QIV) or administered at same time as licensed QIV in a different arm
^{b}qIRV 30 μg (2A, 2B 7.5 μg/strain) administered either as a standalone vaccine or as part of 2 dose regime (21 days apart); 131 out of the 262 participants received two doses
*Only includes participants who received standalone doses of qIRV
**C5261001-A Phase 1/2 Study To Evaluate The Safety, Tolerability, And Immunogenicity Of Combined Modified Rna Vaccine Candidates Against Covid-19 And Influenza In Healthy Individuals
***C5401001-A Study To Evaluate The Safety, Tolerability, And Immunogenicity Of Respiratory Combination Vaccine Candidates In Older Adults

### a) Substudy A (Phase 1)

This was a randomized, observer-blinded (sponsor-unblinded) substudy to evaluate the safety and immunogenicity of mIRV and bIRV encoding both A and B strains in different dose level combinations, and qIRV, in participants 65 through 85 years of age.

Participants were randomized to receive at Vaccination 1 (Visit 1):
mIRV at a dose level of 3.75, 7.5, 15, or 30 μg encoding A strain, or QIV
mIRV at a dose level of 3.75, 7.5, 15, or 30 μg, encoding B strain, or QIV
bIRV in different A+B dose level combinations, encoding both A and B strains, or QIV
qIRV encoding 2 A strains and 2 B strains at a dose level of 7.5 μg per strain, or QIV

The safety and immunogenicity data available from C4781001 Substudy A is described below. Based on this data, the C4781001 protocol included increased doses of influenza modRNA vaccine (Substudy B), with a total dose not exceeding 100 μg. This was supported by an acceptable tolerability profile for a single dose up to 30 μg and opportunity to increase the immune response. Overall, the influenza modRNA vaccines and licensed QIV were generally well tolerated across study groups with no unexpected safety findings.

### b) Immunogenicity

Peak HAI antibody responses were observed at 4 weeks after Vaccination 1 in both influenza modRNA vaccine groups and licensed QIV recipients, with higher doses of influenza modRNA vaccines generally outperforming lower doses for HAI GMTs. When individual strains encoded by mIRV and qIRV were compared at similar doses, mIRV generally induced higher HAI antibody responses than qIRV.

The immunogenicity profile based on HAI GMTs against homologous (ie, vaccine-encoded) strains showed increased titers at postvaccination time points compared to before vaccination across mIRV, bIRV, and qIRV groups. The strains encoded by qIRV and evaluated for homologous immune responses were: A/Wisconsin/588/2019 (H1N1), A/Cambodia/e0826360/2020 (H3N2), B/Phuket/3073/2013 (Yamagata lineage), and B/Washington/02/2019 (Victoria lineage).

Postvaccination GMTs for these homologous strains were increased against influenza A strains, and more modestly increased against B strains. HAI GMTs in the influenza modRNA vaccine groups were generally similar to or higher than those in the licensed QIV control groups. The fold-rises, ratios of titers, and seroconversion results overall reflected this same pattern. Additionally, HAI GMTs against heterologous strains were analyzed at 4 weeks after vaccination for the qIRV group, with results showing that heterologous GMTs for participants who received qIRV were similar to or higher than those who received licensed QIV when evaluating influenza A strains and trended lower than those in the licensed QIV group when evaluating influenza B strains. Comparisons of heterologous titer GMFRs, GMRs versus licensed QIV, and seroconversion rates versus licensed QIV showed a similar trend: qIRV antibody responses were generally similar or higher against influenza A strains, and similar or lower against influenza B strains, which is consistent with observations for homologous HAI GMTs.d T-cell responses were characterized by the induction of IFNg-producing CD8 T cells and a Th1-biased CD4 T-cell response, as evidenced by the induction of CD4 IFNg-producing cells and limited to few IL-4-producing cells, which is associated with effective antiviral cell-mediated immunity along with humoral antibody-mediated responses (ie, HAI titers). In contrast to HAI antibody responses, cell-mediated immune responses to the influenza modRNA vaccines showed T-cell activation as soon as 1 week after Vaccination 1. This Th1-dominant cell-mediated immune response was seen for all 4 influenza strains after vaccination with qIRV, and the magnitude of the T-cell responses trended similar or higher overall in the qIRV group as compared to licensed QIV.

### Substudy B (Phase 1/2)

This is a randomized, single-blinded (sponsor-unblinded) substudy to evaluate the safety and immunogenicity of the influenza vaccination schedules detailed below in participants 65 through 85 years of age and 18 through 64 years of age. Substudy B recruitment was separated into initial and expanded enrollment groups.

### 2-Visit Schedules—2 Vaccine Doses Separated by 21 Days

qIRV (30 lag)/qIRV (30 lag): 2 doses of qIRV (2 A strains and 2 B strains; 7.5 μg per strain) administered 21 days apart
Licensed QIV/bIRV A (30 lag): 1 dose of licensed QIV followed by 1 dose of bIRV (2 A strains; 15 μg per strain) administered 21 days apart
Licensed QIV/bIRV A (60 lag): 1 dose of licensed QIV followed by 1 dose of bIRV (2 A strains; 30 μg per strain) administered 21 days apart
Licensed QIV/Licensed QIV: 2 doses of licensed QIV administered 21 days apart (as a control group). Note: licensed QIV is approved for use as a single dose. 1-Visit Schedules—2 concurrently administered vaccine doses or single vaccine doses
Licensed QIV+bIRV A (30 lag): 1 dose of licensed QIV administered concurrently in the opposite arm with bIRV (2 A strains; 15 μg per strain)
Licensed QIV+bIRV A (60 lag): 1 dose of licensed QIV administered concurrently in the opposite arm with bIRV (2 A strains; 30 μg per strain)
qIRV (60 lag): 1 dose of qIRV (2 A strains and 2 B strains; 15 μg per strain)
Licensed QIV: 1 dose of licensed QIV (as a control group)
1 dose of bIRV (2 A strains; 15 μg per strain) administered concurrently in the opposite arm with bIRV (2 B strains; 15 μg per strain)
1 dose of qIRV (2 A strains and 2 B strains) in dose level combinations of:
   7.5 μg per A strain and 22.5 μg per B strain
   7.5 μg per A strain and 37.5 μg per B strain

Substudy B initial enrollment commenced with participants 65 through 85 years of age, who were randomized concurrently to one of the vaccination schedules described below (1-visit or 2-visit schedule) at Vaccination 1 (Day 1), with a minimum of 2 groups open for randomization at any one time. Approximately 15 participants were planned to be enrolled per group.

Participants were blinded as to their randomized vaccination schedule.

The interim safety and immunogenicity data available as of the data cutoff date from 04781001 Substudy B (ie, at 1 and 4 weeks following vaccination from participants 65 to 85 years of age) are described below.

### Participants 65 to 85 Years of Age

Disposition of initial enrollment groups of participants 65 through 85 years of age is summarized below.

### Initial Enrolment

### 2-Visit Schedule

In total, 61/62 participants (98.4%) randomized to a 2-visit schedule group, received at least 1 study vaccination, as summarized below.
qIRV (30 lag)/qIRV (30 lag): 15 participants were randomized, of whom 1 participant (6.7%) was not vaccinated; 14 participants (93.3%) received Vaccination 1 and 13 participants (86.7%) received Vaccination 2. One participant (6.7%) was withdrawn after Vaccination 1 and did not receive Vaccination 2 due to physician decision.
Licensed QIV/bIRV A (30 lag): 15 participants were randomized, all (100%) of whom were vaccinated; 15 participants (100%) received Vaccination 1 and 13 participants (86.7%) received Vaccination 2. Note: 1 participant randomized to licensed QIV/bIRV A (30 μg) on a 2-visit schedule received licensed QIV+bIRV A (30 μg) on a 1-visit schedule in error (Appendix 16.2.5.2). One participant (6.7%) withdrew after Vaccination 1 due to ‘withdrawal by participant’.
Licensed QIV/bIRV A (60 lag): 16 participants were randomized, all (100%) of whom were vaccinated; 16 participants (100%) received Vaccination 1, and 15 participants (93.8%) received Vaccination 2. Three participants (18.8%) withdrew after vaccination, which included 1 participant (6.3%) due to ‘withdrawal by participant’(after Vaccination 2), 1 participant (6.3%) lost to follow-up (after Vaccination 2), and 1 participant (6.3%) for reasons cited as ‘other’ (after Vaccination 1).
Licensed QIV/Licensed QIV: 16 participants were randomized, all (100%) of whom were vaccinated; all participants (100%) received both Vaccinations 1 and 2. No participants withdrew after vaccination.

### 1-Visit Schedule

In total, 62/62 participants (100%) randomized to a 1-visit schedule group received study vaccination, as summarized below.
Licensed QIV+bIRV A (30 lag): 16 participants were randomized, all (100%) of whom were vaccinated. Note: 1 participant randomized to licensed QIV+bIRV A (30 μg) on a 1-visit schedule received licensed QIV/bIRV A (30 μg) on a 2-visit schedule in error (Appendix 16.2.5.2). One participant (6.3%) withdrew due to ‘withdrawal by participant’.
Licensed QIV+bIRV A (60 lag): 15 participants were randomized, all (100%) of whom were vaccinated. Two participants (13.3%) withdrew due to ‘withdrawal by participant’.
qIRV (60 lag): 16 participants were randomized, all (100%) of whom were vaccinated. No participants withdrew after vaccination.
Licensed QIV: 15 participants were randomized, all (100%) of whom were vaccinated. No participants withdrew after vaccination.

### Expanded Enrollment

Disposition of expanded enrollment groups of participants 65 through 85 years of age is summarized below. In total, 348/349 participants (99.7%) randomized to an expanded enrollment group received study vaccination, as summarized below.
qIRV (30 lag): 117 participants were randomized, of whom 1 participant (0.9%) was not vaccinated and 116 (99.1%) were vaccinated. Six participants (5.1%) withdrew after vaccination, which included 3 participants (2.6%) due to ‘withdrawal by participant’ and 3 participants (2.6%) who were lost to follow-up.
qIRV (60 lag): 117 participants were randomized, all (100%) of whom were vaccinated. Six participants (5.1%) withdrew after vaccination, which included 3 participants (2.6%) due to ‘withdrawal by participant’ and 3 participants (2.6%) who were lost to follow-up.
Licensed QIV: 115 participants were randomized, all (100%) of whom were vaccinated. Six participants (5.2%) withdrew after vaccination, which included 3 participants (2.6%) due to ‘withdrawal by participant’, 2 participants (1.7%) who were lost to follow-up, and 1 participant (0.9%) who refused further study procedures.

### Immunogenicity

Among participants 65 through 85 years of age, across the initial and expanded enrollment groups, vaccination with influenza modRNA-containing vaccines elicited increased HAI GMTs against homologous influenza strains (ie, A/H1N1 and A/H3N2 A strains, and Yamagata and Victoria B lineages) up to 4 weeks after the last vaccination. HAI titers were generally more pronounced against influenza A strains, and more modest against influenza B strains, similarly within the modRNA-containing vaccine groups and licensed QIV control groups. Vaccine-elicited HAI GMTs were consistently higher in the modRNA-containing vaccine groups compared to the licensed Fluzone High-Dose Quadrivalent (QIV) control groups with regard to both A strains, with no consistent pattern identified with regard to both B strains across regimens (ie, 2-visit or 1-visit schedule). There was no clear pattern with regard to HAI GMTS across vaccine groups based on visit schedule (ie, sequential doses given 21 days apart versus concurrently administered doses). Postvaccination fold-rises and seroconversion were observed across all vaccine groups, and were also typically more pronounced for A strains than for B strains.

The initial enrollment groups vaccinated on a 2-visit schedule (ie, received 2 doses separated by 21 days) had comparable HAI GMTs after the second dose relative to after the first dose, which suggests that two doses does not improve vaccine-elicited HAI responses.

In the larger expanded enrollment groups, postvaccination HAI GMTs were higher in the qIRV (60 μg) group compared to the licensed QIV control group for A strains, but lower with regard to titers against B strains, and trended similar or higher compared to the qIRV (30 μg) group with regard to titers against all 4 influenza strains. A similar pattern was seen with other evaluations of antibody response (GMFRs, GMRs, and seroconversion). A single dose of qIRV (30 μg) and a single dose of qIRV (60 μg) elicited IFNγ-producing CD4+ and CD8+ T-cell immune responses to all 4 influenza strains that trended similar to or higher than the licensed QIV control. T-cell responses elicited by qIRV (60 μg) were generally similar or higher compared with qIRV (30 μg). Despite lower antibody responses of qIRV compared with the licensed QIV control for the B strains (as measured by HAI GMTs, GMFRs, GMRs, and seroconversion), higher IFNγ-producing CD4+ and CD8+ T-cell responses were elicited against the B/Phuket and the B/Washington strains at the 60 μg qIRV dose level.

Among participants 18 through 64 years of age, vaccination with qIRV (30 μg) and qIRV (60 μg) elicited increased and similar HAI GMTs against homologous influenza strains at 4 weeks after vaccination. Postvaccination fold-rises and seroconversion were typically more pronounced for A strains than for B strains. A single dose of qIRV (30 μg) and a single dose of qIRV (60 μg) elicited IFNγ-producing CD4+ and CD8+ T-cell immune responses to all the 4 strains. T-cell responses elicited by qIRV (30 μg) were generally similar or higher compared with qIRV (60 μg). Vaccine-elicited T-cell responses were observed across age and vaccine groups and reflected induction of IFNγ-producing CD8+ T-cells and a Th1-type CD4+ T-cell response. This Th1-type cytokine profile, characterized by vaccine-elicited IFNγ production in the absence of IL-4 induction, is associated with effective antiviral immunity, coupled with the humoral response of neutralizing antibody production (ie, HAI titers). Responses were generally similar against both A strains and B strains, in contrast to the difference observed for HAI titers (ie, more robust HAI GMTs against A strains and more modest HAI titers against B strains).

### Participants 18-64 Years of Age

In total, 262/265 participants (98.9%) randomized to an expanded enrollment group received study vaccination, as summarized below.
qIRV (30 lag): 132 participants were randomized, of whom 1 participant (0.8%) was not vaccinated and 131 participants (99.2%) were vaccinated. Three participants (2.3%) withdrew after vaccination, which included 2 participants (1.5%) due to ‘withdrawal by participant’ and 1 participant (0.8%) who was lost to follow-up.
qIRV (60 lag): 133 participants were randomized, of whom 2 participants (1.5%) were not vaccinated and 131 participants (98.5%) were vaccinated. One participant (0.8%) withdrew after vaccination due to being lost to follow-up.

### Immunogenicity

Among participants 18 through 64 years of age, vaccination with qIRV (30 μg) and qIRV (60 μg) elicited increased and similar HAI GMTs against homologous influenza strains at 4 weeks after vaccination. Postvaccination fold-rises and seroconversion were typically more pronounced for A strains than for B strains. A single dose of qIRV (30 μg) and a single dose of qIRV (60 μg) elicited IFN□-producing CD4+ and CD8+ T-cell immune responses to all the 4 strains. T-cell responses elicited by qIRV (30 μg) were generally similar or higher compared with qIRV (60 μg). Vaccine-elicited T-cell responses were observed across age and vaccine groups and reflected induction of IFNγ-producing CD8+ T-cells and a Th1-type CD4+ T-cell response. This Th1-type cytokine profile, characterized by vaccine-elicited IFNγ production in the absence of IL-4 induction, is associated with effective antiviral immunity, coupled with the humoral response of neutralizing antibody production (ie, HAI titers). Responses were generally similar against both A strains and B strains, in contrast to the difference observed for HAI titers (ie, more robust HAI GMTs against A strains and more modest HAI titers against B strains).

### Example 14: Randomized Phase 1 Study to Evaluate the Safety, Tolerability, and Immunogenicity of Different Doses of modRNA-Based Pandemic Influenza Vaccine Candidates

The “pdmFlu” trials refer to the clinical development program currently under initiation (including trial C5561001). This pdmFlu vaccine candidate is a monovalent, modified RNA encoding the HA antigen of A/H5N8 and the platform includes the use of the same LNP as the licensed SARS-CoV-2 COMIRNATY. In the subsequent list of trials (Trials 1A through 2C), the clinical program moves through a series of Phase 1 and Phase 2 trials evaluating the potential of both RNA technologies (modRNA and saRNA), the encoding of both HA and NA of a targeted pandemic influenza strain, and additional LNP formulations that may further optimize dose-sparing regimens and/or extend the duration of immune response to vaccination. These additional studies would ultimately select the optimal next-generation pandemic RNA influenza candidate that retains the same features of rapid production and scale-up that are true of the first generation pdmFlu vaccine candidate.

The randomized Phase 1 study will evaluate the safety, tolerability, and immunogenicity of different doses of modRNA-based pandemic influenza vaccine candidates. The vaccine candidate will be delivered over two scheduled doses and tested in healthy, young adults between the ages of 18 to 49 years of age. This U.S.-based Phase 1 study aims to enroll up to 60 healthy volunteers 18 through 49 years of age. Multiple dose combinations will be investigated. Participants will be randomly assigned to one of four groups. Three of the groups will receive one of three dose levels (30ug, 60ug or 90ug) of the quadrivalent modRNA-based influenza vaccine candidate described herein in two separate vaccinations delivered 20 days apart. As a control, the fourth group will receive one injection of the commercially available quadrivalent influenza vaccine and a placebo injection delivered 20 days later.

The study arms are summarized below:

| Group | Scheduled | Scheduled |
|---|---|---|
| Number | Vaccination 1 | Vaccination 2 |
| 1 | 30 μg Quadrivalent | 30 μg Quadrivalent |
| | modRNA flu vaccine dose | modRNA flu vaccine dose |
| 2 | 60 μg Quadrivalent | 60 μg Quadrivalent |
| | modRNA flu vaccine dose | modRNA flu vaccine dose |
| 3 | 90 μg Quadrivalent | 90 μg Quadrivalent |
| | modRNA flu vaccine dose | modRNA flu vaccine dose |
| 4 | Commercially available flu | Placebo |
| | vaccine dose | |

The vaccine candidate selected for this study will encode the hemagglutinin (HA) antigen of the pandemic influenza strain A/H5N8 (clade 2.3.4.4b), which has been identified as a strain that may provide relevant information about potential responses to a more pathogenic strain. Some novel influenza A viruses are believed to pose a greater pandemic threat because they have already caused serious human illness and have been able to spread in a limited manner from person to person. The vaccine candidate selected for this study will encode the hemagglutinin (HA) antigen of the pandemic influenza strain A/H5N8 (clade 2.3.4.4b), which has been identified as a strain leading to lethal infection in birds and asymptomatic infection in few humans since its identification in 2014.

It is regarded as a strain that may also provide relevant information about potential responses to a more pathogenic strain, A/H5N1 (clade 2.3.4.4b), which has been associated with an unprecedented outbreak in birds and mammals and a limited number of human infections. However, it is anticipated that an mRNA-based influenza vaccine candidate may serve as a platform that can be updated in response to a range of emerging novel pandemic influenza strains. In an emergency, an mRNA-based vaccine could be updated, if needed, to provide a vaccine candidate tailored to the strain responsible for the pandemic threat.

### Phase 1 Dose Finding in Young Healthy Adults

### C5561001: a Study to Evaluate the Safety, Tolerability, and Immunogenicity of Modified RNA Vaccine Candidate Against Pandemic Influenza

Study Design: This Phase 1, randomized, observer-blinded, sponsor-unblinded, dose-ranging study in healthy adults 18 to <50 years of age will evaluate the safety, tolerability, and immunogenicity of different doses of a pdmFlu vaccine candidate against pandemic influenza. Up to 60 participants will be enrolled in a stepwise, dose-escalation manner. Study intervention at each dose level shown will be pdmFlu encoding HA for influenza strain A/H5N8 or licensed quadrivalent influenza vaccine (QIV) as comparator. Enrollment will be conducted in 3 cohorts with participants randomized in a 3:1 (pdmFlu:QIV) ratio as shown in Table 44.

TABLE 44

| Enrollment | Number of | Study |
|---|---|---|
| Cohort | Participants | Intervention |
| 1 | 20 (15 pdmFlu; | 30 μg total mRNA containing |
| | 5 licensed | HA at Vaccination 1 and |
| | QIV) | Vaccination 2 or |
| | | Licensed QIV at Vaccination 1 |
| | | and Placebo at |
| | | Vaccination 2 |
| 2 | 20 (15 pdmFlu; | 60 μg total mRNA containing |
| | 5 licensed | HA at Vaccination 1 and |
| | QIV) | Vaccination 2 or |
| | | Licensed QIV at Vaccination 1 |
| | | and Placebo at |
| | | Vaccination 2 |
| 3 | 20 (15 pdmFlu; | 90 μg total mRNA containing |
| | 5 licensed | HA at Vaccination 1 and |
| | QIV) | Vaccination 2 or |
| | | Licensed QIV at Vaccination 1 |
| | | and Placebo at |
| | | Vaccination 2 |

Two study vaccinations will occur on Days 1 and 21, and vaccine will be administered in an observer-blind fashion. Follow-up visits for safety and collection of blood samples for immunogenicity assessments will occur through 6 months after last dose of study vaccine. The study population may be composed of healthy adults ≥18 to <50 years of age who have not been vaccinated against seasonal influenza in the past 6 months.

Upon completion of appropriate tasks in Phases 2-3, aims to further characterize the safety and immunogenicity of pdmFlu are planned. The first pdmFlu candidate to enter the clinic for evaluation encodes the HA for A/Astrakhan/3212/2020 H5N8, a Glade 2.3.4.4b virus and will be based on the same LNP composition and modRNA manufacturing process used with the licensed COVID-19 vaccine. After completion of Phase 1 study, the pdm Flu candidate will be evaluated in Phase 2 and Phase 3 trials in adults and a Phase 1/2 study in children. Our Phase 2 trial for pdmFlu would include 12 months of follow-up, allowing for assessment of duration of immunogenicity.

Immunogenicity Assays will be developed to be phase-appropriate, either qualified or validated, for measuring the immune response to the homologous vaccine antigen(s) in vaccinated human subjects. Key immunogenicity time points will be identified for the collection and storage of serum samples from vaccinated human subjects. These samples will be tested for immune response and stratified by age and prior seasonal influenza vaccination history. A subset of these samples may be sent to a centralized immunogenicity laboratory for clinical endpoint analyses. Remaining serum samples will be preserved for future use supporting pandemic flu vaccine development, such as for evaluating cross-reactivity to emerging or heterologous influenza strains. An interim analysis will be conducted following the peak immunogenicity time point, utilizing cumulative data on immunogenicity and safety as each subject completes the specified visit. Study database will be monitored, cleaned, and locked. Unblinded group-level data may be prepared.

Phase 2 Dose and Schedule Confirming in Younger and Older Adults (18+, Option for 16+). This is a Phase 2 randomized study designed to identify the optimized dose and schedule of pdmFlu for potential use in a Phase 3 trial. The study also aims to (i) evaluate mix-and-match potential with seasonal influenza vaccines, (ii) assess the impact of a booster dose, and (iii) explore the durability of immune responses.

Study Design: This will be a Phase 2, randomized, observer-blind, double-dummy (if 1 versus 2 dose comparisons are performed), placebo-controlled study in healthy adults ≥18 to <50 years of age (N=210) and ≥65 to <86 years (N=210) (Table 45). The study will evaluate the safety, tolerability, and immunogenicity of different dosages of the pdmFlu vaccine against influenza. Participants will be re-randomized for the administration of pdmFlu vs. an inactive comparator at 6 months post Dose 2. The inclusion of the inactive comparator will allow for the evaluation of the potential benefits of a mix-and-match approach with the seasonal influenza vaccine. Characterizing whether use of a seasonal influenza vaccine as a priming vaccine prior to administration of first dose of pandemic influenza vaccine may offer strategic advantages for consideration in the initial phases of a pandemic outbreak, where pandemic vaccines may be limited but seasonal influenza vaccines are available. The plan to administer a booster dose of pdmFlu will utilize the lower of the two. Study intervention at each dose level shown will encode HA for influenza strain A/H5N8.

TABLE 45
Study Intervention Groups (Phase 2 Adults)

| | | | “Booster” |
|---|---|---|---|
| | | Number of | Vaccine |
| | Schedule | Participants, | 6 Months |
| pdmFlu | (Primary | Age | Post Primary |
| H5N8 | Vaccination) | Cohort | Vaccination |
| pdmFlu | 2 doses pdmFlu | 60, ≥18 to <50 | 30, pdmFlu; 30, |
| vaccine | separated | years | inactive comparator |
| group 1 | by 3 weeks | 60, ≥65 to <86 | 30, pdmFlu; 30, |
| | | years | inactive comparator |
| pdmFlu | 1 dose | 60, ≥18 to <50 | 30, pdmFlu; 30, |
| vaccine | seasonal Flu + | years | inactive comparator |
| group 2 | 1 dose pdmFlu | 60, ≥65 to <86 | 30, pdmFlu; 30, |
| | 3 weeks later | years | inactive comparator |
| pdmFlu | 1 dose pdmFlu + | 60, ≥18 to <50 | 30, pdmFlu; 30, |
| vaccine | 1 dose placebo | years | inactive comparator |
| group 3 | 3 weeks later | 60, ≥65 to <86 | 30, pdmFlu; 30, |
| | | years | inactive comparator |
| Seasonal Flu | 1 dose seasonal | 30, ≥18 to <50 | 15, pdmFlu; 15, |
| | Flu + 1 dose | years | inactive comparator |
| | placebo 3 weeks | 30, ≥65 to <86 | 15, pdmFlu; 15, |
| | later | years | inactive comparator |

Study Population: The study population may be composed of healthy adults ≥18 to <50 years of age (N=210) and ≥65 to <86 years of age (N=210). The lower age limit may also be extended down to 16+ years to align with COVID-19 vaccine trials.

### Phase 3 Pivotal Immunogenicity and Safety Trial in Younger and Older Adults (18+).

This will be a Phase 3 pivotal study designed to confirm the acceptable immunogenicity and safety profile of pdmFlu for intended registration. The study aims to evaluate the safety, tolerability, and immunogenicity of pdmFlu, and assess heterologous HI antibody responses and microneutralization test (MNT) antibody responses.

Study Design: This is a Phase 3, randomized, observer-blind, double-dummy (if 2 dose comparisons are performed), inactive vaccine (seasonal influenza)-controlled study in healthy adults aged ≥18 years (N≥1650) (Table 46). The study will evaluate the safety, tolerability, and immunogenicity of the pdmFlu vaccine against influenza. Study intervention at each dose level shown will encode HA for influenza strain A/H5N8.

TABLE 46
Study Intervention Groups (Phase 3 Adults)

| | Schedule | Sample Size, Age |
|---|---|---|
| pdmFlu H5N8 | (Primary Vaccination) | Cohort |
| pdmFlu | Schedule selected by Phase | <900, ≥18 |
| vaccine | 2 Study (assume 2 doses) | to <65 years |
| | | ≥250, ≥65 years |
| Inactive | 1 dose or 2 doses with 2nd | <900, ≥18 |
| comparator | dose given 3 weeks later, | to <65 years |
| | pending schedule of pdmFlu | ≥250, ≥65 years |

Study Population: The study population may be composed of healthy adults ≥18 years of age (N=1650), excluding individuals who received a seasonal influenza vaccine in the past 6 months. Phase 1/2 Dose Finding, Age-De-escalation in Children (6 Mos to 17 yrs of Age) This will be a Phase 1/2 clinical trial designed to identify one or more doses and schedules of pdmFlu with an acceptable immune response and safety profile in healthy children.

Study Design: This study is a randomized, observer-blind, double-dummy, inactive comparator-controlled study of safety and immunogenicity. The study will use staggered enrollment by age cohort, with stopping and dose adjustment rules applied in each age cohort (Table 47, Table 48). Study intervention at each dose level shown will encode HA for influenza strain A/H5N8.

TABLE 47
Study Intervention Groups (Phase 1/2 Pediatric)

| pdmFlu H5N8 dose vs. Inactive | | Sample size/ |
|---|---|---|
| comparator | Schedule | age cohort |
| Adult dose pdmFlu | 1 or 2 doses separated by | 30 × 2 age |
| | 3 weeks (subject to | cohorts = 60 |
| | change) | |
| Inactive comparator | 1 or 2 doses | 30 × 2 age |
| vaccine | separated by 3 weeks | cohorts = 60 |

TABLE 48
Enrollment Waves

| Enrollment | Enrollment | Enrollment |
|---|---|---|
| Wave 1 | Wave 2 | Wave 3 |
| Adult dose vs. | Adult or half dose | Adult or half |
| inactive | vs. inactive | dose vs. inactive |
| comparator | comparator vaccines | comparator |
| vaccines | 5 to 18 yrs, full | vaccines 6 |
| 9 to 18 yrs, | cohort (n = 50) | mos to <5 yrs |
| sentinel | Adult or half dose | (n = 50) |
| cohort | vs. inactive | |
| (n = 10) | comparator vaccines | |
| | 6 mos to <5 yrs, | |
| | sentinel cohort | |
| | (n = 10) | |

Study Population: The study population will consist of healthy children 6 months to 17 years of age, separated into 2 age cohorts: 6 months to <5 years and 5 to <18 years. A total of 120 participants will be enrolled. Individuals who have received a seasonal influenza vaccine in the past 6 months will be excluded.

Study 1a: Evaluation of HA/NA Ratio in a Pandemic modRNA Vaccine

Study Goal: Induction of NA antibodies may maximize protection against clinical disease, so determining the optimal HA:NA ratio in a pdmFlu vaccine will be conducted. The optimized HA:NA monovalent pdmFlu vaccine will be selected for further use in Study 1 B. Dose selection for Study 1B will be informed by an interim analysis of safety and immunogenicity data gathered up through and including Day 42.

Population: Healthy adults 18 to 49 years of age, no seasonal influenza vaccine in the past 6 months, U.S. center(s)

Study Vaccine Groups: 5 groups—4 HA: NA formulations and comparator vaccine is the pdmFlu HA only H5 candidate selected from 05561001 (Table 49)

TABLE 49
Study 1A Vaccine Groups

| Enrollment | Number of | Study |
|---|---|---|
| Cohort | Participants | Intervention |
| 1 | 30 | HA:NA Ratio 1, H5 monovalent |
| | | mod pdmFlu vaccine |
| 2 | 30 | HA:NA Ratio 2, H5 monovalent |
| | | mod pdmFlu vaccine |
| 3 | 30 | HA:NA Ratio 3, H5 monovalent |
| | | mod pdmFlu vaccine |
| 4 | 30 | HA:NA Ratio 4, H5 monovalent |
| | | mod pdmFlu vaccine |
| 5 | 30 | HA only H5 monovalent vaccine |
| | | selected from Study C5561001 |

Study Design: Approximately 150 participants will be enrolled and randomly assigned to vaccine groups (1:1:1:1:1). All study participants will receive up to 2 doses of study vaccine separated by 3 weeks. Participants will have clinic visits at screening, Day 0, (Day 7), Day 21, (Day 28), Day 42, Day 208 and telephone contacts will occur on Day 3, 24, and Day 118. Visits in brackets refer to participants in the cell mediated immunity (CMI) subset only. At each clinic visit, blood samples will be drawn for HI antibody and MNT testing in all participants and in subgroup of enrolled participants (N=105), additional blood will be taken for CMI testing. Safety assessments will include use of standard e-Diary for 7 days after each vaccination and adverse events will be collected through 6 months following last vaccination (Day 208).

Study 1B: Evaluation of Novel LNPs on modRNA Platform Study Goal: Novel LNPs that have resulted in improved in vitro antigen expression, stability, and/or immunogenicity in preclinical animal models will be evaluated in the clinic to identify one or two formulations for further study in Phase 2 and as a comparator arm(s) in Study 1C. Dose selection for Study 1C will be informed by an interim analysis of safety and immunogenicity data.

Population: Healthy adults 18 to 49 years of age, no seasonal influenza vaccine in the past 6 months, U.S. center(s).

Study Vaccine Groups: 7—three formulations with each novel LNP and comparator vaccine that is the pdmFlu HA only H5 candidate selected from C5561001 (Table 50)

TABLE 50
Study 1B Enrollment Cohorts

| Wave 1 | Wave 2 | Wave 3 |
|---|---|---|
| Low dose | Mid dose | High dose |
| pdmFlu HA/NA + | pdmFlu HA/NA + | pdmFlu HA/NA + |
| LNP1, N = 30 | LNP1, N = 30 | LNP1, N = 30 |
| Low dose | Mid dose | High dose |
| pdmFlu HA/NA + | pdmFlu HA/NA + | pdmFlu HA/NA + |
| LNP2, N = 30 | LNP2, N = 30 | LNP2, N = 30 |
| pdmFlu HA/NA | pdmFlu HA/NA | pdmFlu HA/NA |
| selected from | selected from | selected from |
| Study 1A, | Study 1A, | Study 1A, |
| N = 10 | N = 10 | N = 10 |

Study Design: Approx. 210 participants will be enrolled and randomly assigned to vaccine groups (3:3:1) in a series of 3 dose escalating, staggered waves of enrollment. All study participants will receive up to 2 doses of study vaccine separated by 3 weeks. Participants will have clinic visits at screening, Day 0, (Day 7), Day 21, (Day 28), Day 42, Day 208 and telephone contacts will occur on Day 3, 24, and Day 118. Visits in brackets refer to participants in the CMI subset only. HI antibody testing and safety assessments will be similar to study 1A above.

Study 1C: Evaluation of saRNA and modRNA Pandemic Influenza Candidates Including Further Characterization of Novel LNP for Optimization of saRNA Formulation

Study Goal: saRNA has the potential to be dose-sparing and improve duration of the immune response compared to modRNA, so saRNA candidates will be evaluated to identify one or two formulations for further study in Phase 2. Dose selection for Study 2 will be informed by an interim analysis of safety/immunogenicity data through Day 42.

Population: Healthy adults 18 to 49 years of age, no seasonal influenza vaccine in the past 6 months, U.S. center(s).

Study Vaccine Groups: 7—three formulations with each novel LNP and comparator vaccine that is the pdmFlu HA: NA H5 candidate selected from Study 1B (Table 51)

TABLE 51
Study 1C Enrollment Cohorts

| Wave 1 | Wave 2 | Wave 3 |
|---|---|---|
| Low dose SA | Mid dose SA | High dose SA |
| pdmFlu HA/NA + | pdmFlu HA/NA + | pdmFlu HA/NA + |
| LNP3, N = 30 | LNP3, N = 30 | LNP3, N = 30 |
| Low dose SA | Mid dose SA | High dose SA |
| pdmFlu HA/NA + | pdmFlu HA/NA + | pdmFlu HA/NA + |
| LNP4, N = 30 | LNP4, N = 30 | LNP4, N = 30 |
| Mod pdmFlu HA:NA | Mod pdmFlu HA:NA | Mod pdmFlu HA:NA |
| with LNP of | with LNP of | with LNP of |
| choice based | choice based | choice based |
| on Study 1B, | on Study 1B, | on Study 1B, |
| N = 10 | N = 10 | N = 10 |

Study Design: Approx. 210 participants will be enrolled and randomly assigned to vaccine groups (3:3:1) in a series of 3 dose escalating, staggered waves of enrollment. All study participants will receive up to 2 doses of study vaccine separated by 3 weeks. Participants will have clinic visits at screening, Day 0, (Day 7), Day 21, (Day 28), Day 42, Day 208 and telephone contacts will occur on Day 3, 24, and Day 118. Visits in brackets refer to participants in the CMI subset only. At each clinic visit, blood samples will be drawn for HI antibody and MNT testing in all participants and in approximately half of enrolled participants (N=105), additional blood will be taken for CMI testing. Safety assessments will be similar to study 1A above.

Study 1D: Evaluation of “Mix and Match” Scenarios with saRNA and modRNA Pandemic Influenza Candidates Given with and without Seasonal Influenza Vaccines

Study Goal: To determine the impact of 2 doses of standalone pandemic vaccine candidates versus those of a mixed regimen with seasonal influenza vaccine as priming vaccine versus a single dose of standalone pandemic vaccine. This will be used to inform Phase 2 study designs.

Population: Healthy adults 18 to 49 years of age, no seasonal influenza vaccine in the past 6 months, U.S. center(s).

Study Vaccine Groups: 7-three formulations with each novel LNP and comparator vaccine that is the pdmFlu HA:NA H5 candidate selected from Study 1C (Table 52)

TABLE 52
Study 1D Vaccine Groups

| Enrollment | | |
|---|---|---|
| Cohort | N | Study Intervention |
| 1 | 30 | Mod pdmFlu HA:NA with LNP of choice based |
| | | on Study 1B, N = 30, standalone |
| | | vaccine, one dose followed |
| | | by placebo given 3 weeks later |
| 2 | 30 | Mod pdmFlu HA:NA with LNP of choice |
| | | based on Study 1B, N = 30, standalone |
| | | vaccine, two doses separated by 3 weeks |
| 3 | 30 | Mod pdmFlu HA:NA with LNP of choice based |
| | | on Study 1B, N = 30, given as |
| | | second dose in two dose series 3 weeks after |
| | | vaccination with seasonal influenza vaccine |
| 4 | 30 | SA pdmFlu HA:NA with LNP of choice based |
| | | on Study 1C, N = 30, standalone |
| | | vaccine, one dose followed by |
| | | placebo given 3 weeks later |
| 5 | 30 | SA pdmFlu HA:NA with LNP of choice based |
| | | on Study 1C, N = 30, standalone |
| | | vaccine, two doses separated by 3 weeks |
| 6 | 30 | SA pdmFlu HA:NA with LNP of choice based |
| | | on Study 1C, N = 30, given as |
| | | second dose in two dose series 3 weeks after |
| | | vaccination with seasonal influenza vaccine |
| 7 | 30 | HA only H5 monovalent vaccine selected from |
| | | Study C5561001, two doses separated by 3 weeks |

Study Design: Approx. 210 participants will be enrolled and randomly assigned to vaccine groups (1:1:1:1:1:1:1). All study participants will receive up to 2 doses of study vaccine separated by 3 weeks. Participants will have clinic visits at screening, Day 0, (Day 7), Day 21, (Day 28), Day 42, Day 208 and telephone contacts will occur on Day 3, 24, and Day 118. Visits in brackets refer to participants in the CMI subset only. At each clinic visit, blood samples will be drawn for HI antibody and MNT testing in all participants and in a subgroup of enrolled participants (N=105), additional blood will be taken for CMI testing. Safety assessments will be similar to study 1A above.

Study 2A: Characterization of modRNA HA/NA Candidates with Optimized LNP in Younger and Older Adults

Study Goal: To identify one or two formulations for further study in Phase 2C. Dose selection for Study 2C for younger and older adults will be informed by an interim analysis of safety and immunogenicity after Day 42 in Waves 2 and 3, respectively.

Population: Healthy adults ≥18 to <50 and ≥65 to <86 years of age, no seasonal influenza vaccine in the past 6 months, US center(s). (Table 53)

TABLE 53
Study 2A Enrollment Cohorts

| Wave 1: Adults ≥65 | Wave 2: Adults ≥18 | Wave 3: Adults ≥65 |
|---|---|---|
| to <86 yrs. | to <50 yrs | to <86 yrs |
| Low dose mod pdmFlu | Selected mod pdmFlu HA/NA + | Selected mod pdmFlu HA/NA + |
| HA/NA + LNP from Study | LNP1 from Study 1B, N = 120, | LNP from Wave 1, N = 120, given |
| 1B, N = 30 | given as two doses | as two doses |
| Mid dose mod pdmFlu | Selected mod pdmFlu HA/NA + | Selected mod pdmFlu HA/NA + |
| HA/NA + LNP from Study | LNP2 from Study 1B, N = 120, | LNP formulation #1 from Wave 1, |
| 1B, N = 30 | given as two doses | N = 120, give as one dose + |
| | | placebo |
| High dose mod pdmFlu | Selected mod pdmFlu HA/NA + | |
| HA/NA + LNP from Study | LNP1 from Study 1B, N = 120, | |
| 1B, N = 30 | given as one dose + placebo | |
| | Selected mod pdmFlu HA/NA + | |
| | LNP2 from Study 1B, N = 120, | |
| | given as one dose + placebo | |

Study Design: Approx. 810 participants will be enrolled in two to three waves. The first wave will enroll older adults in a parallel fashion (1:1:1) to each of the three doses previously evaluated in younger adults in Study 1 B. An interim analysis of safety and immunogenicity will be performed after Day 42 to inform dose selection for further study in Wave 3 in the older adult population. In Wave 2, which may be conducted at the same time as Wave 1, participants ≥18 to <50 years of age will be randomly assigned 1:1:1:1 to one of two formulations of mod pdmFlu HA/NA formulation identified from Study 1 B given as a 1 or 2-dose active vaccine schedule. In Wave 3, participants ≥65 to <86 years of age will be randomly assigned 1:1 to one of two dose schedules of the mod pdmFlu HA/NA formulation selected in Wave 1. All study participants will receive up to 2 doses of study vaccine separated by 3 weeks. Participants will have clinic visits at screening, Day 0, (Day 7), Day 21, (Day 28), Day 42, Day 208 and telephone contacts will occur on Day 3, 24, and Day 118. Visits in brackets refer to participants in the CMI subset only. At each clinic visit, blood samples will be drawn for HI antibody and MNT testing in all participants and in older participants in Wave 3 only, approximately 60 participants will have additional blood taken for CMI testing. Safety assessments will be similar to study 1A above.

Study 2B: Characterization of saRNA HA/NA Candidates with Optimized LNP in Younger and Older Adults

Study Goal: To identify one or two formulations for further study in Phase 2C. Dose selection for Study 2C for younger and older adults will be informed by an interim analysis of safety and immunogenicity after Day 42 in Waves 2 and 3, respectively.

Population: Healthy adults ≥18 to <50 and ≥65 to <86 years of age, no seasonal influenza vaccine in the past 6 months, US center(s). (Table 54)

TABLE 54
Study 2B Enrollment Cohorts

| Wave 1: Adults ≥65 | Wave 2: Adults ≥18 | Wave 3: Adults ≥65 |
|---|---|---|
| to <86 yrs | to <50 yrs | to <86 yrs |
| Low dose SA pdmFlu | Selected SA pdmFlu HA/NA + | Selected SA pdmFlu HA/NA + LNP |
| HA/NA + LNP from Study | LNP3 from Study 1C, given as 2 | from Wave 1, N = 120, given as two |
| 1C, N = 30 | doses, N = 120 | doses |
| Mid dose SA pdmFlu | Selected SA pdmFlu HA/NA + | Selected SA pdmFlu HA/NA + LNP |
| HA/NA + LNP from Study | LNP4 from Study 1C, given as 2 | from Wave 1, N = 120, give as one |
| 1C, N = 30 | doses, N = 120 | dose + placebo |
| High dose SA pdmFlu | Selected SA pdmFlu HA/NA + | |
| HA/NA + LNP from Study | LNP3 from Study 1C, given as 1 | |
| 1C, N = 30 | dose + placebo, N = 120 | |
| | Selected SA pdmFlu HA/NA + | |
| | LNP4 from Study 1C, given as 1 | |
| | dose + placebo, N = 120 | |

Approx. 810 participants will be enrolled in two to three waves. The first wave will enroll older adults in a parallel fashion (1:1:1) to each of the three doses previously evaluated in younger adults in Study 1C. An interim analysis of safety and immunogenicity will be performed after Day 42 to inform dose selection for further study in Wave 3 in the older adult population. In Wave 2, which may be conducted at the same time as Wave 1, participants ≥18 to <50 years of age will be randomly assigned 1:1:1:1 to one of two formulations of SA pdmFlu HA/NA formulation identified from Study 1C given as a 1 or 2-dose active vaccine schedule. In Wave 3, participants ≥65 to <86 years of age will be randomly assigned 1:1 to one of two dose schedules of the SA pdmFlu HA/NA formulation selected in Wave 1.

All study participants will receive up to 2 doses of study vaccine separated by 3 weeks. Participants will have clinic visits at screening, Day 0, (Day 7), Day 21, (Day 28), Day 42, Day 208 and telephone contacts will occur on Day 3, 24, and Day 118. Visits in brackets refer to participants in the CMI subset only. At each clinic visit, blood samples will be drawn for HI antibody and MNT testing in all participants and in older participants in Wave 3 only, approx. 60 participants will undergo CMI testing. Safety assessments will be similar to study 1A above.

### Study 2C: Characterization of Top Pandemic RNA Vaccine Candidates in Younger and Older Adults, Including Booster Dose Evaluation in Both Age Cohorts and Mix and Match in Older Adults

Study Goals: To include characterization of optimal dosing schedule for primary and boosting vaccination. This Phase 2 study may be conducted in parallel to the start of Phase 3 in younger adults. As this study includes an evaluation of two different platforms (modRNA and saRNA) in older adults, it is expected that data from an interim analysis conducted after Day 42 would inform formulation selection for the Phase 3 program in this age cohort.

Population: Healthy adults ≥18 to <50 and ≥65 to <86 years of age, no seasonal influenza vaccine in the past 6 months, US center(s). (Table 55)

TABLE 55
Study 2C Vaccine Groups

| | | |
|---|---|---|
| Enrollment | Number of | |
| Cohort | Participants | Study Intervention |

Younger Adults: ≥18 to <50 Yrs of Age

| | | |
|---|---|---|
| 1 | 60 | Selected Candidate and Schedule from Study 1D, no |
| | | booster at 6 months |
| 2 | 60 | Selected Candidate and Schedule from Study 1D, with |
| | | booster at 6 months |

Older Adults: ≥65 to <86 Yrs of Age

| | | |
|---|---|---|
| 1 | 60 | Selected modRNA Candidate from Study 2A given as 1 |
| | | dose 3 weeks after placebo, placebo as booster at 6 |
| | | months |
| 2 | 60 | Selected modRNA Candidate from Study 2A given as 1 |
| | | dose 3 weeks after seasonal Flu vaccine, placebo as |
| | | booster at 6 months |
| 3 | 60 | Selected modRNA Candidate from Study 2A given as 1 |
| | | dose 3 weeks after placebo, pdmFlu booster at 6 |
| | | months |
| 4 | 60 | Selected modRNA Candidate from Study 2A given as 1 |
| | | dose 3 weeks after seasonal Flu vaccine, pdmFlu |
| | | booster at 6 months |
| 5 | 60 | Selected saRNA Candidate from Study 2B given as 1 |
| | | dose 3 weeks after placebo, placebo as booster at 6 |
| | | months |
| 6 | 60 | Selected saRNA Candidate from Study 2B given as 1 |
| | | dose 3 weeks after seasonal flu vaccine, placebo as |
| | | booster at 6 months |
| 7 | 60 | Selected saRNA Candidate from Study 2B given as 1 |
| | | dose 3 weeks after placebo, pdmFlu booster at 6 |
| | | months |
| 8 | 60 | Selected saRNA Candidate from Study 2B given as 1 |
| | | dose 3 weeks after seasonal Flu vaccine, pdmFlu |
| | | booster at 6 months |

Study Design: Approx. 600 participants will be enrolled (120 participants ≥18 to <50 years of age and 480 participants ≥65 to <86 years of age). Younger adults will be randomly assigned 1:1 to the preferred pandemic flu vaccine candidates from Study 1 D given with or without a booster dose of the same vaccine at 6 months. Older adults will be randomly assigned 1:1:1:1:1:1:1:1 to the selected pandemic flu vaccine candidates from Study 2A and Study 2B given with or without a booster dose of the same vaccine at 6 months and also given with or without a priming dose of seasonal influenza vaccine.

All study participants will receive up to 3 doses of study vaccine given at Day 0, Day 21, Day 201. Participants will have clinic visits at screening, Day 0, (Day 7), Day 21, (Day 28), Day 42, Day 201, (Day 208), Day 222, and 381 and telephone contacts will occur on Day 3, 24, 118, 291. Visits in brackets refer to participants in the CMI subset only. At each clinic visit, blood samples will be drawn for HI antibody and MNT testing in all participants. In approximately 30 participants in each vaccine group (n=300) will have additional blood taken for CMI testing. Safety assessments will be similar to study 1A above.

### Pediatric Investigational Plan: Dose Ranging Based on Formulation and Schedule Identified in Younger Adults

Study Goals: To characterize safety and immunogenicity of the pandemic influenza vaccine candidate in a pediatric population in a pre-pandemic setting for potential study in a pandemic.

Population: Healthy, not recently vaccinated children 6 months to 17 years of age

Study Vaccine Groups: 3 study vaccine formulations evaluated in 3 age cohorts (Table 56)

TABLE 56
Pediatric Study Groups

| Age Cohort | Wave 1 | Wave 2 | Wave 3 | Wave 4 | Wave 5 |
|---|---|---|---|---|---|
| 9 to 17 yrs | pdmFlu low | pdmFlu mid | pdmFlu high | | |
| | dose vs. | dose vs. | dose vs. | | |
| | Seasonal Flu | Seasonal Flu | Seasonal Flu | | |
| | vaccine (2:1), | vaccine (2:1), | vaccine (2:1), | | |
| | n = 36 | n = 36 | n = 36 | | |
| 3 to 8 yrs | | pdmFlu low | pdmFlu mid | pdmFlu high | |
| | | dose vs. | dose vs. | dose vs. | |
| | | Seasonal Flu | Seasonal Flu | Seasonal Flu | |
| | | vaccine (2:1), | vaccine (2:1), | vaccine (2:1), | |
| | | n = 36 | n = 36 | n = 36 | |
| 6 mo to 2 | | | pdmFlu low | pdmFlu mid | pdmFlu high |
| yrs | | | dose vs. | dose vs. | dose vs. |
| | | | Seasonal Flu | Seasonal Flu | Seasonal Flu |
| | | | vaccine (2:1), | vaccine (2:1), | vaccine (2:1), |
| | | | n = 36 | n = 36 | n = 36 |

Study Design: Approx. 324 participants will be enrolled and randomly assigned to pandemic vs. seasonal influenza vaccine (2:1) in a series of 3 dose escalating, age deescalating waves of enrollment. Children 9 to 17 years of age will receive 1 or 2 doses of study vaccine depending on final primary dosing schedule identified in adults. Children <9 years of age will receive up to 2 doses of study vaccine separated by 3 weeks. Participants will have clinic visits at screening, Day 0, (Day 7), Day 21, [in less than 9 years of age only: (Day 28), Day 42], [Day 180/208] and telephone contacts will occur on Day 3, 24, and Day [90/118]. Visits in parentheses refer to participants in the CMI subset and the bracketed visit numbers refer to older and younger children visit dates, respectively. At each clinic visit, blood samples will be drawn for HI antibody and MNT testing in all participants and in approximately 12 participants in each age cohort and enrollment wave (n=108 total) additional blood will be taken for CMI testing. Safety assessments will be similar to study 1A above.

### Example 15: Overview of Nonclinical Breadth of Coverage Testing

The investigational pdmFlu vaccine to be developed under IND 30059 is a nucleoside-modified mRNA-LNP vaccine encoding the HA antigen derived from the A/Astrakhan/3212/2020 H5N8, a Glade 2.3.4.4b virus, which is related to the currently predominant highly pathogenic avian influenza viruses of the H5N1 subtype (Glade 2.3.4.4b) that has been associated with an unprecedented outbreak in birds and mammals and a limited number of human infections. Immunogenicity studies conducted in mice using the investigational H5 pdmFlu vaccine demonstrate the ability of the vaccine to induce functional HA-specific antibodies against related H5 avian influenza viruses.

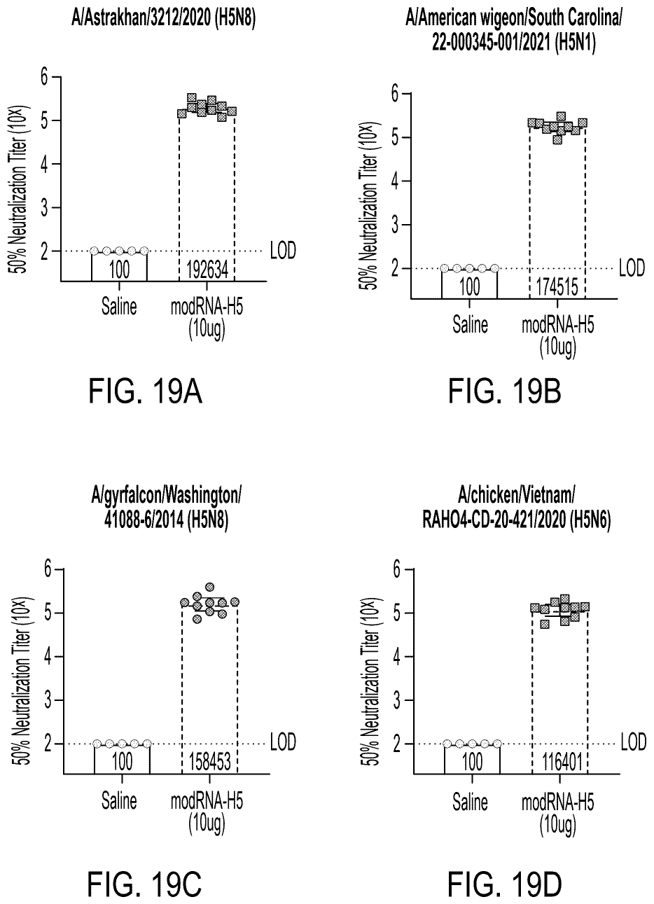

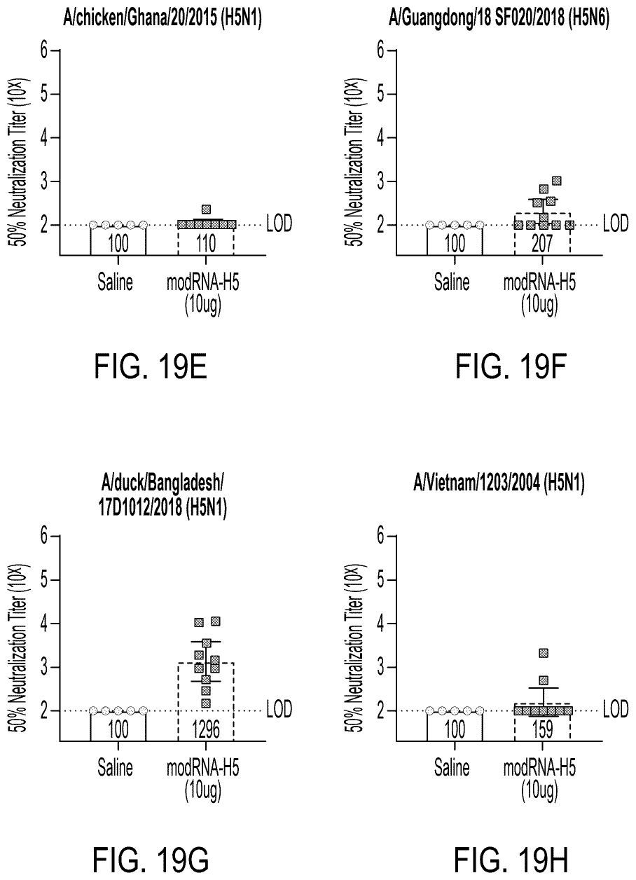

Cross-Neutralization of Heterologous H5Nx Viruses by a Pandemic Influenza modRNA H5 Vaccine in Mice. BALB/c mice were immunized IM with 10 μg of LNP-formulated influenza modRNA vaccine encoding the HA antigen from A/Astrakhan/3212/2020 (H5N8) on Days 0 and 28. To evaluate the breadth of coverage of the vaccine, virus neutralization titers against the matched strain and three related heterologous H5Nx virus strains (Table 57) were measured using sera collected on Day 42 (2 weeks post-dose 2) (FIG. 19A-D). Vaccination with the H5 pdmFlu vaccine induced a robust virus neutralization response to the matched A/Astrakhan/3212/2020 H5N8 strain (Glade 2.3.4.4b) (FIG. 19A). Vaccination induced similar high levels of virus neutralizing antibodies to three heterologous H5 viruses which share 95% or more HA sequence homology with the vaccine strain: A/American wigeon/South Carolina/22-000345-001/2021 H5N1 (Glade 2.3.4.4b), A/gyrfalcon/Washington/41088-6/2014 H5N8 (Glade 2.3.4.4c), and A/chicken/Vietnam/RAHO4-CD-20-421/2020 H5N6 (Glade 2.3.4.4g) (FIG. 19B-D). Vaccination with the H5 pdmFlu vaccine induced a robust virus neutralization response to additional virus strains, as shown in FIG. 19E-H. Accordingly, the H5 and H9 modRNA-LNP vaccines elicited robust virus neutralizing responses in a dose-dependent manner after one and/or two doses. MNT titers were comparable to those elicited by a seasonal H1 modRNA vaccine. Mice vaccinated with 2 doses (10 ug/dose) of modRNA-H5 elicited antibodies that cross-neutralize closely-related heterologous H5Nx viruses. High levels of neutralizing antibodies elicited against heterologous strains with >95% HA sequence identity to the vaccine strain HA. See Table 57 for estimated % sequence Identity to Vaccine Strain A/Astrakhan/3212/2020 (H5N8) HA and NA.

In summary, H5N8 modRNA vaccine elicited a robust virus neutralizing response in a dose-dependent manner. H5N8 modRNA vaccine elicited similar MNT titers compared to the H5 modRNA vaccine alone at both dose levels. H5N8 modRNA vaccine elicited strong HA-specific and NA-specific CD4+ and CD8+ T cell responses. H5N8 modRNA-induced CD4+ T cell responses were higher or similar in magnitude to the H5 or N8 only modRNA groups, suggesting no interference of NA on HA-specific CD4+ T cell responses. H5N8 modRNA vaccine elicited higher H5-specific but lower N8-specific CD8+ T cell responses compared to the H5 or N8 only modRNA groups, suggesting potential interference of HA on NA-specific CD8+ T cell responses. Mice vaccinated with 2 doses (1 ug/dose) of modRNA-N8 elicited antibodies that cross-inhibit NA of a heterologous virus encoding the same NA subtype, but not viruses with heterologous NA subtypes.

TABLE 57
Avian Influenza Viruses for Neutralization Testing

| | | | % seq. Identity | % seq. Identity |
|---|---|---|---|---|
| | | | to Vaccine | to Vaccine |
| Virus Strain | Subtype | Clade | Strain HA | Strain NA |
| A/Astrakhan/3212/2020* | H5N8 | 2.3.4.4b | — | — |
| A/American wigeon/South | H5N1 | 2.3.4.4b | 99.5 | 49.8 |
| Carolina/22-000345-001/2021 | | | | |
| A/gyrfalcon/Washington/41088-6/2014 | H5N8 | 2.3.4.4c | 96.1 | 94.5 |
| A/chicken/Ghana/20/2015 | H5N1 | 2.3.2.1f | 91.4 | 50.3 |
| A/Guangdong/18SF020/2018 | H5N6 | 2.3.4.4h | 93.5 | 41.7 |
| A/chicken/Vietnam/RAHO4-CD-20- | H5N6 | 2.3.4.4g | 95.4 | 41.5 |
| 421/2020 | | | | |
| A/duck/Bangladesh/17D1012/2018 | H5N1 | 2.3.2.1a | 92.1 | 50.5 |
| A/Vietnam/1203/2004 | H5N1 | 1 | 92.3 | 50.3 |

*Vaccine strain

See FIG. 19A-D Virus Neutralization Titers Elicited by Immunization of Mice with a Pandemic Influenza modRNA H5 Vaccine.

TABLE 58
Breadth of coverage testing - modRNA -N8.

| | | | | | Dose | Vacci- |
|---|---|---|---|---|---|---|
| Gp | | | Dosing | Dose | Volume/ | nation |
| # | N/Gr | RNA | Group | (μg) | Route | (Day) |
| 1 | 10 | Saline | — | — | 50 μL/IM | 0, 28 |
| 2 | 10 | pdmFlu | Low | 0.3 μg | | |
| | | modRNA-H5 + | Dose | (0.2 μg HA/ | | |
| | | pdmFlu | | 0.1 μg NA) | | |
| | | modRNA-N8 | | | | |
| | | (2:1) | | | | |
| 3 | 10 | pdmFlu | | 0.4 μg | | |
| | | modRNA-H5 + | | (0.2 μg HA/ | | |
| | | pdmFlu | | 0.2 μg NA) | | |
| | | modRNA-N8 | | | | |
| | | (1:1) | | | | |
| 4 | 10 | pdmFlu | Medium | 1.5 μg | | |
| | | modRNA-H5 + | Dose | (1 μg HA/ | | |
| | | pdmFlu | | 0.5 μg NA) | | |
| | | modRNA-N8 | | | | |
| | | (2:1) | | | | |
| 5 | 10 | pdmFlu | | 2 μg | | |
| | | modRNA-H5 + | | (1 μg HA/ | | |
| | | pdmFlu | | 1 μg NA) | | |
| | | modRNA-N8 | | | | |
| | | (1:1) | | | | |
| 6 | 10 | pdmFlu | — | 0.2 μg | | |
| 7 | 10 | modRNA-H5 | — | 1 μg | | |
| 8 | 10 | pdmFlu | — | 0.2 μg | | |
| 9 | 10 | modRNA-N8 | — | 1 μg | | |
| 10 | 10 | modRNA-H1 + | Low | 0.4 μg | | |
| | | modRNA-N1 | Dose | (0.2 μg HA/ | | |
| | | (1:1) | | 0.2 μg NA) | | |
| 11 | 10 | | Medium | 2 μg | | |
| | | | Dose | (1 μg HA/ | | |
| | | | | 1 μg NA) | | |

### Example 16: Sequence Analysis of PdmFlu modRNA-H5 Vaccine for Predicting Protection Against (2024) HPAI H5N1 Cattle Outbreak Viruses

Preclinical breadth of coverage analysis of the pdmFlu modRNA-H5 vaccine demonstrated that the vaccine induces a robust neutralizing antibody response against heterologous H5Nx viruses that share >95% HA sequence homology with the A/Astrakhan/3212/2020 (H5N8) vaccine strain and have minimal or no mutations in epitopes and/or glycosylation sites which are predicted to impact antigenicity.

The following HA sequence analysis was used to evaluate coverage of the pdmFlu modRNA-H5 vaccine and current licensed H5N1 vaccines against dairy cattle and human isolates from the current 2024 HPAI H5N1 outbreak in cattle, with the following results: PdmFlu modRNA-H5 vaccine shares 98% HA sequence homology with the 2024 dairy cattle and human HPAI H5N1 viruses with no mutations in epitope regions or glycosylation sites that are predicted to impact antigenicity (see Table 59); current licensed H5N1 vaccines share ˜91% HA sequence homology with dairy cattle and human HPAI H5N1 viruses and contain 40+ mutations with multiple mutations in epitope regions and glycosylation sites that are predicted to negatively impact antigenicity (See Table 60).

In some embodiments, the pdmFlu modRNA-H5 vaccine elicits an effective immune response against the 2024 A/dairy cattle/Texas/24-008749-001-original/2024, which has an HA amino acid sequence having 98.24% identity to the HA amino acid sequence of the pdmFlu modRNA-H5 vaccine strain, A/Astrakhan/3212/2020. In some embodiments, the pdmFlu modRNA-H5 vaccine elicits an effective immune response against the 2024 A/Texas/37/2024, which has an HA amino acid sequence having 98.41% identity to the HA amino acid sequence of the pdmFlu modRNA-H5 vaccine strain, A/Astrakhan/3212/2020. In preferred embodiments, the amino acid sequence for the vaccine strain's HA is at least 92% identical to the amino acid sequence for the target influenza strain's HA.

Accordingly, the pdmFlu modRNA-H5 vaccine is expected to provide cross-protection against HPAI H5N1 viruses currently circulating among dairy cattle. More specifically, the pdmFlu modRNA-H5 vaccine candidate comprises an RNA polynucleotide comprising a 5′ UTR comprising SEQ ID NO: 5, a 3′ UTR comprising SEQ ID NO: 8, an open reading frame encoding influenza hemagglutinin of A/Astrakhan/3212/2020, and a polyA tail, encapsulated in a lipid nanoparticle comprising ALC-0315, ALC-0159, cholesterol, and DSPC. In another embodiment, the pdmFlu modRNA vaccine comprises an RNA polynucleotide comprising a 5′ UTR comprising SEQ ID NO: 5, a 3′ UTR comprising SEQ ID NO: 8, an open reading frame encoding influenza hemagglutinin of A/Texas/37/2024, and a polyA tail, encapsulated in a lipid nanoparticle comprising a cationic lipid, pegylated lipid, cholesterol, and phospholipid.

In some embodiments, the open reading frame encodes an influenza hemagglutinin comprising an epitope having the sequence selected from the group consisting of: EFIRVPEW (SEQ ID NO: 35), EFISVPEW (SEQ ID NO: 36), EFTNPEW (SEQ ID NO: 37), and EFINVPEW (SEQ ID NO: 38). In some embodiments, the open reading frame comprises a sequence that has at least 80% (e.g., any number between 80% and 100%, inclusive, e.g., 80%, 85%, 90%, 91%, 92%, 93%, 94%, 95%, 96%, 97%, 98%, 99%, and 100%) identity to an amino acid sequence of the epitope.

In some embodiments, the open reading frame encodes an influenza hemagglutinin comprising an epitope having the sequence selected from the group consisting of: PKSSWPNHETSL (SEQ ID NO: 9), PRSSWPNHETSL (SEQ ID NO: 10), PKRSWSNHTSS (SEQ ID NO: 11), PKDSWSDHEASL (SEQ ID NO: 12), and PKSSWSSHEASL (SEQ ID NO: 13). In some embodiments, the open reading frame comprises a sequence that has at least 80% (e.g., any number between 80% and 100%, inclusive, e.g., 80%, 85%, 90%, 91%, 92%, 93%, 94%, 95%, 96%, 97%, 98%, 99%, and 100%) identity to an amino acid sequence of the epitope.

In some embodiments, the open reading frame encodes an influenza hemagglutinin comprising an epitope having the sequence selected from the group consisting of: SNNAEEQTNLY (SEQ ID NO: 14), SNNAAEQTNLY (SEQ ID NO: 15), SNSAEEQTDLY (SEQ ID NO: 16), PNDEAEQTKLY (SEQ ID NO: 17), PNDEAEQTRLY (SEQ ID NO: 18), and PNDAAEQTKLY (SEQ ID NO: 19). In some embodiments, the open reading frame comprises a sequence that has at least 80% (e.g., any number between 80% and 100%, inclusive, e.g., 80%, 85%, 90%, 91%, 92%, 93%, 94%, 95%, 96%, 97%, 98%, 99%, and 100%) identity to an amino acid sequence of the epitope.

In some embodiments, the open reading frame encodes an influenza hemagglutinin comprising an epitope having the sequence selected from the group consisting of: EFIRVPEW (SEQ ID NO: 20), EFISVPEW (SEQ ID NO: 21), EFTNPEW (SEQ ID NO: 22), EFINVPEW (SEQ ID NO: 23); PKSSWPNHETSL (SEQ ID NO: 24), PRSSWPNHETSL (SEQ ID NO: 25), PKRSWSNHTSS (SEQ ID NO: 26), PKDSWSDHEASL (SEQ ID NO: 27), PKSSWSSHEASL (SEQ ID NO: 28); SNNAEEQTNLY (SEQ ID NO: 29), SNNAAEQTNLY (SEQ ID NO: 30), SNSAEEQTDLY (SEQ ID NO: 31), PNDEAEQTKLY (SEQ ID NO: 32), PNDEAEQTRLY (SEQ ID NO: 33), and PNDAAEQTKLY (SEQ ID NO: 34). In some embodiments, the open reading frame comprises a sequence that has at least 80% (e.g., any number between 80% and 100%, inclusive, e.g., 80%, 85%, 90%, 91%, 92%, 93%, 94%, 95%, 96%, 97%, 98%, 99%, and 100%) identity to an amino acid sequence of the epitope.

Analysis of PdmFlu modRNA-H5 Preclinical Breadth of Coverage Data

Heterologous viruses to which the pdmFlu modRNA-H5 vaccine elicits high neutralizing antibody titers to have similar sequences in the receptor binding domain and epitopes as the vaccine strain.

TABLE 59
Sequence Analysis of pdmFlu modRNA-H5 HA vaccine candidate

| Vaccine | | | Outbreak | | | HA Seq | |
|---|---|---|---|---|---|---|---|
| Strain | GISAD ID | Clade | Isolate | GISAID ID | Clade | ID (%) | |
| A/Astrakhan/ | EPI_ISL_13655139 | 2.3.4.4b | A/dairy cattle/ | EPI_ISL_19014384 | 2.3.4.4b | 98.24 | cattle |
| 3212/2020 | | | Texas/24- | | | | |
| | | | 008749-001- | | | | |
| | | | original/2024 | | | | |
| A/Astrakhan/ | EPI_ISL_13655139 | 2.3.4.4b | A/Texas/37/2024 | EPI_ISL_19027114 | 2.3.4.4b | 98.41 | human |
| 3212/2020 | | | | | | | |

TABLE 60
Sequence Analysis of Licensed H5N1 vaccines

| | | | Outbreak | | | HA Seq |
|---|---|---|---|---|---|---|
| Vaccine Strain | GISAID ID | Clade | Isolate | GISAID ID | Clade | ID (%) |
| A/Vietnam/1203/2004 | EPI_ISL_10656749 | 1 | A/Texas/37/2024 | EPI_ISL_19027114 | 2.3.4.4b | 91.21 |
| (Sanofi) | | | | | | |
| A/Indonesia/05/2005 | EPI_ISL_5729 | 2.1.3.2 | A/Texas/37/2024 | EPI_ISL_19027114 | 2.3.4.4b | 91.56 |
| (GSK) | | | | | | |
| A/turkey/Turkey/1/2005 | EPI_ISL_143438 | 2.2.1 | A/Texas/37/2024 | EPI_ISL_19027114 | 2.3.4.4b | 91.03 |
| (CSL Seqirus) | | | | | | |
| A/Vietnam/1203/2004 | EPI_ISL_10656749 | 1 | A/dairy cattle/ | EPI_ISL_19014384 | 2.3.4.4b | 91.38 |
| (Sanofi) | | | Texas/24- | | | |
| | | | 008749-001- | | | |
| | | | original/2024 | | | |
| A/Indonesia/05/2005 | EPI_ISL_5729 | 2.1.3.2 | A/dairy cattle/ | EPI_ISL_19014384 | 2.3.4.4b | 91.56 |
| (GSK) | | | Texas/24- | | | |
| | | | 008749-001- | | | |
| | | | original/2024 | | | |
| A/turkey/Turkey/1/2005 | EPI_ISL_143438 | 2.2.1 | A/dairy cattle/ | EPI_ISL_19014384 | 2.3.4.4b | 91.20 |
| (CSL Seqirus) | | | Texas/24- | | | |
| | | | 008749-001- | | | |
| | | | original/2024 | | | |

### Example 17: C5561001 Preliminary Summary of Immunogenicity Data from Hemagglutination Inhibition (HAI) Assay for the Detection of Influenza H5N8 Virus Antibodies

Following the study described in Example 14, the following tables provide a preliminary summary of the immunogenicity data.

TABLE 61
GMT and GMR of H5N8 HAI Titer - mITT Population

| | Vaccine Group (as Randomized) | | | | | | | | | | |
|---|---|---|---|---|---|---|---|---|---|---|---|
| | pdm Flu Vaccine | | | | | | | | | | |
| | 30 μg | | | 60 μg | | | 60 μg/30 μg | | Licensed QIV/Placebo | | |
| Visit | n | GMT | (95% CI) | n | GMT | (95% CI) | GMR | (95% CI) | n | GMT | (95% CI) |
| Prevax | 15 | 5 | (NE) | 15 | 5 | (NE) | | | 9 | 5 | (NE) |
| 1-Week | 15 | 5.5 | (4.8, 6.3) | 14 | 7.8 | (5.6, 10.9) | 1.42 | (1.00, 2.03) | 10 | 5 | (NE) |
| Post Vax1 | | | | | | | | | | | |
| Vax2 | 14 | 12.8 | (8.6, 19.2) | 13 | 17.0 | (11.6, 25.1) | 1.33 | (0.78, 2.26) | 9 | 5 | (NE) |
| 4-Week | 14 | 48.8 | (33.8, 70.3) | 7 | 65.6 | (40.4, 106.5) | 1.35 | (0.77, 2.35) | 5 | 5 | (NE) |
| Post Vax2 | | | | | | | | | | | |

LLOQ = 10, results below LLOQ imputed to LLOQ/2
Vax2 = 3-Week Post Vax 1
NE = 95% CI not estimable because results for all participants had equal values

All participants in all 3 vaccine groups had H5N8 HAI titers below LLOQ at baseline.
No immune response elicited by Licensed QIV at any postvaccination visit.
Both doses of pdmFlu vaccine elicited immune response to H5N8, with limited response after Dose 1 and more robust response after Dose 2.
Dose-response trend observed, with higher immune response in the higher dose group.

TABLE 62
GMFR of H5N8 HAI Titer from Prevaccination to
Each Subsequent Timepoint - mITT Population

| | Vaccine Group (as Randomized) | | | | | | | | |
|---|---|---|---|---|---|---|---|---|---|
| | pdmFlu Vaccine | | | | | | | | |
| | 30 μg | | | 60 μg | | | Licensed QIV/Placebo | | |
| Visit | n | GMFR | (95% CI) | n | GMFR | (95% CI) | n | GMFR | (95% CI) |
| 1-Week | 15 | 1 | (NE) | 14 | 1.2 | (1.0, 1.4) | 9 | 1 | (NE) |
| Post Vax1 | | | | | | | | | |
| Vax2 | 14 | 1.5 | (1.1, 2.0) | 13 | 1.7 | (1.2, 2.5) | 8 | 1 | (NE) |
| 4-Week | 14 | 4.9 | (3.4, 7.0) | 7 | 6.6 | (4.0, 10.7) | 4 | 1 | (NE) |
| Post Vax2 | | | | | | | | | |

LLOQ = 10, if baseline value below LLOQ and post-baseline value ≥ LLOQ, baseline value imputed to LLOQ in fold- rise calculation
Vax2 = 3-Week PostVax1
NE = 95% CI not estimable because results for all participants had equal values

GMFRs showed same trend as GMTs

TABLE 63
Percentage of Participants with Seroconversion (4-Fold Rise) to H5N8 - mITT Population

| | Vaccine Group (as Randomized) | | | | | | | | |
|---|---|---|---|---|---|---|---|---|---|
| | pdmFlu Vaccine | | | | | | | | |
| | 30 μg | | | 60 μg | | | Licensed QIV/Placebo | | |
| Visit | % | n/N | (95% CI) | % | n/N | (95% CI) | % | n/N | (95% CI) |
| 1-Week Post Vax 1 | 0 | 0/15 | (0, 21.8) | 0 | 0/14 | (0, 23.2) | 0 | 0/9 | (0, 33.6) |
| Vax2 | 14.3 | 2/14 | (1.8, 42.8) | 15.4 | 2/13 | (1.9, 45.4) | 0 | 0/8 | (0, 36.9) |
| 4-Week Post Vax2 | 85.7 | 12/14 | (57.2, 98.2) | 100 | 7/7 | (59.0, 100) | 0 | 0/4 | (0, 60.2) |

Seroconversion: at least 4-fold rise from baseline. Baseline value below LLOQ imputed to LLOQ in fold-rise calculation.
Vax2 = 3-Week Post Vax1

High seroconversion rates observed in both dose groups of pdmFlu vaccine after Dose 2.
All 7 participants in the 60-μg dose group achieved seroconversion at 4-week post-Dose 2.
No seroconversion in Licensed QIV group.

TABLE 64
Percentage of Participants with H5N8 HAI Titers ≥ LLOQ - mITT Population

| | Vaccine Group (as Randomized) | | | | | | | | |
|---|---|---|---|---|---|---|---|---|---|
| | pdm Flu Vaccine | | | | | | | | |
| | 30 μg | | | 60 μg | | | Licensed QIV/Placebo | | |
| Visit | % | n/N | (95% CI) | % | n/N | (95% CI) | % | n/N | (95% CI) |
| Prevax | 0 | 0/15 | (0, 21.8) | 0 | 0/15 | (0, 21.8) | 0 | 0/9 | (0, 33.6) |
| 1-Week Post Vax1 | 13.3 | 2/15 | (1.7, 40.5) | 42.9 | 6/14 | (17.7, 71.1) | 0 | 0/10 | (0, 30.8) |
| Vax2 | 78.6 | 11/14 | (49.2, 95.3) | 100 | 13/13 | (75.3, 100) | 0 | 0/9 | (0, 33.6) |
| 4-Week Post Vax2 | 100 | 14/14 | (76.8, 100) | 100 | 7/7 | (59.0, 100) | 0 | 0/5 | (0, 52.2) |

LLOQ = 10
Vax2 = 3-Week Post Vax 1

Both dose groups of pdmFlu vaccine showed immune response starting as early as 1-week post-Dose 1 with higher response post-Dose 1 in the higher dose group.
All participants in both pdmFlu dose groups responded at 4-week post-Dose 2.
No response in Licensed QIV group.

The Phase 1 study that has enrolled a younger adult (18-49 yrs) cohort and enrollment in older adults (65-84 yrs) will be initiated. The data demonstrate HI antibody responses to the vaccine-matched A/H5N8 strain and a heterologous A/H5N1 strain (clade 2.3.4.4b) with a closely related HA after one and two administrations of the lowest and middle dose in younger adults. Highest antibody responses were observed after 2 administrations at the middle dose level (in the dose groups with data currently available). The majority/all of reactogenicity following vaccination has been mild or moderate and with average duration 1-5 days. Larger numbers of participants at the highest dose level reported reactogenicity as compared to the middle and lower dose levels. No severe adverse events and no serious adverse events after vaccination with pdmFlu vaccine were observed, there is an overall acceptable safety profile with the vaccine candidate. PdmFlu Phase 1 results in patent is 18-49 yrs of age demonstrated seroconversion and seroprotection for homologous H5N8 strain exceeded FDA guidelines for adults <65 years of age after 2 doses of 30 μg and 60 μg pdmFlu. Wide 95% confidence intervals were observed for all values due to small numbers of participants. Local reactogenicity symptoms were mild to moderate at 30 μg, 60 μg and 90 μg dose levels. Hemagglutinin (HA) encoded in the pdmFlu vaccine is the HA from A/Astrakhan/3212/2020 (H5N8) and is 99.12% homologous to H5N1 dairy cow isolate and 2 human cases.

TABLE 65

| | | Geometric | | |
|---|---|---|---|---|
| | | Mean | Seroconversion | Seroprotection |
| Dose | Timing | Titers | % | % |
| 30 μg | Day 1 Prior to | 6.6 | — | 7.1 |
| pdmFlu | Vaccination 1 | | | |
| N = 14 | 3 weeks after | 11.9 | 28.6 | 35.7 |
| | Vaccination 1 | | | |
| | 4 weeks after | 62.5 | 78.6 | 78.6 |
| | Vaccination 2 | | | |
| 60 μg | Day 1 Prior to | 5.7 | — | 0% |
| pdmFlu | Vaccination 1 | | | |
| N = 13 | 3 weeks after | 17.0 | 38.5 | 38.5 |
| | Vaccination 1 | | | |
| | 4 weeks after | 84.4 | 76.9 | 76.9 |
| | Vaccination 2 | | | |
| QIV/ | Day 1 Prior to | 5.8 | — | 0 |
| Placebo | Vaccination 1 | | | |
| N = 9 | 3 weeks after | 6.1 | 0 | 0 |
| | Vaccination 1 | | | |
| | 4 weeks after | 6.1 | 0 | 0 |
| | Vaccination 2 | | | |

PdmFlu Phase 1 results in 18-49 yrs demonstrated seroconversion and seroprotection for H5N1 strain with similar HA exceeded FDA guidelines2 for adults <65 years of age after 2 doses of 30 μg and 60 μg pdmFlu and 1 dose of 60 μg. Wide 95% confidence intervals were observed for all values due to small numbers of participants. HA from A/American wigeon/South Carolina/22-000345-001/2021 (H5N1) is 99.47% homologous to vaccine encoded HA and 99.65% homologous to H5N1 dairy cow isolate and 2 human cases. The hemagglutinin (HA) encoded in the pdmFlu vaccine is the HA from A/Astrakhan/3212/2020 (H5N8).

TABLE 66

| | | Geometric | | |
|---|---|---|---|---|
| | | Mean | Seroconversion | Seroprotection |
| Dose | Timing | Titers | % | % |
| 30 μg | Day 1 Prior to | 10.8 | — | 28.6 |
| pdmFlu | Vaccination 1 | | | |
| N = 14 | 3 weeks after | 22.1 | 35.7 | 50 |
| | Vaccination 1 | | | |
| | 4 weeks after | 67.3 | 57.1 | 71.4 |
| | Vaccination 2 | | | |
| 60 μg | Day 1 Prior to | 9.7 | — | 23.1 |
| pdmFlu | Vaccination 1 | | | |
| N = 13 | 3 weeks after | 44.5 | 53.8 | 76.9 |
| | Vaccination 1 | | | |
| | 4 weeks after | 164.3 | 76.9 | 92.3 |
| | Vaccination 2 | | | |
| QIV/ | Day 1 Prior to | 12.6 | — | 33.3 |
| Placebo | Vaccination 1 | | | |
| N = 9 | 3 weeks after | 13.1 | 0 | 22.2 |
| | Vaccination 1 | | | |
| | 4 weeks after | 13.1 | 0 | 22.2 |
| | Vaccination 2 | | | |

Pandemic Influenza potential Phase 2/3 design: Objective: To evaluate the immunogenicity and safety responses following vaccination with pdmFlu in healthy younger and older adults in pivotal trial intended for licensure.

Study design will evaluate 1 or 2 dose levels of pdmFlu in larger groups of younger and older adults. The study will continue to characterize immunogenicity and safety after 1 or 2 dose administration of pdmFlu. One or more assays will be used to characterize the immune response of pdmFlu. An adequate sample size will be evaluated for intended licensure of the pdmFlu vaccine in adults.
Purpose: To evaluate the immunogenicity and safety responses following vaccination with pdmFlu in healthy younger and older adults in pivotal trial intended for licensure
Design: Randomized, observer-blind inactive comparator-controlled study of safety and immunogenicity
Dosing Schedule: 2 doses given 21 days apart (Day 1 and Day 22)
Population: Healthy adults, ≥18 years, N=4440-8000
   Size will depend on if the same dose is chosen for both age groups (N=4440) or different dose is chosen (N=8000)
Objectives:
   Primary: Immunogenicity by HAI (homologous strain) (N=1680): HAI seroconversion and HI ≥1:40 endpoints, Safety/Tolerability
   Exploratory: MNT antibody responses (N=1680); Heterologous HI antibody responses in sub-set (N=840 tbc)
   Duration: 7 months/participant (screening to final visit); 1.5 month recruitment period, US only
Number of Visits: 4 [Screening/Rando Day 1, Day 22, Day 42, Day 201].

TABLE 67
Potential Phase 2/3 Study Design - Same dose chosen for both age groups

| | | Ph2 | | Ph3 | |
|---|---|---|---|---|---|
| Age groups | Study Intervention | Wave 1 | Wave 2 | Wave 3 | Total |
| 18-64 yrs | Selected dose pdmFlu × 2 | N = 15 | N = 150 | N = 1500 | N = 1665 |
| | COMIRNATY × 1/placebo × 1 | N = 5 | N = 50 | N = 500 | N = 555 |
| 65-85 yrs | Selected dose pdmFlu × 2 | N = 15 | N = 150 | N = 1500 | N = 1665 |
| | COMIRNATY × 1/placebo × 1 | N = 5 | N = 50 | N = 500 | N = 555 |
| All 18+ yrs | All Interventions | N = 40 | N = 400 | N = 4000 | N = 4400 |

Dose selected from phi
Dose used in younger and older adults is the same
Minimum 3000 safety data across both age groups combined
3:1 randomization pdmFlu to placebo in both age groups and in all enrollment waves
All participants followed for safety
420 participants for immunogenicity in each group=1680
72-hour safety pause after Wave 1 (clinical review only)
7-day safety pause after Wave 2

### Clinical Development Plan: Phase 1 Study (65-84 Yrs of Age)

Purpose: To identify 1 or more doses/schedules of pdmFLU (modified RNA pandemic influenza) with acceptable immune response and safety profile in healthy older adults
Design: Randomized, observer-blind, double dummy, placebo-controlled study of safety and immunogenicity; parallel dosing groups with sentinel wave and stopping rules
Population: Healthy adults, 65-84 years. N=40.
   No seasonal Flu vaccine in the past 6 months. Seasonal flu vaccine allowed after Day 49 visit (1 month post-Vaccination 2)
Objectives:
   Primary: Safety/Tolerability, Safety Laboratory Assessments
   Secondary: Immunogenicity by HI assay (homologous strain)
   Exploratory: Immunogenicity by HI assay (heterologous strains), immunogenicity by MNT assay(homologous), Cell Mediated Immune Responses (homologous), Durability of immune responses (=3 and 6 months); CMIs
Duration: 7 months/participant (screening to final visit), −3 weeks recruitment
Number of Visits: 8
   Screening, Day 1, Day 7, Day 21, Day 28, Day 49, Day 111, Day 201

TABLE 68

| Study | Vaccine | | Number of |
|---|---|---|---|
| Cohort | Group | Schedule | participants |
| Cohort 4 | 60 mcg pdmFlu | 2 doses separated by 3 | 15 |
| | | weeks | |
| | Saline Placebo | 2 vaccinations separated | 5 |
| | | by 3 weeks | |
| Cohort 5 | 90 mcg pdmFlu | 2 doses separated by 3 | 15 |
| | | weeks | |
| | Saline Placebo | 2 vaccinations separated | 5 |
| | | by 3 weeks | |
| | Total | | 40 |

### Clauses

1. An influenza virus vaccine, comprising: at least one ribonucleic acid (RNA) polynucleotide having an open reading frame encoding at least one influenza virus antigenic polypeptide or an immunogenic fragment thereof, formulated in a lipid nanoparticle.
2. The influenza vaccine of clause 1, wherein the RNA further comprises a 5′ cap analog.
3. The influenza vaccine of clause 2, wherein the 5′ cap analog comprises m₂^{7,3′-O}Gppp(m₁^{2′-}°)ApG.
4. The influenza vaccine of clause 1, wherein the RNA further comprises a modified nucleotide.
5. The influenza vaccine of clause 4, wherein the modified nucleotide comprises N1-Methylpseudourodine-5′-triphosphate (m1ψTP).
6. The influenza vaccine of clause 1, wherein the at least one antigenic polypeptide is influenza hemagglutinin 1 (HA1), hemagglutinin 2 (HA2), an immunogenic fragment of HA1 or HA2, or a combination of any two or more of the foregoing.
7. The influenza vaccine of clause 1, wherein at least one antigenic polypeptide is HA1, HA2, or a combination of HA1 and HA2, and at least one antigenic polypeptide is selected from the group consisting of neuraminidase (NA), nucleoprotein (NP), matrix protein 1 (M1), matrix protein 2 (M2), non-structural protein 1 (NS1) and non-structural protein 2 (NS2).
8. The influenza vaccine of clause 1, wherein at least one antigenic polypeptide is HA1, HA2, or a combination of HA1 and HA2, and at least one antigenic polypeptide is neuraminidase (NA).
9. The influenza vaccine of clause 1, wherein the composition comprises a) at least one ribonucleic acid (RNA) polynucleotide having an open reading frame encoding influenza hemagglutinin 1 (HA1); b) at least one ribonucleic acid (RNA) polynucleotide having an open reading frame encoding hemagglutinin 2 (HA2); c) at least one ribonucleic acid (RNA) polynucleotide having an open reading frame encoding at least one antigenic polypeptide is selected from the group consisting of neuraminidase (NA), nucleoprotein (NP), matrix protein 1 (M1), matrix protein 2 (M2), non-structural protein 1 (NS1) and non-structural protein 2 (NS2); and d) at least one ribonucleic acid (RNA) polynucleotide having an open reading frame encoding at least one antigenic polypeptide is selected from the group consisting of neuraminidase (NA), nucleoprotein (NP), matrix protein 1 (M1), matrix protein 2 (M2), non-structural protein 1 (NS1) and non-structural protein 2 (NS2).
10. The influenza vaccine according to clause 5, wherein the open reading frame is codon-optimized.
11. The influenza vaccine of clause 1, wherein the composition further comprises a cationic lipid.
12. The influenza vaccine of clause 1, wherein the composition comprises a lipid nanoparticle encompassing the mRNA molecule.
13. The influenza vaccine of clause 1, wherein the composition comprises a) a lipid nanoparticle encompassing at least one ribonucleic acid (RNA) polynucleotide having an open reading frame encoding influenza hemagglutinin 1 (HA1); b) a lipid nanoparticle encompassing at least one ribonucleic acid (RNA) polynucleotide having an open reading frame encoding hemagglutinin 2 (HA2); c) a lipid nanoparticle encompassing at least one ribonucleic acid (RNA) polynucleotide having an open reading frame encoding at least one antigenic polypeptide is selected from the group consisting of neuraminidase (NA), nucleoprotein (NP), matrix protein 1 (M1), matrix protein 2 (M2), non-structural protein 1 (NS1) and non-structural protein 2 (NS2); and d) a lipid nanoparticle encompassing at least one ribonucleic acid (RNA) polynucleotide having an open reading frame encoding at least one antigenic polypeptide is selected from the group consisting of neuraminidase (NA), nucleoprotein (NP), matrix protein 1 (M1), matrix protein 2 (M2), non-structural protein 1 (NS1) and non-structural protein 2 (NS2).
14. The influenza vaccine of clause 13, wherein the lipid nanoparticle size is at least 40 nm.
15. The influenza vaccine of clause 13, wherein the lipid nanoparticle size is at most 180 nm.
16. The influenza vaccine of clause 13, wherein at least 80% of the total RNA in the composition is encapsulated.
17. The influenza vaccine of clause 1, wherein the composition comprises 18. The influenza vaccine of clause 1, wherein the composition comprises ALC-0315 (4-hydroxybutyl)azanediyl)bis(hexane-6,1-diyl)bis(2-hexyldecanoate).

19. The influenza vaccine of clause 1, wherein the composition comprises ALC-0159 (2-[(polyethylene glycol)-2000]—N,N-ditetradecylacetamide).
20. The influenza vaccine of clause 1, wherein the composition comprises 1,2-Distearoyl-sn-glycero-3-phosphocholine (DSPC).
21. The influenza vaccine of clause 1, wherein the composition comprises cholesterol.
22. The influenza vaccine of clause 1, wherein the composition comprises 0.9-1.85 mg/mL ALC-0315; 0.11-0.24 mg/mL ALC-0159; 0.18-0.41 mg/mL DSPC; and 0.36-0.78 mg/mL cholesterol.
23. The influenza vaccine of clause 1, wherein the composition comprises Tris.
24. The influenza vaccine of clause 1, wherein the composition comprises sucrose.
25. The influenza vaccine of clause 1, wherein the composition does not further comprise sodium chloride.
26. The influenza vaccine of clause 1, wherein the composition comprises 10 mM Tris.
27. The influenza vaccine of clause 1, wherein the composition comprises 300 mM sucrose.
28. The influenza vaccine of clause 1, wherein the composition has a pH 7.4.
29. The influenza vaccine of clause 1, wherein the composition has less than or equal to 12.5 EU/mL of bacterial endotoxins.
30. The influenza vaccine of clause 1, wherein the RNA polynucleotide comprises a 5′ cap, 5′ UTR, 3′ UTR, histone stem-loop and poly-A tail.
31. The influenza vaccine of clause 30, wherein the 5′ UTR comprises the sequence AATAAACTAGTATTCTTCTGGTCCCCACAGACTCAGAGAGAACCC (5′ WHO UTR1) (SEQ ID No: 4).
32. The influenza vaccine of clause 30, wherein the 5′ UTR comprises the sequence GAGAAψAAACψAGψAψψCψψCψGGψCCCCA CAGACψCAGA GAGAACCCGCCACC (SEQ ID NO: 5)
33. The influenza vaccine of clause 30, wherein the 5′ UTR comprises the sequence AGAATAAACTAGTATTCTTCTGGTCCCCACAGACTCAGAGAGAACCC (5′ WHO UTR1). (SEQ ID NO: 6)
34. The influenza vaccine of clause 30, wherein the 3′ UTR comprises the sequence CUCGAGCUGGUACUGCAUGCACGCAAUGCUAGCUGCCCCUUUCCCGUCCUGGGUAC CCCGAGUCUCCCCCGACCUCGGGUCCCAGGUAUGCUCCCACCUCCACCUGCCCCAC UCACCACCUCUGCUAGUUCCAGACACCUCCCAAGCACGCAGCAAUGCAGCUCAAAAC GCUUAGCCUAGCCACACCCCCACGGGAAACAGCAGUGAUUAACCUUUAGCAAUAAAC GAAAGUUUAACUAAGCUAUACUAACCCCAGGGUUGGUCAAUUUCGUGCCAGCCACAC CCUGGAGCUAGC (3′ WHO UTR2). (SEQ ID NO: 7)
35. The influenza vaccine of clause 30, wherein the 3′ UTR comprises the sequence CψCGAGC4JGG4JAC4JGCA4JGCACGCAA4JGCψAGC4JGCCCC4J4J4JCCCG4JCC4JG GG4JACCCCGAG4JCψCCCCCGACCψCGGG4JCCCAGG4JA4JGCψCCCACC4jCCAC C4JGCCCCACψCACCACCψC4JGCψAG4J4JCCAGACACCψCCCAAGCACGCAGCAA 4JGCAGCψCAAAACGC4J4JAGCCψAGCCACACCCCCACGGGAAACAGCAG4JGA4J4J AACC4J4J 4JAGCAA4JAAACGAAAG4J4J4JAACψAAGCψA4JACψAACCCCAGGG4J4JGG 4JCAA4J4P4JCG4JGCCAGCCACACCC4JGGAGC′1 AGC (3′ WHO 4JTR2). (SEQ ID NO: 8).
36. An immunogenic composition comprising: (i) a first ribonucleic acid (RNA) polynucleotide having an open reading frame encoding a first antigen, said antigen comprising at least one influenza virus antigenic polypeptide or an immunogenic fragment thereof, and (ii) a second RNA polynucleotide having an open reading frame encoding a second antigen, said second antigen comprising at least one influenza virus antigenic polypeptide or an immunogenic fragment thereof, wherein the first and second RNA polynucleotides are formulated in a lipid nanoparticle (LNP).
37. The immunogenic composition of clause 36, wherein the first and second antigens comprise hemagglutinin (HA), or an immunogenic fragment or variant thereof.
38. The immunogenic composition of clause 36 or 37 wherein the first antigen comprises an HA from a different subtype of influenza virus to the influenza virus antigenic polypeptide or an immunogenic fragment thereof of the second antigen.
39. The immunogenic composition of any of clause 36-38, wherein the first and second RNA polynucleotides are formulated in a single lipid nanoparticle.
40. The immunogenic composition of any preceding clause further comprising: (iii) a third antigen comprising at least one influenza virus antigenic polypeptide or an immunogenic fragment thereof, wherein the third antigen is from influenza virus but is from a different strain of influenza virus to both the first and second antigens.
41. The immunogenic composition of clause 40, wherein the first, second and third RNA polynucleotides are formulated in a lipid nanoparticle.
42. The immunogenic composition of clause 41, wherein the first, second and third RNA polynucleotides are formulated in a single lipid nanoparticle.
43. The immunogenic composition of any preceding clause further comprising: (iv) a fourth RNA polynucleotide having an open reading frame encoding a fourth antigen, said antigen comprising at least one influenza virus antigenic polypeptide or an immunogenic fragment thereof, wherein the fourth antigen is from influenza virus but is from a different strain of influenza virus to the first, second and third antigens.
44. The immunogenic composition of clause 43, wherein the first, second, third, and fourth RNA polynucleotides are formulated in a lipid nanoparticle.
45. The immunogenic composition of clause 44, wherein the first, second, third, and fourth RNA polynucleotides are formulated in a single lipid nanoparticle.
46. The immunogenic composition of any preceding clause further comprising: (v) a fifth RNA polynucleotide having an open reading frame encoding a fifth antigen, said antigen comprising at least one influenza virus antigenic polypeptide or an immunogenic fragment thereof, wherein the fifth antigen is from influenza virus but is from a different strain of influenza virus to the first, second, third, and fourth antigens.
47. The immunogenic composition of clause 46, wherein the first, second, third, fourth, and fifth RNA polynucleotides are formulated in a lipid nanoparticle.
48. The immunogenic composition of clause 47, wherein the first, second, third, fourth, and fifth RNA polynucleotides are formulated in a single lipid nanoparticle.
49. The immunogenic composition of any preceding clause further comprising: (vi) a sixth RNA polynucleotide having an open reading frame encoding a sixth antigen, said antigen comprising at least one influenza virus antigenic polypeptide or an immunogenic fragment thereof, wherein the sixth antigen is from influenza virus but is from a different strain of influenza virus to the first, second, third, fourth, and fifth antigens.
50. The immunogenic composition of clause 49, wherein the first, second, third, fourth, and fifth RNA polynucleotides are formulated in a lipid nanoparticle.
51. The immunogenic composition of clause 50, wherein the first, second, third, fourth, and fifth RNA polynucleotides are formulated in a single lipid nanoparticle.
52. The immunogenic composition of any preceding clause further comprising: (vii) a seventh RNA polynucleotide having an open reading frame encoding a seventh antigen, said antigen comprising at least one influenza virus antigenic polypeptide or an immunogenic fragment thereof, wherein the seventh antigen is from influenza virus but is from a different strain of influenza virus to the first, second, third, fourth, fifth, and sixth antigens.
53. The immunogenic composition of clause 52, wherein the first, second, third, fourth, fifth, sixth and seventh RNA polynucleotides are formulated in a lipid nanoparticle.
54. The immunogenic composition of clause 53, wherein the first, second, third, fourth, fifth, sixth and seventh RNA polynucleotides are formulated in a single lipid nanoparticle.
55. The immunogenic composition of any preceding clause further comprising: (viii) an eighth RNA polynucleotide having an open reading frame encoding an eighth antigen, said antigen comprising at least one influenza virus antigenic polypeptide or an immunogenic fragment thereof, wherein the eighth antigen is from influenza virus but is from a different strain of influenza virus to the first, second, third, fourth, fifth, sixth and seventh antigens.
56. The immunogenic composition of clause 55, wherein the first, second, third, fourth, fifth, sixth, seventh and eighth RNA polynucleotides are formulated in a lipid nanoparticle.
57. The immunogenic composition of clause 56, wherein the first, second, third, fourth, fifth, sixth, seventh and eighth RNA polynucleotides are formulated in a single lipid nanoparticle.
58. The immunogenic composition of any preceding clause further comprising: (v) a fifth RNA polynucleotide having an open reading frame encoding a fifth antigen, said antigen comprising at least one influenza virus antigenic polypeptide or an immunogenic fragment thereof, wherein the fifth antigen is from influenza virus but is from a different strain of influenza virus to the first, second, third, and fourth antigens.
59. The immunogenic composition of clause 58, wherein the first, second, third, fourth, and fifth RNA polynucleotides are formulated in a lipid nanoparticle.
60. The immunogenic composition of clause 59, wherein the RNA polynucleotides are present in about equal ratios.
61. The immunogenic composition of any preceding clause, wherein each RNA polynucleotide comprises a modified nucleotide.
62. The immunogenic composition of clause 61, wherein the modified nucleotide is selected from the group consisting of pseudouridine, 1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-0-methyl uridine.
63. The immunogenic composition of any preceding clause, wherein each RNA polynucleotide comprises a 5′ terminal cap, a 5′ UTR, a 3′UTR, and a 3′ polyadenylation tail.
64. The immunogenic composition of clause 63, wherein the 5′ terminal cap comprises:

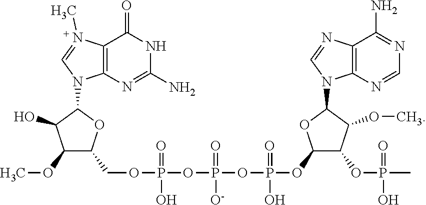

65. The immunogenic composition of clause 63, wherein the 5′ UTR comprises SEQ ID NO: 1.
66. The immunogenic composition of clause 63, wherein the 3′ UTR comprises SEQ ID NO: 2.
67. The immunogenic composition of clause 63, wherein the 3′ polyadenylation tail comprises SEQ ID NO: 3.
68. The immunogenic composition of any preceding clause, wherein the RNA polynucleotide has an integrity greater than 85%.
69. The immunogenic composition of any preceding clause, wherein the RNA polynucleotide has a purity of greater than 85%.
70. The immunogenic composition of any preceding clause, wherein the lipid nanoparticle comprises 20-60 mol % ionizable cationic lipid, 5-25 mol % neutral lipid, 25-55 mol % cholesterol, and 0.5-5 mol % PEG-modified lipid.
71. The immunogenic composition of any preceding clause, wherein the cationic lipid comprises:

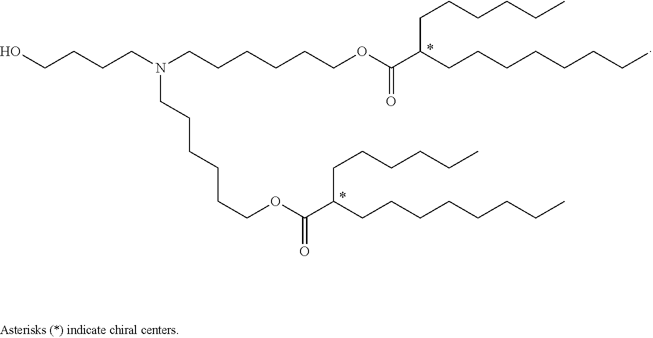

72. The immunogenic composition of any preceding clause, wherein the PEG-modified lipid comprises:

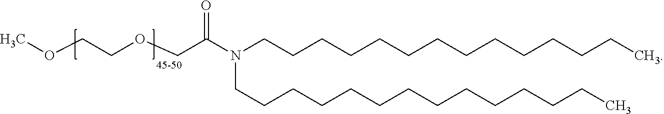

73. The immunogenic composition of any preceding clause, wherein the first antigen is HA from influenza A subtype H1 or an immunogenic fragment or variant thereof and the second antigen is HA from a different H1 strain to the first antigen or an immunogenic fragment or variant thereof.
74. The immunogenic composition of any preceding clause, wherein the first and second antigens are HA from influenza A subtype H3 or an immunogenic fragment or variant thereof and wherein both antigens are derived from different strains of H3 influenza virus.
75. The immunogenic composition of any preceding clause, wherein the first and second antigens are HA from influenza A subtype H1 or an immunogenic fragment or variant thereof and the third and fourth antigens are from influenza A subtype H3 or an immunogenic fragment or variant thereof and wherein the first and second antigens are derived from different strains of H1 virus and the third and fourth antigens are from different strains of H3 influenza virus.
76. The immunogenic composition of any preceding clause, wherein at least the first and second RNA polynucleotides are formulated in a single lipid nanoparticle.
77. The immunogenic composition of any preceding clause, wherein the first and second RNA polynucleotides are formulated in a single lipid nanoparticle.
78. The immunogenic composition of any preceding clause, wherein the first, second, and third RNA polynucleotides are formulated in a single lipid nanoparticle.
79. The immunogenic composition of any preceding clause, wherein the first, second, third, and fourth RNA polynucleotides are formulated in a single LNP.
80. The immunogenic composition of any one of clauses 36-75, wherein each of the RNA polynucleotides is formulated in a single LNP, wherein each single LNP encapsulates the RNA polynucleotide encoding one antigen.
81. The immunogenic composition of clause 80, wherein the first RNA polynucleotide is formulated in a first LNP; and the second RNA polynucleotide is formulated in a second LNP.
82. The immunogenic composition of clause 80, wherein the first RNA polynucleotide is formulated in a first LNP; the second RNA polynucleotide is formulated in a second LNP; and the third RNA polynucleotide is formulated in a third LNP.
83. The immunogenic composition of clause 80, wherein the first RNA polynucleotide is formulated in a first LNP; the second RNA polynucleotide is formulated in a second LNP; the third RNA polynucleotide is formulated in a third LNP; and the fourth RNA polynucleotide is formulated in a fourth LNP.
84. The immunogenic composition of clause 80, wherein the first RNA polynucleotide is formulated in a first LNP; the second RNA polynucleotide is formulated in a second LNP; the third RNA polynucleotide is formulated in a third LNP; the fourth RNA polynucleotide is formulated in a fourth LNP; and the fifth RNA polynucleotide is formulated in a fifth LNP.
85. The immunogenic composition of clause 80, wherein the first RNA polynucleotide is formulated in a first LNP; the second RNA polynucleotide is formulated in a second LNP; the third RNA polynucleotide is formulated in a third LNP; the fourth RNA polynucleotide is formulated in a fourth LNP; the fifth RNA polynucleotide is formulated in a fifth LNP; and the sixth RNA polynucleotide is formulated in a sixth LNP.
86. The immunogenic composition of clause 80, wherein the first RNA polynucleotide is formulated in a first LNP; the second RNA polynucleotide is formulated in a second LNP; the third RNA polynucleotide is formulated in a third LNP; the fourth RNA polynucleotide is formulated in a fourth LNP; the fifth RNA polynucleotide is formulated in a fifth LNP; the sixth RNA polynucleotide is formulated in a sixth LNP; and the seventh RNA polynucleotide is formulated in a seventh LNP.
87. The immunogenic composition of clause 80, wherein the first RNA polynucleotide is formulated in a first LNP; the second RNA polynucleotide is formulated in a second LNP; the third RNA polynucleotide is formulated in a third LNP; the fourth RNA polynucleotide is formulated in a fourth LNP; the fifth RNA polynucleotide is formulated in a fifth LNP; the sixth RNA polynucleotide is formulated in a sixth LNP; the seventh RNA polynucleotide is formulated in a seventh LNP; and the eighth RNA polynucleotide is formulated in an eighth LNP.
88. The immunogenic composition of any preceding clause, for use in the eliciting an immune response against influenza.
89. A method of eliciting an immune response against influenza disease, comprising administering an effective amount of an immunogenic composition according to any one of clauses 1-35.
90. A method of eliciting an immune response against influenza disease, comprising administering an effective amount of an immunogenic composition according to any one of clauses 36-79.
91. The method according to clause 90, wherein said immune response has a vaccine efficacy of greater than 50% after at least one dose of the composition.
92. The method according to any one of clauses 90-91, comprising administering a second composition comprising a RNA polynucleotide comprising an open reading frame encoding at least one influenza virus antigenic polypeptide or an immunogenic fragment thereof, wherein the RNA polynucleotide is formulated in a lipid nanoparticle (LNP), wherein the polypeptide is derived from an influenza virus strain that is associated with a pandemic or has the potential to be associated with a pandemic.
93. The method of clause 90, wherein the RNA polynucleotide in the second composition encodes a hemagglutinin (HA) or an immunogenic fragment thereof.
94. The method of clause 90, wherein the RNA polynucleotide in the second composition encodes a neuraminidase (NA) or an immunogenic fragment or variant thereof.
95. The method according to any one of clauses 92-94, wherein the second composition is administered 1 week to 14 months after the first composition.
96. The method according to any one of clauses 92-95, wherein said composition elicits at least one effect chosen from the group of: (i) a CD4 T-cell immune response; (ii) a B cell memory response; and (iii) a humoral response, against the influenza virus.
97. A method of producing an RNA polynucleotide-encapsulated lipid nanoparticle (LNP), the method comprises purifying an RNA polynucleotide comprising an open reading frame encoding a first antigen, said antigen comprising at least one influenza virus antigenic polypeptide or an immunogenic fragment thereof through ultrafiltration and diafiltration; formulating the purified RNA polynucleotide in an LNP, wherein the LNP is buffer exchanged and concentrated via flat sheet cassette membranes.
98. The method according to clause 97, wherein the method does not comprise a chromatography step or hollow fiber membranes.
99. The method according to clause 97, wherein the purified RNA polynucleotide is substantially free of contaminants comprising short abortive RNA species, long abortive RNA species, double-stranded RNA (dsRNA), residual plasmid DNA, residual in vitro transcription enzymes, residual solvent and/or residual salt.
100. A method for the production of an immunogenic composition for a pandemic situation or a pre-pandemic situation, comprising formulating an RNA polynucleotide in a LNP according to any one of clauses 1-35.
101. The composition according to any one of clauses 1-35, wherein the RNA polynucleotide is a self-amplifying RNA.
102. A method of purifying an RNA polynucleotide synthesized by in vitro transcription,
comprising ultrafiltration and diafiltration.
103. The method according to clause 102, wherein the method does not comprise a chromatography step.
104. The method according to clause 102, wherein the purified RNA polynucleotide is substantially free of contaminants comprising short abortive RNA species, long abortive RNA species, double-stranded RNA (dsRNA), residual plasmid DNA, residual in vitro transcription enzymes, residual solvent and/or residual salt.
105. The method according to clause 102, wherein the residual plasmid DNA is ≤500 ng DNA/mg RNA.
106. The method according to clause 102, wherein purity of the purified mRNA is between about 60% and about 100%.
107. The method according to clause 102, further comprising encapsulating the RNA polynucleotide in a lipid nanoparticle.
108. The method according to clause 107, wherein the LNPs are buffer exchanged and concentrated via flat sheet cassette membranes.
109. The influenza virus vaccine according to any one clause 1-35, wherein the open reading frame comprises a sequence having at least 80% identity to the sequence selected from the group consisting of SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38.
110. An RNA polynucleotide having an open reading frame comprising a sequence having at least 80% identity to the sequence selected from the group consisting of SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38, wherein the RNA further comprises a modified nucleotide, a 5′ UTR, and 3′ UTR.

1. A composition comprising a ribonucleic acid (RNA) polynucleotide comprising an open reading frame encoding at least one influenza virus antigenic polypeptide or an immunogenic fragment thereof, wherein the RNA polynucleotide is formulated in a lipid nanoparticle (LNP), wherein the polypeptide is derived from an influenza virus strain that is associated with a pandemic or has the potential to be associated with a pandemic.

2. The composition of claim 1, wherein the antigen comprises hemagglutinin (HA) or an immunogenic fragment or variant thereof.

3. The composition of claim 1, wherein the pandemic influenza virus strain is selected from the list consisting of: H5N1, H9N2, H7N7, H2N2, H7N1, and H1N1.

4. The composition of claim 1, wherein each RNA polynucleotide comprises a modified nucleotide.

5. The composition of claim 4, wherein the modified nucleotide is selected from the group consisting of pseudouridine, 1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-0-methyl uridine.

6. The composition of claim 1, wherein the RNA polynucleotide comprises a 5′ UTR and a 3′UTR.

7. The composition of claim 8, wherein the 5′ UTR comprises SEQ ID NO: 1.

8. The composition of claim 8, wherein the 3′ UTR comprises SEQ ID NO: 2.

9. The composition of claim 8, wherein the RNA polynucleotide comprises a 5′ terminal cap.

10. The composition of claim 9, wherein the 5′ terminal cap comprises:

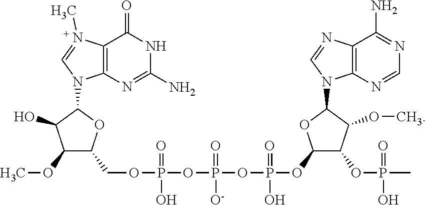

11. The composition of claim 1, wherein the RNA polynucleotide comprises a 3′ polyadenylation tail.

12. The composition of claim 11, wherein the 3′ polyadenylation tail comprises SEQ ID NO: 3.

13. The composition of claim 1, wherein the RNA polynucleotide has an integrity greater than 85%.

14. The composition of claim 1, wherein the RNA polynucleotide has a purity of greater than 85%.

15. The composition of claim 1, wherein the lipid nanoparticle comprises 20-60 mol % ionizable cationic lipid, 5-25 mol % neutral lipid, 25-55 mol % cholesterol, and 0.5-5 mol % PEG-modified lipid.

16. The composition of claim 15, wherein the cationic lipid comprises:

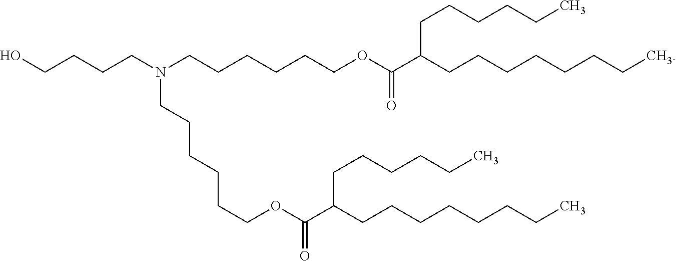 [SMILES: CCCCCCCCC(CCCCCC)C(=O)OCCCCCCN(CCCCO)CCCCCCOC(=O)C(CCCCCC)CCCCCCCC]

17. The composition of claim 15, wherein the PEG-modified lipid comprises:

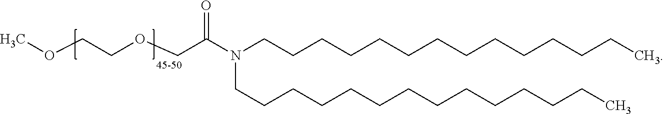

18. The composition of claim 1, wherein the composition comprises a second ribonucleic acid (RNA) polynucleotide comprising an open reading frame encoding a second influenza virus antigenic polypeptide or an immunogenic fragment thereof, wherein the second RNA polynucleotide is formulated in a lipid nanoparticle (LNP).

19. The composition of claim 18, wherein the second influenza virus antigenic polypeptide is neuraminidase (NA).

20. The composition of claim 18, wherein the ratio of the first RNA polynucleotide to the second RNA polynucleotide is 1:1.

21. The composition of claim 18, wherein the ratio of the first RNA polynucleotide to the second RNA polynucleotide is 1: greater than 1.

22. A method of eliciting an immune response against influenza disease in a subject, comprising administering an effective amount of a composition comprising a ribonucleic acid (RNA) polynucleotide comprising an open reading frame encoding at least one influenza virus antigenic polypeptide or an immunogenic fragment thereof, wherein the RNA polynucleotide is formulated in a lipid nanoparticle (LNP), wherein the polypeptide is derived from an influenza virus strain that is associated with a pandemic or has the potential to be associated with a pandemic.
